SEQUENCE LISTING (69 sequences)

SEQ ID NO: 1 - protein, 1732 aa, Homo sapiens
MPEPWGTVYFLGIAQVFSFLFSWWNLEGVMNQADAPRPLNWTIRKLCHAAFLPSVRLLKAQKSWIERAFYKRECVHIIPSTKDPHRCCCGRLIGQHVGLTPSISVLQNEKNESRLSRNDIQSEKWSISKHTQLSPTDAFGTIEFQGGGHSNKAMYVRVSFDTKPDLLLHLMTKEWQLELPKLLISVHGGLQNFELQPKLKQVFGKGLIKAAMTTGAWIFTGGVNTGVIRHVGDALKDHASKSRGKICTIGIAPWGIVENQEDLIGRDVVRPYQTMSNPMSKLTVLNSMHSHFILADNGTTGKYGAEVKLRRQLEKHISLQKINTRCLPFFSLDSRLFYSFWGSCQLDSVGIGQGVPVVALIVEGGPNVISIVLEYLRDTPPVPVVVCDGSGRASDILAFGHKYSEEGGLINESLRDQLLVTIQKTFTYTRTQAQHLFIILMECMKKKELITVFRMGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKGNLPPDYRISLIDIGLVIEYLMGGAYRCNYTRKRFRTLYHNLFGPKRPKALKLLGMEDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAGEETMSPTSPTLMPRMRSHSFYSVNMKDKGGIEKLESIFKERSLSLHRATSSHSVAKEPKAPAAPANTLAIVPDSRRPSSCIDIYVSAMDELHCDIDPLDNSVNILGLGEPSFSTPVPSTAPSSSAYATLAPTDRPPSRSIDFEDITSMDTRSFSSDYTHLPECQNPWDSEPPMYHTIERSKSSRYLATTPFLLEEAPIVKSHSFMFSPSRSYYANFGVPVKTAEYTSITDCIDTRCVNAPQAIADRAAFPGGLGDKVEDLTCCHPEREAELSHPSSDSEENEAKGRRATIAISSQEGDNSERTLSNNITVPKIERANSYSAEEPSAPYAHTRKSFSISDKLDRQRNTASLRNPFQRSKSSKPEGRGDSLSMRRLSRTSAFQSFESKHN

SEQ ID NO: 2 - protein, 1707 aa, Homo sapiens
MPEPWGTVYFLGIAQVFSFLFSWWNLEGVMNQADAPRPLNWTIRKLCHAAFLPSVRLLKAQKSWIERAFYKRECVHIIPSTKDPHRCCCGRLIGQHVGLTPSISVLQNEKNESRLSRNDIQSEKWSISKHTQLSPTDAFGTIEFQGGGHSNKAMYVRVSFDTKPDLLLHLMTKEWQLELPKLLISVHGGLQNFELQPKLKQVFGKGLIKAAMTTGAWIFTGGVNTGVIRHVGDALKDHASKSRGKICTIGIAPWGIVENQEDLIGRDVVRPYQTMSNPMSKLTVLNSMHSHFILADNGTTGKYGAEVKLRRQLEKHISLQKINTRIGQGVPVVALIVEGGPNVISIVLEYLRDTPPVPVVVCDGSGRASDILAFGHKYSEEGGLINESLRDQLLVTIQKTFTYTRTQAQHLFIILMECMKKKELITVFRMGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKGNLPPDYRISLIDIGLVIEYLMGGAYRCNYTRKRFRTLYHNLFGPKRPKALKLLGMEDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAGEETMSPTSPTLMPRMRSHSFYSVNMKDKGGIEKLESIFKERSLSLHRATSSHSVAKEPKAPAAPANTLAIVPDSRRPSSCIDIYVSAMDELHCDIDPLDNSVNILGLGEPSFSTPVPSTAPSSSAYATLAPTDRPPSRSIDFEDITSMDTRSFSSDYTHLPECQNPWDSEPPMYHTIERSKSSRYLATTPFLLEEAPIVKSHSFMFSPSRSYYANFGVPVKTAEYTSITDCIDTRCVNAPQAIADRAAFPGGLGDKVEDLTCCHPEREAELSHPSSDSEENEAKGRRATIAISSQEGDNSERTLSNNITVPKIERANSYSAEEPSAPYAHTRKSFSISDKLDRQRNTASLRNPFQRSKSSKPEGRGDSLSMRRLSRTSAFQSFESKHN

SEQ ID NO: 3 - protein, 1719 aa, Homo sapiens
MPEPWGTVYFLGIAQVFSFLFSWWNLEGVMNQADAPRPLNWTIRKLCHAAFLPSVRLLKAQKSWIERAFYKRECVHIIPSTKDPHRCCCGRLIGQHVGLTPSISVLQNEKNESRLSRNDIQSEKWSISKHTQLSPTDAFGTIEFQGGGHSNKAMYVRVSFDTKPDLLLHLMTKEWQLELPKLLISVHGGLQNFELQPKLKQVFGKGLIKAAMTTGAWIFTGGVNTGVIRHVGDALKDHASKSRGKICTIGIAPWGIVENQEDLIGRDVVRPYQTMSNPMSKLTVLNSMHSHFILADNGTTGKYGAEVKLRRQLEKHISLQKINTRIGQGVPVVALIVEGGPNVISIVLEYLRDTPPVPVVVCDGSGRASDILAFGHKYSEEGGLINESLRDQLLVTIQKTFTYTRTQAQHLFIILMECMKKKELITVFRMGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKREYPGFGWIYFKGNLPPDYRISLIDIGLVIEYLMGGAYRCNYTRKRFRTLYHNLFGPKRPKALKLLGMEDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAGEETMSPTSPTLMPRMRSHSFYSVNMKDKGGIEKLESIFKERSLSLHRATSSHSVAKEPKAPAAPANTLAIVPDSRRPSSCIDIYVSAMDELHCDIDPLDNSVNILGLGEPSFSTPVPSTAPSSSAYATLAPTDRPPSRSIDFEDITSMDTRSFSSDYTHLPECQNPWDSEPPMYHTIERSKSSRYLATTPFLLEEAPIVKSHSFMFSPSRSYYANFGVPVKTAEYTSITDCIDTRCVNAPQAIADRAAFPGGLGDKVEDLTCCHPEREAELSHPSSDSEENEAKGRRATIAISSQEGDNSERTLSNNITVPKIERANSYSAEEPSAPYAHTRKSFSISDKLDRQRNTASLRNPFQRSKSSKPEGRGDSLSMRRLSRTSAFQSFESKHN

SEQ ID NO: 4 - protein, 1697 aa, Homo sapiens
MPEPWGTVYFLGIAQVFSFLFSWWNLEGVMNQADAPRPLNWTIRKLCHAAFLPSVRLLKAQKSWIERAFYKRECVHIIPSTKDPHRCCCGRLIGQHVGLTPSISVLQNEKNESRLSRNDIQSEKWSISKHTQLSPTDAFGTIEFQGGGHSNKAMYVRVSFDTKPDLLLHLMTKEWQLELPKLLISVHGGLQNFELQPKLKQVFGKGLIKAAMTTGAWIFTGGVNTGVIRHVGDALKDHASKSRGKICTIGIAPWGIVENQEDLIGRDVVRPYQTMSNPMSKLTVLNSMHSHFILADNGTTGKYGAEVKLRRQLEKHISLQKINTRIGQGVPVVALIVEGGPNVISIVLEYLRDTPPVPVVVCDGSGRASDILAFGHKYSEEGGLINESLRDQLLVTIQKTFTYTRTQAQHLFIILMECMKKKELITVFRMGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKGNLPPDYRISLIDIGLVIEYLMGGAYRCNYTRKRFRTLYHNLFGPKRDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAGEETMSPTSPTLMPRMRSHSFYSVNMKDKGGIEKLESIFKERSLSLHRATSSHSVAKEPKAPAAPANTLAIVPDSRRPSSCIDIYVSAMDELHCDIDPLDNSVNILGLGEPSFSTPVPSTAPSSSAYATLAPTDRPPSRSIDFEDITSMDTRSFSSDYTHLPECQNPWDSEPPMYHTIERSKSSRYLATTPFLLEEAPIVKSHSFMFSPSRSYYANFGVPVKTAEYTSITDCIDTRCVNAPQAIADRAAFPGGLGDKVEDLTCCHPEREAELSHPSSDSEENEAKGRRATIAISSQEGDNSERTLSNNITVPKIERANSYSAEEPSAPYAHTRKSFSISDKLDRQRNTASLRNPFQRSKSSKPEGRGDSLSMRRLSRTSAFQSFESKHN

SEQ ID NO: 5 - protein, 1709 aa, Homo sapiens
MPEPWGTVYFLGIAQVFSFLFSWWNLEGVMNQADAPRPLNWTIRKLCHAAFLPSVRLLKAQKSWIERAFYKRECVHIIPSTKDPHRCCCGRLIGQHVGLTPSISVLQNEKNESRLSRNDIQSEKWSISKHTQLSPTDAFGTIEFQGGGHSNKAMYVRVSFDTKPDLLLHLMTKEWQLELPKLLISVHGGLQNFELQPKLKQVFGKGLIKAAMTTGAWIFTGGVNTGVIRHVGDALKDHASKSRGKICTIGIAPWGIVENQEDLIGRDVVRPYQTMSNPMSKLTVLNSMHSHFILADNGTTGKYGAEVKLRRQLEKHISLQKINTRIGQGVPVVALIVEGGPNVISIVLEYLRDTPPVPVVVCDGSGRASDILAFGHKYSEEGGLINESLRDQLLVTIQKTFTYTRTQAQHLFIILMECMKKKELITVFRMGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKREYPGFGWIYFKGNLPPDYRISLIDIGLVIEYLMGGAYRCNYTRKRFRTLYHNLFGPKRDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAGEETMSPTSPTLMPRMRSHSFYSVNMKDKGGIEKLESIFKERSLSLHRATSSHSVAKEPKAPAAPANTLAIVPDSRRPSSCIDIYVSAMDELHCDIDPLDNSVNILGLGEPSFSTPVPSTAPSSSAYATLAPTDRPPSRSIDFEDITSMDTRSFSSDYTHLPECQNPWDSEPPMYHTIERSKSSRYLATTPFLLEEAPIVKSHSFMFSPSRSYYANFGVPVKTAEYTSITDCIDTRCVNAPQAIADRAAFPGGLGDKVEDLTCCHPEREAELSHPSSDSEENEAKGRRATIAISSQEGDNSERTLSNNITVPKIERANSYSAEEPSAPYAHTRKSFSISDKLDRQRNTASLRNPFQRSKSSKPEGRGDSLSMRRLSRTSAFQSFESKHN

SEQ ID NO: 6 - protein, 1719 aa, Homo sapiens
MPEPWGTVYFLGIAQVFSFLFSWWNLEGVMNQADAPRPLNWTIRKLCHAAFLPSVRLLKAQKSWIERAFYKRECVHIIPSTKDPHRCCCGRLIGQHVGLTPSISVLQNEKNESRLSRNDIQSEKWSISKHTQLSPTDAFGTIEFQGGGHSNKAMYVRVSFDTKPDLLLHLMTKEWQLELPKLLISVHGGLQNFELQPKLKQVFGKGLIKAAMTTGAWIFTGGVNTGVIRHVGDALKDHASKSRGKICTIGIAPWGIVENQEDLIGRDVVRPYQTMSNPMSKLTVLNSMHSHFILADNGTTGKYGAEVKLRRQLEKHISLQKINTRIGQGVPVVALIVEGGPNVISIVLEYLRDTPPVPVVVCDGSGRASDILAFGHKYSEEGGLINESLRDQLLVTIQKTFTYTRTQAQHLFIILMECMKKKELITVFRMGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKGNLPPDYRISLIDIGLVIEYLMGGAYRCNYTRKRFRTLYHNLFGPKRPKALKLLGMEDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDRKQVYDSHTPKSAPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAGEETMSPTSPTLMPRMRSHSFYSVNMKDKGGIEKLESIFKERSLSLHRATSSHSVAKEPKAPAAPANTLAIVPDSRRPSSCIDIYVSAMDELHCDIDPLDNSVNILGLGEPSFSTPVPSTAPSSSAYATLAPTDRPPSRSIDFEDITSMDTRSFSSDYTHLPECQNPWDSEPPMYHTIERSKSSRYLATTPFLLEEAPIVKSHSFMFSPSRSYYANFGVPVKTAEYTSITDCIDTRCVNAPQAIADRAAFPGGLGDKVEDLTCCHPEREAELSHPSSDSEENEAKGRRATIAISSQEGDNSERTLSNNITVPKIERANSYSAEEPSAPYAHTRKSFSISDKLDRQRNTASLRNPFQRSKSSKPEGRGDSLSMRRLSRTSAFQSFESKHN

SEQ ID NO: 7 - protein, 1722 aa, Homo sapiens
MPEPWGTVYFLGIAQVFSFLFSWWNLEGVMNQADAPRPLNWTIRKLCHAAFLPSVRLLKAQKSWIERAFYKRECVHIIPSTKDPHRCCCGRLIGQHVGLTPSISVLQNEKNESRLSRNDIQSEKWSISKHTQLSPTDAFGTIEFQGGGHSNKAMYVRVSFDTKPDLLLHLMTKEWQLELPKLLISVHGGLQNFELQPKLKQVFGKGLIKAAMTTGAWIFTGGVNTGVIRHVGDALKDHASKSRGKICTIGIAPWGIVENQEDLIGRDVVRPYQTMSNPMSKLTVLNSMHSHFILADNGTTGKYGAEVKLRRQLEKHISLQKINTRCLPFFSLDSRLFYSFWGSCQLDSVGIGQGVPVVALIVEGGPNVISIVLEYLRDTPPVPVVVCDGSGRASDILAFGHKYSEEGGLINESLRDQLLVTIQKTFTYTRTQAQHLFIILMECMKKKELITVFRMGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKGNLPPDYRISLIDIGLVIEYLMGGAYRCNYTRKRFRTLYHNLFGPKRDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAGEETMSPTSPTLMPRMRSHSFYSVNMKDKGGIEKLESIFKERSLSLHRATSSHSVAKEPKAPAAPANTLAIVPDSRRPSSCIDIYVSAMDELHCDIDPLDNSVNILGLGEPSFSTPVPSTAPSSSAYATLAPTDRPPSRSIDFEDITSMDTRSFSSDYTHLPECQNPWDSEPPMYHTIERSKSSRYLATTPFLLEEAPIVKSHSFMFSPSRSYYANFGVPVKTAEYTSITDCIDTRCVNAPQAIADRAAFPGGLGDKVEDLTCCHPEREAELSHPSSDSEENEAKGRRATIAISSQEGDNSERTLSNNITVPKIERANSYSAEEPSAPYAHTRKSFSISDKLDRQRNTASLRNPFQRSKSSKPEGRGDSLSMRRLSRTSAFQSFESKHN

SEQ ID NO: 8 - protein, 1679 aa, Homo sapiens
MPEPWGTVYFLGIAQVFSFLFSWWNLEGVMNQADAPRPLNWTIRKLCHAAFLPSVRLLKAQKSWIERAFYKRECVHIIPSTKDPHRCCCGRLIGQHVGLTPSISVLQNEKNESRLSRNDIQSEKWSISKHTQLSPTDAFGTIEFQGGGHSNKAMYVRVSFDTKPDLLLHLMTKEWQLELPKLLISVHGGLQNFELQPKLKQVFGKGLIKAAMTTGAWIFTGGVNTGVIRHVGDALKDHASKSRGKICTIGIAPWGIVENQEDLIGRDVVRPYQTMSNPMSKLTVLNSMHSHFILADNGTTGKYGAEVKLRRQLEKHISLQKINTRIGQGVPVVALIVEGGPNVISIVLEYLRDTPPVPVVVCDGSGRASDILAFGHKYSEEGGLINESLRDQLLVTIQKTFTYTRTQAQHLFIILMECMKKKELITVFRMGSEGHQDIDLAILTALLKGANASAPDQLSLALAWNRVDIARSQIFIYGQQWPVGSLEQAMLDALVLDRVDFVKLLIENGRHGPSNTLYHLVRDVKKGNLPPDYRISLIDIGLVIEYLMGGAYRCNYTRKRFRTLYHNLFGPKRDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAGEETMSPTSPTLMPRMRSHSFYSVNMKDKGGIEKLESIFKERSLSLHRATSSHSVAKEPKAPAAPANTLAIVPDSRRPSSCIDIYVSAMDELHCDIDPLDNSVNILGLGEPSFSTPVPSTAPSSSAYATLAPTDRPPSRSIDFEDITSMDTRSFSSDYTHLPECQNPWDSEPPMYHTIERSKSSRYLATTPFLLEEAPIVKSHSFMFSPSRSYYANFGVPVKTAEYTSITDCIDTRCVNAPQAIADRAAFPGGLGDKVEDLTCCHPEREAELSHPSSDSEENEAKGRRATIAISSQEGDNSERTLSNNITVPKIERANSYSAEEPSAPYAHTRKSFSISDKLDRQRNTASLRNPFQRSKSSKPEGRGDSLSMRRLSRTSAFQSFESKHN

SEQ ID NO: 9 - protein, 1325 aa, Homo sapiens
MPEPWGTVYFLGIAQVFSFLFSWWNLEGVMNQADAPRPLNWTIRKLCHAAFLPSVRLLKAQKSWIERAFYKRECVHIIPSTKDPHRCCCGRLIGQHVGLTPSISVLQNEKNESRLSRNDIQSEKWSISKHTQLSPTDAFGTIEFQGGGHSNKAMYVRVSFDTKPDLLLHLMTKEWQLELPKLLISVHGGLQNFELQPKLKQVFGKGLIKAAMTTGAWIFTGGVNTGVIRHVGDALKDHASKSRGKICTIGIAPWGIVENQEDLIGRDVVRPYQTMSNPMSKLTVLNSMHSHFILADNGTTGKYGAEVKLRRQLEKHISLQKINTRIGQGVPVVALIVEGGPNVISIVLEYLRDTPPVPVVVCDGSGRASDILAFGHKYSEEGGLINESLRDQLLVTIQKTFTYTRTQAQHLFIILMECMKKKELITVFRMGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKGNLPPDYRISLIDIGLVIEYLMGGAYRCNYTRKRFRTLYHNLFGPKRPKALKLLGMEDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAEHPLYSV

SEQ ID NO: 10 - protein, 1734 aa, Homo sapiens
MGKKWRDAAEMERGCSDREDNAESRRRSRSASRGRFAESWKRLSSKQGSTKRSGLPSQQTPAQKSWIERAFYKRECVHIIPSTKDPHRCCCGRLIGQHVGLTPSISVLQNEKNESRLSRNDIQSEKWSISKHTQLSPTDAFGTIEFQGGGHSNKAMYVRVSFDTKPDLLLHLMTKEWQLELPKLLISVHGGLQNFELQPKLKQVFGKGLIKAAMTTGAWIFTGGVNTGVIRHVGDALKDHASKSRGKICTIGIAPWGIVENQEDLIGRDVVRPYQTMSNPMSKLTVLNSMHSHFILADNGTTGKYGAEVKLRRQLEKHISLQKINTRCLPFFSLDSRLFYSFWGSCQLDSVGIGQGVPVVALIVEGGPNVISIVLEYLRDTPPVPVVVCDGSGRASDILAFGHKYSEEGGLINESLRDQLLVTIQKTFTYTRTQAQHLFIILMECMKKKELITVFRMGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKGNLPPDYRISLIDIGLVIEYLMGGAYRCNYTRKRFRTLYHNLFGPKRPKALKLLGMEDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAGEETMSPTSPTLMPRMRSHSFYSVNMKDKGGIEKLESIFKERSLSLHRATSSHSVAKEPKAPAAPANTLAIVPDSRRPSSCIDIYVSAMDELHCDIDPLDNSVNILGLGEPSFSTPVPSTAPSSSAYATLAPTDRPPSRSIDFEDITSMDTRSFSSDYTHLPECQNPWDSEPPMYHTIERSKSSRYLATTPFLLEEAPIVKSHSFMFSPSRSYYANFGVPVKTAEYTSITDCIDTRCVNAPQAIADRAAFPGGLGDKVEDLTCCHPEREAELSHPSSDSEENEAKGRRATIAISSQEGDNSERTLSNNITVPKIERANSYSAEEPSAPYAHTRKSFSISDKLDRQRNTASLRNPFQRSKSSKPEGRGDSLSMRRLSRTSAFQSFESKHN

SEQ ID NO: 11 - protein, 1709 aa, Homo sapiens
MGKKWRDAAEMERGCSDREDNAESRRRSRSASRGRFAESWKRLSSKQGSTKRSGLPSQQTPAQKSWIERAFYKRECVHIIPSTKDPHRCCCGRLIGQHVGLTPSISVLQNEKNESRLSRNDIQSEKWSISKHTQLSPTDAFGTIEFQGGGHSNKAMYVRVSFDTKPDLLLHLMTKEWQLELPKLLISVHGGLQNFELQPKLKQVFGKGLIKAAMTTGAWIFTGGVNTGVIRHVGDALKDHASKSRGKICTIGIAPWGIVENQEDLIGRDVVRPYQTMSNPMSKLTVLNSMHSHFILADNGTTGKYGAEVKLRRQLEKHISLQKINTRIGQGVPVVALIVEGGPNVISIVLEYLRDTPPVPVVVCDGSGRASDILAFGHKYSEEGGLINESLRDQLLVTIQKTFTYTRTQAQHLFIILMECMKKKELITVFRMGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKGNLPPDYRISLIDIGLVIEYLMGGAYRCNYTRKRFRTLYHNLFGPKRPKALKLLGMEDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAGEETMSPTSPTLMPRMRSHSFYSVNMKDKGGIEKLESIFKERSLSLHRATSSHSVAKEPKAPAAPANTLAIVPDSRRPSSCIDIYVSAMDELHCDIDPLDNSVNILGLGEPSFSTPVPSTAPSSSAYATLAPTDRPPSRSIDFEDITSMDTRSFSSDYTHLPECQNPWDSEPPMYHTIERSKSSRYLATTPFLLEEAPIVKSHSFMFSPSRSYYANFGVPVKTAEYTSITDCIDTRCVNAPQAIADRAAFPGGLGDKVEDLTCCHPEREAELSHPSSDSEENEAKGRRATIAISSQEGDNSERTLSNNITVPKIERANSYSAEEPSAPYAHTRKSFSISDKLDRQRNTASLRNPFQRSKSSKPEGRGDSLSMRRLSRTSAFQSFESKHN

SEQ ID NO: 12 - protein, 1339 aa, Homo sapiens
MGKKWRDAAEMERGCSDREDNAESRRRSRSASRGRFAESWKRLSSKQGSTKRSGLPSQQTPAQKSWIERAFYKRECVHIIPSTKDPHRCCCGRLIGQHVGLTPSISVLQNEKNESRLSRNDIQSEKWSISKHTQLSPTDAFGTIEFQGGGHSNKAMYVRVSFDTKPDLLLHLMTKEWQLELPKLLISVHGGLQNFELQPKLKQVFGKGLIKAAMTTGAWIFTGGVNTGVIRHVGDALKDHASKSRGKICTIGIAPWGIVENQEDLIGRDVVRPYQTMSNPMSKLTVLNSMHSHFILADNGTTGKYGAEVKLRRQLEKHISLQKINTRIGQGVPVVALIVEGGPNVISIVLEYLRDTPPVPVVVCDGSGRASDILAFGHKYSEEGGLINESLRDQLLVTIQKTFTYTRTQAQHLFIILMECMKKKELITVFRMGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKREYPGFGWIYFKGNLPPDYRISLIDIGLVIEYLMGGAYRCNYTRKRFRTLYHNLFGPKRPKALKLLGMEDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAEHPLYSV

SEQ ID NO: 13 - protein, 1327 aa, Homo sapiens
MGKKWRDAAEMERGCSDREDNAESRRRSRSASRGRFAESWKRLSSKQGSTKRSGLPSQQTPAQKSWIERAFYKRECVHIIPSTKDPHRCCCGRLIGQHVGLTPSISVLQNEKNESRLSRNDIQSEKWSISKHTQLSPTDAFGTIEFQGGGHSNKAMYVRVSFDTKPDLLLHLMTKEWQLELPKLLISVHGGLQNFELQPKLKQVFGKGLIKAAMTTGAWIFTGGVNTGVIRHVGDALKDHASKSRGKICTIGIAPWGIVENQEDLIGRDVVRPYQTMSNPMSKLTVLNSMHSHFILADNGTTGKYGAEVKLRRQLEKHISLQKINTRIGQGVPVVALIVEGGPNVISIVLEYLRDTPPVPVVVCDGSGRASDILAFGHKYSEEGGLINESLRDQLLVTIQKTFTYTRTQAQHLFIILMECMKKKELITVFRMGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKGNLPPDYRISLIDIGLVIEYLMGGAYRCNYTRKRFRTLYHNLFGPKRPKALKLLGMEDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAEHPLYSV

SEQ ID NO: 14 - protein, 1337 aa, Homo sapiens
MPEPWGTVYFLGIAQVFSFLFSWWNLEGVMNQADAPRPLNWTIRKLCHAAFLPSVRLLKAQKSWIERAFYKRECVHIIPSTKDPHRCCCGRLIGQHVGLTPSISVLQNEKNESRLSRNDIQSEKWSISKHTQLSPTDAFGTIEFQGGGHSNKAMYVRVSFDTKPDLLLHLMTKEWQLELPKLLISVHGGLQNFELQPKLKQVFGKGLIKAAMTTGAWIFTGGVNTGVIRHVGDALKDHASKSRGKICTIGIAPWGIVENQEDLIGRDVVRPYQTMSNPMSKLTVLNSMHSHFILADNGTTGKYGAEVKLRRQLEKHISLQKINTRIGQGVPVVALIVEGGPNVISIVLEYLRDTPPVPVVVCDGSGRASDILAFGHKYSEEGGLINESLRDQLLVTIQKTFTYTRTQAQHLFIILMECMKKKELITVFRMGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKREYPGFGWIYFKGNLPPDYRISLIDIGLVIEYLMGGAYRCNYTRKRFRTLYHNLFGPKRPKALKLLGMEDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAEHPLYSV

SEQ ID NO: 15 - protein, 1744 aa, Homo sapiens
MPEPWGTVYFLGIAQVFSFLFSWWNLEGVMNQADAPRPLNWTIRKLCHAAFLPSVRLLKAQKSWIERAFYKRECVHIIPSTKDPHRCCCGRLIGQHVGLTPSISVLQNEKNESRLSRNDIQSEKWSISKHTQLSPTDAFGTIEFQGGGHSNKAMYVRVSFDTKPDLLLHLMTKEWQLELPKLLISVHGGLQNFELQPKLKQVFGKGLIKAAMTTGAWIFTGGVNTGVIRHVGDALKDHASKSRGKICTIGIAPWGIVENQEDLIGRDVVRPYQTMSNPMSKLTVLNSMHSHFILADNGTTGKYGAEVKLRRQLEKHISLQKINTRCLPFFSLDSRLFYSFWGSCQLDSVGIGQGVPVVALIVEGGPNVISIVLEYLRDTPPVPVVVCDGSGRASDILAFGHKYSEEGGLINESLRDQLLVTIQKTFTYTRTQAQHLFIILMECMKKKELITVFRMGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKREYPGFGWIYFKGNLPPDYRISLIDIGLVIEYLMGGAYRCNYTRKRFRTLYHNLFGPKRPKALKLLGMEDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAGEETMSPTSPTLMPRMRSHSFYSVNMKDKGGIEKLESIFKERSLSLHRATSSHSVAKEPKAPAAPANTLAIVPDSRRPSSCIDIYVSAMDELHCDIDPLDNSVNILGLGEPSFSTPVPSTAPSSSAYATLAPTDRPPSRSIDFEDITSMDTRSFSSDYTHLPECQNPWDSEPPMYHTIERSKSSRYLATTPFLLEEAPIVKSHSFMFSPSRSYYANFGVPVKTAEYTSITDCIDTRCVNAPQAIADRAAFPGGLGDKVEDLTCCHPEREAELSHPSSDSEENEAKGRRATIAISSQEGDNSERTLSNNITVPKIERANSYSAEEPSAPYAHTRKSFSISDKLDRQRNTASLRNPFQRSKSSKPEGRGDSLSMRRLSRTSAFQSFESKHN

SEQ ID NO: 16 - protein, 1352 aa, Homo sapiens
MPEPWGTVYFLGIAQVFSFLFSWWNLEGVMNQADAPRPLNWTIRKLCHAAFLPSVRLLKAQKSWIERAFYKRECVHIIPSTKDPHRCCCGRLIGQHVGLTPSISVLQNEKNESRLSRNDIQSEKWSISKHTQLSPTDAFGTIEFQGGGHSNKAMYVRVSFDTKPDLLLHLMTKEWQLELPKLLISVHGGLQNFELQPKLKQVFGKGLIKAAMTTGAWIFTGGVNTGVIRHVGDALKDHASKSRGKICTIGIAPWGIVENQEDLIGRDVVRPYQTMSNPMSKLTVLNSMHSHFILADNGTTGKYGAEVKLRRQLEKHISLQKINTRCLPFFSLDSRLFYSFWGSCQLDSVGIGQGVPVVALIVEGGPNVISIVLEYLRDTPPVPVVVCDGSGRASDILAFGHKYSEEGGLINESLRDQLLVTIQKTFTYTRTQAQHLFIILMECMKKKELITVFRMGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKREYPGFGWIYFKGNLPPDYRISLIDIGLVIEYLMGGAYRCNYTRKRFRTLYHNLFGPKRDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAEHPLYSV

SEQ ID NO: 17 - protein, 1315 aa, Homo sapiens
MPEPWGTVYFLGIAQVFSFLFSWWNLEGVMNQADAPRPLNWTIRKLCHAAFLPSVRLLKAQKSWIERAFYKRECVHIIPSTKDPHRCCCGRLIGQHVGLTPSISVLQNEKNESRLSRNDIQSEKWSISKHTQLSPTDAFGTIEFQGGGHSNKAMYVRVSFDTKPDLLLHLMTKEWQLELPKLLISVHGGLQNFELQPKLKQVFGKGLIKAAMTTGAWIFTGGVNTGVIRHVGDALKDHASKSRGKICTIGIAPWGIVENQEDLIGRDVVRPYQTMSNPMSKLTVLNSMHSHFILADNGTTGKYGAEVKLRRQLEKHISLQKINTRIGQGVPVVALIVEGGPNVISIVLEYLRDTPPVPVVVCDGSGRASDILAFGHKYSEEGGLINESLRDQLLVTIQKTFTYTRTQAQHLFIILMECMKKKELITVFRMGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKGNLPPDYRISLIDIGLVIEYLMGGAYRCNYTRKRFRTLYHNLFGPKRDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAEHPLYSV

SEQ ID NO: 18 - protein, 1362 aa, Homo sapiens
MPEPWGTVYFLGIAQVFSFLFSWWNLEGVMNQADAPRPLNWTIRKLCHAAFLPSVRLLKAQKSWIERAFYKRECVHIIPSTKDPHRCCCGRLIGQHVGLTPSISVLQNEKNESRLSRNDIQSEKWSISKHTQLSPTDAFGTIEFQGGGHSNKAMYVRVSFDTKPDLLLHLMTKEWQLELPKLLISVHGGLQNFELQPKLKQVFGKGLIKAAMTTGAWIFTGGVNTGVIRHVGDALKDHASKSRGKICTIGIAPWGIVENQEDLIGRDVVRPYQTMSNPMSKLTVLNSMHSHFILADNGTTGKYGAEVKLRRQLEKHISLQKINTRCLPFFSLDSRLFYSFWGSCQLDSVGIGQGVPVVALIVEGGPNVISIVLEYLRDTPPVPVVVCDGSGRASDILAFGHKYSEEGGLINESLRDQLLVTIQKTFTYTRTQAQHLFIILMECMKKKELITVFRMGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKREYPGFGWIYFKGNLPPDYRISLIDIGLVIEYLMGGAYRCNYTRKRFRTLYHNLFGPKRPKALKLLGMEDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAEHPLYSV

SEQ ID NO: 19 - protein, 1184 aa, Homo sapiens
MYVRVSFDTKPDLLLHLMTKEWQLELPKLLISVHGGLQNFELQPKLKQVFGKGLIKAAMTTGAWIFTGGVNTGVIRHVGDALKDHASKSRGKICTIGIAPWGIVENQEDLIGRDVVRPYQTMSNPMSKLTVLNSMHSHFILADNGTTGKYGAEVKLRRQLEKHISLQKINTRIGQGVPVVALIVEGGPNVISIVLEYLRDTPPVPVVVCDGSGRASDILAFGHKYSEEGGLINESLRDQLLVTIQKTFTYTRTQAQHLFIILMECMKKKELITVFRMGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKREYPGFGWIYFKGNLPPDYRISLIDIGLVIEYLMGGAYRCNYTRKRFRTLYHNLFGPKRPKALKLLGMEDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAEHPLYSV

SEQ ID NO: 20 - protein, 1554 aa, Homo sapiens
MYVRVSFDTKPDLLLHLMTKEWQLELPKLLISVHGGLQNFELQPKLKQVFGKGLIKAAMTTGAWIFTGGVNTGVIRHVGDALKDHASKSRGKICTIGIAPWGIVENQEDLIGRDVVRPYQTMSNPMSKLTVLNSMHSHFILADNGTTGKYGAEVKLRRQLEKHISLQKINTRIGQGVPVVALIVEGGPNVISIVLEYLRDTPPVPVVVCDGSGRASDILAFGHKYSEEGGLINESLRDQLLVTIQKTFTYTRTQAQHLFIILMECMKKKELITVFRMGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKGNLPPDYRISLIDIGLVIEYLMGGAYRCNYTRKRFRTLYHNLFGPKRPKALKLLGMEDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAGEETMSPTSPTLMPRMRSHSFYSVNMKDKGGIEKLESIFKERSLSLHRATSSHSVAKEPKAPAAPANTLAIVPDSRRPSSCIDIYVSAMDELHCDIDPLDNSVNILGLGEPSFSTPVPSTAPSSSAYATLAPTDRPPSRSIDFEDITSMDTRSFSSDYTHLPECQNPWDSEPPMYHTIERSKSSRYLATTPFLLEEAPIVKSHSFMFSPSRSYYANFGVPVKTAEYTSITDCIDTRCVNAPQAIADRAAFPGGLGDKVEDLTCCHPEREAELSHPSSDSEENEAKGRRATIAISSQEGDNSERTLSNNITVPKIERANSYSAEEPSAPYAHTRKSFSISDKLDRQRNTASLRNPFQRSKSSKPEGRGDSLSMRRLSRTSAFQSFESKHN

SEQ ID NO: 21 - protein, 1566 aa, Homo sapiens
MYVRVSFDTKPDLLLHLMTKEWQLELPKLLISVHGGLQNFELQPKLKQVFGKGLIKAAMTTGAWIFTGGVNTGVIRHVGDALKDHASKSRGKICTIGIAPWGIVENQEDLIGRDVVRPYQTMSNPMSKLTVLNSMHSHFILADNGTTGKYGAEVKLRRQLEKHISLQKINTRIGQGVPVVALIVEGGPNVISIVLEYLRDTPPVPVVVCDGSGRASDILAFGHKYSEEGGLINESLRDQLLVTIQKTFTYTRTQAQHLFIILMECMKKKELITVFRMGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKREYPGFGWIYFKGNLPPDYRISLIDIGLVIEYLMGGAYRCNYTRKRFRTLYHNLFGPKRPKALKLLGMEDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAGEETMSPTSPTLMPRMRSHSFYSVNMKDKGGIEKLESIFKERSLSLHRATSSHSVAKEPKAPAAPANTLAIVPDSRRPSSCIDIYVSAMDELHCDIDPLDNSVNILGLGEPSFSTPVPSTAPSSSAYATLAPTDRPPSRSIDFEDITSMDTRSFSSDYTHLPECQNPWDSEPPMYHTIERSKSSRYLATTPFLLEEAPIVKSHSFMFSPSRSYYANFGVPVKTAEYTSITDCIDTRCVNAPQAIADRAAFPGGLGDKVEDLTCCHPEREAELSHPSSDSEENEAKGRRATIAISSQEGDNSERTLSNNITVPKIERANSYSAEEPSAPYAHTRKSFSISDKLDRQRNTASLRNPFQRSKSSKPEGRGDSLSMRRLSRTSAFQSFESKHN

SEQ ID NO: 22 - protein, 1544 aa, Homo sapiens
MYVRVSFDTKPDLLLHLMTKEWQLELPKLLISVHGGLQNFELQPKLKQVFGKGLIKAAMTTGAWIFTGGVNTGVIRHVGDALKDHASKSRGKICTIGIAPWGIVENQEDLIGRDVVRPYQTMSNPMSKLTVLNSMHSHFILADNGTTGKYGAEVKLRRQLEKHISLQKINTRIGQGVPVVALIVEGGPNVISIVLEYLRDTPPVPVVVCDGSGRASDILAFGHKYSEEGGLINESLRDQLLVTIQKTFTYTRTQAQHLFIILMECMKKKELITVFRMGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKGNLPPDYRISLIDIGLVIEYLMGGAYRCNYTRKRFRTLYHNLFGPKRDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAGEETMSPTSPTLMPRMRSHSFYSVNMKDKGGIEKLESIFKERSLSLHRATSSHSVAKEPKAPAAPANTLAIVPDSRRPSSCIDIYVSAMDELHCDIDPLDNSVNILGLGEPSFSTPVPSTAPSSSAYATLAPTDRPPSRSIDFEDITSMDTRSFSSDYTHLPECQNPWDSEPPMYHTIERSKSSRYLATTPFLLEEAPIVKSHSFMFSPSRSYYANFGVPVKTAEYTSITDCIDTRCVNAPQAIADRAAFPGGLGDKVEDLTCCHPEREAELSHPSSDSEENEAKGRRATIAISSQEGDNSERTLSNNITVPKIERANSYSAEEPSAPYAHTRKSFSISDKLDRQRNTASLRNPFQRSKSSKPEGRGDSLSMRRLSRTSAFQSFESKHN

SEQ ID NO: 23 - protein, 1556 aa, Homo sapiens
MYVRVSFDTKPDLLLHLMTKEWQLELPKLLISVHGGLQNFELQPKLKQVFGKGLIKAAMTTGAWIFTGGVNTGVIRHVGDALKDHASKSRGKICTIGIAPWGIVENQEDLIGRDVVRPYQTMSNPMSKLTVLNSMHSHFILADNGTTGKYGAEVKLRRQLEKHISLQKINTRIGQGVPVVALIVEGGPNVISIVLEYLRDTPPVPVVVCDGSGRASDILAFGHKYSEEGGLINESLRDQLLVTIQKTFTYTRTQAQHLFIILMECMKKKELITVFRMGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKREYPGFGWIYFKGNLPPDYRISLIDIGLVIEYLMGGAYRCNYTRKRFRTLYHNLFGPKRDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAGEETMSPTSPTLMPRMRSHSFYSVNMKDKGGIEKLESIFKERSLSLHRATSSHSVAKEPKAPAAPANTLAIVPDSRRPSSCIDIYVSAMDELHCDIDPLDNSVNILGLGEPSFSTPVPSTAPSSSAYATLAPTDRPPSRSIDFEDITSMDTRSFSSDYTHLPECQNPWDSEPPMYHTIERSKSSRYLATTPFLLEEAPIVKSHSFMFSPSRSYYANFGVPVKTAEYTSITDCIDTRCVNAPQAIADRAAFPGGLGDKVEDLTCCHPEREAELSHPSSDSEENEAKGRRATIAISSQEGDNSERTLSNNITVPKIERANSYSAEEPSAPYAHTRKSFSISDKLDRQRNTASLRNPFQRSKSSKPEGRGDSLSMRRLSRTSAFQSFESKHN

SEQ ID NO: 24 - protein, 1579 aa, Homo sapiens
MYVRVSFDTKPDLLLHLMTKEWQLELPKLLISVHGGLQNFELQPKLKQVFGKGLIKAAMTTGAWIFTGGVNTGVIRHVGDALKDHASKSRGKICTIGIAPWGIVENQEDLIGRDVVRPYQTMSNPMSKLTVLNSMHSHFILADNGTTGKYGAEVKLRRQLEKHISLQKINTRCLPFFSLDSRLFYSFWGSCQLDSVGIGQGVPVVALIVEGGPNVISIVLEYLRDTPPVPVVVCDGSGRASDILAFGHKYSEEGGLINESLRDQLLVTIQKTFTYTRTQAQHLFIILMECMKKKELITVFRMGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKGNLPPDYRISLIDIGLVIEYLMGGAYRCNYTRKRFRTLYHNLFGPKRPKALKLLGMEDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAGEETMSPTSPTLMPRMRSHSFYSVNMKDKGGIEKLESIFKERSLSLHRATSSHSVAKEPKAPAAPANTLAIVPDSRRPSSCIDIYVSAMDELHCDIDPLDNSVNILGLGEPSFSTPVPSTAPSSSAYATLAPTDRPPSRSIDFEDITSMDTRSFSSDYTHLPECQNPWDSEPPMYHTIERSKSSRYLATTPFLLEEAPIVKSHSFMFSPSRSYYANFGVPVKTAEYTSITDCIDTRCVNAPQAIADRAAFPGGLGDKVEDLTCCHPEREAELSHPSSDSEENEAKGRRATIAISSQEGDNSERTLSNNITVPKIERANSYSAEEPSAPYAHTRKSFSISDKLDRQRNTASLRNPFQRSKSSKPEGRGDSLSMRRLSRTSAFQSFESKHN

SEQ ID NO: 25 - protein, 1569 aa, Homo sapiens
MYVRVSFDTKPDLLLHLMTKEWQLELPKLLISVHGGLQNFELQPKLKQVFGKGLIKAAMTTGAWIFTGGVNTGVIRHVGDALKDHASKSRGKICTIGIAPWGIVENQEDLIGRDVVRPYQTMSNPMSKLTVLNSMHSHFILADNGTTGKYGAEVKLRRQLEKHISLQKINTRCLPFFSLDSRLFYSFWGSCQLDSVGIGQGVPVVALIVEGGPNVISIVLEYLRDTPPVPVVVCDGSGRASDILAFGHKYSEEGGLINESLRDQLLVTIQKTFTYTRTQAQHLFIILMECMKKKELITVFRMGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKGNLPPDYRISLIDIGLVIEYLMGGAYRCNYTRKRFRTLYHNLFGPKRDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAGEETMSPTSPTLMPRMRSHSFYSVNMKDKGGIEKLESIFKERSLSLHRATSSHSVAKEPKAPAAPANTLAIVPDSRRPSSCIDIYVSAMDELHCDIDPLDNSVNILGLGEPSFSTPVPSTAPSSSAYATLAPTDRPPSRSIDFEDITSMDTRSFSSDYTHLPECQNPWDSEPPMYHTIERSKSSRYLATTPFLLEEAPIVKSHSFMFSPSRSYYANFGVPVKTAEYTSITDCIDTRCVNAPQAIADRAAFPGGLGDKVEDLTCCHPEREAELSHPSSDSEENEAKGRRATIAISSQEGDNSERTLSNNITVPKIERANSYSAEEPSAPYAHTRKSFSISDKLDRQRNTASLRNPFQRSKSSKPEGRGDSLSMRRLSRTSAFQSFESKHN

SEQ ID NO: 26 - protein, 1699 aa, Homo sapiens
MGKKWRDAAEMERGCSDREDNAESRRRSRSASRGRFAESWKRLSSKQGSTKRSGLPSQQTPAQKSWIERAFYKRECVHIIPSTKDPHRCCCGRLIGQHVGLTPSISVLQNEKNESRLSRNDIQSEKWSISKHTQLSPTDAFGTIEFQGGGHSNKAMYVRVSFDTKPDLLLHLMTKEWQLELPKLLISVHGGLQNFELQPKLKQVFGKGLIKAAMTTGAWIFTGGVNTGVIRHVGDALKDHASKSRGKICTIGIAPWGIVENQEDLIGRDVVRPYQTMSNPMSKLTVLNSMHSHFILADNGTTGKYGAEVKLRRQLEKHISLQKINTRIGQGVPVVALIVEGGPNVISIVLEYLRDTPPVPVVVCDGSGRASDILAFGHKYSEEGGLINESLRDQLLVTIQKTFTYTRTQAQHLFIILMECMKKKELITVFRMGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKGNLPPDYRISLIDIGLVIEYLMGGAYRCNYTRKRFRTLYHNLFGPKRDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAGEETMSPTSPTLMPRMRSHSFYSVNMKDKGGIEKLESIFKERSLSLHRATSSHSVAKEPKAPAAPANTLAIVPDSRRPSSCIDIYVSAMDELHCDIDPLDNSVNILGLGEPSFSTPVPSTAPSSSAYATLAPTDRPPSRSIDFEDITSMDTRSFSSDYTHLPECQNPWDSEPPMYHTIERSKSSRYLATTPFLLEEAPIVKSHSFMFSPSRSYYANFGVPVKTAEYTSITDCIDTRCVNAPQAIADRAAFPGGLGDKVEDLTCCHPEREAELSHPSSDSEENEAKGRRATIAISSQEGDNSERTLSNNITVPKIERANSYSAEEPSAPYAHTRKSFSISDKLDRQRNTASLRNPFQRSKSSKPEGRGDSLSMRRLSRTSAFQSFESKHN

SEQ ID NO: 27 - protein, 1711 aa, Homo sapiens
MGKKWRDAAEMERGCSDREDNAESRRRSRSASRGRFAESWKRLSSKQGSTKRSGLPSQQTPAQKSWIERAFYKRECVHIIPSTKDPHRCCCGRLIGQHVGLTPSISVLQNEKNESRLSRNDIQSEKWSISKHTQLSPTDAFGTIEFQGGGHSNKAMYVRVSFDTKPDLLLHLMTKEWQLELPKLLISVHGGLQNFELQPKLKQVFGKGLIKAAMTTGAWIFTGGVNTGVIRHVGDALKDHASKSRGKICTIGIAPWGIVENQEDLIGRDVVRPYQTMSNPMSKLTVLNSMHSHFILADNGTTGKYGAEVKLRRQLEKHISLQKINTRIGQGVPVVALIVEGGPNVISIVLEYLRDTPPVPVVVCDGSGRASDILAFGHKYSEEGGLINESLRDQLLVTIQKTFTYTRTQAQHLFIILMECMKKKELITVFRMGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKREYPGFGWIYFKGNLPPDYRISLIDIGLVIEYLMGGAYRCNYTRKRFRTLYHNLFGPKRDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAGEETMSPTSPTLMPRMRSHSFYSVNMKDKGGIEKLESIFKERSLSLHRATSSHSVAKEPKAPAAPANTLAIVPDSRRPSSCIDIYVSAMDELHCDIDPLDNSVNILGLGEPSFSTPVPSTAPSSSAYATLAPTDRPPSRSIDFEDITSMDTRSFSSDYTHLPECQNPWDSEPPMYHTIERSKSSRYLATTPFLLEEAPIVKSHSFMFSPSRSYYANFGVPVKTAEYTSITDCIDTRCVNAPQAIADRAAFPGGLGDKVEDLTCCHPEREAELSHPSSDSEENEAKGRRATIAISSQEGDNSERTLSNNITVPKIERANSYSAEEPSAPYAHTRKSFSISDKLDRQRNTASLRNPFQRSKSSKPEGRGDSLSMRRLSRTSAFQSFESKHN

SEQ ID NO: 28 - protein, 1721 aa, Homo sapiens
MGKKWRDAAEMERGCSDREDNAESRRRSRSASRGRFAESWKRLSSKQGSTKRSGLPSQQTPAQKSWIERAFYKRECVHIIPSTKDPHRCCCGRLIGQHVGLTPSISVLQNEKNESRLSRNDIQSEKWSISKHTQLSPTDAFGTIEFQGGGHSNKAMYVRVSFDTKPDLLLHLMTKEWQLELPKLLISVHGGLQNFELQPKLKQVFGKGLIKAAMTTGAWIFTGGVNTGVIRHVGDALKDHASKSRGKICTIGIAPWGIVENQEDLIGRDVVRPYQTMSNPMSKLTVLNSMHSHFILADNGTTGKYGAEVKLRRQLEKHISLQKINTRIGQGVPVVALIVEGGPNVISIVLEYLRDTPPVPVVVCDGSGRASDILAFGHKYSEEGGLINESLRDQLLVTIQKTFTYTRTQAQHLFIILMECMKKKELITVFRMGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKREYPGFGWIYFKGNLPPDYRISLIDIGLVIEYLMGGAYRCNYTRKRFRTLYHNLFGPKRPKALKLLGMEDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAGEETMSPTSPTLMPRMRSHSFYSVNMKDKGGIEKLESIFKERSLSLHRATSSHSVAKEPKAPAAPANTLAIVPDSRRPSSCIDIYVSAMDELHCDIDPLDNSVNILGLGEPSFSTPVPSTAPSSSAYATLAPTDRPPSRSIDFEDITSMDTRSFSSDYTHLPECQNPWDSEPPMYHTIERSKSSRYLATTPFLLEEAPIVKSHSFMFSPSRSYYANFGVPVKTAEYTSITDCIDTRCVNAPQAIADRAAFPGGLGDKVEDLTCCHPEREAELSHPSSDSEENEAKGRRATIAISSQEGDNSERTLSNNITVPKIERANSYSAEEPSAPYAHTRKSFSISDKLDRQRNTASLRNPFQRSKSSKPEGRGDSLSMRRLSRTSAFQSFESKHN

SEQ ID NO: 29 - protein, 1734 aa, Homo sapiens
MPEPWGTVYFLGIAQVFSFLFSWWNLEGVMNQADAPRPLNWTIRKLCHAAFLPSVRLLKAQKSWIERAFYKRECVHIIPSTKDPHRCCCGRLIGQHVGLTPSISVLQNEKNESRLSRNDIQSEKWSISKHTQLSPTDAFGTIEFQGGGHSNKAMYVRVSFDTKPDLLLHLMTKEWQLELPKLLISVHGGLQNFELQPKLKQVFGKGLIKAAMTTGAWIFTGGVNTGVIRHVGDALKDHASKSRGKICTIGIAPWGIVENQEDLIGRDVVRPYQTMSNPMSKLTVLNSMHSHFILADNGTTGKYGAEVKLRRQLEKHISLQKINTRCLPFFSLDSRLFYSFWGSCQLDSVGIGQGVPVVALIVEGGPNVISIVLEYLRDTPPVPVVVCDGSGRASDILAFGHKYSEEGGLINESLRDQLLVTIQKTFTYTRTQAQHLFIILMECMKKKELITVFRMGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKREYPGFGWIYFKGNLPPDYRISLIDIGLVIEYLMGGAYRCNYTRKRFRTLYHNLFGPKRDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAGEETMSPTSPTLMPRMRSHSFYSVNMKDKGGIEKLESIFKERSLSLHRATSSHSVAKEPKAPAAPANTLAIVPDSRRPSSCIDIYVSAMDELHCDIDPLDNSVNILGLGEPSFSTPVPSTAPSSSAYATLAPTDRPPSRSIDFEDITSMDTRSFSSDYTHLPECQNPWDSEPPMYHTIERSKSSRYLATTPFLLEEAPIVKSHSFMFSPSRSYYANFGVPVKTAEYTSITDCIDTRCVNAPQAIADRAAFPGGLGDKVEDLTCCHPEREAELSHPSSDSEENEAKGRRATIAISSQEGDNSERTLSNNITVPKIERANSYSAEEPSAPYAHTRKSFSISDKLDRQRNTASLRNPFQRSKSSKPEGRGDSLSMRRLSRTSAFQSFESKHN

SEQ ID NO: 30 - protein, 1268 aa, Homo sapiens
MGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKGNLPPDYRISLIDIGLVIEYLMGGAYRCNYTRKRFRTLYHNLFGPKRDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAGEETMSPTSPTLMPRMRSHSFYSVNMKDKGGIEKLESIFKERSLSLHRATSSHSVAKEPKAPAAPANTLAIVPDSRRPSSCIDIYVSAMDELHCDIDPLDNSVNILGLGEPSFSTPVPSTAPSSSAYATLAPTDRPPSRSIDFEDITSMDTRSFSSDYTHLPECQNPWDSEPPMYHTIERSKSSRYLATTPFLLEEAPIVKSHSFMFSPSRSYYANFGVPVKTAEYTSITDCIDTRCVNAPQAIADRAAFPGGLGDKVEDLTCCHPEREAELSHPSSDSEENEAKGRRATIAISSQEGDNSERTLSNNITVPKIERANSYSAEEPSAPYAHTRKSFSISDKLDRQRNTASLRNPFQRSKSSKPEGRGDSLSMRRLSRTSAFQSFESKHN

SEQ ID NO: 31 - protein, 896 aa, Homo sapiens
MGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKGNLPPDYRISLIDIGLVIEYLMGGAYRCNYTRKRFRTLYHNLFGPKRPKALKLLGMEDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAEHPLYSV

SEQ ID NO: 32 - protein, 1278 aa, Homo sapiens
MGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKGNLPPDYRISLIDIGLVIEYLMGGAYRCNYTRKRFRTLYHNLFGPKRPKALKLLGMEDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAGEETMSPTSPTLMPRMRSHSFYSVNMKDKGGIEKLESIFKERSLSLHRATSSHSVAKEPKAPAAPANTLAIVPDSRRPSSCIDIYVSAMDELHCDIDPLDNSVNILGLGEPSFSTPVPSTAPSSSAYATLAPTDRPPSRSIDFEDITSMDTRSFSSDYTHLPECQNPWDSEPPMYHTIERSKSSRYLATTPFLLEEAPIVKSHSFMFSPSRSYYANFGVPVKTAEYTSITDCIDTRCVNAPQAIADRAAFPGGLGDKVEDLTCCHPEREAELSHPSSDSEENEAKGRRATIAISSQEGDNSERTLSNNITVPKIERANSYSAEEPSAPYAHTRKSFSISDKLDRQRNTASLRNPFQRSKSSKPEGRGDSLSMRRLSRTSAFQSFESKHN

SEQ ID NO: 33 - protein, 898 aa, Homo sapiens
MGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKREYPGFGWIYFKGNLPPDYRISLIDIGLVIEYLMGGAYRCNYTRKRFRTLYHNLFGPKRDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAEHPLYSV

SEQ ID NO: 34 - protein, 1280 aa, Homo sapiens
MGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKREYPGFGWIYFKGNLPPDYRISLIDIGLVIEYLMGGAYRCNYTRKRFRTLYHNLFGPKRDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAGEETMSPTSPTLMPRMRSHSFYSVNMKDKGGIEKLESIFKERSLSLHRATSSHSVAKEPKAPAAPANTLAIVPDSRRPSSCIDIYVSAMDELHCDIDPLDNSVNILGLGEPSFSTPVPSTAPSSSAYATLAPTDRPPSRSIDFEDITSMDTRSFSSDYTHLPECQNPWDSEPPMYHTIERSKSSRYLATTPFLLEEAPIVKSHSFMFSPSRSYYANFGVPVKTAEYTSITDCIDTRCVNAPQAIADRAAFPGGLGDKVEDLTCCHPEREAELSHPSSDSEENEAKGRRATIAISSQEGDNSERTLSNNITVPKIERANSYSAEEPSAPYAHTRKSFSISDKLDRQRNTASLRNPFQRSKSSKPEGRGDSLSMRRLSRTSAFQSFESKHN

SEQ ID NO: 35 - protein, 908 aa, Homo sapiens
MGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKREYPGFGWIYFKGNLPPDYRISLIDIGLVIEYLMGGAYRCNYTRKRFRTLYHNLFGPKRPKALKLLGMEDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAEHPLYSV

SEQ ID NO: 36 - protein, 1290 aa, Homo sapiens
MGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKREYPGFGWIYFKGNLPPDYRISLIDIGLVIEYLMGGAYRCNYTRKRFRTLYHNLFGPKRPKALKLLGMEDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDPPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAGEETMSPTSPTLMPRMRSHSFYSVNMKDKGGIEKLESIFKERSLSLHRATSSHSVAKEPKAPAAPANTLAIVPDSRRPSSCIDIYVSAMDELHCDIDPLDNSVNILGLGEPSFSTPVPSTAPSSSAYATLAPTDRPPSRSIDFEDITSMDTRSFSSDYTHLPECQNPWDSEPPMYHTIERSKSSRYLATTPFLLEEAPIVKSHSFMFSPSRSYYANFGVPVKTAEYTSITDCIDTRCVNAPQAIADRAAFPGGLGDKVEDLTCCHPEREAELSHPSSDSEENEAKGRRATIAISSQEGDNSERTLSNNITVPKIERANSYSAEEPSAPYAHTRKSFSISDKLDRQRNTASLRNPFQRSKSSKPEGRGDSLSMRRLSRTSAFQSFESKHN

SEQ ID NO: 37 - protein, 1566 aa, Homo sapiens
MYVRVSFDTKPDLLLHLMTKEWQLELPKLLISVHGGLQNFELQPKLKQVFGKGLIKAAMTTGAWIFTGGVNTGVIRHVGDALKDHASKSRGKICTIGIAPWGIVENQEDLIGRDVVRPYQTMSNPMSKLTVLNSMHSHFILADNGTTGKYGAEVKLRRQLEKHISLQKINTRIGQGVPVVALIVEGGPNVISIVLEYLRDTPPVPVVVCDGSGRASDILAFGHKYSEEGGLINESLRDQLLVTIQKTFTYTRTQAQHLFIILMECMKKKELITVFRMGSEGHQDIDLAILTALLKGANASAPDQLSLALAWNRVDIARSQIFIYGQQWPVGSLEQAMLDALVLDRVDFVKLLIENGVSMHRFLTISRLEELYNTRHGPSNTLYHLVRDVKKGNLPPDYRISLIDIGLVIEYLMGGAYRCNYTRKRFRTLYHNLFGPKRPKALKLLGMEDDIPLRRGRKTTKKREEEVDIDLDDPEINHFPFPFHELMVWAVLMKRQKMALFFWQHGEEAMAKALVACKLCKAMAHEASENDMVDDISQELNHNSRDFGQLAVELLDQSYKQDEQLAMKLLTYELKNWSNATCLQLAVAAKHRDFIAHTCSQMLLTDMWMGRLRMRKNSGLKVILGILLPPSILSLEFKNKDDMPYMSQAQEIHLQEKEAEEPEKPTKEKEEEDMELTAMLGRNNGESSRKKDEEEVQSKHRLIPLGRKIYEFYNAPIVKFWFYTLAYIGYLMLFNYIVLVKMERWPSTQEWIVISYIFTLGIEKMREILMSEPGKLLQKVKVWLQEYWNVTDLIAILLFSVGMILRLQDQPFRSDGRVIYCVNIIYWYIRLLDIFGVNKYLGPYVMMIGKMMIDMMYFVIIMLVVLMSFGVARQAILFPNEEPSWKLAKNIFYMPYWMIYGEVFADQIDRKQVYDSHTPKSAPCGQNETREDGKIIQLPPCKTGAWIVPAIMACYLLVANILLVNLLIAVFNNTFFEVKSISNQVWKFQRYQLIMTFHERPVLPPPLIIFSHMTMIFQHLCCRWRKHESDPDERDYGLKLFITDDELKKVHDFEEQCIEEYFREKDDRFNSSNDERIRVTSERVENMSMRLEEVNEREHSMKASLQTVDIRLAQLEDLIGRMATALERLTGLERAESNKIRSRTSSDCTDAAYIVRQSSFNSQEGNTFKLQESIDPAGEETMSPTSPTLMPRMRSHSFYSVNMKDKGGIEKLESIFKERSLSLHRATSSHSVAKEPKAPAAPANTLAIVPDSRRPSSCIDIYVSAMDELHCDIDPLDNSVNILGLGEPSFSTPVPSTAPSSSAYATLAPTDRPPSRSIDFEDITSMDTRSFSSDYTHLPECQNPWDSEPPMYHTIERSKSSRYLATTPFLLEEAPIVKSHSFMFSPSRSYYANFGVPVKTAEYTSITDCIDTRCVNAPQAIADRAAFPGGLGDKVEDLTCCHPEREAELSHPSSDSEENEAKGRRATIAISSQEGDNSERTLSNNITVPKIERANSYSAEEPSAPYAHTRKSFSISDKLDRQRNTASLRNPFQRSKSSKPEGRGDSLSMRRLSRTSAFQSFESKHN

SEQ ID NO: 38 - DNA, 298 bp, Homo sapiens
  source 1..298: mol_type = genomic DNA
ggcaaaggcagcgccaaccgagagccgcgcgcaggctggagggagacccgcggcgaggagccagcgagagcgctcggcgctgggctgtttcccggccgagggaggcgaacttctcatggggaagaagtggagggatgcggcggaaatggagcggggctgctccgaccgcgaggacaacgcggagagccgcagacgcagccggagcgccagccggggcaggtttgccgagtcgtggaaaaggttaagttccaagcaggggtccaccaaacgctcgggactcccgtcgcagcagacgccg

SEQ ID NO: 39 - DNA, 444 bp, Homo sapiens
  source 1..444: mol_type = genomic DNA
attctggagcaggctgctttgactccgaccacaggctgttttgtgcaggctgtccctcttcttcaaaatcgtgcatcccctccccgaagcagcaggcagtgtgcctccattcagccacatttggtatgcatgagcacggctgcagagagaggggaggtggctgttttaagaaggttcaggggctcaggcaaggctacttgactagtcttccaagttccaggaagcctctgccctaatggaatttgcaggtgtggagatgaccatgggatgccagagccgtgggggaccgtttattttctaggcattgctcaggttttcagtttcttgttttcctggtggaatttggaaggggtcatgaatcaggctgatgctcctcgacccctaaactggaccatccggaagctgtgccacgcagcctttcttccatctgtcagacttctgaag

SEQ ID NO: 40 - DNA, 67 bp, Homo sapiens
  source 1..67: mol_type = genomic DNA
agcaaaatgaaagcaacaggagctgctccggggactgcttttgccagtacccagaatcagtgctcag

SEQ ID NO: 41 - DNA, 80 bp, Homo sapiens
  source 1..80: mol_type = genomic DNA
gctcagaaatcctggatagaaagagcattttataaaagagaatgtgtccacatcatacccagcaccaaagacccccatag

SEQ ID NO: 42 - DNA, 205 bp, Homo sapiens
  source 1..205: mol_type = genomic DNA
gtgttgctgtgggcgtctgataggccagcatgttggcctcacccccagtatctccgtgcttcagaatgagaaaaatgaaagtcgcctctcccgaaatgacatccagtctgaaaagtggtccatcagcaaacacactcaactcagccctacggatgcttttgggaccattgagttccaaggaggtggccattccaacaaagccatg

SEQ ID NO: 43 - DNA, 214 bp, Homo sapiens
  source 1..214: mol_type = genomic DNA
tatgtgcgagtatcttttgatacaaaacctgatctcctcttacacctgatgaccaaggaatggcagttggagcttcccaagcttctcatctctgtccatgggggcctgcagaactttgaactccagccaaaactcaagcaagtctttgggaaagggctcatcaaagcagcaatgacaactggagcgtggatattcactggaggggttaacacag

SEQ ID NO: 44 - DNA, 125 bp, Homo sapiens
  source 1..125: mol_type = genomic DNA
gtgttattcgtcatgttggcgatgccttgaaggatcatgcctctaagtctcgaggaaagatatgcaccataggtattgccccctggggaattgtggaaaaccaggaggacctcattggaagagat

SEQ ID NO: 45 - DNA, 172 bp, Homo sapiens
  source 1..172: mol_type = genomic DNA
gttgtccggccataccagaccatgtccaatcccatgagcaagctcactgttctcaacagcatgcattcccacttcattctggctgacaacgggaccactggaaaatatggagcagaggtgaaacttcgaagacaactggaaaagcatatttcactccagaagataaacacaa

SEQ ID NO: 46 - DNA, 75 bp, Homo sapiens
  source 1..75: mol_type = genomic DNA
gatgcctgccgtttttctctcttgactcccgcttgttttattcattttggggtagttgccagttagactcagttg

SEQ ID NO: 47 - DNA, 175 bp, Homo sapiens
  source 1..175: mol_type = genomic DNA
gaatcggtcaaggtgttcctgtggtggcactcatagtggaaggaggacccaatgtgatctcgattgttttggagtaccttcgagacacccctcccgtgccagtggttgtctgtgatgggagtggacgggcatcggacatcctggcctttgggcataaatactcagaagaaggcgg

SEQ ID NO: 48 - DNA, 124 bp, Homo sapiens
  source 1..124: mol_type = genomic DNA
actgataaatgaatctttgagggaccagctgttggtgactatacagaagactttcacatacactcgaacccaagctcagcatctgttcatcatcctcatggagtgcatgaagaagaaggaattg

SEQ ID NO: 49 - DNA, 175 bp, Homo sapiens
  source 1..175: mol_type = genomic DNA
gcagaatctcaccaggcctgcctaaactacccatccctggctgacctcagtgccccctagtctctccattccagttgaaacaggacagctggatgactcccattaaggagcaactctcataatgtaacctcccacccaatcggaaacccacattaccctcgctgtgcttcccaag

SEQ ID NO: 50 - DNA, 73 bp, Homo sapiens
  source 1..73: mol_type = genomic DNA
attacggtatttcggatgggatcagaaggacaccaggacattgatttggctatcctgacagctttactcaaag

SEQ ID NO: 51 - DNA, 101 bp, Homo sapiens
  source 1..101: mol_type = genomic DNA
gagccaatgcctcggccccagaccaactgagcttagctttagcctggaacagagtcgacatcgctcgcagccagatctttatttacgggcaacagtggccg

SEQ ID NO: 52 - DNA, 135 bp, Homo sapiens
  source 1..135: mol_type = genomic DNA
gtgggatctctggagcaagccatgttggatgccttagttctggacagagtggattttgtgaaattactcatagagaatggagtaagcatgcaccgttttctcaccatctccagactagaggaattgtacaatacg

SEQ ID NO: 53 - DNA, 51 bp, Homo sapiens
  source 1..51: mol_type = genomic DNA
agacatgggccctcaaatacattgtaccacttggtcagggatgtcaaaaag

SEQ ID NO: 54 - DNA, 36 bp, Homo sapiens
  source 1..36: mol_type = genomic DNA
cgagagtatccaggtttcggttggatctattttaag

SEQ ID NO: 55 - DNA, 141 bp, Homo sapiens
  source 1..141: mol_type = genomic DNA
gggaacctgcccccagactacagaatcagcctgattgacatcggcctggtgatcgagtacctgatgggcggggcttatcgctgcaactacacgcgcaagcgcttccggaccctctaccacaacctcttcggccccaagagg

SEQ ID NO: 56 - DNA, 30 bp, Homo sapiens
  source 1..30: mol_type = genomic DNA
cccaaagccttgaaactgctgggaatggag

SEQ ID NO: 57 - DNA, 290 bp, Homo sapiens
  source 1..290: mol_type = genomic DNA
gatgatattcccttgaggcgaggaagaaagacaaccaagaaacgtgaagaagaggtggacattgacttggatgatcctgagatcaaccacttccccttccctttccatgagctcatggtgtgggctgttctcatgaagcggcagaagatggccctgttcttctggcagcacggtgaggaggccatggccaaggccctggtggcctgcaagctctgcaaagccatggctcatgaggcctctgagaacgacatggttgacgacatttcccaggagctgaatcacaattccag

SEQ ID NO: 58 - DNA, 229 bp, Homo sapiens
  source 1..229: mol_type = genomic DNA
agactttggccagctggctgtggagctcctggaccagtcctacaagcaggacgaacagctggccatgaaactgctgacgtatgagctgaagaactggagcaacgccacgtgcctgcagcttgccgtggctgccaaacaccgcgacttcatcgcgcacacgtgcagccagatgctgctcaccgacatgtggatgggccggctccgcatgcgcaagaactcaggcctcaag

SEQ ID NO: 59 - DNA, 168 bp, Homo sapiens
  source 1..168: mol_type = genomic DNA
gtaattctgggaattctacttcctccttcaattctcagcttggagttcaagaacaaagacgacatgccctatatgtctcaggcccaggaaatccacctccaagagaaggaggcagaagaaccagagaagcccacaaaggaaaaagaggaagaggacatggagctcaca

SEQ ID NO: 60 - DNA, 141 bp, Homo sapiens
  source 1..141: mol_type = genomic DNA
gcaatgttgggacgaaacaacggggagtcctccaggaagaaggatgaagaggaagttcagagcaagcaccggttaatccccctcggcagaaaaatctatgaattctacaatgcacccatcgtgaagttctggttctacaca

SEQ ID NO: 61 - DNA, 129 bp, Homo sapiens
  source 1..129: mol_type = genomic DNA
ctggcgtatatcggatacctgatgctcttcaactatatcgtgttagtgaagatggaacgctggccgtccacccaggaatggatcgtaatctcctatattttcaccctgggaatagaaaagatgagagag

SEQ ID NO: 62 - DNA, 252 bp, Homo sapiens
  source 1..252: mol_type = genomic DNA
attctgatgtcagagccagggaagttgctacagaaagtgaaggtatggctgcaggagtactggaatgtcacggacctcatcgccatccttctgttttctgtcggaatgatccttcgtctccaagaccagcccttcaggagtgacgggagggtcatctactgcgtgaacatcatttactggtatatccgtctcctagacatcttcggcgtgaacaagtatttgggcccgtatgtaatgatgattggaaaaatg

SEQ ID NO: 63 - DNA, 175 bp, Homo sapiens
  source 1..175: mol_type = genomic DNA
atgatagacatgatgtactttgtcatcattatgctggtggttctgatgagctttggggtcgccaggcaagccatcctttttcccaatgaggagccatcatggaaactggccaagaacatcttctacatgccctattggatgatttatggggaagtgtttgcggaccagatagacc

SEQ ID NO: 64 - DNA, 151 bp, Homo sapiens
  source 1..151: mol_type = genomic DNA
ctccctgtggacagaatgagacccgagaggatggtaaaataatccagctgcctccctgcaagacaggagcttggatcgtgccggccatcatggcctgctacctcttagtggcaaacatcttgctggtcaacctcctcattgctgtctttaa

SEQ ID NO: 65 - DNA, 200 bp, Homo sapiens
  source 1..200: mol_type = genomic DNA
caatacattttttgaagtaaaatcgatatccaaccaagtctggaagtttcagaggtatcagctcatcatgactttccatgaaaggccagttctgcccccaccactgatcatcttcagccacatgaccatgatattccagcacctgtgctgccgatggaggaaacacgagagcgacccggatgaaagggactacggcctga

SEQ ID NO: 66 - DNA, 133 bp, Homo sapiens
  source 1..133: mol_type = genomic DNA
aactcttcataaccgatgatgagctcaagaaagtacatgactttgaagagcaatgcatagaagaatacttcagagaaaaggatgatcggttcaactcatctaatgatgagaggatacgggtgacttcagaaag

SEQ ID NO: 67 - DNA, 8343 bp, Homo sapiens
  source 1..8343: mol_type = genomic DNA
ggtggagaacatgtctatgcggctggaggaagtcaacgagagagagcactccatgaaggcttcactccagaccgtggacatccggctggcgcagctggaagaccttatcgggcgcatggccacggccctggagcgcctgacaggtctggagcgggccgagtccaacaaaatccgctcgaggacctcgtcagactgcacggacgccgcctacattgtccgtcagagcagcttcaacagccaggaagggaacaccttcaagctccaagagagtatagaccctgcaggtgaggagaccatgtccccaacttctccaaccttaatgccccgtatgcgaagccattctttctattcggtcaatatgaaagacaaaggtggtatagaaaagttggaaagtatttttaaagaaaggtccctgagcctacaccgggctactagttcccactctgtagcaaaagaacccaaagctcctgcagcccctgccaacaccttggccattgttcctgattccagaagaccatcatcgtgtatagacatctatgtctctgctatggatgagctccactgtgatatagaccctctggacaattccgtgaacatccttgggctgggcgagccaagcttttcaactccagtaccttccacagccccttcaagtagtgcctatgcaacacttgcacccacagacagacctccaagccggagcattgattttgaggacatcacctccatggacactagatctttttcttcagactacacccacctcccagaatgccaaaacccctgggactcagagcctccgatgtaccacaccattgagcgttccaaaagtagccgctacctagccaccacaccctttcttctagaagaggctcccattgtgaaatctcatagctttatgttttccccctcaaggagctattatgccaactttggggtgcctgtaaaaacagcagaatacacaagtattacagactgtattgacacaaggtgtgtcaatgcccctcaagcaattgcggacagagctgccttccctggaggtcttggagacaaagtggaggacttaacttgctgccatccagagcgagaagcagaactgagtcaccccagctctgacagtgaggagaatgaggccaaaggccgcagagccaccattgcaatatcctcccaggagggtgataactcagagagaaccctgtccaacaacatcactgttcccaagatagagcgcgccaacagctactcggcagaggagccaagtgcgccatatgcacacaccaggaagagcttctccatcagtgacaaactcgacaggcagcggaacacagcaagcctgcgaaatcccttccagagaagcaagtcctccaagccggagggccgaggggacagcctgtccatgaggagactgtccagaacatcggctttccaaagctttgaaagcaagcacaactaaaccttcttaatatccgccacagaaggctcaagaatccagccctaaaattctctccaactccagtttttcccctttccttgaatcatacctgctttattcttagctgagcaaaacaagcaatgctttgggaggtgttaactcaaaggtgacttctgggccacagatcaagaaagcatttgatctgacccagtgccagacacaggggatttaaggcatgttcacacttgctgggcagggagggggaagagagggagaaggaagggttagagatgaatgtgtatccgcagtcacagcagaaagccatgagagcaggggaaacaaggggcttcgagcacgctccatgccaggaggcatctgttgatttctgaccattatcaagagttgtaggatgcagggctaaattgcaaaataaaataaaatagccagcgtacacaatgagatattctaaacttccattctgttttcttttcacattggctccatcactggtgactgatgaagagcatcctctttattcagtataagccggcagcaagcagttctacctaacgtcccacatccttctcatgccaacacttctgtaattgatcattataaagaaaaaacaaggtaacagtcatagttcacctgtctcttatctattcacttctggtgccacaactgtttatccttttttgaagaaaataagggaacagaaatgcctttttgtattgcaatcgaaatgaaagaagagttgatgttaaaaaaacaaaagtcaagtgatttattatatacagtgggcgttcaagtctagtcgagcaagctcaggagaatgtaattaaataattttatattttttaatttattttgtatctcacctgtcatggatgaattcattcactgaatatgtaatattgaacttaaaaaaaaaaaaaaaaaaacagtggacagaactagcctgaaattgccatgtgggatgttctgttctcaccgttcatgtcgtgtgttgtgctgctgtgtgaccgtcatcatttgcatggctccaccacgtcctcctgaacacctccaagacgtaactgccaatttttcacaccactcatcacatgaccattttgtatatgaatatatggttatatgtggatattttcatacctagagttttgccatctaatctgagctcagcataaatttaattggagtttttcaagggctggtatcttttctatggaaatgttccaaagtactttttaaggaagtttcaaaactataaataaccttagatttgactgggagtttggtatcaacccaaagccatcagttgcaagcataccatgaatatttttcttatagacagagctataaaaaaagatacagtagaccaaaatagaaagcaaataaaccatataaagagttctatagtatatgccacttatacacatgtatatgcatttatgcagagacacatatttatatataaaataaatggacataaagagctttcttaagaggcttggacttttctatcaccttgaagtataatcaagattttttttaatatacaagaagttccattgaaagagttcattatactattagaccaaatgttgaaattatgaattttttaaattcctatgacttttcctcatatcaaactagagttgtcaaaatgactcactgctcgccttctgcccttttttgtctgaaatcactcactatcaggacatctcatttagtctcgcttttcctaaacaaagtttttacaccacgtacaatctgaatagacacgcaactttaaaccagctagtgttacagactctaaagtagaggcataatcttctagatcagaatctggattggaagtgggataaagaaatcttgaccccaaatctttggattcctatttggctcattaacgtttaaaaaagaataaagcctttaaggaaaacaatctattgttcctggcctgagatactacctggttcttttgtttaattataattgaaaacaccaagttttactttattttgtgaaatcaagtaaatacatggcctttaaatataagataaaaaacaaaataaaaactcttctgaataagcatttcaggtataaaaattgtgatgaggtcacatatgtaaagaattaaacaattcctttttcactccttggcatttaggtgaattgctaacaaacacccagactcccttatttacagctgttctagaaacacttaacattttattgggccccacgctggctatttccctctcagcaaacatgcactgggtctgagaataaatcagtcgttcttaaacttccttgagtcatgaatccctacgaggctctacgataactctaggctctctctccagaaagaaaaaaaaaatgtacataatgcaaagttgcaatataacttgaaagggttcatgagcccctggttctatattgattatgagggaaaggaaccatgtttgctcgtagttaatgcagttgtaataggcactttacatttgagaccatctctctgtgattctcaggaaggccaaatctcagcaccagagtttgaacaaagattttgttatttttgttctttaaattagatgttgccaatttaatgcacttacatcctaacatgttgatgattttaagtatcaagcacaatcacaatgctgaagtctccctgaagggtctatagaggaatccaagaaggaggtgaaaacattattcattgacatcaaacacctccaaggcctcattctgggccagattggaagtgagggaaagaaattttcagctcaagtgtttccattcatcgtgatttcagagagaaaaggataggaactgggaaacgaaatactcaacagtcctgatcttgcaattacctgattcttttgttgtgcatgaaaattaagttctaacgtctgtgaaacgtgtgaaatatataagccaaacttaaatgactgtagacacaaactgtgtaacttccatgagccatttccttcctgtgcacatgtatgtgtgttatttttcagtccatttccttccatttctgggttctctgtgcagcaatgtggcattgattctgtggccttgtgcagtcagcttatgctcagcccattgattccaccacatccctgatcgaggggacgttaattacctgtctttgtgaattaagcagtttatgagcagcaagttactgaggaccgtaaataaagtcatagagagctcaaggaagtaatagcttcggtttaatcatctgcttcaaaggtgccaatttttacatactactatatgtaatgatttgttctaaattgcttgttccttttgaaaaaaatttaaggatgttttatgaaaatgaccatggaggtctcctgcttaaccaatatactttatatttatagttagagggcccctccttgatagatttgaatgaggagctggaaagcttgggtacaattttcatgactagtttgtattatctctgcaagccaaaaggtgtattagcagtcagaatacagcactttacagaactggattttttaaatgtataagtttccaggtctaggatattatttagcactttgaaagttccccttttaaaatatatatataagaataatcaaacgagaaaaatgtactcataaaaacacaaaatgtgtttcttctgaatgtttcaaatgtattctaaaatatggtttctatagataatattatggtagataactggatagtacttttttactttctgagtctgtacagtctaatcttcctgaatgtttgggctgtttaatttctttaaagatgataaatattacccggtcatccatcatgctctatctataggctataccattcatatgttgggcaatgctgtattataaaacacatgtaaacatgtatgtaacccatgtttaacccagcgtagttgtagcatgtggttgtattaatgaacgttacaggagagctttataatcatttataaatctgtaacattcaataaagaaacacatgttgcaatgattaattatgttcccaattccatgttatttagcatctttgtgaaccgggcatttttgttcctgatctgcttgatgcttttcatgttcctttggaccactgtaaaatatgtgtgtaagctgatgtatatcctctaatgagtcctgcgagctgtggtaggcaagttgtgattttgtgaaagatgaactggcatgttatgcaaaagactatatgacaaatgaacttaaaatgatattttaaactccagttgaattagctgtttacatctgccaaccccatgcagaagcagtgtacagtattttataaatatgttcatttagtaacactatgttgtttctagataaagtgatactagtgtgtaggtggttatatatttggcctagatattgcattataactcactatccttgaacaataacaatgtgactgaaatgaagtaatctctctccaaacctggtcttaattttgtgtcaacaaaatgcatgcactgctttatgcatttcttttgacttttctaaggcacttaacattttactattgcctgctgtgacgcttaagccagttaagaattcactcaaacagttgggcttcttactatggggattatgttcagttttgaaaaattaaaaacaaaaaaaaagtaaccaagggatttttaaatcttttttttaagcactgaacaaataatatgaaataaagtattaactgcaggttgtatagccatgggacccagaggtggcaagccaacacctcttgggtctgccaaggtctaggtgttgtgcatctgtgatatatgcaagacacatttctgattcttcagagctactgggtgcaaattgctaagccggagatgagagcgaaaagtccatttgtgatagtgcagaagtgcgcagatcgctgtgaatgtgtgatccaatgatatgacaagagacctccctccttgtcttgtggcttcttcaccttgggggtgggaggcgtgtgtttgggtgtctgatgaagcactgggtggttctaaagagtttactcttttagaaaatcatcattactagaagccattcaacatctgttaaagagaggagccaatttgcacagttttgaaaatgactgctcctaaaatgagtagacaaaactttcctctcagcctgcattgacctaagctttctcttctttttacaagttggatattctgtctctcagattggagtcaaccaaaagaaaggctggttccaattaaggaaaagcaatttttttcttttaattcctctcagaaggtagttgtgttaccttttctctgatgatttccttgcactcatcactgctaatgaaagcaacgtgactgtttctacaggtcatctaagagaggattttggtgaccaggtaatccagatggcaataagaatgaggtgaattttcccttccttaccttacctagtctaccagcctccttaaacctgcaggcccttgtgcgtttgcacaatgtttgtgtgctggaaactgaaaagacatgtgtatggctagaagtccataggctttgtaaagagtgtcagtgccaatcaggacccatgccttcctctggaggagacaggtggctgaggcctatggggcaaagggggaaattcagccttgtgcctggatgtgcggctttctttctcccctcttcttgcttctcttctcttagtctaagactagctgctttttcaccagaccaacagacccttgcccacattacctttataccttgttgaccctcgcacagtaagctgaaacagcggccgcttggaaaaaggcaaacatttagactttgtcccaggcctgatcttcccaccaacttagcctcctcctgcctctaccccggctggtgttcacattgacctcgtccaatttttgcctctctaaatctcaagagtcctacacatcttcaccgcagcttcccacacatcattaaaaccagccatctgcacatagactgagaggctgcaaattcaaacacatggaggggccggatccagaaaacacaaatgtgtgaactggactggtgaaagtcaacagaagtagggtttgtaagtaaggcatgacagaatacacatcccaccacaaacaccttcaaattcagctttccaaacactgtcctagccaaacaaacatgtctgccagcagcattcagtcccaaaggcaatagtttgggacctctgatttagagccaacaggatcagattcttgatagtgacttggtgagtgtacacacaaaatacactgagaaccgagaggggggaaaagaaactcgccttattcagagttactcttgttctgaatgatatatgcatgtatatttgttagccacataattctaaacttgaacacagagtctttgtgagagagattcagaagaagagtaccatagcatggtatgaagacttacctgcttcctgtcactactgcgtctgacctgagtttatattgctgctaccgctgtgtaaaaacagtacgtgcagtatactgtactcatagccatttcataatataggacctagactctcatttgtaaacgttctaaccctagtttataatgggggaaattattcagatatcatttttaaatgaattcctacaaatacaatggttttaaaattgggtttgctgaatatatatacatcttttccttctttgtgtatggatggaccacggtatgcatatctatcgacactataaatatatataaatccttagaagaattgttcttttttaaaggtctattattggattcacaatgcactttgagtttgattgtttagatatacaattcagagcagaagttggcagataccacgggaaaaactttctgaaatttctgtaacaaatgttttgcaataaaaaaaaaaactcagtagtttaaaaca

SEQ ID NO: 68 - DNA, 284 bp, Homo sapiens
  source 1..284: mol_type = genomic DNA
ggtggagaacatgtctatgcggctggaggaagtcaacgagagagagcactccatgaaggcttcactccagaccgtggacatccggctggcgcagctggaagaccttatcgggcgcatggccacggccctggagcgcctgacaggtctggagcgggccgagtccaacaaaatccgctcgaggacctcgtcagactgcacggacgccgcctacattgtccgtcagagcagcttcaacagccaggaagggaacaccttcaagctccaagagagtatagaccctgcag

SEQ ID NO: 69 - DNA, 6392 bp, Homo sapiens
  source 1..6392: mol_type = genomic DNA
agcatcctctttattcagtataagccggcagcaagcagttctacctaacgtcccacatccttctcatgccaacacttctgtaattgatcattataaagaaaaaacaaggtaacagtcatagttcacctgtctcttatctattcacttctggtgccacaactgtttatccttttttgaagaaaataagggaacagaaatgcctttttgtattgcaatcgaaatgaaagaagagttgatgttaaaaaaacaaaagtcaagtgatttattatatacagtgggcgttcaagtctagtcgagcaagctcaggagaatgtaattaaataattttatattttttaatttattttgtatctcacctgtcatggatgaattcattcactgaatatgtaatattgaacttaaaaaaaaaaaaaaaaaaacagtggacagaactagcctgaaattgccatgtgggatgttctgttctcaccgttcatgtcgtgtgttgtgctgctgtgtgaccgtcatcatttgcatggctccaccacgtcctcctgaacacctccaagacgtaactgccaatttttcacaccactcatcacatgaccattttgtatatgaatatatggttatatgtggatattttcatacctagagttttgccatctaatctgagctcagcataaatttaattggagtttttcaagggctggtatcttttctatggaaatgttccaaagtactttttaaggaagtttcaaaactataaataaccttagatttgactgggagtttggtatcaacccaaagccatcagttgcaagcataccatgaatatttttcttatagacagagctataaaaaaagatacagtagaccaaaatagaaagcaaataaaccatataaagagttctatagtatatgccacttatacacatgtatatgcatttatgcagagacacatatttatatataaaataaatggacataaagagctttcttaagaggcttggacttttctatcaccttgaagtataatcaagattttttttaatatacaagaagttccattgaaagagttcattatactattagaccaaatgttgaaattatgaattttttaaattcctatgacttttcctcatatcaaactagagttgtcaaaatgactcactgctcgccttctgcccttttttgtctgaaatcactcactatcaggacatctcatttagtctcgcttttcctaaacaaagtttttacaccacgtacaatctgaatagacacgcaactttaaaccagctagtgttacagactctaaagtagaggcataatcttctagatcagaatctggattggaagtgggataaagaaatcttgaccccaaatctttggattcctatttggctcattaacgtttaaaaaagaataaagcctttaaggaaaacaatctattgttcctggcctgagatactacctggttcttttgtttaattataattgaaaacaccaagttttactttattttgtgaaatcaagtaaatacatggcctttaaatataagataaaaaacaaaataaaaactcttctgaataagcatttcaggtataaaaattgtgatgaggtcacatatgtaaagaattaaacaattcctttttcactccttggcatttaggtgaattgctaacaaacacccagactcccttatttacagctgttctagaaacacttaacattttattgggccccacgctggctatttccctctcagcaaacatgcactgggtctgagaataaatcagtcgttcttaaacttccttgagtcatgaatccctacgaggctctacgataactctaggctctctctccagaaagaaaaaaaaaatgtacataatgcaaagttgcaatataacttgaaagggttcatgagcccctggttctatattgattatgagggaaaggaaccatgtttgctcgtagttaatgcagttgtaataggcactttacatttgagaccatctctctgtgattctcaggaaggccaaatctcagcaccagagtttgaacaaagattttgttatttttgttctttaaattagatgttgccaatttaatgcacttacatcctaacatgttgatgattttaagtatcaagcacaatcacaatgctgaagtctccctgaagggtctatagaggaatccaagaaggaggtgaaaacattattcattgacatcaaacacctccaaggcctcattctgggccagattggaagtgagggaaagaaattttcagctcaagtgtttccattcatcgtgatttcagagagaaaaggataggaactgggaaacgaaatactcaacagtcctgatcttgcaattacctgattcttttgttgtgcatgaaaattaagttctaacgtctgtgaaacgtgtgaaatatataagccaaacttaaatgactgtagacacaaactgtgtaacttccatgagccatttccttcctgtgcacatgtatgtgtgttatttttcagtccatttccttccatttctgggttctctgtgcagcaatgtggcattgattctgtggccttgtgcagtcagcttatgctcagcccattgattccaccacatccctgatcgaggggacgttaattacctgtctttgtgaattaagcagtttatgagcagcaagttactgaggaccgtaaataaagtcatagagagctcaaggaagtaatagcttcggtttaatcatctgcttcaaaggtgccaatttttacatactactatatgtaatgatttgttctaaattgcttgttccttttgaaaaaaatttaaggatgttttatgaaaatgaccatggaggtctcctgcttaaccaatatactttatatttatagttagagggcccctccttgatagatttgaatgaggagctggaaagcttgggtacaattttcatgactagtttgtattatctctgcaagccaaaaggtgtattagcagtcagaatacagcactttacagaactggattttttaaatgtataagtttccaggtctaggatattatttagcactttgaaagttccccttttaaaatatatatataagaataatcaaacgagaaaaatgtactcataaaaacacaaaatgtgtttcttctgaatgtttcaaatgtattctaaaatatggtttctatagataatattatggtagataactggatagtacttttttactttctgagtctgtacagtctaatcttcctgaatgtttgggctgtttaatttctttaaagatgataaatattacccggtcatccatcatgctctatctataggctataccattcatatgttgggcaatgctgtattataaaacacatgtaaacatgtatgtaacccatgtttaacccagcgtagttgtagcatgtggttgtattaatgaacgttacaggagagctttataatcatttataaatctgtaacattcaataaagaaacacatgttgcaatgattaattatgttcccaattccatgttatttagcatctttgtgaaccgggcatttttgttcctgatctgcttgatgcttttcatgttcctttggaccactgtaaaatatgtgtgtaagctgatgtatatcctctaatgagtcctgcgagctgtggtaggcaagttgtgattttgtgaaagatgaactggcatgttatgcaaaagactatatgacaaatgaacttaaaatgatattttaaactccagttgaattagctgtttacatctgccaaccccatgcagaagcagtgtacagtattttataaatatgttcatttagtaacactatgttgtttctagataaagtgatactagtgtgtaggtggttatatatttggcctagatattgcattataactcactatccttgaacaataacaatgtgactgaaatgaagtaatctctctccaaacctggtcttaattttgtgtcaacaaaatgcatgcactgctttatgcatttcttttgacttttctaaggcacttaacattttactattgcctgctgtgacgcttaagccagttaagaattcactcaaacagttgggcttcttactatggggattatgttcagttttgaaaaattaaaaacaaaaaaaaagtaaccaagggatttttaaatcttttttttaagcactgaacaaataatatgaaataaagtattaactgcaggttgtatagccatgggacccagaggtggcaagccaacacctcttgggtctgccaaggtctaggtgttgtgcatctgtgatatatgcaagacacatttctgattcttcagagctactgggtgcaaattgctaagccggagatgagagcgaaaagtccatttgtgatagtgcagaagtgcgcagatcgctgtgaatgtgtgatccaatgatatgacaagagacctccctccttgtcttgtggcttcttcaccttgggggtgggaggcgtgtgtttgggtgtctgatgaagcactgggtggttctaaagagtttactcttttagaaaatcatcattactagaagccattcaacatctgttaaagagaggagccaatttgcacagttttgaaaatgactgctcctaaaatgagtagacaaaactttcctctcagcctgcattgacctaagctttctcttctttttacaagttggatattctgtctctcagattggagtcaaccaaaagaaaggctggttccaattaaggaaaagcaatttttttcttttaattcctctcagaaggtagttgtgttaccttttctctgatgatttccttgcactcatcactgctaatgaaagcaacgtgactgtttctacaggtcatctaagagaggattttggtgaccaggtaatccagatggcaataagaatgaggtgaattttcccttccttaccttacctagtctaccagcctccttaaacctgcaggcccttgtgcgtttgcacaatgtttgtgtgctggaaactgaaaagacatgtgtatggctagaagtccataggctttgtaaagagtgtcagtgccaatcaggacccatgccttcctctggaggagacaggtggctgaggcctatggggcaaagggggaaattcagccttgtgcctggatgtgcggctttctttctcccctcttcttgcttctcttctcttagtctaagactagctgctttttcaccagaccaacagacccttgcccacattacctttataccttgttgaccctcgcacagtaagctgaaacagcggccgcttggaaaaaggcaaacatttagactttgtcccaggcctgatcttcccaccaacttagcctcctcctgcctctaccccggctggtgttcacattgacctcgtccaatttttgcctctctaaatctcaagagtcctacacatcttcaccgcagcttcccacacatcattaaaaccagccatctgcacatagactgagaggctgcaaattcaaacacatggaggggccggatccagaaaacacaaatgtgtgaactggactggtgaaagtcaacagaagtagggtttgtaagtaaggcatgacagaatacacatcccaccacaaacaccttcaaattcagctttccaaacactgtcctagccaaacaaacatgtctgccagcagcattcagtcccaaaggcaatagtttgggacctctgatttagagccaacaggatcagattcttgatagtgacttggtgagtgtacacacaaaatacactgagaaccgagaggggggaaaagaaactcgccttattcagagttactcttgttctgaatgatatatgcatgtatatttgttagccacataattctaaacttgaacacagagtctttgtgagagagattcagaagaagagtaccatagcatggtatgaagacttacctgcttcctgtcactactgcgtctgacctgagtttatattgctgctaccgctgtgtaaaaacagtacgtgcagtatactgtactcatagccatttcataatataggacctagactctcatttgtaaacgttctaaccctagtttataatgggggaaattattcagatatcatttttaaatgaattcctacaaatacaatggttttaaaattgggtttgctgaatatatatacatcttttccttctttgtgtatggatggaccacggtatgcatatctatcgacactataaatatatataaatccttagaagaattgttcttttttaaaggtctattattggattcacaatgcactttgagtttgattgtttagatatacaattcagagcagaagttggcagataccacgggaaaaactttctgaaatttctgtaacaaatgttttgcaataaaaaaaaaaactcagtagtttaaaacatga

(19) United States
(12) Patent Application Publication
(10) Pub. No.: US 2023/0220037 A1
(43) Pub. Date: Jul. 13, 2023

(54) NOVEL USE

(71) Applicants: GlaxoSmithKline Intellectual Property (No.3) Limited, Brenrford (GB); 23andMe, Inc., Sunnyvale, CA (US)

(72) Inventors: Janet Mary KUMAR, Collegeville, PA (US); Colin Houston McPHEE, Collegeville, PA (US); Adam Mohamed NAGUIB, Sunnyvale, CA (US); Lea SAROV-BLAT, Collegeville, PA (US); Claire Yvonne Marie TOWNSEND, Collegeville, PA (US); Paul Bryan WREN, Collegeville, PA (US); Clint Ernest YOUNG, Collegeville, PA (US); Alexander George Steven PRESTON, Stevenage (GB); David Andrew HALL, Stevenage (GB)

(73) Assignees: GlaxoSmithKline Intellectual Property (No.3) Limited, Brentford (GB); 23andMe, Inc., Sunnyvale, CA (US)

(21) Appl. No.: 17/812,299

(22) Filed: Jul. 13, 2022

Related U.S. Application Data
(63) Continuation-in-part of application No. 17/790,218, filed on Jan. 1, 0001.

(30) Foreign Application Priority Data
Jan. 12, 2022 (WO) PCT/EP2022/050481

(51) Int. Cl. C07K 14/705; A61K 31/404; A61K 45/06; A61P 25/06

(52) U.S. Cl. CPC C07K 14/705; A61K 31/404; A61K 45/06; A61P 25/06

(57) ABSTRACT

The present disclosure relates to an inhibitor of human TRPM3 for use in the treatment or prevention of migraine, including in subjects whose migraines are not responsive to CGRP inhibition or whose migraines are responsive to triptans. Combination therapies are also described. In other aspects, the present disclosure provides methods for identifying suitable patients, methods for identifying inhibitors of human TRPM3, cell lines and agonists for use in such methods and a method for measuring PACAP release.

### CROSS REFERENCE TO PRIOR APPLICATIONS

This application is a Continuation-in-Part (CIP) of U.S. application Ser. No. 17/790,218 titled “NOVEL USE” filed 30 Jun. 2022 which is a 371 of PCT application serial number PCT/EP2022/050481 titled “NOVEL USE” filed 12 Jan. 2022 which claims the benefit of U.S. Provisional Application No. 63/137,373 titled “NOVEL USE” filed 14 Jan. 2021 and also claims the benefit of U.S. Provisional Application No. 63/229,222 titled “NOVEL USE” filed 4 Aug. 2021; which are all incorporated herein by reference in their entirety.

### FIELD OF THE INVENTION

The present disclosure relates to an inhibitor of human TRPM3 for use in the treatment or prevention of migraine, including in subjects whose migraines are not responsive to CGRP inhibition or whose migraines are responsive to triptans. Combination therapies are also described. In other aspects, the present disclosure provides methods for identifying suitable patients, methods for identifying inhibitors of human TRPM3, cell lines and agonists for use in such methods and a method for measuring PACAP release.

### BACKGROUND TO THE INVENTION

Migraine headaches are a common cause of disability in the United States, affecting approximately 27 million American adults, or 17.1% of women and 5.6% of men. Chronic migraine, which affects 3.2 million Americans (2%), is defined as having migraine symptoms for at least 15 days per month, lasting at least 4 hours, and for longer than 3 months in duration. This is in contrast to episodic migraine, which causes symptoms on fewer than 15 days per month. Current treatment for migraine is divided into acute, abortive agents and medications that will prevent migraine onset. Opioid analgesics and triptans are commonly prescribed for migraine. Whilst these types of drugs provide acute relief, regular use can result in increased headache severity, and progression of headache from an episodic to a chronic state. This form of medication overuse headache is difficult to treat and new treatment options are eagerly awaited.

Calcitonin gene-related peptide (CGRP) is a peptide that is released by peripheral neurons, including somatosensory neurons of the dorsal root, vagal and trigeminal ganglia where it is reported to act as a neurotransmitter, a vasodilator and as a local mediator of inflammation. Its short half-life (7 mins) normally results in localised effects. However, CGRP levels are increased in the cranial circulation during migraine and cluster headache attacks, and intravenous administration of CGRP triggers migraine attacks in migraineurs suggesting that it has a prominent role in migraine. Four monoclonal antibody antagonists of CGRP function and two small molecule CGRP receptor antagonists have been approved by the FDA for migraine prevention in addition to an oral CGRP receptor antagonist approved for symptomatic treatment of migraine. Evidence suggests that the increased levels of CGRP during migraine are produced in the trigeminal ganglion. CGRP release from the trigeminal ganglion is also implicated in the pathophysiology of trigeminal neuralgia and cluster headaches.

Activation of TRPV1 and TRPA1 has previously been demonstrated to result in release of CGRP from trigeminal neurons derived from rats and mice. TRPV1 and TRPA1 are members of the transient receptor potential (TRP) family of channels. The TRP gene family comprises at least 28 mammalian genes divided into seven subfamilies. Most of the encoded proteins exhibit common structural features including six predicted transmembrane (TM) domains with a putative pore loop between TM5 and TM6 and the so-called “TRPbox” after TM6. Members of the TRPM subfamily have unusually long cytoplasmic tails at both ends of the channel domain, and some of the family members have an enzyme domain in the C-terminal region. Despite their similarities of structure, TRPMs have different ion-conductive properties, activation mechanisms, and putative biological functions.

The human TRPM3 gene is comprised of 30 exons and maps to human chromosome 9q-21.12. TRPM3 isoforms vary from 1184 to 1744 amino acids in length and possess the characteristic six transmembrane domain of the TRP family. However, unlike some other TRPM family members, TRPM3 does not contain an enzyme domain in the C-terminal cytoplasmic region. Several alternative spliced transcript variants encoding different isoforms have been identified in mice and humans. The isoforms appear to have very different physiological roles given that one mouse isoform, Trpm3α1 preferentially conducts monovalent cation influx, while another, Trpm3α2 strongly favours divalent ion entry. The mouse Trpm3a2 form is the best studied. It has been reported to be a calcium-permeable nonselective cation channel that can be activated by several stimuli, including ligands, such as pregnenolone sulfate, nifedipine and CIM0216, and heat.

Human TRPM3 expression is detectable in kidney, brain, ovary, and pancreas. Northern blotting of mouse tissues resulted in strong signals in brain, whereas in kidney no signal could be detected. Within brain subregions in the mouse, the highest levels of expression were found in the cerebellum, choroid plexus, the locus coeruleus, the posterior hypothalamus, and the substantia nigra. using a Trpm3-specific antisense RNA probe yielded a positive Trpm3 hybridization signal in 82%±5% of mouse trigeminal neurons. The most abundant isoform in the human dorsal root ganglion as assessed by RNA expression has the UNIPROT ID: Q9HCF6-2.

The prepro-peptide of pituitary adenylate cyclase activating polypeptide (PACAP) is encoded by ADCYAP1 gene and is proteolytically processed into two forms of PACAP containing either 27 or 38 amino acids with PACAP-38 being the more prevalent, representing 90% of PACAP forms in mammalian tissues. Like CGRP, PACAP is a multifunctional vasodilatory peptide that has been implicated in migraine pathogenesis. It has been shown that plasma levels of PACAP are elevated during a migraine attack compared to their interictal levels. Peripheral injection of PACAP in migraineurs resulted in 11 out of 12 subjects experiencing an initial headache, and 7 out of 12 subjects experienced a migraine-like headache that was delayed by 2-11 h. Interestingly, treatment with sumatriptan decreased PACAP-38 levels suggesting that sumatriptan may mediate its effects on migraine via PACAP In addition to its role in migraine pathogenesis, PACAP has been implicated in spontaneous headache conditions. For example, elevated PACAP-38 levels have been observed during the attack phase in episodic cluster headache patients. It has also been demonstrated to relieve opioid induced hyperalgesia in animal models. For example, Pradhan and colleagues have demonstrated that PACAP blockade reduced periorbital allodynia in a mouse model of opioid induced hyperalgesia, and that PACAP blockade reduced cephalic allodynia in mice treated with combined morphine and nitroglycerin.

Currently there is no clinically available PACAP-specific treatment. A PAC1 receptor monoclonal antibody, AMG 301 was observed to offer no benefit over placebo for migraine prevention despite inhibiting nociceptive activity in the trigeminocervical complex to the same extent as sumatriptan in preclinical trials. A monoclonal antibody targeting PACAP-38, ALD1910, however, remains in clinical development for the treatment of migraine patients.

The trigeminal ganglion has been examined to detect neuropeptides including calcitonin gene-related peptide (CGRP) and PACAP. In the human trigeminal ganglion nearly half of the neurons were found to be CGRP immunoreactive. PACAP-38 is present in the trigeminal ganglion, and plasma PACAP-38-like immunoreactivity is increased after electrical stimulation of the trigeminal ganglion.

Vriens et al. (Neuron, 2011, 70: 482-494) reported that pregnenolone sulfate induced an inward current in trigeminal neurons from mice which they attributed to TRPM3.

Held et al. (PNAS, 2015, E1363-E1372) reports an experiment in which isolated mouse hind paw skin was induced to release CGRP by treatment with a TRPM3 agonist CIM0216. CGRP release was largely reduced in the presence of a TRPM3 antagonist, isosakuranetin.

### SUMMARY OF THE INVENTION

In a first aspect, the invention provides an inhibitor of human TRPM3 for use in the treatment or prevention of a disorder selected from: migraine, trigeminal neuralgia, cluster headache, postherpetic neuralgia, chemotherapy-induced neuropathy, complex regional pain syndrome, HIV sensory neuropathy, peripheral nerve injury and phantom limb pain.

The invention also provides use of an inhibitor of human TRPM3 in the manufacture of a medicament for the treatment or prevention of a disorder selected from: migraine, trigeminal neuralgia, cluster headache, postherpetic neuralgia, chemotherapy-induced neuropathy, complex regional pain syndrome, HIV sensory neuropathy, peripheral nerve injury and phantom limb pain.

The invention also provides a method of treatment or prevention of a disorder selected from migraine, trigeminal neuralgia, cluster headache, postherpetic neuralgia, chemotherapy-induced neuropathy, complex regional pain syndrome, HIV sensory neuropathy, peripheral nerve injury and phantom limb pain, which comprises administering a subject in need thereof a therapeutically acceptable amount of an inhibitor of human TRPM3. In one embodiment, the subject is human.

In another aspect, the invention provides an inhibitor of human TRPM3 for use in the treatment or prevention of migraine in a human subject whose migraines are not responsive to CGRP inhibition. In one particular embodiment, the migraines are not responsive to CGRP therapy and are responsive to therapy with a triptan.

In this context, migraines that are “not responsive to CGRP inhibition” are migraines that are not adequately treated by CGRP inhibition. Migraines that are “responsive to therapy with a triptan” are migraines that are adequately treated by a triptan.

In yet another aspect, the invention provides an inhibitor of human TRPM3 for use in the treatment or prevention of migraine in a human subject whose migraines are responsive to therapy with a triptan.

In a further aspect, the invention provides an inhibitor of human TRPM3 for use in the treatment or prevention of cluster headache in a human subject whose headaches are not responsive to CGRP inhibition. In one particular embodiment, the headaches are not responsive to CGRP therapy and are responsive to therapy with a triptan.

In another aspect, the invention provides an inhibitor of human TRPM3 for use in the treatment of medication overuse headache in a human subject.

In specific embodiments, the invention provides combination therapies and pharmaceutical compositions of the inhibitor of human TRPM3.

Further aspects of the invention provide methods for identifying whether a patient is a candidate for treatment with an inhibitor of human TRPM3, methods for identifying an inhibitor of human TRPM3 and a cell line and agonist for use in these methods.

In another aspect, the invention provides a method for identifying whether a patient diagnosed with migraine is a candidate for treatment with an inhibitor of human TRPM3, comprising:
a) sequencing the human TRPM3 gene in the patient diagnosed with migraine;
b) comparing the sequence with the sequences of the human TRPM3 exons set out in SEQ ID NOs: 38-69 and identifying whether changes would modify the amino acid sequence of any isoform;

wherein, if a change in amino acid sequence is identified, the patient is a candidate for treatment with an inhibitor of human TRPM3.

In a further aspect, the invention provides a method for identifying an inhibitor of human TRPM3, comprising measuring release of CGRP from dorsal root ganglia or trigeminal ganglia, or from primary cultures of cells isolated from dorsal root ganglia or trigeminal ganglia, following challenge with an agonist of human TRPM3 in the presence or absence of a test inhibitor, wherein the test inhibitor is identified as an inhibitor for human TRPM3 if CGRP production is reduced in the presence of the test inhibitor compared to CGRP production in the absence of the test inhibitor.

In another aspect, the invention provides a method for measuring PACAP in a sample comprising incubating a cell line expressing the PAC1 receptor with the sample and measuring cAMP signalling in the cell line.

In a further aspect, the invention provides a method for identifying an inhibitor of human TRPM3, comprising measuring release of PACAP from dorsal root ganglia or trigeminal ganglia, or from primary cultures of cells isolated from dorsal root ganglia or trigeminal ganglia, following challenge with an agonist of human TRPM3 in the presence or absence of a test inhibitor, wherein the test inhibitor is identified as an inhibitor for human TRPM3 if PACAP production is reduced in the presence of the test inhibitor compared to PACAP production in the absence of the test inhibitor. In particular embodiments, PACAP production is measured according to the method of the invention.

In a further aspect, the invention provides a cell line expressing a recombinant human TRPM3 variant protein comprising one or more amino acid substitutions at residues selected from the group consisting of R1670, R1457, D602, K774, S1678, Y378, V990 and P1090 (numbering based on SEQ ID NO:2).

In another aspect, the invention provides a cell line expressing a recombinant human TRPM3 variant protein having the sequence set out in SEQ ID NO: 2, or a processed version of this lacking the initial methionine residue, or a variant of these sequences comprising the amino acid substitution R1670Q (numbering based on SEQ ID NO. 2).

In a further aspect, the invention provides for use of the cell lines of the invention in the identification of an inhibitor of human TRPM3.

In a further aspect, the invention provides a method for identifying an inhibitor of human TRPM3, comprising:
a) contacting a cell line according to the invention which cells contain an intracellular calcium indicator with an agonist of human TRPM3 in the presence and absence of a test inhibitor; and
b) measuring a change in intracellular calcium concentrations by measuring a change in the intracellular calcium indicator;

wherein the test inhibitor is identified as an inhibitor for human TRPM3 if intracellular calcium concentrations are reduced in the presence of the test inhibitor compared to those achieved in the absence of the test inhibitor.

In another aspect, the invention provides a method for identifying an inhibitor of human TRPM3, comprising measuring current increases in a cell line according to the invention following challenge with an agonist of human TRPM3 in the presence and absence of a test inhibitor, wherein the test inhibitor is identified as an inhibitor for human TRPM3 if the increase in current is reduced in the presence of the test inhibitor compared to the increase achieved in the absence of the test inhibitor.

In a further aspect, the invention provides a method for identifying an inhibitor of human TRPM3 which comprises a step of dural sensitisation with a TRPM3 agonist in the presence or absence of the test inhibitor, followed by assessment of facial allodynia, wherein the test inhibitor is identified as an inhibitor for human TRPM3 if the level of facial allodynia is lower in the presence of the test inhibitor compared to that observed in the absence of the test inhibitor.

In yet another aspect, the invention provides (R)-2-(3,4-dihydroquinolin-1(2H)-yl)-N-(5-methylisoxazol-3-yl)propenamide or a salt thereof.

### DESCRIPTION OF DRAWINGS/FIGURES

FIGS. 1A and 1B demonstrate that TRPM3 agonists, CIM0216 (0.37-10 PM) and pregnenolone sulfate (100 μM), induce release of CGRP from dorsal root ganglia neurons. The CIM0216 response was inhibited by TRPM3 blocker isosakuranetin (10 PM) (mean±SEM; n=3 or mean±range; n=2). The pregnenolone sulfate response was inhibited with isosakuranetin (10 μM) (mean±SEM; n=3 or mean±range; n=2). FIG. 1C is a graph showing that isosakuranetin (10 μM) reduced CIM0216 (10 μM) induced CGRP released from DRG neurons, data from separate experiments (bars represent mean±SEM, n=2 or 3 wells)

FIG. 2. demonstrates that isosakuranetin inhibits release of CGRP from dorsal root ganglia neurons following activation with 6 μM CIM0216 (A) or 10 μM CIM0216 (B) in a dose dependent manner (mean±SEM; n=3 or mean±range; n=2). Isosakuranetin reduced the release of CGRP to below basal levels in the presence of 6 μM CIM0216.

FIGS. 3A and 3B demonstrates that the TRPM3 agonists, CIM0216 and pregnenolone sulfate, stimulate release of CGRP from trigeminal ganglia (TG) neurons in a concentration dependent manner (mean±SEM; n=2 or 3). FIGS. 3A and 3B also show that CGRP responses were inhibited with isosakuranetin (10 μM). FIG. 3C is a graph showing that isosakuranetin (10 μM) reduced CIM0216 (10 μM) induced CGRP release from TG neurons in separate experiments (bars represent mean±SEM, n=2 or 3 wells).

FIG. 4 shows a LocusZoom plot for the association between migraine defined using the ‘migraine_diagnosis’ classification and genetic variants at the TRPM3 locus. The x-axis displays position on chromosome 9 using GRCh37/hg19 as the human reference genome build. The y-axis is the −log₁₀(p-value) from a logistic regression testing for an association between migraine case-control status and genotype. Plus symbols (+) denote variants for which genotype was imputed; circle symbols (o) denote variants for which genotype calls were used; x symbols (x) denote imputed coding variants; diamond symbols (⋄) denote genotyped coding variants. The horizontal line represents the genome-wide significance threshold of 5×10⁻⁸. The credible set track displays the number and location of variants in the 99% credible set, which is likely to contain the causal variant. The gene track displays genes in the locus with thick bars representing exons and thin lines representing introns.

FIG. 5 demonstrates the effect of isosakuranetin on the pregnenolone sulfate dose response curve in HEK293-TRPM3 cells in a calcium mobilisation assay (mean±range; n=2).

FIG. 6 demonstrates the effect of isosakuranetin on the CIM0216 dose response curve in HEK293-TRPM3 cells in a calcium mobilisation assay (mean±range; n=2).

FIG. 7 demonstrates the concentration dependent inhibition of the pregnenolone sulfate response by isosakuranetin in HEK293-TRPM3 cells in a calcium mobilisation assay (mean±range; n=2).

FIG. 8A-D shows the response of Wild Type (WT) and Trpm3 knock out (KO) dorsal root ganglion neurons (FIGS. 8A and 8B), and trigeminal ganglion neurons (FIGS. 8C and 8D) to pregnenolone sulfate (PS), CIM0216 and capsaicin in a CGRP release assay (mean±SEM; n=3 or mean±range; n=2). The PS and CIM0216 dose-dependent release of CGRP observed in WT neurons was lacking in Trpm3 deficient neurons. Capsaicin evoked CGRP release from Trpm3 deficient neurons.

FIG. 9 shows the response of HEK MSR II cells expressing canonical TRPM3 (SEQ NO: 2) and a variant of SEQID NO: 2 having the R1670Q mutation to pregnenolone sulfate in a calcium mobilisation assay (mean±SEM; n=10).

FIG. 10A demonstrates that dural administration of TRPM3 agonists, Pregnenolone sulphate (5 mM) and CIM0216 (215 μM), induce mechanical allodynia in the periorbital region of rats. FIG. 10B reveals that at these concentrations the agonists do not cause overt adverse effects, represented by healthy increases in body weight. Data points represent von frey thresholds or percentage change of body weight (mean±SEM; n=3 or 4), which evoked a response from rats treated with Vehicle ( [symbol] ), Pregnenolone sulphate ( [symbol] ) and CIM0216 ( [symbol] ).

FIG. 11A demonstrates CIM0216 induces a concentration-dependent release of PACAP from trigeminal ganglia cultures and its inhibition by isosakuranetin. FIG. 11B shows concentration-dependent inhibition by isosakuranetin (mean±range; n=2).

FIG. 12A-D shows the response of Wild Type (WT) and Trpm3 knock out (KO) dorsal root ganglion cells (FIGS. 12A and 12B), and trigeminal ganglion cells (FIGS. 12C and 12D) to pregnenolone sulfate (PS), CIM0216 and capsaicin and the effect of isosakuranetin on the TRPM3 agonists in a PACAP release assay (mean±range; n=2).

FIG. 13 shows the agonist activity of the isomers of CIM0216 in a calcium mobilisation assay. R-CIM0126 is (R)-2-(3,4-dihydroquinolin-1(2H)-yl)-N-(5-methylisoxazol-3-yl)propenamide and S-CIM0216 is (S)-2-(3,4-dihydroquinolin-1(2H)-yl)-N-(5-methylisoxazol-3-yl)propenamide.

### DETAILED DESCRIPTION OF THE INVENTION

### Statement of the Invention

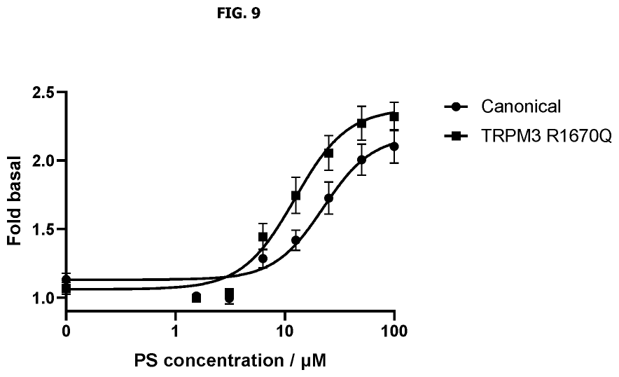

Example 1 demonstrates that a SNP in the human TRPM3 gene, R1670Q shows a strong genetic association with migraine. Example 3 (FIG. 9) shows that the potency of pregnenolone sulfate is 1.8-fold greater at the R1670Q variant than at canonical form, and the maximal fold change in fluorescence in a calcium mobilisation assay was 26% larger. Thus pregnenolone sulfate is more able to activate the TRPM3 variant associated with increased likelihood of migraine diagnosis than the canonical form of the channel.

Further evidence that TRPM3 activation is causative of migraine comes from the five-day rat dural infusion migraine model. Historical data in this model using an inflammatory soup (2 mM histamine, bradykinin, serotonin, 0.2 mM prostaglandin E2) infusion shows mechanical nociception sensitivity (Oshinsky & Gomoncharconsiri, (2007). Episodic dural stimulation in awake rats: a model for recurrent headache. Headache: The Journal of Head and Face Pain, 47(7), 1026-1036). This sensitivity is alleviated with sumatriptan and anti-CGRP therapies, current standard of care compounds for migraine, suggesting this model has clinical translation. Example 4 shows that Pregnenolone sulphate and CIM0216 (TRPM3 agonists) also trigger mechanical allodynia in the periorbital region When comparing significant differences in sensitivity, both Pregnenolone sulphate and CIM0216 were effective in increasing sensitivity to periorbital von Frey (VF) stimulation as early as Day 5 (after 4 infusions) when compared to vehicle treated animals. In other words, Example 4 provides further evidence of the link between TRPM3 activation and migraine.

In addition, the examples demonstrate that inhibition of human TRPM3 in sensory ganglion, including the trigeminal ganglion, reduces production of CGRP. Release of CGRP from the trigeminal ganglion is known in the art to be key to the pathophysiology of migraine, trigeminal neuralgia and cluster headache. Release of CGRP from sensory ganglion is strongly associated with postherpetic neuralgia, chemotherapy-induced neuropathy, complex regional pain syndrome, HIV sensory neuropathy, peripheral nerve injury and phantom limb pain. Accordingly, in a first aspect, the invention provides an inhibitor of human TRPM3 for use in the treatment or prevention of a disorder selected from: migraine, trigeminal neuralgia, cluster headache, postherpetic neuralgia, chemotherapy-induced neuropathy, complex regional pain syndrome, HIV sensory neuropathy, peripheral nerve injury and phantom limb pain. In one embodiment, the disorder is selected from: migraine, trigeminal neuralgia, cluster headache, postherpetic neuralgia, chemotherapy-induced neuropathy, complex regional pain syndrome and HIV sensory neuropathy. In one embodiment, the disorder is migraine, trigeminal neuralgia and cluster headache. In one embodiment, the disorder is migraine. In another embodiment, the disorder is trigeminal neuralgia. In another embodiment, the disorder is cluster headache.

Example 2 also demonstrates that inhibition of human TRPM3 in sensory ganglion, including the trigeminal ganglion, reduces production of PACAP. In other words, inhibition of human TRPM3 in sensory ganglia reduce production of both CGRP and PACAP. Both neuropeptides are known to cause migraine and therapies inhibiting CGRP and PACAP signalling have been developed although as of the date of filing, only CGRP blocking therapies have been approved. Trpm3 inhibitors have the ability to treat migraine mediated by CGRP and PACAP. It is known that there are classes of patients for whom the approved CGRP therapies are ineffective. It is believed trpm3 inhibition may be an effective therapeutic in such patients due to the impact of trpm3 inhibition upon PACAP. Example 5 shows that this patient group is significant, comprising over 10% of migraine patients, and includes patients with and without mutations in trpm3. It is believed that trpm3 inhibition may be an effective therapeutic in patients that are not responsive to PACAP blockade due to the impact of trpm3 inhibition upon CGRP. Given that triptans may mediate their effects on migraine via PACAP, it is plausible that trpm3 inhibition may be an effective therapy in a human subject whose migraines are responsive to therapy with triptans. For the same reasons, it is also plausible that trpm3 inhibition may be an effective therapy for cluster headache in a human subject whose headaches are not responsive to CGRP therapies, and/or whose headaches are responsive to therapy with triptans

As discussed in the section entitled Background to the invention, PACAP receptor antagonism has also been demonstrated to relieve opioid induced hyperalgesia in an animal model. Accordingly, the demonstration in the examples that trpm3 inhibition reduces PACAP shows that trpm3 inhibitors are suitable for the treatment of medication overuse headache, including opioid induced hyperalgesia . . . .

In the context of this invention, the term migraine refers to a condition that satisfies the diagnostic criteria for migraine according to the International Classification of Headache Disorders (ICHD) of the HIS. This definition is periodically updated.

In the context of this invention, the term trigeminal neuralgia refers to a condition that satisfies the diagnostic criteria for trigeminal neuralgia according to the International Classification of Headache Disorders (ICHD) of the HIS. This definition is periodically updated.

In the context of this invention, the term cluster headache refers to a condition that satisfies the diagnostic criteria for cluster headache according to the International Classification of Headache Disorders (ICHD) of the HIS. This definition is periodically updated.

In the context of this invention, the term medication overuse headache refers to a condition that satisfies the diagnostic criteria for medication overuse headache according to the International Classification of Headache Disorders (ICHD) of the HIS. This definition is periodically updated.

Human Trpm3

Human TRPM3 refers to a protein product of the TRPM3 gene present on chromosome 9q-21.12. Allelic variants including those encoded by SNPs associated with migraine are included within this definition. In addition, the definition covers all isoforms of human TRPM3 that may be generated from any allelic variant.

In one embodiment, human TRPM3 refers to the hTRPM3 variant having the amino acid sequence set out in any one of sequences set out as: SEQ ID NO: 1, SEQ ID NO: 2, SEQ ID NO: 3, SEQ ID NO: 4, SEQ ID NO. 5, SEQ ID NO: 6, SEQ ID NO: 7, SEQ ID NO: 8, SEQ ID NO: 9, SEQ ID NO: 10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO: 25, SEQ ID NO:26, SEQ ID NO: 27, SEQ ID NO: 28, SEQ ID NO:29, SEQ ID NO:30, SEQ ID NO:31, SEQ ID NO: 32, SEQ ID NO: 33, SEQ ID NO: 34, SEQ ID NO: 35, SEQ ID NO: 36 and SEQ ID NO: 37 or a processed version of any one of SEQ ID Nos. 1-37 lacking the initial methionine residue.

In one embodiment, human TRPM3 refers to the TRPM3 variant having the amino acid sequence set out in SEQ ID NO: 2, or a processed version of this variant lacking the initial methionine residue.

In one embodiment, human TRPM3 is a human TRPM3 having one or more mutations compared with the sequences set out in SEQ ID NO: 1 to SEQ ID NO:37. In one embodiment, human TRPM3 has an amino acid substitution at one or more of the following positions R1670, A1645, R1457, D602, K774, S1678, Y378, V990 and P1090 (numbering based on SEQ ID NO:2). In one embodiment, human TRPM3 has one or more of the following substitutions R1670Q, A1645V, R1457Q, D602V, K774R, S1678F, Y378C, V990M and P1090Q (numbering based on SEQ ID NO:2). The skilled person will appreciate that, although the substitutions are described in relation to SEQ ID NO: 2, the substitution could occur in any isoform, and the invention is intended to encompass the corresponding variants with amino acid substitutions in each of SEQ ID NO:1 or SEQ ID NOS: 3-37 (or processed forms of these sequences lacking the initial methionine).

In one embodiment, human TRPM3 is a human TRPM3 having a gain of function mutation. A gain of function is one that results in constitutive activity (i.e., calcium influx in the absence of a stimulus), or increased sensitivity to stimuli (calcium influx at a lower concentration of agonist, or a larger calcium mobilisation at the same agonist concentration) as determined in a calcium mobilisation assay. In one embodiment, the human TRPM3 has one or more of the following amino acid substitutions R1670Q, A1645V, V990M and P10900 (numbering based on SEQ ID NO: 2). In one embodiment, the human TRPM3 has the amino acid substitution R1670Q (numbering based on SEQ ID NO: 2).

Conventional sequencing techniques can be used to identify changes in the nucleotide sequence of human TRPM3. Changes in nucleotide sequences that give rise to changes in the amino sequence can be readily identified. The nucleotide sequences of the exons of the human TRPM3 are set out as SEQ ID NO: 38 to 69.

The term human TRPM3 channel encompasses a channel composed of at least one monomer of human TRPM3 as outlined above. The term therefore encompasses heterotetrameric channels formed from mixtures of human TRPM3 variants and homotetrameric channels formed from a single human TRPM3 variant. In one embodiment, the term human TRPM3 channel refers to a homotetrameric channel.

Inhibitor of Human Trpm3

An inhibitor of human TRPM3 has one or more of the following properties:
1) inhibiting Ca²⁺ influx in a cell line expressing human TRPM3 channels following challenge with an agonist of a human TRPM3 channel;
2) reducing current increases in a cell line expressing human TRPM3 channels following challenge with an agonist of a human TRPM3 channel;
3) inhibiting release of CGRP from dorsal root ganglia or trigeminal ganglia following challenge with an agonist of a human TRPM3 channel;
4) inhibiting release of PACAP from dorsal root gangia or trigeminal ganglia following challenge with an agonist of a human TRPM3 channel;
5) reduces facial allodynia following dural sensitisation with a TRPM3 agonist.

Property 1 may be measured in a calcium mobilisation assay. An inhibitor of human TRPM3 reduces fluorescence emissions in a calcium mobilisation assay compared to the negative control (agonist challenge/no inhibitor). In various embodiments, the fluorescence is reduced by at least 10%, at least 20%, at least 30%, at least 40%, at least 50%, at least 60%, at least 70%, at least 80% or at least 90%. In one embodiment, an inhibitor of human TRPM3 reduces fluorescence by at least as much as a blocking concentration of isosakuranetin ((2S)-5,7-dihydroxy-2-(4-methoxyphenyl)-2,3-dihydrochromen-4-one).

Property 2 may be measured in an electrophysiological assay. An inhibitor of human TRPM3 reduces current increases compared to the negative control (agonist/no inhibitor).

An inhibitor of human TRPM3 reduces current increases by at least 10%, at least 20%, at least 30%, at least 40%, at least 50%, at least 60%, at least 70%, at least 80% or at least 90% compared to the negative control. In one embodiment, an inhibitor of human TRPM3 reduces current increases by at least as much as a blocking concentration of isosakuranetin ((2S)-5,7-dihydroxy-2-(4-methoxyphenyl)-2,3-dihydrochromen-4-one).

Property 3 may be measured by the CGRP release assay. An inhibitor of human TRPM3 reduces CGRP levels in the media by at least 10%, at least 20%, at least 30%, at least 40%, at least 50%, at least 60%, at least 70%, at least 80% or at least 90%. In one embodiment, an inhibitor of human TRPM3 reduces CGRP levels by at least as much as a blocking concentration of isosakuranetin ((2S)-5,7-dihydroxy-2-(4-methoxyphenyl)-2,3-dihydrochrornen-4-one).

Property 4 may be measured by the PACAP release assay. An inhibitor of human TRPM3 reduces PACAP levels in the media by at least 10%, at least 20%, at least 30%, at least 40%, at least 50%, at least 60%, at least 70%, at least 80% or at least 90%. In one embodiment, an inhibitor of human TRPM3 reduces PACAP levels by at least as much as a blocking concentration of isosakuranetin ((2S)-5,7-dihydroxy-2-(4-methoxyphenyl)-2,3-dihydrochrornen-4-one).

Property 5 is assessed in a suitable model, for example the five-day rat dural infusion migraine model described by Oshinsky et al., supra, using a TRPM3 agonist in place of inflammatory soup. The level of facial allodynia is lower in the presence of an inhibitor of human TRPM3 compared to that observed in the absence of the inhibitor of human TRPM3. In a particular embodiment, the reduction in facial allodynia is measured using von Frey filaments. In particular embodiments, an inhibitor of human TRPM3 reduces the von Frey threshold on a particular day following infusion by 0.5 g, 1 g, 1.5 g, or 2 g. In certain embodiment, the reduction is measured from day 0 to day 14 post the completion of infusion. In particular embodiments, the reduction is measured on day 0, day 3, day 6, day 9 or day 12.

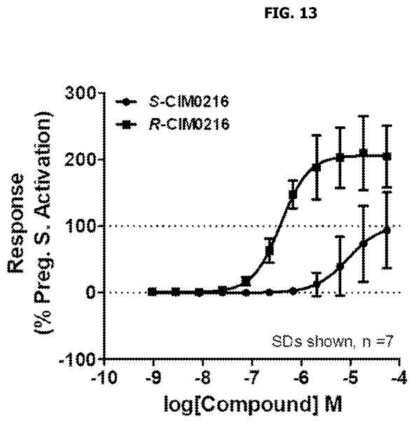

In the assays used to assess these properties, any compound capable of promoting calcium ion influx in a cell line expressing human TRPM3 may be used as the agonist. In one embodiment, the calcium ion influx is mediated by human TRPM3. Typically, the agonist is used at a concentration causing a response between 50% and 80% of the maximal response (i.e. between EC50-EC80) for the assays used to assess properties 1 to 4. In one embodiment, the agonist is pregnenolone sulfate or CIM0216 (racernate of 2-(3,4-dihydroquinolin-1(2H)-yl)-N-(5-methylisoxazol-3-yl)-2-phenylacetamide). In one embodiment, the agonist is (R)-2-(3,4-dihydroquinolin-1(2H)-yl)-N-(5-methylisoxazol-3-yl)propenamide. In another embodiment, the agonist is (S)-2-(3,4-dihydroquinolin-1(2H)-yl)-N-(5-methylisoxazol-3-yl)propenamide. FIG. 13 shows that (R)-2-(3,4-dihydroquinolin-1(2H)-yl)-N-(5-methylisoxazol-3-yl)propenamide is a more potent agonist than the (S)-isomer. Further details of the conduct of these assays are set out in the section entitled “IDENTIFICATION OF INHIBITORS OF HUMAN TRPM3”.

Accordingly, in one embodiment, the invention provides (R)-2-(3,4-dihydroquinolin-1(2H)-yl)-N-(5-methylisoxazol-3-yl)propenamide or a salt thereof. In a more particular embodiment, the invention provides (R)-2-(3,4-dihydroquinolin-1(2H)-yl)-N-(5-methylisoxazol-3-yl)propenamide (free base).

The nature of an inhibitor of human TRPM3 is not limited, and could be a chemical compound (i.e. a compound), an oligonucleotide, a peptide, a polypeptide, a protein, an antibody or an alternative antibody format. In one embodiment, the inhibitor of human TRPM3 is a compound.

The term “antibody” is used herein in the broadest sense to refer to molecules with an immunoglobulin-like domain (for example IgG, IgM, IgA, IgD or IgE) and includes monoclonal, recombinant, synthetic, polyclonal, chimeric, human, humanised, multispecific antibodies, including bispecific antibodies, and heteroconjugate antibodies; a single variable domain, antigen binding antibody fragments (e.g. Fab, F(ab′)2, Fv, disulphide linked Fv, single chain Fv, disulphide-linked scFv, diabodies, TANDAB™, etc.) and modified versions of any of the foregoing.

The term “domain” refers to a folded protein structure which retains its tertiary structure independent of the rest of the protein. Generally, domains are responsible for discrete functional properties of proteins and in many cases may be added, removed or transferred to other proteins without loss of function of the remainder of the protein and/or of the domain. The term “single variable domain” refers to a folded polypeptide domain comprising sequences characteristic of antibody variable domains. It therefore includes complete antibody variable domains such as VH, VHH and VL and modified antibody variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain. A single variable domain that is capable of binding an antigen or epitope independently of a different variable region or domain may be referred to as a “domain antibody” or “dAb(™)”. A single variable domain may be a human single variable domain, but also includes single variable domains from other species such as rodent, nurse shark and Camelid VHH dAbs™. Camelid VHH are immunoglobulin single variable domain polypeptides that are derived from species including camel, llama, alpaca, dromedary, and guanaco, which produce heavy chain antibodies naturally devoid of light chains. Such VHH domains may be humanised according to standard techniques available in the art, and such domains are considered to be “single variable domains”. As used herein VH includes camelid VHH domains.

Alternative antibody formats are those where the CDRs are arranged onto a suitable non-immunoglobulin protein scaffold or skeleton. The non-immunoglobulin scaffold may be a derived from the group consisting of CTLA-4, lipocalin, Protein A derived molecules such as Z-domain of Protein A (Affibody, SpA), A-domain (Avimer/Maxibody); heat shock proteins such as GroEl and GroES; transferrin (trans-body); ankyrin repeat protein (DARPin); peptide aptamer; C-type lectin domain (Tetranectin); human γ-crystallin and human ubiquitin (affilins); PDZ domains; LDL receptor class A domains; EGF domains; scorpion toxin kunitz type domains of human protease inhibitors; and fibronectin/adnectin.

Inhibitors of human TRPM3 are known in the art.

Inhibitors of human TRPM3 include those disclosed in WO2022112345 and WO2022112352. In one embodiment, an inhibitor of human TRPM3 is a compound of formula (I), or a pharmaceutically acceptable salt thereof:

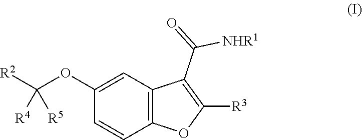

wherein:

R¹ is selected from the group consisting of:
a 4-6 membered saturated heterocyclylic ring, which 4-6 membered heterocyclic ring is optionally substituted by one, two or three groups independently selected from oxo, fluoro, C1-3fluoroalkyl and C₁₋₃ alkyl, which C₁₋₃alkyl group may optionally by substituted by one hydroxy group; and
a group of formula —CR⁶R⁷CONH₂, wherein R⁶ is optionally H or methyl and R⁷ is methyl substituted by one hydroxy group or ethyl substituted by one hyroxy group;
R² is phenyl or a 5-6 membered nitrogen containing heteroaryl ring, which phenyl or 5-6 membered nitrogen containing heteroaryl ring is optionally substituted by one two or three substituents independently selected from the group consisting of halo, cyclopropyl, cyclopropyloxy, methoxy and C₁₋₃alkyl, which C₁₋₃alkyl group is optionally substituted by one, two or three substitutents selected from fluoro and cyclopropyl; and

R³ is selected from cycloalkyl and methyl, which methyl group is optionally substituted by one, two or three fluoro groups.

R⁴ and R⁵ are independently selected from H, or methyl, which methyl group is optionally substituted with a group consisting of hydroxy, methoxy and N(CH₃)₂.

In one embodiment, R₁ is a group of formula —CR⁶R7CONH₂, wherein R⁶ is optionally H or methyl and R⁷ is methyl substituted by one hydroxy group or ethyl substituted by one hydroxy group. In a more particular embodiment, R¹ is a group of formula —CH(CH₂OH)CONH₂.

In one embodiment, R³ is methyl.

In one embodiment, R⁴ and R⁵ are each H. In another embodiment, R⁴ is H, and R⁵is methyl, which methyl group is optionally substituted with a group consisting of hydroxy and methoxy.

Compounds of formula (I) can be made as described in WO2022112352 or WO2022112345. It will be appreciated that certain compounds of formula (I) may exist in alternative tautomeric forms. For the avoidance of doubt, all tautomers of a compound of formula (I) are encompassed.

In one embodiment, the inhibitor of human TRPM3 is a compound selected from the group consisting of:
2-(difluoromethyl)-N-(3,3-difluoropiperidin-4-yl)-5-((4-methylthiazol-5-yl)methoxy)benzofuran-3-carboxamide;
N-(3,3-difluoropiperidin-4-yl)-2-methyl-5-((4-methylthiazol-5-yl)methoxy)benzofuran-3-carboxamide;
(S)—N-(1-amino-3-hydroxy-1-oxopropan-2-yl)-2-(difluoromethyl)-5-((4-methylthiazol-5-yl)methoxy)benzofuran-3-carboxamide;
(S)—N-(1-amino-3-hydroxy-1-oxopropan-2-yl)-2-cyclopropyl-5-((4-methylthiazol-5-yl)methoxy)benzofuran-3-carboxamide;
(S)-5-(benzyloxy)-2-methyl-N—(pyrrolidin-3-yl)benzofuran-3-carboxamide;
(S)-5-((2-fluorobenzyl)oxy)-2-methyl-N—(pyrrolidin-3-yl)benzofuran-3-carboxamide;
5-(benzyloxy)-N-(3,3-difluoropiperidin-4-yl)-2-methylbenzofuran-3-carboxamide;
N-(3,3-difluoropiperidin-4-yl)-5-((2-fluorobenzyl)oxy)-2-methylbenzofuran-3-carboxamide;
(S)—N-(1-amino-1-oxobutan-2-yl)-2-methyl-5-((4-methylthiazol-5-yl)methoxy)benzofuran-3-carboxamide;
(S)—N-(1-amino-1-oxobutan-2-yl)-5-(benzyloxy)-2-methylbenzofuran-3-carboxamide;
(S)—N-(1-amino-1-oxobutan-2-yl)-5-((2-fluorobenzyl)oxy)-2-methylbenzofuran-3-carboxamide;
(S)-2-methyl-5-((4-methylthiazol-5-yl)methoxy)-N-(pyrrolidin-3-yl)benzofuran-3-carboxamide;
5-(benzyloxy)-2-(difluoromethyl)-N-(3,3-difluoropiperidin-4-yl)benzofuran-3-carboxamide;
2-(difluoromethyl)-N-(3,3-difluoropiperidin-4-yl)-5-((2-fluorobenzyl)oxy)benzofuran-3-carboxamide;
(S)—N-(1-amino-3-hydroxy-1-oxopropan-2-yl)-5-(benzyloxy)-2-(difluoromethyl)benzofuran-3-carboxamide;
(S)—N-(1-amino-3-hydroxy-1-oxopropan-2-yl)-2-(difluoromethyl)-5-((2-fluorobenzyl)oxy)benzofuran-3-carboxamide;
(S)-2-(difluoromethyl)-5-((4-methylthiazol-5-yl)methoxy)-N-(pyrrolidin-3-yl)benzofuran-3-carboxamide;
(S)-5-(benzyloxy)-2-(difluoromethyl)-N-(pyrrolidin-3-yl)benzofuran-3-carboxamide;
(S)-2-(difluoromethyl)-5-((2-fluorobenzyl)oxy)-N-(pyrrolidin-3-yl)benzofuran-3-carboxamide;
(S)-2-cyclopropyl-5-((4-methylthiazol-5-yl)methoxy)-N-(pyrrolidin-3-yl)benzofuran-3-carboxamide;
(S)-5-(benzyloxy)-2-cyclopropyl-N-(pyrrolidin-3-yl)benzofuran-3-carboxamide;
(S)-2-cyclopropyl-5-((2-fluorobenzyl)oxy)-N-(pyrrolidin-3-yl)benzofuran-3-carboxamide;
(S)—N-(1-amino-3-hydroxy-1-oxopropan-2-yl)-5-(benzyloxy)-2-cyclopropylbenzofuran-3-carboxamide;
(S)—N-(1-amino-3-hydroxy-1-oxopropan-2-yl)-2-cyclopropyl-5-((2-fluorobenzyl)oxy)benzofuran-3-carboxamide;
2-cyclopropyl-N-(3,3-difluoropiperidin-4-yl)-5-((4-methylthiazol-5-yl)methoxy)benzofuran-3-carboxamide;
5-(benzyloxy)-2-cyclopropyl-N-(3,3-difluoropiperidin-4-yl)benzofuran-3-carboxamide; and
2-cyclopropyl-N-(3,3-difluoropiperidin-4-yl)-5-((2-fluorobenzyl)oxy)benzofuran-3-carboxamide; or a pharmaceutically acceptable salt thereof.

Additional inhibitors of human TRPM3 are reviewed in Held et al., (2015, Temperature 2: 201-13). Inhibitors include, for example, primidone (5-ethyldihydro-5-phenyl-4,6(1 H,5H)-pyrimidinedione), diclofenac, ononetin, econazole, the calmodulin antagonist W-7, the PPARy agonists rosiglitazone, troglitazone and pioglitazone, the flavonoid derivatives disclosed in Straub et al. (Mol Pharmacol, 2013, 84(5):736-50), the fenamate derivates disclosed in Klose et al. (Br J Pharmacol., 2011, 162(8): 1757-1769), and the TRPM3-specific polyclonal antibody (TM3E3).

The properties that define an inhibitor of human TRPM3 are determined experimentally. The skilled person will appreciate that this permits the identification of additional inhibitors of human TRPM3. This is discussed in the section entitled “IDENTIFICATION OF INHIBITORS OF HUMAN TRPM3”.

In particular embodiments, the inhibitor of human TRPM3 exhibits selectivity for inhibition of human TRPM3 channels in comparison to other TRP channels. In particular, the inhibitor of human TRPM3 exhibits selectivity for inhibition of human TRPM3 over one or more of TRPM1, TRPV1, TRPV4 and TRPM8. The skilled person will readily understand that selectivity can be assessed in binding assays for example, an assay using a labelled ligand, or where the TRP channel also acts to increase intracellular calcium, in a calcium mobilisation assay.

The inhibitor of human TRPM3 may be an inhibitor of a mutated version of human TRPM3, such as those disclosed supra, for example a mutated version of human TRPM3 having a gain of function mutation. For the avoidance of doubt, an inhibitor of a mutated version of human TRPM3 is an inhibitor of the mutated version, and does not require selectivity over the corresponding wild type variant of TRPM3, although selectivity may be required in particular embodiments. For example, in one embodiment, the inhibitor of a mutated version of human TRPM3 having a gain of function mutation is an inhibitor that is selective for the mutated version over the corresponding wild type TRPM3 variant.

In one embodiment, the inhibitor of human TRPM3 exhibits selectivity for the TRPM3 having the sequence set out in SEQ ID NO: 2 (or a processed version of SEQ ID NO:2 lacking the initial methionine residue) over other TRPM3 variants. Similarly to the above, the skilled person would understand that selectivity for this isoform can be assessed in binding assays or using a calcium mobilisation assay using channels that are homotetrameric for SEQ ID NO:2 (or a processed version of SEQ ID NO:2 lacking the initial methionine residue) and channels that are homotetrameric for the reference TRPM3 variant.

In a further embodiment, the inhibitor of human TRPM3 selectively inhibits the response to a particular stimulus. As explained in the Background, human TRPM3 is polymodally activated. In one embodiment, inhibition is selective to agonism by pregnenolone sulfate over heat. In another embodiment, inhibition is selective to agonism by pregnenolone sulfate over other agonists (e.g., nifedipine, D-erythrosphingosine, CIM2016). A functional assay such as a calcium mobilisation assay may be used to assess selectivity to particular stimuli.

In the above embodiments in which selectivity is assessed using a binding assay, the Kd for human TRPM3 channels or channels homotetrameric for a particular isoform of human TRPM3 is at least 10 fold lower compared to the reference channel. In a more particular embodiment, the Kd for the human TRPM3 channels or channels homotetrameric for a particular isoform of human TRPM3 is at least 100 fold lower compared to the reference channel. In embodiments above in which selectivity is assessed using a calcium mobilisation assay, the IC50 or Kd derived therefrom for human TRPM3 channels or channels homotetrameric for a particular isoform of human TRPM3 is at least 10 fold lower compared to the reference channel. In a particular embodiment above in which selectivity is assessed using a calcium mobilisation assay, the IC50 or Kd derived therefrom for human TRPM3 channels or channels homotetrameric for a particular isoform of human TRPM3 is at least 100 fold lower compared to the reference channel.

Identification of Inhibitors of Human Trpm3

As discussed supra, the properties that define an inhibitor are determined experimentally in assays. These assays can be used to identify additional inhibitors of human TRPM3.

Property 1 is measured using a calcium mobilisation assay in which changes in intracellular calcium ion levels are detected by changes in calcium indicator compounds. Currently, over one hundred chemically synthesized and genetically encoded calcium indicators are available.

Accordingly, in one aspect, the invention provides a method for identifying an inhibitor of human TRPM3, comprising:
a) contacting a cell line expressing human TRPM3 which cells contain an intracellular calcium indicator with an agonist of human TRPM3 in the presence and absence of a test inhibitor; and
b) measuring a change in intracellular calcium concentrations by measuring a change in the intracellular calcium indicator;

wherein the test inhibitor is identified as an inhibitor for human TRPM3 if intracellular calcium concentrations are reduced in the presence of the test inhibitor compared to those achieved in the absence of test inhibitor.

In one embodiment, the intracellular calcium indicator is a synthetic calcium indicator, and step a) is preceded by a step of loading the cells with the indicator. Several synthetic calcium indicator compounds are available commercially, for example, the FLUO calcium indicators (Invitrogen). Synthetic calcium indicators may be loaded into cells using methods known in the art. For example, water soluble salts of synthetic calcium indicators may be loaded into cells by well known methods including microinjection, by addition to patch pipette solutions, or by use of pinocytosis, for example using the INFLUX pinocytotic cell loading reagent. Cell permeant AM esters of calcium indicators may be loaded into cells by addition to the media, typically in the presence of a non-ionic detergent such as PLURONIC F-127, and an anion transport inhibitor such as probenecid or sulfinpyrazone, and incubation at 20-37° C. for a suitable period of time (e.g., between 15 minutes and 4 hours). Following cell loading, cells may be washed and background fluorescence due to indicator leakage could be quenched by addition of, for example, an anti-fluorescein antibody, although this is not essential. Where cell permeant AM esters were used, incubation for a further 30 minutes after loading permits de-esterification of the intracellular AM esters prior to the assay being conducted.

In an alternative embodiment, the intracellular calcium indicator is a genetically encoded calcium indicator. Several genetically encoded calcium indicators are known in the art, including the GCaMPs, pericams, GECOs, carngaroos. Constructs for transfecting cell lines with these genetically encoded calcium indicators are known in the art, for example, the GCaMP6s-P2A-Bsr construct (commercially available as Addgene plasmid #40753). Clonal cell lines based upon this construct showed bright, uniform cytoplasmic staining. Transient and stable cell line production using this construct is described in Wu et al., 2019 (2019, Sci Rep, 9: 12692).

In one embodiment, DNA encoding the genetically encoded calcium indicator is transfected into a cell line expressing human TRPM3 before step a). In an alternative embodiment, the cell line is a cell line stably expressing the genetically encoded calcium indicator.

Calcium mobilisation assays using cells loaded with calcium indicators are well known in the art. In one embodiment, the calcium indicator is an indicator whose fluorescence changes in the presence of calcium ions. In such embodiments, calcium mobilisation assays involve measuring levels of fluorescence following excitation with an appropriate wavelength for the calcium indicator following the addition of an agonist and inhibitor. The inhibitor may be added to the cells either before (e.g. 60 minutes before) or at the same time as the agonist. Fluoresence may be measured using a fluorescent imaging plate reader (several are commercially available, for example FLIPR TETRA), or by FACS analysis. Typically, negative and positive controls and standards are included in each experiment. Negative controls lack agonist, positive controls lack inhibitor and a standard uses a blocking concentration of a known inhibitor, for example, isosakuranetin.

A suitable calcium mobilisation assay in 96 well plates has been described by Zhao and colleagues, (eLife 2020; 9: e55634).

In a calcium mobilisation assay, an inhibitor of human TRPM3 is a compound that reduces fluorescence emissions compared to the positive control. In various embodiments, the fluorescence is reduced by at least 10%, at least 20%, at least 30%, at least 40%, at least 50%, at least 60%, at least 70%, at least 80% or at least 90%. In one embodiment, an inhibitor is a compound that reduces fluorescence by at least as much as a blocking concentration of isosakuranetin. In one embodiment, an inhibitor is a compound that reduces fluorescence by at least as much as a saturating concentration of isosakuranetin (i.e. maximal inhibition with isosakuranetin).

Property 2 is measured in an electrophysiological assay such as patch clamp or using an automated electrophysiology platform. These assays are well known in the art and several automated electrophysiology platforms are available commercially, for example the IONWORKS platforms, PATCHXPRESS, IONFLUX, QPATCH HT/HTX, PATCHLINER and SYNCHROPATCH platforms.

Accordingly, an inhibitor of human TRPM3 can be identified by a method comprising measuring the current increases in a cell line expressing human TRPM3 following challenge with an agonist of human TRPM3 in the presence and absence of a test inhibitor, wherein the test inhibitor is identified as an inhibitor for human TRPM3 if the increase in current is reduced in the presence of the test inhibitor compared to the increase achieved in the absence of the test inhibitor.

In one embodiment, the change in current is measured at a membrane potential of between +/−80 mV. In particular embodiments, the change in current is measured at a membrane potential of −80 mV, −70 mV, −60 mV, −50 mV, −40 mV, −30 mV, −20 mV, −10 mV, 0 mV, +10 mV, +20 mV, +30 mV, +40 mV, +50 mV, +60 mV, +70 mV or +80 mV. In one embodiment, the change in current is measured at a membrane potential of −80 mV. In an alternative embodiment, the change in current is measured at a membrane potential of +80 mV. In certain embodiments, mean changes in current are measured.

The assays to assess properties 1 and 2 utilise cell lines expressing human TRPM3. Primary or immortalised cell lines endogenously expressing human TRPM3 may be used. In one embodiment, the SHSY-5Y cell line may be used. In another embodiment, transient and stable cell lines expressing recombinant human TRPM3 may be prepared by conventional means. In one embodiment, the invention provides a cell line stably expressing recombinant human TRPM3.

The production of a HEK293 cell line expressing the human TRPM3 variant i (SEQ ID NO: 11) is described in by Zhao and colleagues supra. Further cell lines expressing TRPM3 variants are disclosed in WO200526317.

In one aspect, the invention provides a cell line expressing a recombinant human TRPM3 variant comprising one or more amino acid substitutions at residues selected from the group consisting of R1670, A1645, R1457, D602, K774, S1678, Y378, V990 and P1090 (numbering based on SEQ ID NO:2). In one embodiment, the invention provides a cell line expressing a recombinant human TRPM3 variant having one, two or three of the following substitutions R1670Q, A1645V, R1457Q, D602V, K774R, S1678F, Y378C, V990M and P1090Q (numbering based on SEQ ID NO:2). In one embodiment, the invention provides a cell line expressing a recombinant human TRPM3 variant comprising the amino acid substitution R1670Q (numbering based on SEQ ID NO:2).

In another aspect, the invention provides a cell line expressing recombinant human TRPM3 variant having the sequence set out in SEQ ID NO: 2, a processed version of SEQ ID NO:2 lacking the initial methionine residue, or a variant of SEQ ID NO: 2 or the processed version of SEQ ID NO: 2 comprising one two or three amino acid substitutions compared to the sequence set out in SEQ ID NO:2. In one embodiment, the invention provides a cell line expressing recombinant human TRPM3 variant having the sequence set out in SEQ ID NO: 2, a processed version of SEQ ID NO:2 lacking the initial methionine, or a variant of SEQ ID NO: 2 or the processed version of SEQ ID NO: 2 which has one, two or three amino acid substitutions at residues selected from the group consisting of R1670, A1645, R1457, D602, K774, S1678, Y378, V990 and P1090 (numbering based on SEQ ID NO:2). In one embodiment, the invention provides a cell line expressing recombinant human TRPM3 variant having the sequence set out in SEQ ID NO: 2, a processed version of SEQ ID NO:2, or a variant of SEQ ID NO: 2 or the processed version of SEQ ID NO: 2 which has one, two or three of the following substitutions R1670Q, A1645V, R1457Q, D602V, K774R, S1678F, Y378C, V990M and P1090Q (numbering based on SEQ ID NO:2). In a more particular embodiment, the cell line is formed by transient transfection of DNA encoding the human TRPM3 variant having the sequence set out in SEQ ID NO: 2, a processed version of SEQ ID NO:2 lacking the initial methionine, or a variant of SEQ ID NO:2 or the processed version of SEQ ID NO: 2 comprising the amino acid substitution R1670Q. In one embodiment, the cell line is formed by transient transfection of DNA encoding the human TRPM3 variant having the sequence set out in SEQ ID NO: 2, In another embodiment, the cell line is formed by transient transfection of DNA encoding the human TRPM3 variant having the sequence of SEQ ID NO:2 comprising the amino acid substitution R1670Q. In another embodiment, the cell line stably expresses the human TRPM3 variant having the sequence set out in SEQ ID NO: 2, a processed version of SEQ ID NO:2 lacking the initial methionine or a variant of SEQ ID NO:2 or the processed version of SEQ ID NO: 2 comprising the amino acid substitution R1670Q. In one embodiment, the cell line stably expresses the human TRPM3 variant having the sequence set out in SEQ ID NO: 2 or a processed version of SEQ ID NO: 2 lacking the initial methionine. In one embodiment, the cell line stably expresses the human TRPM3 variant that has the sequence of SEQ ID NO:2 or the processed form of SEQ ID NO:2 lacking the initial methionine, comprising the amino acid substitution R1670Q.

In further embodiments of the above cell lines, the cell line also stably expresses a genetically encoded calcium indicator. In one embodiment, the cell line is a human cell line. In one embodiment, the assays described above are conducted in this cell line. The cell lines described herein may be used in the identification of an inhibitor of human TRPM3.

Property 3) may be measured in freshly extracted dorsal root ganglia or trigeminal ganglia from rats or mice and measuring CGRP in the incubation fluid. The CGRP content of the incubation fluid may be measured by methods known in the art. A suitable enzyme immunoassay kits with a detection threshold of 5 μg/mL is commercially available (Bertin Pharma) which permits the media to be photometrically analyzed. Example 2 exemplifies a suitable assay.

In one aspect, the invention provides a method for identifying an inhibitor of human TRPM3, comprising measuring release of CGRP from dorsal root ganglia or trigeminal ganglia, or from primary cultures of cells isolated from dorsal root ganglia or trigeminal ganglia, following challenge with an agonist of human TRPM3 in the presence or absence of a test inhibitor, wherein the test inhibitor is identified as an inhibitor for human TRPM3 if CGRP production is reduced in the presence of the test inhibitor compared to CGRP production in the absence of the test inhibitor.

Property 4) may be measured in freshly extracted dorsal root ganglia or trigeminal ganglia from rats or mice and measuring PACAP. The PACAP content of the incubation fluid is measured indirectly. The conditioned media is incubated with PAC1-receptor expressing CHO cells, binding of PACAP to PAC1 receptor induces concentration dependent cAMP production that may be measured using a homogeneous time-resolved fluorescence resonance energy transfer (TR-FRET) immunoassay. A suitable TR-FRET cAMP assay is commercially available from (Perkin Elmer). Example 2 exemplifies a suitable assay.

In one aspect, the invention provides a method for identifying an inhibitor of human TRPM3, comprising measuring release of PACAP from dorsal root ganglia or trigeminal ganglia, or from primary cultures of cells isolated from dorsal root ganglia or trigeminal ganglia, following challenge with an agonist of human TRPM3 in the presence or absence of a test inhibitor, wherein the test inhibitor is identified as an inhibitor for human TRPM3 if PACAP production is reduced in the presence of the test inhibitor compared to PACAP production in the absence of the test inhibitor.

In one embodiment, the methods used to measure property 3) or 4) use primary cultures of cells isolated from dorsal root ganglia or trigeminal ganglia (i.e., cells isolated from dorsal root ganglia or trigeminal ganglia and placed into culture). In one embodiment, the method uses primary cultures of cells isolated from trigeminal ganglia. In one embodiment, the method uses primary cultures of cells in multiwell plates.

In one embodiment, an inhibitor of human TRPM3 reduces PACAP levels by at least 10%, at least 20%, at least 30%, at least 40%, at least 50%, at least 60%, at least 70%, at least 80% or at least 90%. In one embodiment, an inhibitor of human TRPM3 reduces PACAP levels by at least as much as a blocking concentration of isosakuranetin.

Property 5) may be assessed in a suitable animal model, for example the five-day rat dural infusion migraine model described in Example 4. The level of facial allodynia is lower in the presence of an inhibitor of human TRPM3 compared to that observed in the absence of the inhibitor of human TRPM3. In a particular embodiment, the reduction in facial allodynia is measured using von Frey filaments. In particular embodiments, an inhibitor of humant TRPM3 reduces the von Frey threshold on a particular day following infusion by 0.5 g, 1 g, 1.5 g, or 2 g. In certain embodiment, the reduction is measured from day 0 to day 14 post the completion of infusion. In particular embodiments, the reduction is measured on day 0, day 3, day 6, day 9 or day 12.

Any compound capable of promoting calcium ion influx in a cell line expressing human TRPM3 may be used as the agonist in the assays described supra. In one embodiment, the influx is mediated by human TRPM3. In one embodiment, the agonist is pregnenolone sulfate or CIM0216 (racemate of 2-(3,4-dihydroquinolin-1 (2H)-yl)-N-(5-methylisoxazol-3-yl)-2-phenylacetamide). In a particular embodiment, pregnenolone sulfate is used at a concentration in the range from 1 to 300 μM in the assays measuring properties 1-4. In a particular embodiment, pregnenolone sulfate is used at a concentration of about 100 μM in the assays measuring properties 1-4. In another embodiment, CIM0216 is used at a concentration in the range from 0.1 to 30 μM, more particularly in the range from 6 to 10 μM in the assays measuring properties 1-4. In one embodiment, CIM0216 or the R or S isomers thereof are used at a concentration of about 6 μM in the assays measuring properties 1-4. In another embodiment, CIM0216 or the R or S isomers thereof are used at a concentration of about 10 μM in the assays measuring properties 1-4. For dural infusion to measure property 4, pregnenolone sulfate is used at 5 mM/rat/day or CIM0216 or the R or S isomers thereof are used at 215 μM/rat/day. In another embodiment, CIM0216 or the R or S isomers thereof are used at a concentration of 215 μM/rat/day for dural infusion to measure property 4.

Therapeutic Use

In one embodiment, the invention provides an inhibitor of human TRPM3 as discussed herein for use in the treatment of migraine. In this context, treatment of migraine refers to the symptomatic treatment of acute migraine. Migraines may present with or without aura or visual disturbances. In one embodiment, the invention provides an inhibitor of human TRPM3 for use in the treatment of migraine with aura or visual disturbances. In an alternative embodiment, the invention provides an inhibitor of human TRPM3 for use in the treatment of migraine without aura or visual disturbances.

In a more particular embodiment, the invention provides an inhibitor of human TRPM3 as discussed herein for use in the treatment of migraine in a human subject whose migraines are not responsive to CGRP inhibition.

For the avoidance of doubt, human subjects whose migraines are not responsive to CGRP inhibition is not limited to human subjects that have previously been prescribed a CGRP inhibitor and found it to lack efficacy, and additionally encompasses individuals that have not previously been prescribed a CGRP inhibitor, but, whose migraines would nonetheless not respond to such treatment options. Example 5 suggests that this includes over 10% of all migraineurs. However, in one embodiment, the invention comprises treating human subjects that have previously failed treatment with an antagonist of CGRP. Such patients can be readily by identified by a review of their clinical history. A suitable questionnaire to identify CGRP non responders is given in Example 5.

In another embodiment, the invention provides an inhibitor of human TRPM3 as discussed herein for use in the treatment of migraine in a human subject whose migraines are responsive to therapy with a triptan.

For the avoidance of doubt, human subjects whose migraines are responsive to a triptan is not limited to human subjects that have previously been prescribed and responded to a triptan, but additionally encompasses individuals that have not previously been prescribed a triptan, but, whose migraines would nonetheless respond to such treatment. However, in one embodiment, the invention comprises treating human subjects that have previously been treated with a triptan and self reported a clinical response. Such patients can be readily by identified by a review of their clinical history.

In one embodiment, the triptan is a triptan selected from the group consisting of: almotriptan, eletriptan, frovatriptan, naratriptan, rizatriptan, sumatriptan and zolmitriptan. In a more particular embodiment, the triptan is sumatriptan.

In particular embodiments, the invention provides an inhibitor of human TRPM3 as discussed herein for use in the treatment of migraine in a human subject whose migraines are not responsive to CGRP inhibition, but are responsive to therapy with a triptan.

In one embodiment, a therapeutically effective amount of an inhibitor of human TRPM3 is administered to the human subject. In this context, the term “therapeutically effective amount” refers to the quantity of the inhibitor of human TRPM3 that is required for symptomatic treatment of acute migraine. It may vary depending on the compound, migraine severity and the age and weight of the subject to be treated.

In one embodiment, treatment of acute symptomatic migraine refers to the situation where the percentage of patients that are pain free 2 hours after administration of the inhibitor of human TRPM3 is higher for a population of patients receiving the inhibitor of human TRPM3 compared to a population of patients receiving placebo. In this context, “pain free” is a patient reported measure.

In one embodiment, the percentage of patients that are pain free 2 hours after administration of the inhibitor of human TRPM3 is 10% or higher (compared to placebo).

In another embodiment, treatment of acute symptomatic migraine refers to the situation where the percentage of patients that have no headache pain 2 hours after administration of the inhibitor of human TRPM3 and have no relapse of headache pain within 24 hours after administration of the inhibitor of human TRPM3 is higher compared to a population of patients receiving placebo. Headache pain is a patient reported measure.

In one embodiment, the percentage of patients that have no headache pain 2 hours after administration of the inhibitor of human TRPM3 and have no relapse of headache pain within 24 hours after administration of the inhibitor of human TRPM3 is 10% or higher (compared to placebo).

In another embodiment, treatment of acute symptomatic migraine refers to the situation where the percentage of patients that have no headache pain 2 hours after administration of the inhibitor of human TRPM3 and have no relapse of headache pain within 48 hours in a population of patients receiving the inhibitor of human TRPM3 is higher compared to a population of patients receiving placebo. Again, headache pain is a patient reported measure.

In one embodiment, the percentage of patients that have no headache pain 2 hours after administration of the inhibitor of human TRPM3 and have no relapse of headache pain within 48 hours after administration of the inhibitor of human TRPM3 is 10% or higher (compared to placebo).

In one embodiment, the inhibitor of human TRPM3 is administered in combination with at least one other therapeutic agent selected from: a triptan, an ergot, a non-steroidal anti-inflammatory drug, an acetaminophen containing product, a butalbital containing product, an anti-emetic, caffeine, dexamethasone, ubrogepant, rimegepant and lasmiditan.

In one embodiment, the inhibitor of human TRPM3 is administered in combination with a triptan selected from the group consisting of: almotriptan, eletriptan, frovatriptan, naratriptan, rizatriptan, sumatriptan and zolmitriptan. In a more particular embodiment, the inhibitor of human TRPM3 is administered in combination with sumatriptan. In a more particular embodiment, the inhibitor of human TRPM3 is administered in combination with oral sumatriptan at a maximum dose of 200 mg/day.

In one embodiment, the inhibitor of human TRPM3 is administered in combination with a non-steroidal anti-inflammatory drug selected from the group consisting of diclofenac, ibuprofen, naproxen and ketorolac.

In one embodiment, the inhibitor of human TRPM3 is administered in combination with an anti-emetic selected from the group consisting of: promethazine, prochlorperazine, metoclopramide, trimethobenzamide and ondansetron.

The inhibitor of human TRPM3 and any other therapeutic agent(s) may be administered together or separately and, when administered separately, administration may occur simultaneously or sequentially, in any order. The amounts of inhibitor of human TRPM3 of the present invention and the other therapeutic agent(s) and the relative timings of administration will be selected in order to achieve the desired combined therapeutic effect. Simultaneous administration may be achieved by administration of (1) a unitary pharmaceutical composition including the therapeutic agents; or (2) simultaneous administration of separate pharmaceutical compositions each including one of the therapeutic agents. Alternatively, the combination may be administered separately in a sequential manner wherein one treatment agent is administered first and the other second or vice versa. Such sequential administration may be close in time or remote in time. The amounts of the inhibitor of human TRPM3 of the invention and the other therapeutic agent(s) and the relative timings of administration will be selected in order to achieve the desired combined therapeutic effect.

In one embodiment, the invention provides an inhibitor of human TRPM3 as discussed herein for use in the treatment of trigeminal neuralgia.

In one embodiment, the invention provides an inhibitor of human TRPM3 as discussed herein for use in the treatment of cluster headache.

In one embodiment, the invention provides an inhibitor of human TRPM3 for use in the treatment of postherpetic neuralgia.

In one embodiment, the invention provides an inhibitor of human TRPM3 for use in the treatment of chemotherapy-induced neuropathy.

In one embodiment, the invention provides an inhibitor of human TRPM3 for use in the treatment of, complex regional pain syndrome.

In one embodiment, the invention provides an inhibitor of human TRPM3 for use in the treatment of HIV sensory neuropathy.

In one embodiment, the invention provides an inhibitor of human TRPM3 for use in the treatment of peripheral nerve injury.

In one embodiment, the invention provides an inhibitor of human TRPM3 for use in the treatment of phantom limb pain.

In one embodiment, the invention provides a method of treating acute symptomatic migraine in a human subject comprising the steps of:
a) identifying a human subject whose migraines are not responsive to CGRP inhibition; and
b) administering a therapeutically effective amount of an inhibitor of human TRPM3 as defined herein to the subject;

whereby the acute symptomatic migraine in the subject is treated.

In one embodiment, the invention provides a method of treating acute symptomatic migraine in a human subject comprising the steps of:
a) identifying a human subject whose migraines are responsive to therapy with a triptan; and
b) administering a therapeutically effective amount of an inhibitor of human TRPM3 as defined herein to the subject;

whereby the acute symptomatic migraine in the subject is treated.

In one embodiment, the invention provides a method for decreasing the level of calcitonin gene related peptide in cranial blood in a subject comprising the steps of:

a) identifying a subject with pain selected from the group consisting of migraine, trigeminal neuralgia and cluster headache; and

b) administering a therapeutically effective amount of an inhibitor of human TRPM3 as defined herein to the subject;

whereby the calcitonin gene related peptide level in the cranial blood of the subject is decreased.

In another embodiment, the invention provides a method for decreasing calcitonin gene related peptide level in the systemic circulation of a subject comprising the steps of:

a) identifying a subject with pain selected from the group consisting of postherpetic neuralgia, chemotherapy-induced neuropathy, complex regional pain syndrome, HIV sensory neuropathy, peripheral nerve injury and phantom limb pain; and

b) administering a therapeutically effective amount of an inhibitor of human TRPM3 as defined herein to the subject;

whereby the calcitonin gene related peptide level in the systemic circulation of the subject is decreased.

Certain embodiments of the methods for decreasing calcitonin gene related peptide level may further comprise the steps of:

a) making a first measurement of calcitonin gene related peptide level in a relevant blood sample;

b) making a second measurement of calcitonin gene related peptide level in a relevant blood sample after administering to the subject a therapeutically effective amount of the inhibitor of human TRPM3; and

c) comparing the first measurement and second measurement.

In certain embodiments of the methods for decreasing calcitonin gene related peptide level, the subject is a subject that carries a mutated version of human TRPM3 wherein the mutated verion of human TRPM3 has one or more of the following amino acid substitutions: R1670Q, A1645V, V990M and P1090Q (numbering based on SEQ ID NO: 2).

Certain embodiments of the methods for decreasing calcitonin gene related peptide level may further comprise the steps of: determining whether the subject carries a mutated version of human TRPM3 wherein the mutated verion of human TRPM3 has one or more of the following amino acid substitutions: R1670Q, A1645V, V990M and P1090Q (numbering based on SEQ ID NO: 2). These steps may take place before step (a) in the above methods.

In one embodiment, the invention provides a method for decreasing the level of PACAP in cranial blood in a subject comprising the steps of:

a) identifying a subject with migraine; and

b) administering a therapeutically effective amount of an inhibitor of human TRPM3 as defined herein to the subject;

whereby the level of PACAP in the cranial blood of the subject is decreased.

In another embodiment, the invention provides a method for decreasing the level of PACAP in the systemic circulation of a subject comprising the steps of: a) identifying a subject with migraine; and

b) administering a therapeutically effective amount of an inhibitor of human TRPM3 as defined herein to the subject;

whereby the level of PACAP in the systemic circulation of the subject is decreased.

Certain embodiments of the methods for decreasing PACAP may further comprise the steps of:

a) making a first measurement of the level of PACAP in a relevant blood sample;

b) making a second measurement of the level of PACAP in a relevant blood sample after administering to the subject a therapeutically effective amount of the inhibitor of human TRPM3; and

c) comparing the first measurement and second measurement.

In certain embodiments of the methods for decreasing the level of PACAP, the subject is a subject that carries a mutated version of human TRPM3 wherein the mutated verion of human TRPM3 has one or more of the following amino acid substitutions: R1670Q, A1645V, V990M and P1090Q (numbering based on SEQ ID NO: 2).

Certain embodiments of the methods for decreasing the level of PACAP may further comprise the steps of: determining whether the subject carries a mutated version of human TRPM3 wherein the mutated verion of human TRPM3 has one or more of the following amino acid substitutions: R1670Q, A1645V, V990M and P10900 (numbering based on SEQ ID NO: 2). These steps may take place before step (a) in the above methods.

Prophylactic Use

In one aspect of the invention, the invention provides an inhibitor of human TRPM3 for use in the prevention of migraine. In one embodiment, the invention provides an inhibitor of human TRPM3 for use in the prevention of chronic migraine.

In one embodiment, the invention provides an inhibitor of human TRPM3 for use in the prevention of migraine in a human subject whose migraines are not responsive to CGRP inhibition. In one embodiment, the invention provides an inhibitor of human TRPM3 for use in the prevention of chronic migraine in a human subject whose migraines are not responsive to CGRP inhibition.

For the avoidance of doubt, human subjects whose migraines are not responsive to CGRP inhibition is not limited to human subjects that have previously been prescribed a CGRP inhibitor and found it to lack efficacy, and additionally encompasses individuals that have not previously been prescribed a CGRP inhibitor, but, whose migraines would nonetheless not respond to such treatment options. Example 5 suggests that this includes over 10% of all migraineurs. However, in one embodiment, the invention comprises treating human subjects that have previously failed treatment with an antagonist of CGRP. Such patients can be readily by identified by a review of their clinical history. A suitable questionnaire to identify CGRP non responders is given in Example 5.

In one embodiment, the invention provides an inhibitor of human TRPM3 as discussed herein for use in the prevention of migraine in a human subject whose migraines are responsive to therapy with a triptan. In one embodiment, the invention provides an inhibitor of human TRPM3 for use in the prevention of chronic migraine in a human subject whose migraines are responsive to therapy with a triptan.

For the avoidance of doubt, human subjects whose migraines are responsive to a triptan is not limited to human subjects that have previously been prescribed and responded to a triptan, but additionally encompasses individuals that have not previously been prescribed a triptan, but, whose migraines would nonetheless respond to such treatment. However, in one embodiment, the invention comprises treating human subjects that have previously been treated with a triptan and self reported a clinical response. Such patients can be readily by identified by a review of their clinical history.

In one embodiment, the triptan is a triptan selected from the group consisting of: almotriptan, eletriptan, frovatriptan, naratriptan, rizatriptan, sumatriptan and zolmitriptan. In a more particular embodiment, the triptan is sumatriptan.

In particular embodiments, the invention provides an inhibitor of human TRPM3 as discussed herein for use in the prevention of migraine in a human subject whose migraines are not responsive to CGRP inhibition, but are responsive to therapy with a triptan.

In one embodiment, a therapeutically effective amount of an inhibitor of human TRPM3 is administered to the human subject. In this context, the term “therapeutically effective amount” refers to the quantity of the inhibitor of human TRPM3 that is required for migraine prevention (e.g. prevention of chronic migraine). It may vary depending on the compound, the mean number of migraine days/month, and the age and weight of the subject to be treated.

In one embodiment, prevention of migraine refers to the situation where the reduction in mean monthly migraine days is greater for a population of patients receiving the inhibitor of TRPM3 compared to placebo.

In one embodiment, the reduction in mean monthly migraine days in a population of patients receiving the inhibitor of TRPM3 is at least 1, in a further embodiment, at least 2, in a further embodiment, at least 3 and in a further embodiment, at least 4.

In one embodiment, prevention of migraine refers to the situation where the 50% responder rate is higher for a population of patients receiving the inhibitor of TRPM3 compared to placebo.

In one embodiment, the 50% responder rate in patients receiving the inhibitor of TRPM3 is 20% higher for a population of patients receiving the inhibitor of TRPM3 compared to placebo. In a further embodiment, the 50% responder rate in patients receiving the inhibitor of TRPM3 is 25% higher for a population of patients receiving the inhibitor of TRPM3 compared to placebo.

In one embodiment, the invention provides a method of preventing migraine in a human subject comprising the steps of:
a) identifying a human subject whose migraines are not responsive to CGRP inhibition; and
   b) administering a therapeutically effective amount of an inhibitor of human TRPM3
   as defined herein to the subject;

whereby migraine is prevented in the subject.

In one embodiment, the invention provides a method of preventing migraine in a human subject comprising the steps of:
a) identifying a human subject whose migraines are responsive to therapy with a triptan; and
b) administering a therapeutically effective amount of an inhibitor of human TRPM3 as defined herein to the subject;

whereby migraine is prevented in the subject.

In one embodiment, the inhibitor of human TRPM3 is administered in combination with at least one other therapeutic agent selected from: botulinum toxin A, a CGRP inhibitor, an anticonvulsant, a p-blocker, an antidepressant and a non-steroidal anti-inflammatory drug.

In one embodiment, the inhibitor of human TRPM3 is administered in combination with at least one other therapeutic agent selected from: valproate, divalproex sodium, amitriptyline, topiramate, venlafaxine, metoprolol, propranolol and timolol.

Identification of Patients for Prophylactic or Therapeutic Treatment

In one aspect, the invention provides a method for identifying whether a patient diagnosed with migraine is a candidate for treatment with an inhibitor of human TRPM3, comprising:
a) sequencing the human TRPM3 gene in the patient diagnosed with migraine; and
b) comparing the sequence with the sequences of the human TRPM3 exons set out in SEQ ID NOs: 38-69 and identifying whether changes would modify the amino acid sequence of any isoform;

wherein, if a change in amino acid sequence is identified, the patient is a candidate for treatment with an inhibitor of human TRPM3.

In one embodiment, the patient has migraines that are not responsive to CGRP inhibition.

In one embodiment, the patient has migraines that are responsive to a triptan.

In one embodiment, the patient has migraines that are not responsive to CGRP inhibition and that are responsive to a triptan.

In one embodiment, the changes to the amino acid sequence are selected the group consisting of: R1670Q, A1645V, V990M and P10900 (numbering based on SEQ ID NO: 2). In one embodiment, the change to the amino acid sequence is R1670Q (numbering based on SEQ ID NO: 2).

In particular embodiments, the treatment will be acute symptomatic treatment of migraine. In another embodiment, the treatment will be for the prevention of migraine.

In related aspects, the invention provides an inhibitor of human TRPM3 for use in the treatment or prevention of migraine in a candidate for treatment with an inhibitor of human TRPM3. The invention also provides use of an inhibitor of human TRPM3 for use in the manufacture of a medicament for use in the treatment or prevention of migraine in a candidate for treatment with an inhibitor of human TRPM3. The invention also provides a method for treating or preventing migraine in a patient in need thereof, comprising administering an inhibitor of human TRPM3 to a human subject that is identified as a candidate for treatment with an inhibitor of human TRPM3.

Accordingly, the invention provides a method for migraine prevention, which comprises the following steps:
a) sequencing the human TRPM3 gene in the patient diagnosed with migraine;
b) comparing the sequence with the sequences of the human TRPM3 exons set out in SEQ ID NOs: 38-69 and identifying whether changes would modify the amino acid sequence of any isoform wherein, if a change in amino acid sequence is identified, the patient is a candidate for treatment with an inhibitor of human TRPM3;
c) administering to the patient that is a candidate for treatment with an inhibitor of human TRPM3 a therapeutically effective amount of an inhibitor of human TRPM3.

In one embodiment, the patient has migraines that are not responsive to CGRP inhibition.

In one embodiment, the patient has migraines that are responsive to a triptan.

In one embodiment, the patient has migraines that are not responsive to CGRP inhibition and that are responsive to a triptan.

In one embodiment, the patient is human.

In one embodiment, the changes to the amino acid sequence are selected the group consisting of: R1670Q, A1645V, V990M and P10900 (numbering based on SEQ ID NO: 2). In one embodiment, the change to the amino acid sequence is R1670Q (numbering based on SEQ ID NO: 2).

Combination Therapy

The inhibitor of human TRPM3 and any other therapeutic agent(s) may be administered together or separately and, when administered separately, administration may occur simultaneously or sequentially, in any order. The amounts of inhibitor of human TRPM3 of the present invention and the other therapeutic agent(s) and the relative timings of administration will be selected in order to achieve the desired combined therapeutic effect. Simultaneous administration may be achieved by administration of (1) a unitary pharmaceutical composition including the therapeutic agents; or (2) simultaneous administration of separate pharmaceutical compositions each including one of the therapeutic agents. Alternatively, the combination may be administered separately in a sequential manner wherein one treatment agent is administered first and the other second or vice versa. Such sequential administration may be close in time or remote in time. The amounts of the inhibitor of human TRPM3 of the invention and the other therapeutic agent(s) and the relative timings of administration will be selected in order to achieve the desired combined therapeutic effect.

Trigeminal Neuralgia and Cluster Headache

In one aspect of the invention, the invention provides an inhibitor of human TRPM3 for use in the prevention of trigeminal neuralgia. In one embodiment, the inhibitor of human TRPM3 is administered in combination with at least one other therapeutic agent selected from: carbamazepine an oxcarbazepine.

Cluster Headache

In one aspect of the invention, the invention provides an inhibitor of human TRPM3 for use in the prevention of cluster headache in a human subject.

In one embodiment, the human subject is a subject whose headaches are not responsive to CGRP inhibition.

In another embodiment, the human subject is a subject whose headaches are responsive to therapy with a triptan.

For the avoidance of doubt, human subjects whose headaches are not responsive to CGRP inhibition is not limited to human subjects that have previously been prescribed a CGRP inhibitor and found it to lack efficacy, and additionally encompasses individuals that have not previously been prescribed a CGRP inhibitor, but, whose headaches would nonetheless not respond to such treatment options. However, in one embodiment, the invention comprises treating human subjects that have previously failed treatment with an antagonist of CGRP. Such patients can be readily identified by a review of their clinical history.

For the avoidance of doubt, human subjects whose headaches are responsive to therapy with a triptan is not limited to human subjects that have previously been prescribed and responded to a triptan, but additionally encompasses individuals that have not previously been prescribed a triptan, but, whose headaches would nonetheless respond to such treatment. However, in one embodiment, the invention comprises treating human subjects that have previously been treated with a triptan and self reported a clinical response. Such patients can be readily by identified by a review of their clinical history.

In one particular embodiment, the headaches are not responsive to CGRP therapy and are responsive to therapy with a triptan.

In one embodiment, the triptan is a triptan selected from the group consisting of: almotriptan, eletriptan, frovatriptan, naratriptan, rizatriptan, sumatriptan and zolmitriptan. In a more particular embodiment, the triptan is sumatriptan.

In one embodiment, the inhibitor of human TRPM3 is for use in the treatment of cluster headache. In another embodiment, the inhibitor of human TRPM3 is for use in the prevention of cluster headache.

In one embodiment, the human subject has a TRPM3 allele with a gain of function mutation. In a more particular embodiment, the mutated version of human TRPM3 has one or more of the following amino acid substitutions is R1670Q, A1645V, V990M and P1090Q (numbering based on SEQ ID NO: 2).

Medication Overuse Headache

In one aspect, the invention provides an inhibitor of human TRPM3 for use in the treatment of medication overuse headache in a human subject. In a particular embodiment, the medication overuse headache is opioid induced medication overuse headache. This condition may alternatively be referred to as opioid induced hyperalgesia. In a more particular embodiment, the opioid induced medication overuse headache/opioid induced hyperalgesia is induced by opioids acting at the mu opioid receptor. In another embodiment, the medication overuse headache is triptan induced medication overuse headache. In a more particular embodiment, the triptan is sumatriptan.

In one embodiment, the human subject has a TRPM3 allele with a gain of function mutation. In a more particular embodiment, the mutated version of human TRPM3 has one or more of the following amino acid substitutions is R1670Q, A1645V, V990M and P10900 (numbering based on SEQ ID NO: 2).

Pharmaceutical Compositions/Routes of Administration/Dosages

An inhibitor of human TRPM3 may be administered by any convenient route. In particular embodiments, the inhibitor of human TRPM3 may be administered by orally, parenterally, intranasally or by inhalation. In one embodiment, the inhibitor of human TRPM3 is administered in a pharmaceutical composition. In one embodiment, the inhibitor of human TRPM3 is formulated in a pharmaceutical composition adapted for oral or parenteral administration, or for administration intranasally or by inhalation. Appropriate doses will readily be appreciated by those skilled in the art.

According to one aspect, the invention provides a pharmaceutical composition comprising an inhibitor of human TRPM3 and a pharmaceutically acceptable excipient. According to another aspect, the invention provides a process for the preparation of a pharmaceutical composition comprising admixing an inhibitor of human TRPM3 with a pharmaceutically acceptable excipient.

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Pharmaceutical formulations adapted for nasal administration can comprise a coarse powder having a particle size for example in the range 20 to 500 microns which is administered in the manner in which snuff is taken, i.e., by rapid inhalation through the nasal passage from a container of the powder held close up to the nose. Suitable formulations wherein the carrier is a liquid, for administration as a nasal spray or as nasal drops, include aqueous or oil solutions of the inhibitor of human TRPM3.

Pharmaceutical formulations adapted for administration by inhalation include fine particle dusts or mists, which may be generated by means of various types of metered, dose pressurized aerosols, nebulizers or insufflators.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations described herein may include other agents conventional in the art having regard to the type of formulation in question, for example those suitable for oral administration may include flavouring agents.

The present invention also provides unitary pharmaceutical compositions in which the inhibitor of human TRPM3 one or more other therapeutic agent(s) may be administered together. When an inhibitor of human TRPM3 is used in combination with a second therapeutic agent, the dose of each therapeutic agent may differ from the dose of that therapeutic agent when used alone.

In one embodiment, the invention provides a pharmaceutical composition comprising an inhibitor of human TRPM3, at least one other therapeutic agent selected from: a triptan, an ergot, a non-steroidal anti-inflammatory drug, an acetaminophen containing product, a butalbital containing product, an anti-emetic, caffeine, dexamethasone, ubrogepant and lasmiditan, and a pharmaceutically acceptable excipient. In a more particular embodiment, the pharmaceutical composition comprises an inhibitor of human TRPM3, a triptan selected from the group consisting of: almotriptan, eletriptan, frovatriptan, naratriptan, rizatriptan, sumatriptan and zolmitriptan and a pharmaceutically acceptable excipient. In a more particular embodiment, the pharmaceutical composition comprises an inhibitor of human TRPM3, sumatriptan and a pharmaceutically acceptable excipient.

In another embodiment, the invention provides a pharmaceutical composition comprising an inhibitor of human TRPM3, a non-steroidal anti-inflammatory drug selected from the group consisting of diclofenac, ibuprofen, naproxen and ketorolac and a pharmaceutically acceptable excipient.

In a further embodiment, the invention provides a pharmaceutical composition comprising an inhibitor of human TRPM3, an anti-emetic selected from the group consisting of: promethazine, prochlorperazine, metoclopramide, trimethobenzamide and ondansetron and a pharmaceutically acceptable excipient.

In one embodiment, the invention provides a pharmaceutical composition comprising an inhibitor of human TRPM3, at least one other therapeutic agent selected from: botulinum toxin A, a CGRP inhibitor, an anticonvulsant, a p-blocker, an antidepressant and a non-steroidal anti-inflammatory drug, and a pharmaceutically acceptable excipient.

In one embodiment, the invention provides a pharmaceutical composition comprising an inhibitor of human TRPM3, at least one other therapeutic agent selected from: valproate, divalproex sodium, amitriptyline, topiramate, venlafaxine, metoprolol, propranolol and timolol, and a pharmaceutically acceptable excipient.

In one embodiment, the invention provides a pharmaceutical composition comprising an inhibitor of human TRPM3, at least one other therapeutic agent selected from: carbamazepine and oxcarbazepine, and a pharmaceutically acceptable excipient.

### Numbered Embodiments—Set 1

1. An inhibitor of human TRPM3 for use in the treatment or prevention of a disorder selected from: migraine, trigeminal neuralgia, cluster headache, postherpetic neuralgia, chemotherapy-induced neuropathy, complex regional pain syndrome, HIV sensory neuropathy, peripheral nerve injury and phantom limb pain.
2. Use of an inhibitor of human TRPM3 in the manufacture of a medicament for the treatment or prevention of a disorder selected from: migraine, trigeminal neuralgia, cluster headache, postherpetic neuralgia, chemotherapy-induced neuropathy, complex regional pain syndrome, HIV sensory neuropathy, peripheral nerve injury and phantom limb pain.
3. A method of treatment or prevention of a disorder selected from migraine, trigeminal neuralgia, cluster headache, postherpetic neuralgia, chemotherapy-induced neuropathy, complex regional pain syndrome, HIV sensory neuropathy, peripheral nerve injury and phantom limb pain, which comprises administering a subject in need thereof a therapeutically acceptable amount of an inhibitor of human TRPM3.
4. A method according to embodiment 3, wherein the subject is human.
5. An inhibitor of human TRPM3 for use according to embodiment 1, use according to embodiment 2 or a method according to embodiments 3 or 4, wherein the inhibitor of human TRPM3 is an inhibitor of a human TRPM3 variant having the sequence set out in SEQ ID NO:2, or a processed version of this variant lacking the initial methionine residue.
6. An inhibitor of human TRPM3 for use according to embodiment 1 or embodiment 5, use according to embodiment 2 or embodiment 5 or a method according to any one of embodiments 3 to 5, wherein the inhibitor of human TRPM3 is an inhibitor of a mutated version of human TRPM3 having a gain of function mutation.
7. An inhibitor of human TRPM3 for use according to embodiment 6, use according to embodiment 6 or a method according to embodiment 6, wherein the mutated version of human TRPM3 has one or more of the following amino acid substitutions is R1670Q, A1645V, V990M and P1090Q (numbering based on SEQ ID NO: 2).
8. An inhibitor of human TRPM3 for use according to embodiment 1 or any one of embodiments 5 to 7, use according to embodiment 2 or any one of embodiments 5 to 7 or a method according to any one of embodiments 3 to 7, wherein the use is the treatment of migraine.
9. An inhibitor of human TRPM3 for use according to embodiment 8, use according to embodiment 8 or a method according to embodiment 8, wherein the percentage of patients that are pain free 2 hours after administration of the inhibitor of human TRPM3 is higher for a population of patients receiving the inhibitor of human TRPM3 compared to a population of patients receiving placebo.
10. An inhibitor of human TRPM3 for use according to embodiment 8, use according to embodiment 8 or a method according to embodiment 8, wherein the percentage of patients that are pain free 2 hours after administration of the inhibitor of human TRPM3 and do not use rescue medication or relapse within 24 hours after administration of the inhibitor of human TRPM3 is higher compared to a population of patients receiving placebo.
11. An inhibitor of human TRPM3 for use according to any one of embodiments 8 to 10, use according to any one of embodiments 8 to 10 or a method according to any one of embodiments 8 to 10, wherein the inhibitor of human TRPM3 is administered in combination with at least one other therapeutic agent selected from: a triptan, an ergot, a non-steroidal anti-inflammatory drug, an acetaminophen containing product, a butalbital containing product, an anti-emetic, caffeine, dexamethasone, ubrogepant and lasmiditan.
12. An inhibitor of human TRPM3 for use according to any one of embodiments 8 to 10, use according to any one of embodiments 8 to 10 or a method according to any one of embodiments 8 to 10, wherein the inhibitor of human TRPM3 is administered in combination with a triptan and a non-steroidal anti-inflammatory drug.
13. An inhibitor of human TRPM3 for use according to any one of embodiments 8 to 10, use according to any one of embodiments 8 to 10 or a method according to any one of embodiments 8 to 10, wherein the inhibitor of human TRPM3 is administered in combination with sumatriptan.
14. An inhibitor of human TRPM3 for use according to any one of embodiments 8 to 10, use according to any one of embodiments 8 to 10 or a method according to any one of embodiments 8 to 10, wherein the inhibitor of human TRPM3 is administered in combination with a non-steroidal anti-inflammatory drug.
15. An inhibitor of human TRPM3 for use according to any one of embodiments 1 or any one of embodiments 5 to 7, use according to embodiment 2 or any one of embodiments 5 to 7 or a method according to any one of embodiments 3 to 7, wherein the use is the prevention of migraine.
16. An inhibitor of human TRPM3 for use according to embodiment 15, use according to embodiment 15 or a method according to embodiment 15, wherein the reduction in mean monthly migraine days is greater for a population of patients receiving the inhibitor of TRPM3 compared to placebo.
17. An inhibitor of human TRPM3 for use according to embodiment 15, use according to embodiment 15 or a method according to embodiment 15, wherein the 50% responder rate is higher for a population of patients receiving the inhibitor of TRPM3 compared to placebo.
18. An inhibitor of human TRPM3 for use according to any one of embodiments 15 to 17, use according to any one of embodiments 15 to 17 or a method according to any one of embodiments 15 to 17, wherein the inhibitor of human TRPM3 is administered in combination with at least one other therapeutic agent selected from: botulinum toxin A, a CGRP inhibitor, an anticonvulsant, a p-blocker, an antidepressant and a non-steroidal anti-inflammatory drug.
19. An inhibitor of human TRPM3 for use according to any one of embodiments 15 to 17, use according to any one of embodiments 15 to 17 or a method according to any one of embodiments 15 to 17, wherein the inhibitor of human TRPM3 is administered in combination with a therapeutic agent selected from: valproate, divalproex sodium, amitriptyline, topiramate, venlafaxine, metoprolol, propranolol and timolol.
20. A method for identifying whether a patient diagnosed with migraine is a candidate for treatment with an inhibitor of human TRPM3, comprising:
   a) sequencing the human TRPM3 gene in the patient diagnosed with migraine;
   b) comparing the sequence with the sequences of the human TRPM3 exons set out in SEQ ID NOs: 38-69 and identifying whether changes would modify the amino acid sequence of any isoform;
   wherein, if a change in amino acid sequence is identified, the patient is a candidate for treatment with an inhibitor of human TRPM3.
21. A method for identifying an inhibitor of human TRPM3, comprising measuring release of CGRP from dorsal root ganglia or trigeminal ganglia, or from primary cultures of cells isolated from dorsal root ganglia or trigeminal ganglia, following challenge with an agonist of human TRPM3 in the presence or absence of a test inhibitor, wherein the test inhibitor is identified as an inhibitor for human TRPM3 if CGRP production is reduced in the presence of the test inhibitor compared to CGRP production in the absence of the test inhibitor.
22. A cell line expressing a recombinant human TRPM3 variant protein comprising one or more amino acid substitutions at residues selected from the group consisting of R1670, A1645, R1457, D602, K774, S1678, Y378, V990 and P1090 (numbering based on SEQ ID NO:2).
23. A cell line expressing a recombinant human TRPM3 isoform having the sequence set out in SEQ ID NO: 2, or a processed version of this lacking the initial methionine residue, or a variant of these sequences comprising the amino acid substitution R1670Q (numbering based on SEQ ID NO. 2).
24. Use of the cell line defined in embodiment 22 or 23 in the identification of an inhibitor of human TRPM3.
25. A method for identifying an inhibitor of human TRPM3, comprising:
   a) contacting a cell line as defined in embodiment 22 or embodiment 23, which cell line contains an intracellular calcium indicator with an agonist of human TRPM3 in the presence and absence of a test inhibitor; and
   b) measuring a change in intracellular calcium concentrations by measuring a change in the intracellular calcium indicator;
   wherein the test inhibitor is identified as an inhibitor for human TRPM3 if intracellular calcium concentrations are reduced in the presence of the test inhibitor compared to those achieved in the absence of the test inhibitor.
26. A method according to embodiment 25, wherein the cell line expresses a genetically encoded calcium indicator.
27. A method for identifying an inhibitor of human TRPM3, comprising measuring current increases in a cell line as defined in embodiment 22 or embodiment 23 following challenge with an agonist of human TRPM3 in the presence and absence of a test inhibitor, wherein the test inhibitor is identified as an inhibitor for human TRPM3 if the increase in current is reduced in the presence of the test inhibitor compared to the increase achieved in the absence of the test inhibitor.
28. A method for identifying an inhibitor of human TRPM3, comprising a step of dural sensitisation with a TRPM3 agonist in the presence or absence of the test inhibitor, followed by assessment of facial allodynia, wherein the test inhibitor is identified as an inhibitor for human TRPM3 if the level of facial allodynia is lower in the presence of the test inhibitor compared to that observed in the absence of the test inhibitor.
29. A method according to embodiment 21 or any one of embodiments 25 to 28, wherein the agonist of human TRPM3 is pregnenolone sulfate or a racemate of 2-(3,4-dihydroquinolin-1 (2H)-yl)-N-(5-methylisoxazol-3-yl)-2-phenylacetamide.

### Numbered Embodiments—Set 2

1. An inhibitor of human TRPM3 for use in the treatment or prevention of migraine in a human subject whose migraines are not responsive to CGRP inhibition.
2. Use of an inhibitor of human TRPM3 in the manufacture of a medicament for the treatment or prevention of migraine in a human subject whose migraines are not responsive to CGRP inhibition.
3. A method of treatment, or prevention, of migraine in a human subject whose migraines are not responsive to CGRP inhibition, which comprises administering a subject in need thereof a therapeutically effective amount of an inhibitor of human TRPM3.
4. An inhibitor of human TRPM3 for use according to embodiment 1, use according to embodiment 2 or a method according to embodiment 3, wherein the human subject has failed treatment with an antagonist of CGRP.
5. An inhibitor of human TRPM3 for use according to embodiment 1 or embodiment 4, use according to embodiment 2 or embodiment 4, or a method according to embodiment 3 or embodiment 4, wherein the human subject has migraines that are responsive to therapy with a triptan.
6. An inhibitor of human TRPM3 for use according to embodiment 5, use according to embodiment 5 or a method according to embodiment 5, wherein the human subject has migraines that have previously responded to treatment with a triptan.
7. An inhibitor of human TRPM3 for use in the treatment or prevention of migraine in a human subject whose migraines are responsive to therapy with a triptan.
8. Use of an inhibitor of human TRPM3 in the manufacture of a medicament for the treatment or prevention of migraine in a human subject whose migraines are responsive to therapy with a triptan.
9. A method of treatment or prevention of migraine in a human subject whose migraines are responsive to therapy with a triptan, which comprises administering a subject in need thereof a therapeutically acceptable amount of an inhibitor of human TRPM3.
10. An inhibitor of human TRPM3 for use according to embodiment 7, use according to embodiment 8 or a method according to embodiment 9, wherein the human subject has migraines that have previously responded to treatment with a triptan.
11. An inhibitor of human TRPM3 for use according to embodiment 6 or embodiment 10, use according to embodiment 6 or embodiment 10 or a method according to embodiment 6 or embodiment 10, wherein the triptan is selected from the group consisting of: almotriptan, eletriptan, frovatriptan, naratriptan, rizatriptan, sumatriptan and zolmitriptan.
12. An inhibitor of human TRPM3 for use according to any one of embodiments 1, 4, 5, 6, 7, 10 and 11, use according to any one of embodiments 2, 4, 5, 6, 8, 10 and 11 or a method according to any one of embodiments 3, 4, 5, 9, 10 and 11, wherein the human subject has a TRPM3 allele with a gain of function mutation.
13. An inhibitor of human TRPM3 for use according to embodiment 12, use according to embodiment 12 or a method according to embodiment 12, wherein the mutated version of human TRPM3 has one or more of the following amino acid substitutions is R1670Q, A1645V, V990M and P1090Q (numbering based on SEQ ID NO: 2).
14. An inhibitor of human TRPM3 for use according to any one of embodiments 1, 4, 5, 6, 7, 10, 11, 12 and 13, use according to any one of embodiments 2, 4, 5, 6, 8,10,11, 12 and 13 or a method according to any one of embodiments 3, 4, 5, 6, 9, 10, 11, 12 and 13, wherein the use is the treatment of migraine.
15. An inhibitor of human TRPM3 for use according to embodiment 14, use according to embodiment 14, or a method according to embodiment 14, wherein the percentage of patients that are pain free 2 hours after administration of the inhibitor of human TRPM3 is higher for a population of patients receiving the inhibitor of human TRPM3 compared to a population of patients receiving placebo.
16. An inhibitor of human TRPM3 for use according to embodiment 14, use according to embodiment 14 or a method according to embodiment 14, wherein, following administration to a population of patients, the percentage of patients that are pain free 2 hours after administration of the inhibitor of human TRPM3 and do not use rescue medication or relapse within 24 hours after administration of the inhibitor of human TRPM3 is higher compared to a population of patients receiving placebo.
17. An inhibitor of human TRPM3 for use according to any one of embodiments 1, 4, 5, 6, 7, 10, 11, 12 and 13, use according to any one of embodiments 2, 4, 5, 6, 8,10,11, 12 and 13 or a method according to any one of embodiments 3, 4, 5, 6, 9, 10, 11, 12 and 13, wherein the use is the prevention of migraine.
18. An inhibitor of human TRPM3 for use according to embodiment 17, use according to embodiment 17, or a method according to embodiment 17, wherein, following administration to a population of patients, the reduction in mean monthly migraine days is greater for a population of patients receiving the inhibitor of TRPM3 compared to placebo.
19. An inhibitor of human TRPM3 for use according to embodiment 17, use according to embodiment 17, or a method according to embodiment 17, wherein the 50% responder rate is higher for a population of patients receiving the inhibitor of TRPM3 compared to placebo.
20. An inhibitor of human TRPM3 for use in the treatment or prevention of cluster headache in a human subject whose headaches are not responsive to CGRP inhibition.
21. Use of an inhibitor of human TRPM3 in the manufacture of a medicament for the treatment or prevention of cluster headache in a human subject whose headaches are not responsive to CGRP inhibition.
22. A method of treatment or prevention of cluster headache in a human subject whose headaches are not responsive to CGRP inhibition, which comprises administering a subject in need thereof a therapeutically acceptable amount of an inhibitor of human TRPM3.
23. An inhibitor of human TRPM3 for use according to embodiment 20, use according to embodiment 21 or a method according to embodiment 23, wherein the human subject has failed treatment with an antagonist of CGRP.
24. An inhibitor of human TRPM3 for use according to embodiment 20 or embodiment 23, use according to embodiment 21 or embodiment 23, or a method according to embodiment 22 or embodiment 23, wherein the human subject has headaches that are responsive to therapy with a triptan.
25. An inhibitor of human TRPM3 for use according to embodiment 24, use according to embodiment 24 or a method according to embodiment 24, wherein the human subject has headaches that have previously responded to treatment with a triptan.
26. An inhibitor of human TRPM3 for use according to embodiment 25, use according to embodiment 25 or a method according to embodiment 25, wherein the triptan is selected from the group consisting of: almotriptan, eletriptan, frovatriptan, naratriptan, rizatriptan, sumatriptan and zolmitriptan.
27. An inhibitor of human TRPM3 for use according to any one of embodiments 20 or 23-26, use according to any one of embodiments 21 or 23-26, or a method according to any one of embodiments 22-26, wherein the human subject has a TRPM3 allele with a gain of function mutation.
28. An inhibitor of human TRPM3 for use according to embodiment 27, use according to embodiment 27 or a method according to embodiment 27, wherein the mutated version of human TRPM3 has one or more of the following amino acid substitutions is R1670Q, A1645V, V990M and P1090Q (numbering based on SEQ ID NO: 2),
29. An inhibitor of human TRPM3 for use in the treatment of medication overuse headache in a human subject.
30. Use of an inhibitor of human TRPM3 in the manufacture of a medicament for the treatment of medication overuse headache in a human subject.
31. A method of treatment of medication overuse headache in a human subject, which comprises administering a subject in need thereof a therapeutically acceptable amount of an inhibitor of human TRPM3.
32. An inhibitor of human TRPM3 for use according to embodiment 29, use according to embodiment 30 or a method according to embodiment 31, wherein the human subject has a TRPM3 allele with a gain of function mutation.
33. An inhibitor of human TRPM3 for use according to embodiment 32, use according to embodiment 32 or a method according to embodiment 32, wherein the mutated version of human TRPM3 has one or more of the following amino acid substitutions is R1670Q, A1645V, V990M and P1090Q (numbering based on SEQ ID NO: 2).
34. A method for measuring PACAP in a sample comprising incubating a cell line expressing the PAC1 receptor with the sample and measuring cAMP signalling in the cell line.
35. A method for identifying an inhibitor of human TRPM3, comprising measuring release of PACAP from dorsal root ganglia or trigeminal ganglia, or from primary cultures of cells isolated from dorsal root ganglia or trigeminal ganglia, following challenge with an agonist of human TRPM3 in the presence or absence of a test inhibitor, wherein the test inhibitor is identified as an inhibitor for human TRPM3 if PACAP production is reduced in the presence of the test inhibitor compared to PACAP production in the absence of the test inhibitor, and wherein PACAP production is measured according to the method defined in embodiment 34.
36. A method according to embodiment 35, wherein the agonist of human TRPM3 is pregnenolone sulfate, a racemate of 2-(3,4-dihydroquinolin-1(2H)-y)-N(5-methylisoxazol-3-yl)-2-phenylacetamide, (R)-2-(3,4-dihydroquinolin-1(2H)-yl)-N-(5-methylisoxazol-3-yl)propenamide or (S)-2-(3,4-dihydroquinolin-1(2H)-yl)-N-(5-methylisoxazol-3-yl)propenamide.
37. (R)-2-(3,4-dihydroquinolin-1(2H)-yl)-N-(5-methylisoxazol-3-yl)propenamide or a salt thereof.

### Examples

### Example 1: Genome Wide Association Study in Individuals with Migraine

Genome wide association study (GWAS) of individuals diagnosed with migraine identified a genetic region in the TRPM3 locus reaching the accepted genome-wide statistical threshold of p<5×10⁻⁸ on human chromosome 9.

GWAS Analysis

We computed association test results for the genotyped and the imputed SNPs. We performed association testing using logistic regression, assuming additive allelic effects. For tests using imputed data, we use the imputed dosages rather than best-guess genotypes. We included covariates for age, sex, and the top five principal components to account for residual population structure. The association test P value was computed using a likelihood ratio test, which in our experience is better behaved than a Wald test on the regression coefficient.

All individuals included in the analyses provided informed consent and answered surveys online according to our human subject protocol, which was reviewed and approved by Ethical & Independent Review Services, a private institutional review board (http://www.eandireview.com).

Genotyping and SNP Imputation

DNA extraction and genotyping were performed on saliva samples by CLIA-certified and CAP-accredited clinical laboratories of Laboratory Corporation of America. Samples were genotyped on one of five genotyping platforms. The V1 and V2 μlatforms were variants of the Illumina HumanHap550+ BeadChip and contained a total of about 560,000 SNPs, including about 25,000 custom SNPs selected by 23andMe. The V3 μlatform was based on the Illumina OmniExpress+ BeadChip and contained a total of about 950,000 SNPs and custom content to improve the overlap with our V2 array. The V4 μlatform is a fully custom array and includes a lower redundancy subset of V2 and V3 SNPs with additional coverage of lower-frequency coding variation, and about 570,000 SNPs. The V5 platform, in current use, is an Illumina Infinium Global Screening Array of about 640,000 SNPs supplemented with about 50,000 SNPs of custom content. Samples that failed to reach 98.5% call rate were re-analyzed. Individuals whose analyses failed repeatedly were re-contacted by 23andMe customer service to provide additional samples, as is done for all 23andMe customers.

Variants were imputed using two separate imputation reference panels. One included a larger number of samples, but did not include insertion or deletion variants. The other included a smaller number of individuals, but included insertion and deletion variants. Phased participant data was generated using an internally-developed tool based on Beagle (Browning, S. R. & Browning, B. L., Rapid and accurate Haplotype Phasing and Missing Data Interference for Whole Genome Association Studies Using Localized Haplotype Clustering, Am. J. Hum. Genet., 81: 1084-1097 (2007)) or a new phasing algorithm, Eagle2 (Loh, P. r. et a.I, Fast and accurate Long-Range Phasing ina UK Biobank Cohort, Nature Genetics, 48: 811-6 (2016)). We imputed phased participant data against both reference panels using Minimac3 (Das, S. et al., Next-generation genotype Imputation Service and Methods, Nat. Genet., 48: 1284-1287 (2016)) and then built a merged imputation dataset by combining the two sets of imputed data. A simple merging rule was applied: if a variant was imputed in the larger panel, the imputed results from the larger panel were included in the merged imputed dataset. For the remaining variants not present in the larger panel, the imputed results from the smaller panel were added to the merged dataset.

Results

We identified an independent association between migraine diagnosis and several SNPs near the TRPM3 C-terminus coding sequence. The sentinel SNP for migraine associations is the missense SNP rs6560142 (P=5.9×10⁻¹⁰, OR=1.020).

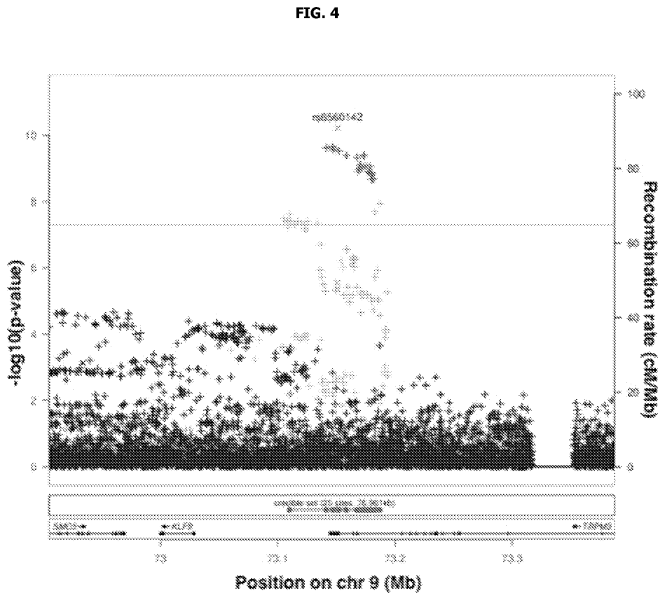

FIG. 4 is a regional association plot for migraine diagnosis on chromosome 9 where the x-axis shows physical positions on human genome build GRCh37/hg19 and the y-axes shows the −log 10 of the p-value for association with migraine diagnosis. Each point in the depicted plots represents a genetic variant tested for association in the region. The grey horizontal line represents the genome wide significance threshold of 5×10⁻⁸. Human genes in the region are depicted on the lower panel. These GWAS data indicate that the locus (genetic region) shown is implicated in susceptibility to migraine diagnosis in humans. In FIG. 4, the trait analyzed was “migraine diagnosis” where “the cases” are individuals with a migraine diagnosis and “the controls” are individuals who did not have a migraine diagnosis.

A common genetic variant changing the amino acid sequence of the TRPM3 protein was found to be the lead SNP for this locus. This variant rs6560142 (dbSNP build 154 identifier) is located on chromosome 9 at position 73150984 (of the human genome build GRCh37/hg19) and the observed frequency of the migraine diagnosis risk allele “T” is 0.56 in the research participant population with predominantly European ancestry (the protective allele “C” has a frequency of 0.44). This variant can also be described at the amino acid level in TRPM3, for example: Arg1670Gln, (numbering based on SEQ ID NO: 2). The GWAS analysis identified the rs6560142 missense SNP in the TRPM3 coding region as the SNP in this locus with the lowest P value. Consequently, these data imply that TRPM3 has the highest probability of being the gene that is functionally responsible for the association between this locus and migraine diagnosis. Therefore, these novel GWAS data indicates that the TRPM3 protein and its functions contribute to migraine pathophysiology in humans.

### Example 2—Neuropeptide Release from Sensory Ganglia Dorsal Root and Trigeminal Ganglion Neuron Cultures

Cultures were prepared from 8-13 week Trpm3^{+/+} (Wild type), Trpm3^{-/-} (knockout) C57BL/6NJ mice and Sprague Dawley rats. Following cervical dislocation and cardiac cessation, trigeminal ganglia (TG) and dorsal root ganglia (DRG) were removed and collected in ice cold dissociation media containing LEBOVITZ L-15 Medium, GLUTAMAX (Gibco). DRG and TG were incubated with 1 mg/ml type 1A collagenase (Sigma-Aldrich) for 15-30 min at 37° C. DRG and TG were then incubated with 1 mg/ml trypsin solution (Sigma-Aldrich) for 30-45 min. Digested ganglia were mechanically dissociated by trituration with a 1 ml pipette. Dissociated DRG were centrifuged at 200 g for 5 min. The cell pellet was resuspended in L15 culture media and plated onto poly-D Lysine 96 well plates (Greiner bio-one) coated with 10 μg/ml laminin (Sigma-Aldrich) and cultured at 37° C., 5% CO₂. Dissociated TG were passed through a 70 μm cell strainer and overlaid onto 4% (w/v) BSA. Cells were collected after centrifugation and plated onto poly-D Lysine-laminin coated 96 well plates and cultured at 37° C., 5% CO₂. CGRP and PACAP release experiments were conducted after 17-24 h in culture.

Neuropeptide Release Assay

To stimulate neuropeptide release, cells were incubated with TRPM3 agonist, either CIM0216 or pregnenolone sulfate (PS) for 30 min at 37° C. For inhibitor studies, cells were pre-incubated with TRPM3 inhibitor, isosakuranetin for 30 min prior to addition of agonist. At the end of the incubation period, conditioned medium was collected for the detection of CGRP and PACAP.

Measurement of CGRP

CGRP concentrations in conditioned media collected from TG and DRG neuron cultures were determined using an ELISA (Bertin bioreagent).

Measurement of PACAP

PACAP concentrations in conditioned medium collected from TG and DRG neuron cultures were determined indirectly. Conditioned medium was incubated with Chinese Hamster Ovary cells expressing the pituitary adenylate cyclase-activating polypeptide type I (PAC1) receptor (generated in house). Binding of PACAP to the PAC1 receptor induced cAMP production that was measured using a homogeneous time-resolved fluorescence resonance energy transfer (TR-FRET) immunoassay (Perkin Elmer). The TR-FRET signal was calibrated against a PACAP-38 standard curve.

Results—CGRP and PACAP Release from DRG and TG Cultures is Modulated by TRPM3 Ligands

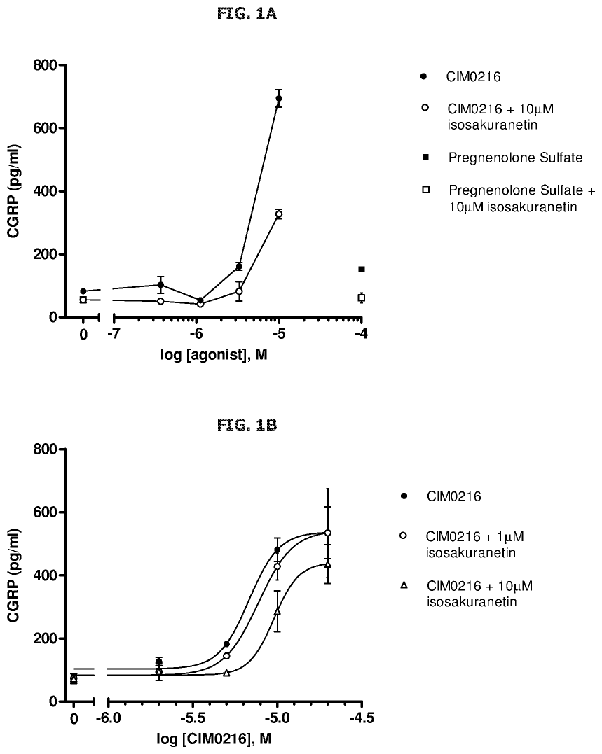

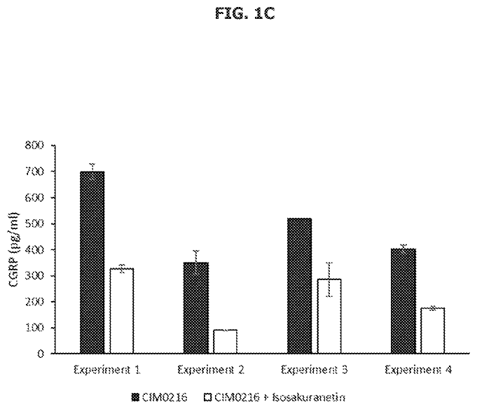

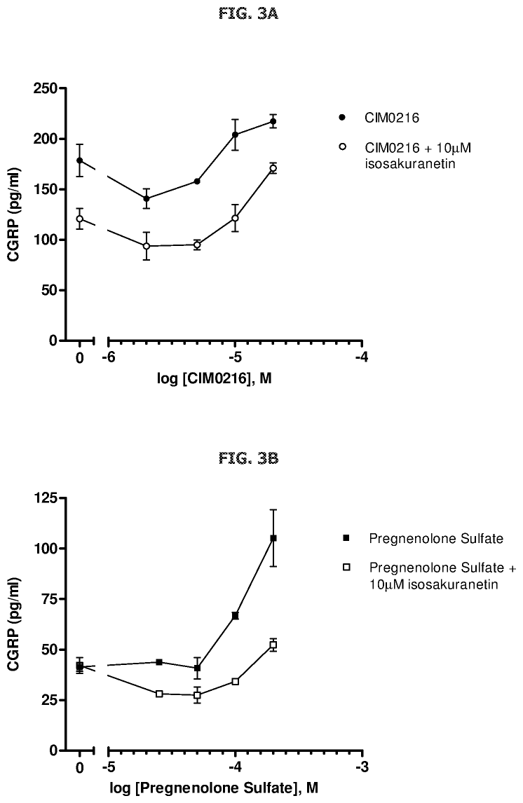

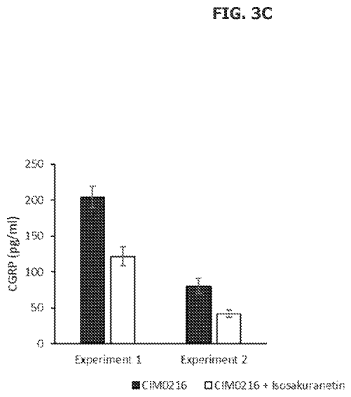

To test whether activation of TRPM3 leads to neuropeptide release, cultured DRG and TG neurons were incubated with TRPM3 agonists CIM0216 or pregnenolone sulfate. CIM0216 induced a concentration dependent release of CGRP from DRG (FIGS. 1A and B) and TG (FIG. 3A) neurons. DRG and TG cultures were also responsive to pregnenolone sulfate, resulting in an increased release of CGRP; while a single concentration of PS (100 μM) was tested in DRG cultures (FIG. 1A), the response was demonstrated to be concentration dependent in TG cultures (FIG. 3B).

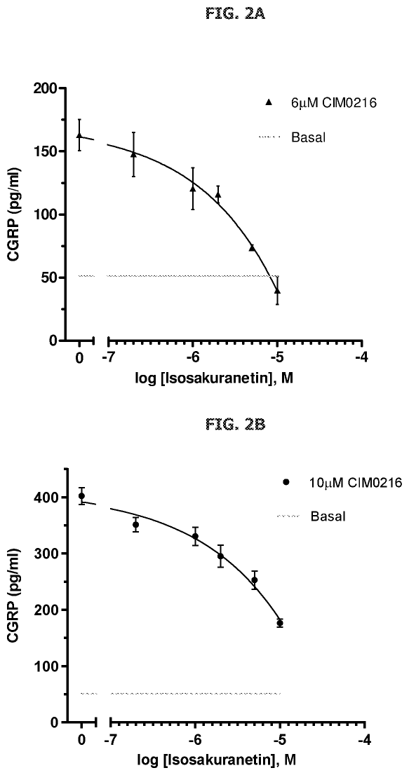

The ability of TRPM3 inhibitor isosakuranetin to antagonise CGRP release induced by CIM0216 and PS was evaluated. Pre-incubation with isosakuranetin inhibited the response to both agonists in DRG (FIGS. 1A, B and C), and TG neuron cultures (FIGS. 3A, B and C). TRPM3 activation in DRG neurons with either 6 μM or 10 μM CIM0216 (FIG. 2A, 2B respectively) was antagonised by isosakuranetin in a concentration dependent manner, resulting in reduced release of CGRP. In addition, isosakuranetin, 10 PM, reduced the release of CGRP to basal levels or below in the presence of 6 μM CIM0216 (FIG. 2A) or 100 μM PS (FIG. 1A) in DRG cultures, or in the presence of CIM0216 (5100 PM) or PS (5100 μM) in TG cultures (FIGS. 3A and B). These results demonstrate that activation of TRPM3 in DRG and TG neurons with synthetic ligand CIM0216 or neurosteroid pregnenolone sulfate leads to release of CGRP.

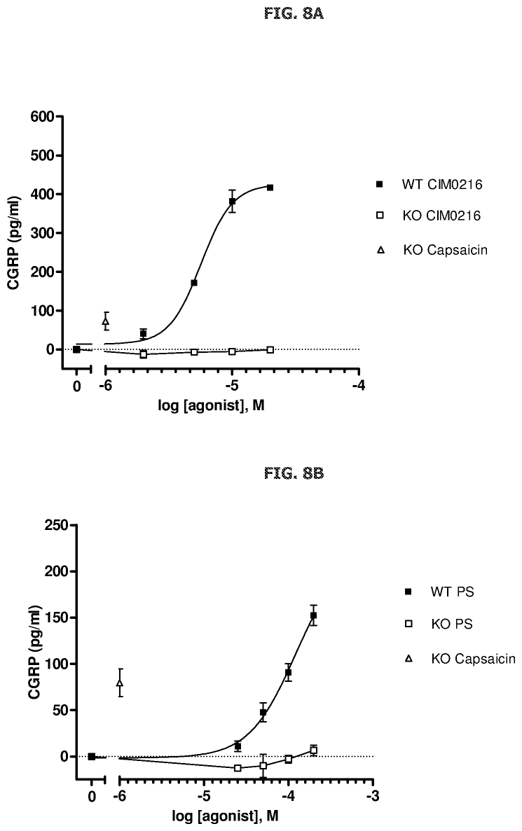

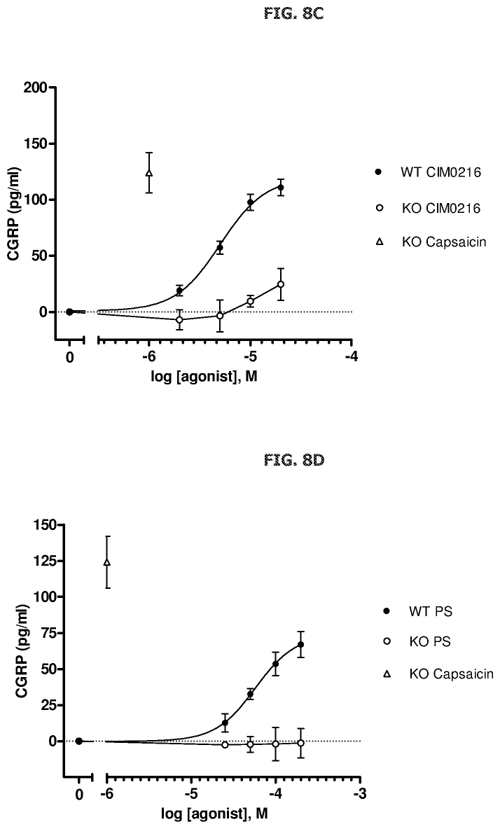

The dose-dependent release of CGRP observed in wild type sensory neurons in response to pregnenolone sulfate and CIM0216 was absent from neurons derived from TRPM3 deficient animals (FIG. 8A-D). CGRP release from these neurons could be induced by capsaicin, a TRPV1 agonist, showing that CGRP release to stimuli of other TRP channels is maintained. These data demonstrate that CGRP release induced by PS and CIM0216 is dependent on TRPM3.

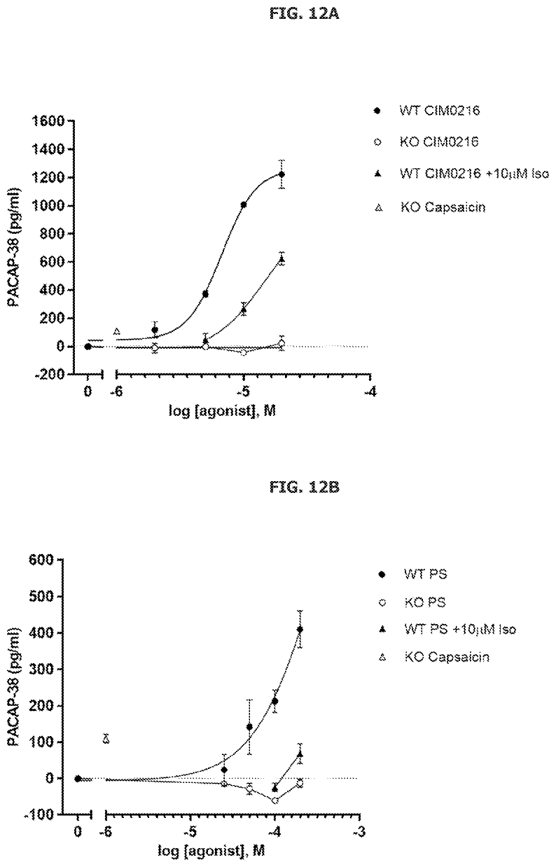

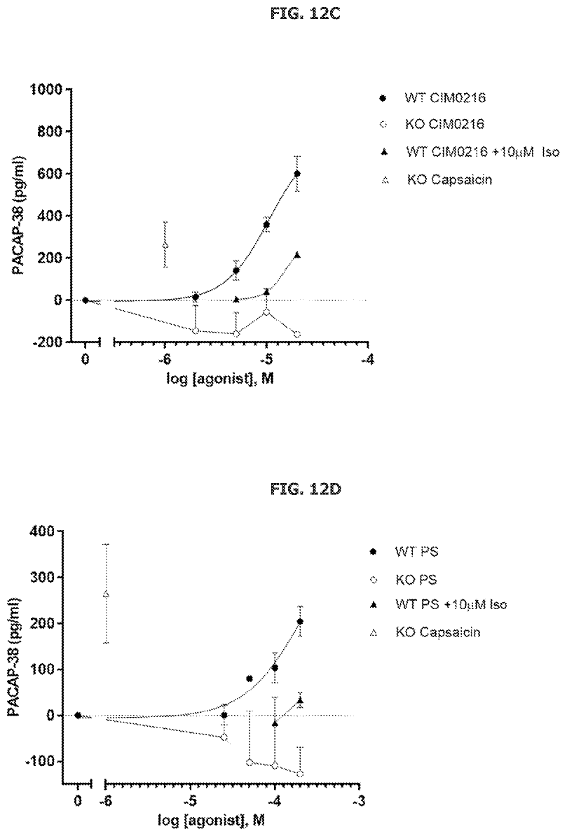

CIM0216 induced a concentration-dependent release of PACAP from rat TG cells (FIG. 11A). FIG. 11B demonstrates concentration-dependent inhibition of the CIM0216 response by TRPM3 antagonist, isosakuranetin. FIG. 12 shows activation of TRPM3 results in the release of PACAP from mouse isolated DRG (FIG. 12A, 12B) and TG cells (FIG. 12C, 12D). These responses were absent from TRPM3 knockout mouse cells and inhibited by the TRPM3 antagonist isosakuranetin (FIG. 12A-D). Cells derived from Trpm3 knockout (FIG. 12, A-D) animals were still responsive to the TRPV1 agonist, capsaicin, showing that PACAP release to stimuli of other TRP channels is maintained.

These data demonstrate that PACAP release induced by pregnenolone sulfate and CIM0216 is dependent on TRPM3.

Example 3—Calcium mobilisation assay and cell lines Calcium mobilisation assays were performed in HEK MSR II cells loaded with the calcium indicator dye Fluo4. The cells were induced to express TRPM3 (SEQ ID NO: 2) or mutants thereof by transducing them with Bacmam virus containing the codon optimised cDNA sequence for the required TRPM3 variant at a multiplicity of infection of approximately 40 for 48 hours prior to the experiment. Cells were incubated in the presence of FLUO4-AM and TRPM3 inhibitors for approx 1.5 hrs prior to transfer to a FLIPR where the cells were treated with TRPM3 agonists to induce calcium mobilisation. Fluo4 fluorescence was monitored for 10 min. As a positive control the cells were then treated with the calcium ionophore ionomycin and the fluorescence monitored for a further 3 min.

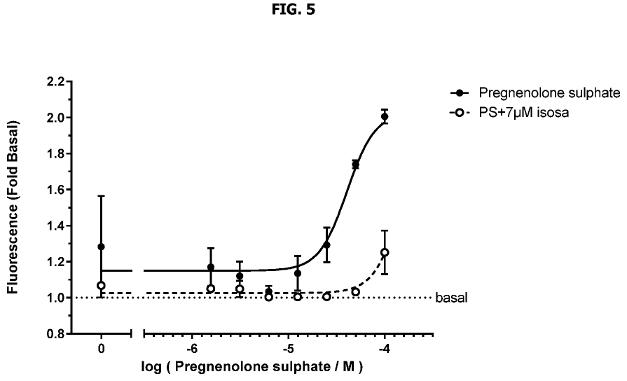

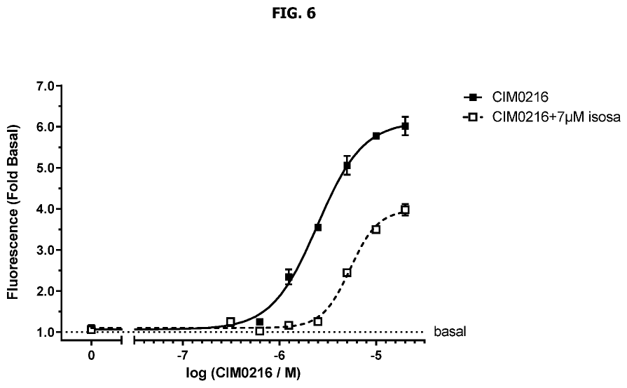

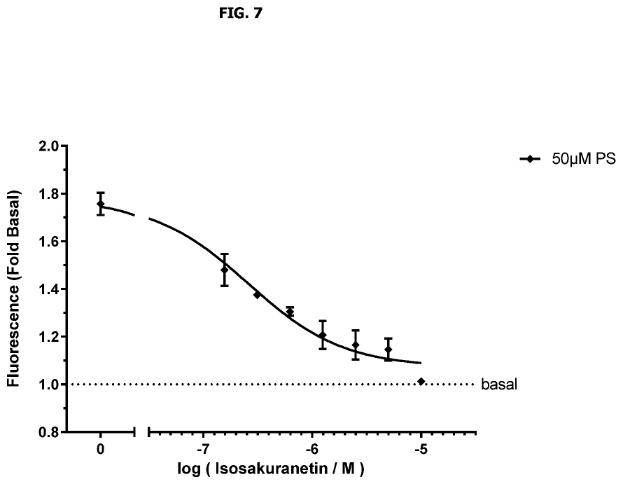

FIGS. 5, 6, 7 show results in calcium mobilisation assays using pregnenelone sulfate (FIGS. 5 and 7) and CIM0216 (FIG. 6) as TRPM3 agonists, and isosakuranetin as a TRPM3 inhibitor.

FIG. 9 shows pregnenolone sulfate induced concentration-dependent increases in FLUO4 fluorescence in cells expressing canonical TRPM3 (SEQ NO: 2) and a variant of SEQID NO: 2 having the R1670Q mutation. When transduced with equal multiplicities of infection of the relevant Bacmam viruses, the potency of pregnenolone sulfate was 1.8-fold greater at the R1670Q variant than at canonical and the maximal fold change in fluorescence was 26% larger. Thus pregnenolone sulfate is more able to activate the TRPM3 variant associated with increased likelihood of migraine diagnosis than the canonical form of the channel.

FIG. 13 shows the agonist effect of the isomers of CIM0216 in a calcium mobilisation assay. The results show that the R-isomer ((R)-2-(3,4-dihydroquinolin-1(2H)-yl)-N-(5-methylisoxazol-3-yl)propenamide) is a more potent agonist compared to the S-isomer ((S)-2-(3,4-dihydroquinolin-1(2H)-yl)-N-(5-methylisoxazol-3-yl)propenamide).

### Example 4-5 Day Rat Dural Infusion Migraine Model

The five-day rat Dural infusion migraine model is based on repeated inflammatory dural stimulation to mimic the repeated activation of dural afferents believed to occur in patients with recurrent migraine headache. In this model, rats are tested in the periorbital region for mechanical allodynia (a pain response to stimuli which are not normally painful) using von Frey fibres, which are small calibrated fibres which deliver a calibrated amount of force. Historical data in this model shows mechanical nociception sensitivity, which is alleviated with sumatriptan and anti-CGRP therapies, current standard of care compounds, suggesting this model has clinical translation.

The surgery, infusion, and testing followed the modification of the method of Oshinsky & Gomoncharconsiri, 2007, supra. Briefly, rats were habituated and trained for 1-2 weeks and then fitted with a stainless-steel cannula (22GA, Plastics One) under isoflurane anaesthesia. A craniotomy was performed using lambda as a reference (the junction of the superior sagittal sinus and transverse sinus) by advancing 1.5 mm right of lambda and 1 mm anterior towards bregma via a 1 mm hole that was made in the skull to expose the dura. Special attention was given not to disturb the dura. A custom flange guide cannula (22GA, Plastics One) was inserted into the hole (cut 0.5 mm below pedestal). The cannula was fixed to the bone with small screws and dental cement. A dummy that extended just past the end of the cannula was inserted to prevent scar tissue from forming, and thus clogging the cannula. Animals were allowed 1-2 weeks of recovery before testing and infusions began.

Periorbital thresholds were monitored during the recovery period to ensure the thresholds returned to pre-surgery baselines. If animals did not return to baseline, they were excluded from the study. Extra animals were included to account for any post-surgery animal that needed to be excluded.

After recovering from surgery, animals were infused supra-durally with treatments according to Table 1 for 5 consecutive days. Under isoflurane, the animal's nose was place in the nose cone of the anaesthesia machine. Using a haemostat, the base of the flange cannula was clasped, and the dummy cannula was removed. A custom cannula injector was inserted into the flange cannula. With a Hamilton syringe and infusion pump at a rate of 1.759 μl/min 15 μl of formulations was delivered supra-durally.

Animals were randomly assigned to a treatment group (A-C) using a software generated randomization scheme. From the first day of sensitization, each animal was tested routinely (every 2-3 days) for changes in periorbital sensitivity by a blinded investigator.

Pre-infusion sensory testing occurred on Day 1 of the testing schedule to provide a point of comparison for subsequent testing. Sensory testing occurred according to the testing schedule established in Table 1, and prior to infusion when applicable. Sensory testing utilized von Frey filaments with reproducible calibrated buckling forces varying from 0.4-10 g utilizing the Chaplan up and down method. Allodynia was tested by perpendicularly touching the periorbital region causing slight buckling of the filament for approximately 5 seconds. Based on the response pattern and the force of the final filament, the periorbital threshold (g) was calculated (Chaplan et al., 1994, Quantitative assessment of tactile allodynia in the rat paw. Journal of neuroscience methods, 53(1), 55-63). The supra-dural infusions were above the dura in the right brain hemisphere; therefore, the right periorbital threshold data only was recorded. A positive response was characterized by several behavioural criteria: stroking the face with a forepaw, head withdrawal from the stimulus, and head shaking.

| | | FINAL | DOSE | | | | EVALU- |
|---|---|---|---|---|---|---|---|
| | TREAT- | GROUP | (MG/ | DAYS OF | DOSE | | ATIONS/ |
| GROUP | MENT | SIZE | ML) | INFUSIONS | VOL | ROUTE | ENDPOINTS |
| 1 | Vehicle | 4 | 0 | 5 (Days 1-5) | 15 μl | Supra- | Periorbital von |
| | (PEG400: | | | | | dural | Frey at: |
| | KLEPTOSE) | | | | | | Pre-Surgery |
| | 0.5:99.5 | | | | | | Post-Surgery |
| | (v/v) | | | | | | During |
| | PEG400:10% | | | | | | Infusion Series: |
| | (w/v) | | | | | | Days |
| | KLEPTOSE | | | | | | 1*, 3*, 5*, 8, |
| | in saline | | | | | | 10, 12, 15, 17, |
| | | | | | | | 19. |
| 2 | Pregnenolone | 4 | 2 | | 15 μl | | * VF testing |
| | sulfate | | | | | | occurred prior |
| 3 | CIM0216 | 3* | 0.075 | | 15 μl | | to infusion |

*one animal was omitted due to cannula loss post-surgery.

Rats that received dural infusions of the vehicle for five days, maintained sensitivity to von Frey thresholds similar to the pre infusion baseline apart from an approximate 18% reduction on day five. This was a transient occurrence and sensitivity quickly returned to baseline levels by day 10.

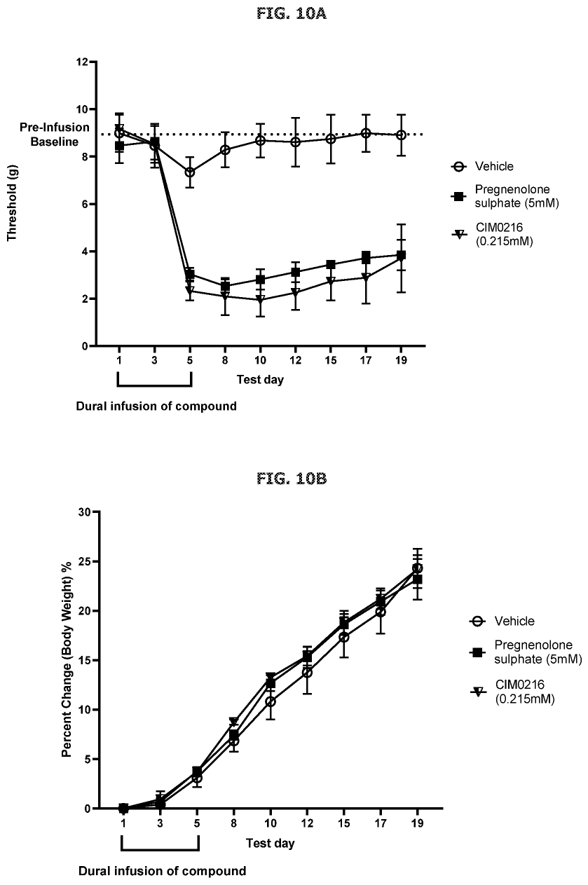

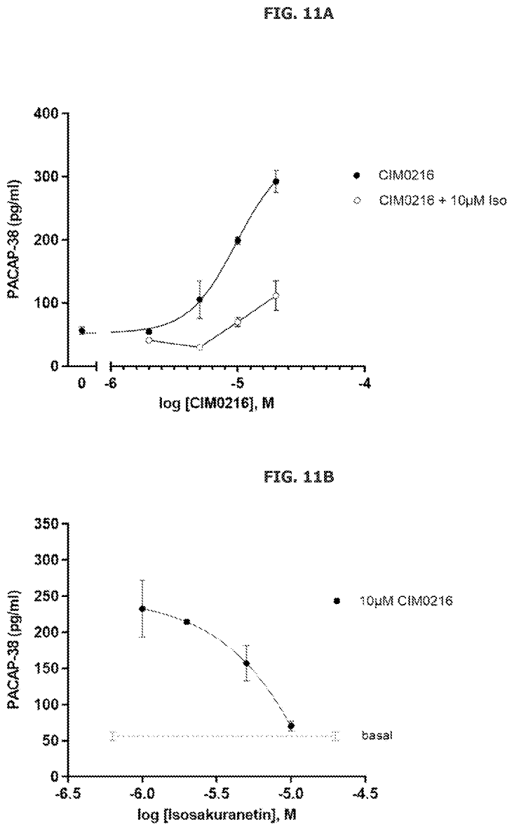

TRPM3 agonists when administered durally for five days evoked an allodynic response to von Frey filaments in rats. Rats treated with the TRPM3 agonists at lower thresholds than in vehicle treated rats (FIG. 10A). This was sustained and did not return to baseline levels for the duration of the experiment (19 days). Animals showed no overt, lasting adverse effects to the agonists and were feeding and grooming normally. This can be seen in FIG. 10B as TRPM3 agonist treated groups increase in body weight with the same trend as vehicle treated animals.

Sensitivity to non-noxious stimuli is referred to as allodynia and has been shown as a potential clinical correlate in migraine patients. Patients with chronic or transformed migraine exhibit facial allodynia even on days when they do not have a headache (Cooke et al., (2007). Cutaneous allodynia in transformed migraine patients. Headache: The Journal of Head and Face Pain, 47(4), 531-539). Example 4 is evidence that repeated activation by TRPM3 agonists can induce sensitisation in the rodent to a mechanical stimulus, that is similar to a pain assessment used in migraine patients.

### Example 5—Patient Population (Non-Responders to CGRP Treatment)

Methods: Data Collection, CGRP-Responder Status by TRPM3 Genotype

23andMe customers, who are genotyped using SNP arrays such that their TRPM3 genetic status at amino acid 1670 is known, were invited by email to complete a Headache and Migraine survey. 23andMe's “Headache and Migraine” survey asks questions on self-reported calcitonin gene-related peptide (CGRP) medication efficacy as follows—A single, check-box question that reads: “Have you ever taken any of the following calcitonin gene-related peptide (CGRP) medications to relieve or prevent your headache or migraine pain?Please check all that apply.”
[ ] Aimovig® or erenumab
[ ] Emgality® or galcanezumab
[ ] Ajovy® or fremanezumab
[ ] Nurtec® or rimegepant
[ ] Vyepti® or eptinezumab
[ ] Ubrelvy® or ubrogepant
[ ] Qulipta® or atogepant
[ ] Other
[ ] None of the above
[ ] Not sure
For each of the 8 medications (7 listed by name in addition to ‘Other’) listed in the previous question, the survey respondent would see an efficacy question that reads: “How effective was _<medication X>_ in helping to relieve or prevent your headache or migraine pain?”
   Not at all
   Slightly
   Moderately
   Very
   Extremely

In order to assess CGRP medication efficacy, data were pulled from only the baseline instance of the “Headache and Migraine” survey. Analysis was limited to individuals who answered at least one of the 8 CGRP medication efficacy questions, have measured SNP dosage data (i.e. TRPM3 status is known), and have consented to participate in 23andMe research. Those who answered more than one CGRP medication efficacy question were assigned an overall efficacy level equal to their maximum response (‘extremely’ >‘very’ >‘moderately’ >‘slightly’ >‘not at all’).

CGRP medication efficacy responses were binarized into “responder(+)” and “never-responder(−)”. “NEVER respond to anti-CGRP medication” individuals were defined as follows:
Responder(+): response was one of the following: ‘Slightly’, ‘moderately’, ‘very’, ‘extremely’
Non-responder(−): response was ‘Not at all’
Individuals were then stratified by TRPM3 status. The number of ‘migraine high-risk’ alleles in each individual (corresponding to the coding variant for 1670Gln) was scored as 0, 1 or 2 depending on allele dosage. Data were compiled in a pivot table, with aggregate data of CGRP responders stratified by TRPM3 status.

Results:

| Anti-CGRP responder vs. | Anti-CGRP Other | Anti-CGRP *Never* |
|---|---|---|
| TRPM3 status | than ‘never’ respond | respond |
| TRPM3 “high risk” allele | 101 | 8 |
| count 0 | | |
| TRPM3 “high risk” allele | 260 | 28 |
| count 1 | | |
| TRPM3 “high risk” allele | 159 | 22 |
| count 2 | | |

Example 5 is evidence that a population exists that does not respond to CGRP antagonism medication. These individuals could be a target population for a TRPM3 blocker therapy. Of the identified population, 50/58 have at least one migraine high-risk allele, suggesting a higher likelihood of responding to anti-TRPM3 migraine therapy.

SEQUENCE LISTING

SEQ ID NO: 1: Amino acid sequence of human TRPM3 isoform X11
MPEPWGTVYFLGIAQVFSFLFSWWNLEGVMNQADAPRPLNWTIRKLCHAAFLPSVRLLK
AQKSWIERAFYKRECVHIIPSTKDPHRCCCGRLIGQHVGLTPSISVLQNEKNESRLSRNDI
QSEKWSISKHTQLSPTDAFGTIEFQGGGHSNKAMYVRVSFDTKPDLLLHLMTKEWQLEL
PKLLISVHGGLQNFELQPKLKQVFGKGLIKAAMTTGAWIFTGGVNTGVIRHVGDALKDHA
SKSRGKICTIGIAPWGIVENQEDLIGRDVVRPYQTMSNPMSKLTVLNSMHSHFILADFiNG
TTGKYGAEVKLRRQLEKHISLQKINTRCLPFFSLDSRLFYSFWGSCQLDSVGIGQGVPVV
ALIVEGGPNVISIVLEYLRDTPPVPVVVCDGSGRASDILAFGHKYSEEGGLINESLRDQLLV
TIQKTFTYTRTQAQHLFIILMECMKKKELITVFRMGSEGHQDIDLAILTALLKGANASAPDQ
LSLALAWNRVDIARSQIFIYGQQWPVGSLEQAMLDALVLDRVDFVKLLIENGVSMHRFLTI
SRLEELYNTRHGPSNTLYHLVRDVKKGNLPPDYRISLIDIGLVIEYLMGGAYRCNYTRKRF
RTLYHNLFGPKRPKALKLLGMEDDIPLRRGRKTTKKREEEVDIDLDDPEINHFPFPFHELM
VWAVLMKRQKMALFFWQHGEEAMAKALVACKLCKAMAHEASENDMVDDISQELNHNS
RDFGQLAVELLDQSYKQDEQLAMKLLTYELKNWSNATCLQLAVAAKHRDFIAHTCSQML
LTDMWMGRLRMRKNSGLKVILGILLPPSILSLEFKNKDDMPYMSQAQEIHLQEKEAEEPE
KPTKEKEEEDMELTAMLGRNNGESSRKKDEEEVQSKHRLIPLGRKIYEFYNAPIVKFWFY
TLAYIGYLMLFNYIVLVKMERWPSTQEWIVISYIFTLGIEKMREILMSEPGKLLQKVKVWLQ
EYWNVTDLIAILLFSVGMILRLQDQPFRSDGRVIYCVNIIYWYIRLLDIFGVNKYLGPYVMMI
GKMMIDMMYFVIIMLVVLMSFGVARQAILFPNEEPSWKLAKNIFYMPYWMIYGEVFADQI
DPPCGQNETREDGKIIQLPPCKTGAWIVPAIMACYLLVANILLVNLLIAVFNNTFFEVKSISN
QVWKFQRYQLIMTFHERPVLPPPLIIFSHMTMIFQHLCCRWRKHESDPDERDYGLKLFIT
DDELKKVHDFEEQCIEEYFREKDDRFNSSNDERIRVTSERVENMSMRLEEVNEREHSMK
ASLQTVDIRLAQLEDLIGRMATALERLTGLERAESNKIRSRTSSDCTDAAYIVRQSSFNSQ
EGNTFKLQESIDPAGEETMSPTSPTLMPRMRSHSFYSVNMKDKGGIEKLESIFKERSLSL
HRATSSHSVAKEPKAPAAPANTLAIVPDSRRPSSCIDIYVSAMDELHCDIDPLDNSVNILGL
GEPSFSTPVPSTAPSSSAYATLAPTDRPPSRSIDFEDITSMDTRSFSSDYTHLPECQNPW
DSEPPMYHTIERSKSSRYLATTPFLLEEAPIVKSHSFMFSPSRSYYANFGVPVKTAEYTSI
TDCIDTRCVNAPQAIADRAAFPGGLGDKVEDLTCCHPEREAELSHPSSDSEENEAKGRR
ATIAISSQEGDNSERTLSNNITVPKIERANSYSAEEPSAPYAHTRKSFSISDKLDRQRNTAS
LRNPFQRSKSSKPEGRGDSLSMRRLSRTSAFQSFESKHN
SEQ ID NO: 2: Amino acid sequence of human TRPM3 isoform k
MPEPWGTVYFLGIAQVFSFLFSWWNLEGVMNQADAPRPLNWTIRKLCHAAFLPSVRLLK
AQKSWIERAFYKRECVHIIPSTKDPHRCCCGRLIGQHVGLTPSISVLQNEKNESRLSRNDI
QSEKWSISKHTQLSPTDAFGTIEFQGGGHSNKAMYVRVSFDTKPDLLLHLMTKEWQLEL
PKLLISVHGGLQNFELQPKLKQVFGKGLIKAAMTTGAWIFTGGVNTGVIRHVGDALKDHA
SKSRGKICTIGIAPWGIVENQEDLIGRDVVRPYQTMSNPMSKLTVLNSMHSHFILADNGTT
GKYGAEVKLRRQLEKHISLQKINTRIGQGVPVVALIVEGGPNVISIVLEYLRDTPPVPVVVC
DGSGRASDILAFGHKYSEEGGLINESLRDQLLVTIQKTFTYTRTQAQHLFIILMECMKKKEL
ITVFRMGSEGHQDIDLAILTALLKGANASAPDQLSLALAWNRVDIARSQIFIYGQQWPVGS
LEQAMLDALVLDRVDFVKLLIENGVSMHRFLTISRLEELYNTRHGPSNTLYHLVRDVKKG
NLPPDYRISLIDIGLVIEYLMGGAYRCNYTRKRFRTLYHNLFGPKRPKALKLLGMEDDIPLR
RGRKTTKKREEEVDIDLDDPEINHFPFPFHELMVWAVLMKRQKMALFFWQHGEEAMAK
ALVACKLCKAMAHEASENDMVDDISQELNHNSRDFGQLAVELLDQSYKQDEQLAMKLLT
YELKNWSNATCLQLAVAAKHRDFIAHTCSQMLLTDMWMGRLRMRKNSGLKVILGILLPP
SILSLEFKNKDDMPYMSQAQEIHLQEKEAEEPEKPTKEKEEEDMELTAMLGRNNGESSR
KKDEEEVQSKHRLIPLGRKIYEFYNAPIVKFWFYTLAYIGYLMLFNYIVLVKMERWPSTQE
WIVISYIFTLGIEKMREILMSEPGKLLQKVKVWLQEYWNVTDLIAILLFSVGMILRLQDQPFR
SDGRVIYCVNIIYWYIRLLDIFGVNKYLGPYVMMIGKMMIDMMYFVIIMLVVLMSFGVARQA
ILFPNEEPSWKLAKNIFYMPYWMIYGEVFADQIDPPCGQNETREDGKIIQLPPCKTGAWIV
PAIMACYLLVANILLVNLLIAVFNNTFFEVKSISNQVWKFQRYQLIMTFHERPVLPPPLIIFS
HMTMIFQHLCCRWRKHESDPDERDYGLKLFITDDELKKVHDFEEQCIEEYFREKDDRFN
SSNDERIRVTSERVENMSMRLEEVNEREHSMKASLQTVDIRLAQLEDLIGRMATALERLT
GLERAESNKIRSRTSSDCTDAAYIVRQSSFNSQEGNTFKLQESIDPAGEETMSPTSPTLM
PRMRSHSFYSVNMKDKGGIEKLESIFKERSLSLHRATSSHSVAKEPKAPAAPANTLAIVP
DSRRPSSCIDIYVSAMDELHCDIDPLDNSVNILGLGEPSFSTPVPSTAPSSSAYATLAPTD
RPPSRSIDFEDITSMDTRSFSSDYTHLPECQNPWDSEPPMYHTIERSKSSRYLATTPFLL
EEAPIVKSHSFMFSPSRSYYANFGVPVKTAEYTSITDCIDTRCVNAPQAIADRAAFPGGLG
DKVEDLTCCHPEREAELSHPSSDSEENEAKGRRATIAISSQEGDNSERTLSNNITVPKIER
ANSYSAEEPSAPYAHTRKSFSISDKLDRQRNTASLRNPFQRSKSSKPEGRGDSLSMRRL
SRTSAFQSFESKHN
SEQ ID NO: 3: Amino acid sequence of human TRPM3 isoform m
MPEPWGTVYFLGIAQVFSFLFSWWNLEGVMNQADAPRPLNWTIRKLCHAAFLPSVRLLK
AQKSWIERAFYKRECVHIIPSTKDPHRCCCGRLIGQHVGLTPSISVLQNEKNESRLSRNDI
QSEKWSISKHTQLSPTDAFGTIEFQGGGHSNKAMYVRVSFDTKPDLLLHLMTKEWQLEL
PKLLISVHGGLQNFELQPKLKQVFGKGLIKAAMTTGAWIFTGGVNTGVIRHVGDALKDHA
SKSRGKICTIGIAPWGIVENQEDLIGRDVVRPYQTMSNPMSKLTVLNSMHSHFILADNGTT
GKYGAEVKLRRQLEKHISLQKINTRIGQGVPVVALIVEGGPNVISIVLEYLRDTPPVPVVVC
DGSGRASDILAFGHKYSEEGGLINESLRDQLLVTIQKTFTYTRTQAQHLFIILMECMKKKEL
ITVFRMGSEGHQDIDLAILTALLKGANASAPDQLSLALAWNRVDIARSQIFIYGQQWPVGS
LEQAMLDALVLDRVDFVKLLIENGVSMHRFLTISRLEELYNTRHGPSNTLYHLVRDVKKRE
YPGFGWIYFKGNLPPDYRISLIDIGLVIEYLMGGAYRCNYTRKRFRTLYHNLFGPKRPKAL
KLLGMEDDIPLRRGRKTTKKREEEVDIDLDDPEINHFPFPFHELMVWAVLMKRQKMALFF
WQHGEEAMAKALVACKLCKAMAHEASENDMVDDISQELNHNSRDFGQLAVELLDQSYK
QDEQLAMKLLTYELKNWSNATCLQLAVAAKHRDFIAHTCSQMLLTDMWMGRLRMRKNS
GLKVILGILLPPSILSLEFKNKDDMPYMSQAQEIHLQEKEAEEPEKPTKEKEEEDMELTAM
LGRNNGESSRKKDEEEVQSKHRLIPLGRKIYEFYNAPIVKFWFYTLAYIGYLMLFNYIVLVK
MERWPSTQEWIVISYIFTLGIEKMREILMSEPGKLLQKVKVWLQEYWNVTDLIAILLFSVG
MILRLQDQPFRSDGRVIYCVNIIYWYIRLLDIFGVNKYLGPYVMMIGKMMIDMMYFVIIMLV
VLMSFGVARQAILFPNEEPSWKLAKNIFYMPYWMIYGEVFADQIDPPCGQNETREDGKII
QLPPCKTGAWIVPAIMACYLLVANILLVNLLIAVFNNTFFEVKSISNQVWKFQRYQLIMTFH
ERPVLPPPLHFSHMTMIFQHLCCRWRKHESDPDERDYGLKLFITDDELKKVHDFEEQCIE
EYFREKDDRFNSSNDERIRVTSERVENMSMRLEEVNEREHSMKASLQTVDIRLAQLEDLI
GRMATALERLTGLERAESNKIRSRTSSDCTDAAYIVRQSSFNSQEGNTFKLQESIDPAGE
ETMSPTSPTLMPRMRSHSFYSVNMKDKGGIEKLESIFKERSLSLHRATSSHSVAKEPKAP
AAPANTLAIVPDSRRPSSCIDIYVSAMDELHCDIDPLDNSVNILGLGEPSFSTPVPSTAPSS
SAYATLAPTDRPPSRSIDFEDITSMDTRSFSSDYTHLPECQNPWDSEPPMYHTIERSKSS
RYLATTPFLLEEAPIVKSHSFMFSPSRSYYANFGVPVKTAEYTSITDCIDTRCVNAPQAIAD
RAAFPGGLGDKVEDLTCCHPEREAELSHPSSDSEENEAKGRRATIAISSQEGDNSERTL
SNNITVPKIERANSYSAEEPSAPYAHTRKSFSISDKLDRQRNTASLRNPFQRSKSSKPEG
RGDSLSMRRLSRTSAFQSFESKHN
SEQ ID NO: 4: Amino acid sequence of human TRPM3 isoform X19
MPEPWGTVYFLGIAQVFSFLFSWWNLEGVMNQADAPRPLNWTIRKLCHAAFLPSVRLLK
AQKSWIERAFYKRECVHIIPSTKDPHRCCCGRLIGQHVGLTPSISVLQNEKNESRLSRNDI
QSEKWSISKHTQLSPTDAFGTIEFQGGGHSNKAMYVRVSFDTKPDLLLHLMTKEWQLEL
PKLLISVHGGLQNFELQPKLKQVFGKGLIKAAMTTGAWIFTGGVNTGVIRHVGDALKDHA
SKSRGKICTIGIAPWGIVENQEDLIGRDVVRPYQTMSNPMSKLTVLNSMHSHFILADNGTT
GKYGAEVKLRRQLEKHISLQKINTRIGQGVPVVALIVEGGPNVISIVLEYLRDTPPVPVVVC
DGSGRASDILAFGHKYSEEGGLINESLRDQLLVTIQKTFTYTRTQAQHLFIILMECMKKKEL
ITVFRMGSEGHQDIDLAILTALLKGANASAPDQLSLALAWNRVDIARSQIFIYGQQWPVGS
LEQAMLDALVLDRVDFVKLLIENGVSMHRFLTISRLEELYNTRHGPSNTLYHLVRDVKKG
NLPPDYRISLIDIGLVIEYLMGGAYRCNYTRKRFRTLYHNLFGPKRDDIPLRRGRKTTKKR
EEEVDIDLDDPEINHFPFPFHELMVWAVLMKRQKMALFFWQHGEEAMAKALVACKLCKA
MAHEASENDMVDDISQELNHNSRDFGQLAVELLDQSYKQDEQLAMKLLTYELKNWSNA
TCLQLAVAAKHRDFIAHTCSQMLLTDMWMGRLRMRKNSGLKVILGILLPPSILSLEFKNKD
DMPYMSQAQEIHLQEKEAEEPEKPTKEKEEEDMELTAMLGRNNGESSRKKDEEEVQSK
HRLIPLGRKIYEFYNAPIVKFWFYTLAYIGYLMLFNYIVLVKMERWPSTQEWIVISYIFTLGIE
KMREILMSEPGKLLQKVKVWLQEYWNVTDLIAILLFSVGMILRLQDQPFRSDGRVIYCVNII
YWYIRLLDIFGVNKYLGPYVMMIGKMMIDMMYFVIIMLVVLMSFGVARQAILFPNEEPSWK
LAKNIFYMPYWMIYGEVFADQIDPPCGQNETREDGKIIQLPPCKTGAWIVPAIMACYLLVA
NILLVNLLIAVFNNTFFEVKSISNQVWKFQRYQLIMTFHERPVLPPPLIIFSHMTMIFQHLCC
RWRKHESDPDERDYGLKLFITDDELKKVHDFEEQCIEEYFREKDDRFNSSNDERIRVTSE
RVENMSMRLEEVNEREHSMKASLQTVDIRLAQLEDLIGRMATALERLTGLERAESNKIRS
RTSSDCTDAAYIVRQSSFNSQEGNTFKLQESIDPAGEETMSPTSPTLMPRMRSHSFYSV
NMKDKGGIEKLESIFKERSLSLHRATSSHSVAKEPKAPAAPANTLAIVPDSRRPSSCIDIYV
SAMDELHCDIDPLDNSVNILGLGEPSFSTPVPSTAPSSSAYATLAPTDRPPSRSIDFEDITS
MDTRSFSSDYTHLPECQNPWDSEPPMYHTIERSKSSRYLATTPFLLEEAPIVKSHSFMFS
PSRSYYANFGVPVKTAEYTSITDCIDTRCVNAPQAIADRAAFPGGLGDKVEDLTCCHPER
EAELSHPSSDSEENEAKGRRATIAISSQEGDNSERTLSNNITVPKIERANSYSAEEPSAPY
AHTRKSFSISDKLDRQRNTASLRNPFQRSKSSKPEGRGDSLSMRRLSRTSAFQSFESKH
N
SEQ ID NO: 5: Amino acid sequence of human TRPM3 isoform q
MPEPWGTVYFLGIAQVFSFLFSWWNLEGVMNQADAPRPLNWTIRKLCHAAFLPSVRLLK
AQKSWIERAFYKRECVHIIPSTKDPHRCCCGRLIGQHVGLTPSISVLQNEKNESRLSRNDI
QSEKWSISKHTQLSPTDAFGTIEFQGGGHSNKAMYVRVSFDTKPDLLLHLMTKEWQLEL
PKLLISVHGGLQNFELQPKLKQVFGKGLIKAAMTTGAWIFTGGVNTGVIRHVGDALKDHA
SKSRGKICTIGIAPWGIVENQEDLIGRDVVRPYQTMSNPMSKLTVLNSMHSHFILADNGTT
GKYGAEVKLRRQLEKHISLQKINTRIGQGVPVVALIVEGGPNVISIVLEYLRDTPPVPVVVC
DGSGRASDILAFGHKYSEEGGLINESLRDQLLVTIQKTFTYTRTQAQHLFIILMECMKKKEL
ITVFRMGSEGHQDIDLAILTALLKGANASAPDQLSLALAWNRVDIARSQIFIYGQQWPVGS
LEQAMLDALVLDRVDFVKLLIENGVSMHRFLTISRLEELYNTRHGPSNTLYHLVRDVKKRE
YPGFGWIYFKGNLPPDYRISLIDIGLVIEYLMGGAYRCNYTRKRFRTLYHNLFGPKRDDIPL
RRGRKTTKKREEEVDIDLDDPEINHFPFPFHELMVWAVLMKRQKMALFFWQHGEEAMA
KALVACKLCKAMAHEASENDMVDDISQELNHNSRDFGQLAVELLDQSYKQDEQLAMKLL
TYELKNWSNATCLQLAVAAKHRDFIAHTCSQMLLTDMWMGRLRMRKNSGLKVILGILLPP
SILSLEFKNKDDMPYMSQAQEIHLQEKEAEEPEKPTKEKEEEDMELTAMLGRNNGESSR
KKDEEEVQSKHRLIPLGRKIYEFYNAPIVKFWFYTLAYIGYLMLFNYIVLVKMERWPSTQE
WIVISYIFTLGIEKMREILMSEPGKLLQKVKVWLQEYWNVTDLIAILLFSVGMILRLQDQPFR
SDGRVIYCVNIIYWYIRLLDIFGVNKYLGPYVMMIGKMMIDMMYFVIIMLVVLMSFGVARQA
ILFPNEEPSWKLAKNIFYMPYWMIYGEVFADQIDPPCGQNETREDGKIIQLPPCKTGAWIV
PAIMACYLLVANILLVNLLIAVFNNTFFEVKSISNQVWKFQRYQLIMTFHERPVLPPPLIIFS
HMTMIFQHLCCRWRKHESDPDERDYGLKLFITDDELKKVHDFEEQCIEEYFREKDDRFN
SSNDERIRVTSERVENMSMRLEEVNEREHSMKASLQTVDIRLAQLEDLIGRMATALERLT
GLERAESNKIRSRTSSDCTDAAYIVRQSSFNSQEGNTFKLQESIDPAGEETMSPTSPTLM
PRMRSHSFYSVNMKDKGGIEKLESIFKERSLSLHRATSSHSVAKEPKAPAAPANTLAIVP
DSRRPSSCIDIYVSAMDELHCDIDPLDNSVNILGLGEPSFSTPVPSTAPSSSAYATLAPTD
RPPSRSIDFEDITSMDTRSFSSDYTHLPECQNPWDSEPPMYHTIERSKSSRYLATTPFLL
EEAPIVKSHSFMFSPSRSYYANFGVPVKTAEYTSITDCIDTRCVNAPQAIADRAAFPGGLG
DKVEDLTCCHPEREAELSHPSSDSEENEAKGRRATIAISSQEGDNSERTLSNNITVPKIER
ANSYSAEEPSAPYAHTRKSFSISDKLDRQRNTASLRNPFQRSKSSKPEGRGDSLSMRRL
SRTSAFQSFESKHN
SEQ ID NO: 6: Amino acid sequence of human TRPM3 isoform Q9HCF6-6
MPEPWGTVYFLGIAQVFSFLFSWWNLEGVMNQADAPRPLNWTIRKLCHAAFLPSVRLLK
AQKSWIERAFYKRECVHIIPSTKDPHRCCCGRLIGQHVGLTPSISVLQNEKNESRLSRNDI
QSEKWSISKHTQLSPTDAFGTIEFQGGGHSNKAMYVRVSFDTKPDLLLHLMTKEWQLEL
PKLLISVHGGLQNFELQPKLKQVFGKGLIKAAMTTGAWIFTGGVNTGVIRHVGDALKDHA
SKSRGKICTIGIAPWGIVENQEDLIGRDVVRPYQTMSNPMSKLTVLNSMHSHFILADNGTT
GKYGAEVKLRRQLEKHISLQKINTRIGQGVPVVALIVEGGPNVISIVLEYLRDTPPVPVVVC
DGSGRASDILAFGHKYSEEGGLINESLRDQLLVTIQKTFTYTRTQAQHLFIILMECMKKKEL
ITVFRMGSEGHQDIDLAILTALLKGANASAPDQLSLALAWNRVDIARSQIFIYGQQWPVGS
LEQAMLDALVLDRVDFVKLLIENGVSMHRFLTISRLEELYNTRHGPSNTLYHLVRDVKKG
NLPPDYRISLIDIGLVIEYLMGGAYRCNYTRKRFRTLYHNLFGPKRPKALKLLGMEDDIPLR
RGRKTTKKREEEVDIDLDDPEINHFPFPFHELMVWAVLMKRQKMALFFWQHGEEAMAK
ALVACKLCKAMAHEASENDMVDDISQELNHNSRDFGQLAVELLDQSYKQDEQLAMKLLT
YELKNWSNATCLQLAVAAKHRDFIAHTCSQMLLTDMWMGRLRMRKNSGLKVILGILLPP
SILSLEFKNKDDMPYMSQAQEIHLQEKEAEEPEKPTKEKEEEDMELTAMLGRNNGESSR
KKDEEEVQSKHRLIPLGRKIYEFYNAPIVKFWFYTLAYIGYLMLFNYIVLVKMERWPSTQE
WIVISYIFTLGIEKMREILMSEPGKLLQKVKVWLQEYWNVTDLIAILLFSVGMILRLQDQPFR
SDGRVIYCVNIIYWYIRLLDIFGVNKYLGPYVMMIGKMMIDMMYFVIIMLVVLMSFGVARQA
ILFPNEEPSWKLAKNIFYMPYWMIYGEVFADQIDRKQVYDSHTPKSAPCGQNETREDGKI
IQLPPCKTGAWIVPAIMACYLLVANILLVNLLIAVFNNTFFEVKSISNQVWKFQRYQLIMTFH
ERPVLPPPLHFSHMTMIFQHLCCRWRKHESDPDERDYGLKLFITDDELKKVHDFEEQCIE
EYFREKDDRFNSSNDERIRVTSERVENMSMRLEEVNEREHSMKASLQTVDIRLAQLEDLI
GRMATALERLTGLERAESNKIRSRTSSDCTDAAYIVRQSSFNSQEGNTFKLQESIDPAGE
ETMSPTSPTLMPRMRSHSFYSVNMKDKGGIEKLESIFKERSLSLHRATSSHSVAKEPKAP
AAPANTLAIVPDSRRPSSCIDIYVSAMDELHCDIDPLDNSVNILGLGEPSFSTPVPSTAPSS
SAYATLAPTDRPPSRSIDFEDITSMDTRSFSSDYTHLPECQNPWDSEPPMYHTIERSKSS
RYLATTPFLLEEAPIVKSHSFMFSPSRSYYANFGVPVKTAEYTSITDCIDTRCVNAPQAIAD
RAAFPGGLGDKVEDLTCCHPEREAELSHPSSDSEENEAKGRRATIAISSQEGDNSERTL
SNNITVPKIERANSYSAEEPSAPYAHTRKSFSISDKLDRQRNTASLRNPFQRSKSSKPEG
RGDSLSMRRLSRTSAFQSFESKHN
SEQ ID NO: 7: Amino acid sequence of human TRPM3 isoform X12
MPEPWGTVYFLGIAQVFSFLFSWWNLEGVMNQADAPRPLNWTIRKLCHAAFLPSVRLLK
AQKSWIERAFYKRECVHIIPSTKDPHRCCCGRLIGQHVGLTPSISVLQNEKNESRLSRNDI
QSEKWSISKHTQLSPTDAFGTIEFQGGGHSNKAMYVRVSFDTKPDLLLHLMTKEWQLEL
PKLLISVHGGLQNFELQPKLKQVFGKGLIKAAMTTGAWIFTGGVNTGVIRHVGDALKDHA
SKSRGKICTIGIAPWGIVENQEDLIGRDVVRPYQTMSNPMSKLTVLNSMHSHFILADNGTT
GKYGAEVKLRRQLEKHISLQKINTRCLPFFSLDSRLFYSFWGSCQLDSVGIGQGVPVVALI
VEGGPNVISIVLEYLRDTPPVPVVVCDGSGRASDILAFGHKYSEEGGLINESLRDQLLVTI
QKTFTYTRTQAQHLFIILMECMKKKELITVFRMGSEGHQDIDLAILTALLKGANASAPDQLS
LALAWNRVDIARSQIFIYGQQWPVGSLEQAMLDALVLDRVDFVKLLIENGVSMHRFLTISR
LEELYNTRHGPSNTLYHLVRDVKKGNLPPDYRISLIDIGLVIEYLMGGAYRCNYTRKRFRT
LYHNLFGPKRDDIPLRRGRKTTKKREEEVDIDLDDPEINHFPFPFHELMVWAVLMKRQKM
ALFFWQHGEEAMAKALVACKLCKAMAHEASENDMVDDISQELNHNSRDFGQLAVELLD
QSYKQDEQLAMKLLTYELKNWSNATCLQLAVAAKHRDFIAHTCSQMLLTDMWMGRLRM
RKNSGLKVILGILLPPSILSLEFKNKDDMPYMSQAQEIHLQEKEAEEPEKPTKEKEEEDME
LTAMLGRNNGESSRKKDEEEVQSKHRLIPLGRKIYEFYNAPIVKFWFYTLAYIGYLMLFNY
IVLVKMERWPSTQEWIVISYIFTLGIEKMREILMSEPGKLLQKVKVWLQEYWNVTDLIAILL
FSVGMILRLQDQPFRSDGRVIYCVNIIYWYIRLLDIFGVNKYLGPYVMMIGKMMIDMMYFVI
IMLVVLMSFGVARQAILFPNEEPSWKLAKNIFYMPYWMIYGEVFADQIDPPCGQNETRED
GKIIQLPPCKTGAWIVPAIMACYLLVANILLVNLLIAVFNNTFFEVKSISNQVWKFQRYQLIM
TFHERPVLPPPLIIFSHMTMIFQHLCCRWRKHESDPDERDYGLKLFITDDELKKVHDFEEQ
CIEEYFREKDDRFNSSNDERIRVTSERVENMSMRLEEVNEREHSMKASLQTVDIRLAQLE
DLIGRMATALERLTGLERAESNKIRSRTSSDCTDAAYIVRQSSFNSQEGNTFKLQESIDPA
GEETMSPTSPTLMPRMRSHSFYSVNMKDKGGIEKLESIFKERSLSLHRATSSHSVAKEPK
APAAPANTLAIVPDSRRPSSCIDIYVSAMDELHCDIDPLDNSVNILGLGEPSFSTPVPSTAP
SSSAYATLAPTDRPPSRSIDFEDITSMDTRSFSSDYTHLPECQNPWDSEPPMYHTIERSK
SSRYLATTPFLLEEAPIVKSHSFMFSPSRSYYANFGVPVKTAEYTSITDCIDTRCVNAPQAI
ADRAAFPGGLGDKVEDLTCCHPEREAELSHPSSDSEENEAKGRRATIAISSQEGDNSER
TLSNNITVPKIERANSYSAEEPSAPYAHTRKSFSISDKLDRQRNTASLRNPFQRSKSSKPE
GRGDSLSMRRLSRTSAFQSFESKHN
SEQ ID NO: 8: Amino acid sequence of human TRPM3 isoform Q9HCF6-8
MPEPWGTVYFLGIAQVFSFLFSWWNLEGVMNQADAPRPLNWTIRKLCHAAFLPSVRLLK
AQKSWIERAFYKRECVHIIPSTKDPHRCCCGRLIGQHVGLTPSISVLQNEKNESRLSRNDI
QSEKWSISKHTQLSPTDAFGTIEFQGGGHSNKAMYVRVSFDTKPDLLLHLMTKEWQLEL
PKLLISVHGGLQNFELQPKLKQVFGKGLIKAAMTTGAWIFTGGVNTGVIRHVGDALKDHA
SKSRGKICTIGIAPWGIVENQEDLIGRDVVRPYQTMSNPMSKLTVLNSMHSHFILADNGTT
GKYGAEVKLRRQLEKHISLQKINTRIGQGVPVVALIVEGGPNVISIVLEYLRDTPPVPVVVC
DGSGRASDILAFGHKYSEEGGLINESLRDQLLVTIQKTFTYTRTQAQHLFIILMECMKKKEL
ITVFRMGSEGHQDIDLAILTALLKGANASAPDQLSLALAWNRVDIARSQIFIYGQQWPVGS
LEQAMLDALVLDRVDFVKLLIENGRHGPSNTLYHLVRDVKKGNLPPDYRISLIDIGLVIEYL
MGGAYRCNYTRKRFRTLYHNLFGPKRDDIPLRRGRKTTKKREEEVDIDLDDPEINHFPFP
FHELMVWAVLMKRQKMALFFWQHGEEAMAKALVACKLCKAMAHEASENDMVDDISQE
LNHNSRDFGQLAVELLDQSYKQDEQLAMKLLTYELKNWSNATCLQLAVAAKHRDFIAHT
CSQMLLTDMWMGRLRMRKNSGLKVILGILLPPSILSLEFKNKDDMPYMSQAQEIHLQEKE
AEEPEKPTKEKEEEDMELTAMLGRNNGESSRKKDEEEVQSKHRLIPLGRKIYEFYNAPIV
KFWFYTLAYIGYLMLFNYIVLVKMERWPSTQEWIVISYIFTLGIEKMREILMSEPGKLLQKV
KVWLQEYWNVTDLIAILLFSVGMILRLQDQPFRSDGRVIYCVNIIYWYIRLLDIFGVNKYLG
PYVMMIGKMMIDMMYFVIIMLVVLMSFGVARQAILFPNEEPSWKLAKNIFYMPYWMIYGE
VFADQIDPPCGQNETREDGKIIQLPPCKTGAWIVPAIMACYLLVANILLVNLLIAVFNNTFFE
VKSISNQVWKFQRYQLIMTFHERPVLPPPLIIFSHMTMIFQHLCCRWRKHESDPDERDYG
LKLFITDDELKKVHDFEEQCIEEYFREKDDRFNSSNDERIRVTSERVENMSMRLEEVNER
EHSMKASLQTVDIRLAQLEDLIGRMATALERLTGLERAESNKIRSRTSSDCTDAAYIVRQS
SFNSQEGNTFKLQESIDPAGEETMSPTSPTLMPRMRSHSFYSVNMKDKGGIEKLESIFKE
RSLSLHRATSSHSVAKEPKAPAAPANTLAIVPDSRRPSSCIDIYVSAMDELHCDIDPLDNS
VNILGLGEPSFSTPVPSTAPSSSAYATLAPTDRPPSRSIDFEDITSMDTRSFSSDYTHLPE
CQNPWDSEPPMYHTIERSKSSRYLATTPFLLEEAPIVKSHSFMFSPSRSYYANFGVPVKT
AEYTSITDCIDTRCVNAPQAIADRAAFPGGLGDKVEDLTCCHPEREAELSHPSSDSEENE
AKGRRATIAISSQEGDNSERTLSNNITVPKIERANSYSAEEPSAPYAHTRKSFSISDKLDR
QRNTASLRNPFQRSKSSKPEGRGDSLSMRRLSRTSAFQSFESKHN
SEQ ID NO: 9: Amino acid sequence of human TRPM3 isoform s
MPEPWGTVYFLGIAQVFSFLFSWWNLEGVMNQADAPRPLNWTIRKLCHAAFLPSVRLLK
AQKSWIERAFYKRECVHIIPSTKDPHRCCCGRLIGQHVGLTPSISVLQNEKNESRLSRNDI
QSEKWSISKHTQLSPTDAFGTIEFQGGGHSNKAMYVRVSFDTKPDLLLHLMTKEWQLEL
PKLLISVHGGLQNFELQPKLKQVFGKGLIKAAMTTGAWIFTGGVNTGVIRHVGDALKDHA
SKSRGKICTIGIAPWGIVENQEDLIGRDVVRPYQTMSNPMSKLTVLNSMHSHFILADNGTT
GKYGAEVKLRRQLEKHISLQKINTRIGQGVPVVALIVEGGPNVISIVLEYLRDTPPVPVVVC
DGSGRASDILAFGHKYSEEGGLINESLRDQLLVTIQKTFTYTRTQAQHLFIILMECMKKKEL
ITVFRMGSEGHQDIDLAILTALLKGANASAPDQLSLALAWNRVDIARSQIFIYGQQWPVGS
LEQAMLDALVLDRVDFVKLLIENGVSMHRFLTISRLEELYNTRHGPSNTLYHLVRDVKKG
NLPPDYRISLIDIGLVIEYLMGGAYRCNYTRKRFRTLYHNLFGPKRPKALKLLGMEDDIPLR
RGRKTTKKREEEVDIDLDDPEINHFPFPFHELMVWAVLMKRQKMALFFWQHGEEAMAK
ALVACKLCKAMAHEASENDMVDDISQELNHNSRDFGQLAVELLDQSYKQDEQLAMKLLT
YELKNWSNATCLQLAVAAKHRDFIAHTCSQMLLTDMWMGRLRMRKNSGLKVILGILLPP
SILSLEFKNKDDMPYMSQAQEIHLQEKEAEEPEKPTKEKEEEDMELTAMLGRNNGESSR
KKDEEEVQSKHRLIPLGRKIYEFYNAPIVKFWFYTLAYIGYLMLFNYIVLVKMERWPSTQE
WIVISYIFTLGIEKMREILMSEPGKLLQKVKVWLQEYWNVTDLIAILLFSVGMILRLQDQPFR
SDGRVIYCVNIIYWYIRLLDIFGVNKYLGPYVMMIGKMMIDMMYFVIIMLVVLMSFGVARQA
ILFPNEEPSWKLAKNIFYMPYWMIYGEVFADQIDPPCGQNETREDGKIIQLPPCKTGAWIV
PAIMACYLLVANILLVNLLIAVFNNTFFEVKSISNQVWKFQRYQLIMTFHERPVLPPPLIIFS
HMTMIFQHLCCRWRKHESDPDERDYGLKLFITDDELKKVHDFEEQCIEEYFREKDDRFN
SSNDERIRVTSERVENMSMRLEEVNEREHSMKASLQTVDIRLAQLEDLIGRMATALERLT
GLERAESNKIRSRTSSDCTDAAYIVRQSSFNSQEGNTFKLQESIDPAEHPLYSV
SEQ ID NO: 10: Amino acid sequence of human TRPM3 isoform Q9HCF6-11
MGKKWRDAAEMERGCSDREDNAESRRRSRSASRGRFAESWKRLSSKQGSTKRSGLP
SQQTPAQKSWIERAFYKRECVHIIPSTKDPHRCCCGRLIGQHVGLTPSISVLQNEKNESR
LSRNDIQSEKWSISKHTQLSPTDAFGTIEFQGGGHSNKAMYVRVSFDTKPDLLLHLMTKE
WQLELPKLLISVHGGLQNFELQPKLKQVFGKGLIKAAMTTGAWIFTGGVNTGVIRHVGDA
LKDHASKSRGKICTIGIAPWGIVENQEDLIGRDVVRPYQTMSNPMSKLTVLNSMHSHFILA
DNGTTGKYGAEVKLRRQLEKHISLQKINTRCLPFFSLDSRLFYSFWGSCQLDSVGIGQGV
PVVALIVEGGPNVISIVLEYLRDTPPVPVVVCDGSGRASDILAFGHKYSEEGGLINESLRD
QLLVTIQKTFTYTRTQAQHLFIILMECMKKKELIIVFRMGSEGHQDIDLAILIALLKGANASA
PDQLSLALAWNRVDIARSQIFIYGQQWPVGSLEQAMLDALVLDRVDFVKLLIENGVSMHR
FLTISRLEELYNTRHGPSNTLYHLVRDVKKGNLPPDYRISLIDIGLVIEYLMGGAYRCNYTR
KRFRTLYHNLFGPKRPKALKLLGMEDDIPLRRGRKTTKKREEEVDIDLDDPEINHFPFPFH
ELMVWAVLMKRQKMALFFWQHGEEAMAKALVACKLCKAMAHEASENDMVDDISQELN
HNSRDFGQLAVELLDQSYKQDEQLAMKLLTYELKNWSNATCLQLAVAAKHRDFIAHTCS
QMLLTDMWMGRLRMRKNSGLKVILGILLPPSILSLEFKNKDDMPYMSQAQEIHLQEKEAE
EPEKPTKEKEEEDMELTAMLGRNNGESSRKKDEEEVQSKHRLIPLGRKIYEFYNAPIVKF
WFYTLAYIGYLMLFNYIVLVKMERWPSTQEWIVISYIFTLGIEKMREILMSEPGKLLQKVKV
WLQEYWNVTDLIAILLFSVGMILRLQDQPFRSDGRVIYCVNIIYWYIRLLDIFGVNKYLGPY
VMMIGKMMIDMMYFVIIMLVVLMSFGVARQAILFPNEEPSWKLAKNIFYMPYWMIYGEVF
ADQIDPPCGQNETREDGKIIQLPPCKTGAWIVPAIMACYLLVANILLVNLLIAVFNNTFFEVK
SISNQVWKFQRYQLIMTFHERPVLPPPLIIFSHMTMIFQHLCCRWRKHESDPDERDYGLK
LFITDDELKKVHDFEEQCIEEYFREKDDRFNSSNDERIRVTSERVENMSMRLEEVNEREH
SMKASLQTVDIRLAQLEDLIGRMATALERLTGLERAESNKIRSRTSSDCTDAAYIVRQSSF
NSQEGNTFKLQESIDPAGEETMSPTSPTLMPRMRSHSFYSVNMKDKGGIEKLESIFKERS
LSLHRATSSHSVAKEPKAPAAPANTLAIVPDSRRPSSCIDIYVSAMDELHCDIDPLDNSVNI
LGLGEPSFSTPVPSTAPSSSAYATLAPTDRPPSRSIDFEDITSMDTRSFSSDYTHLPECQ
NPWDSEPPMYHTIERSKSSRYLATTPFLLEEAPIVKSHSFMFSPSRSYYANFGVPVKTAE
YTSITDCIDTRCVNAPQAIADRAAFPGGLGDKVEDLTCCHPEREAELSHPSSDSEENEAK
GRRATIAISSQEGDNSERTLSNNITVPKIERANSYSAEEPSAPYAHTRKSFSISDKLDRQR
NTASLRNPFQRSKSSKPEGRGDSLSMRRLSRTSAFQSFESKHN
SEQ ID NO: 11 Amino acid sequence of human TRPM3 isoform i
MGKKWRDAAEMERGCSDREDNAESRRRSRSASRGRFAESWKRLSSKQGSTKRSGLP
SQQTPAQKSWIERAFYKRECVHIIPSTKDPHRCCCGRLIGQHVGLTPSISVLQNEKNESR
LSRNDIQSEKWSISKHTQLSPTDAFGTIEFQGGGHSNKAMYVRVSFDTKPDLLLHLMTKE
WQLELPKLLISVHGGLQNFELQPKLKQVFGKGLIKAAMTTGAWIFTGGVNTGVIRHVGDA
LKDHASKSRGKICTIGIAPWGIVENQEDLIGRDVVRPYQTMSNPMSKLTVLNSMHSHFILA
DNGTTGKYGAEVKLRRQLEKHISLQKINTRIGQGVPVVALIVEGGPNVISIVLEYLRDTPPV
PVVVCDGSGRASDILAMGHKYSEEGGLINESLRDQLLVTIQKTFTYTRTQAQHLFIILMEC
QWPVGSLEQAMLDALVLDRVDFVKLLIENGVSMHRFLTISRLEELYNTRHGPSNTLYHLV
RDVKKGNLPPDYRISLIDIGLVIEYLMGGAYRCNYTRKRFRTLYHNLFGPKRPKALKLLGM
EDDIPLRRGRKTTKKREEEVDIDLDDPEINHFPFPFHELMVWAVLMKRQKMALFFWQHG
EEAMAKALVACKLCKAMAHEASENDMVDDISQELNHNSRDFGQLAVELLDQSYKQDEQ
LAMKLLTYELKNWSNATCLQLAVAAKHRDFIAHTCSQMLLTDMWMGRLRMRKNSGLKVI
LGILLPPSILSLEFKNKDDMPYMSQAQEIHLQEKEAEEPEKPTKEKEEEDMELTAMLGRN
NGESSRKKDEEEVQSKHRLIPLGRKIYEFYNAPIVKFWFYTLAYIGYLMLFNYIVLVKMER
WPSTQEWMSYIFTLGIEKMREILMSEPGKLLQKVKVWLQEYWNVTDLIAILLFSVGMILR
LQDQPFRSDGRVIYCVNIIYWYIRLLDIFGVNKYLGPYVMMIGKMMIDMMYFVIIMLVVLMS
FGVARQAILFPNEEPSWKLAKNIFYMPYWMIYGEVFADQIDPPCGQNETREDGKIIQLPP
CKTGAWIVPAIMACYLLVANILLVNLLIAVFNNTFFEVKSISNQVWKFQRYQLIMTFHERPV
LPPPLHFSHMTMIFQHLCCRWRKHESDPDERDYGLKLFITDDELKKVHDFEEQCIEEYFR
EKDDRFNSSNDERIRVTSERVENMSMRLEEVNEREHSMKASLQTVDIRLAQLEDLIGRM
ATALERLTGLERAESNKIRSRTSSDCTDAAYIVRQSSFNSQEGNTFKLQESIDPAGEETM
SPTSPTLMPRMRSHSFYSVNMKDKGGIEKLESIFKERSLSLHRATSSHSVAKEPKAPAAP
ANTLAIVPDSRRPSSCIDIYVSAMDELHCDIDPLDNSVNILGLGEPSFSTPVPSTAPSSSAY
ATLAPTDRPPSRSIDFEDITSMDTRSFSSDYTHLPECQNPWDSEPPMYHTIERSKSSRYL
ATTPFLLEEAPIVKSHSFMFSPSRSYYANFGVPVKTAEYTSITDCIDTRCVNAPQAIADRA
AFPGGLGDKVEDLTCCHPEREAELSHPSSDSEENEAKGRRATIAISSQEGDNSERTLSN
NITVPKIERANSYSAEEPSAPYAHTRKSFSISDKLDRQRNTASLRNPFQRSKSSKPEGRG
DSLSMRRLSRTSAFQSFESKHN
SEQ ID NO: 12 Amino acid sequence of human TRPM3 isoform j
MGKKWRDAAEMERGCSDREDNAESRRRSRSASRGRFAESWKRLSSKQGSTKRSGLP
SQQTPAQKSWIERAFYKRECVHIIPSTKDPHRCCCGRLIGQHVGLTPSISVLQNEKNESR
LSRNDIQSEKWSISKHTQLSPTDAFGTIEFQGGGHSNKAMYVRVSFDTKPDLLLHLMTKE
WQLELPKLLISVHGGLQNFELQPKLKQVFGKGLIKAAMTTGAWIFTGGVNTGVIRHVGDA
LKDHASKSRGKICTIGIAPWGIVENQEDLIGRDVVRPYQTMSNPMSKLTVLNSMHSHFILA
DNGTTGKYGAEVKLRRQLEKHISLQKINTRIGQGVPVVALIVEGGPNVISIVLEYLRDTPPV
PVVVCDGSGHASDILAFGHKYSEEGGLINESLHDQLLVTIQKTFTYTRTQAQHLFIILMEC
MKKKELITVFRMGSEGHQDIDLAILTALLKGANASAPDQLSLALAWNRVDIARSQIFIYGQ
QWPVGSLEQAMLDALVLDRVDFVKLLIENGVSMHRFLTISRLEELYNTRHGPSNTLYHLV
RDVKKREYPGFGWIYFKGNLPPDYRISLIDIGLVIEYLMGGAYRCNYTRKRFRTLYHNLFG
PKRPKALKLLGMEDDIPLRRGRKTTKKREEEVDIDLDDPEINHFPFPFHELMVWAVLMKR
QKMALFFWQHGEEAMAKALVACKLCKAMAHEASENDMVDDISQELNHNSRDFGQLAVE
LLDQSYKQDEQLAMKLLTYELKNWSNATCLQLAVAAKHRDFIAHTCSQMLLTDMWMGR
LRMRKNSGLKVILGILLPPSILSLEFKNKDDMPYMSQAQEIHLQEKEAEEPEKPTKEKEEE
DMELTAMLGRNNGESSRKKDEEEVQSKHRLIPLGRKIYEFYNAPIVKFWFYTLAYIGYLML
FNYIVLVKMERWPSTQEWIVISYIFTLGIEKMREILMSEPGKLLQKVKVWLQEYWNVTDLI
AILLFSVGMILRLQDQPFRSDGRVIYCVNIIYWYIRLLDIFGVNKYLGPYVMMIGKMMIDMM
YFVIIMLVVLMSFGVARQAILFPNEEPSWKLAKNIFYMPYWMIYGEVFADQIDPPCGQNET
REDGKIIQLPPCKTGAWIVPAIMACYLLVANILLVNLLIAVFNNTFFEVKSISNQVWKFQRY
QLIMTFHERPVLPPPLIIFSHMTMIFQHLCCRWRKHESDPDERDYGLKLFITDDELKKVHD
FEEQCIEEYFREKDDRFNSSNDERIRVTSERVENMSMRLEEVNEREHSMKASLQTVDIRL
AQLEDLIGRMATALERLTGLERAESNKIRSRTSSDCTDAAYIVRQSSFNSQEGNTFKLQE
SIDPAEHPLYSV
SEQ ID NO: 13 Amino acid sequence of human TRPM3 isoform w
MGKKWRDAAEMERGCSDREDNAESRRRSRSASRGRFAESWKRLSSKQGSTKRSGLP
SQQTPAQKSWIERAFYKRECVHIIPSTKDPHRCCCGRLIGQHVGLTPSISVLQNEKNESR
LSRNDIQSEKWSISKHTQLSPTDAFGTIEFQGGGHSNKAMYVRVSFDTKPDLLLHLMTKE
WQLELPKLLISVHGGLQNFELQPKLKQVFGKGLIKAAMTTGAWIFTGGVNTGVIRHVGDA
LKDHASKSRGKICTIGIAPWGIVENQEDLIGRDVVRPYQTMSNPMSKLTVLNSMHSHFILA
DNGTTGKYGAEVKLRRQLEKHISLQKINTRIGQGVPVVALIVEGGPNVISIVLEYLRDTPPV
VVVCDGSGHASDILAFGHKYSEEGGLINESLHDQLLVTIQKTFTYTRTQAQHLFIILMEC
MKKKELITVFRMGSEGHQDIDLAILTALLKGANASAPDQLSLALAWNRVDIARSQIFIYGQ
QWPVGSLEQAMLDALVLDRVDFVKLLIENGVSMHRFLTISRLEELYNTRHGPSNTLYHLV
RDVKKGNLPPDYRISLIDIGLVIEYLMGGAYRCNYTRKRFRTLYHNLFGPKRPKALKLLGM
EDDIPLRRGRKTTKKREEEVDIDLDDPEINHFPFPFHELMVWAVLMKRQKMALFFWQHG
EEAMAKALVACKLCKAMAHEASENDMVDDISQELNHNSRDFGQLAVELLDQSYKQDEQ
LAMKLLTYELKNWSNATCLQLAVAAKHRDFIAHTCSQMLLTDMWMGRLRMRKNSGLKVI
LGILLPPSILSLEFKNKDDMPYMSQAQEIHLQEKEAEEPEKPTKEKEEEDMELTAMLGRN
NGESSRKKDEEEVQSKHRLIPLGRKIYEFYNAPIVKFWFYTLAYIGYLMLFNYIVLVKMER
WPSTQEWMSYIFTLGIEKMREILMSEPGKLLQKVKVWLQEYWNVTDLIAILLFSVGMILR
LQDQPFRSDGRVIYCVNIIYWYIRLLDIFGVNKYLGPYVMMIGKMMIDMMYFVIIMLVVLMS
FGVARQAILFPNEEPSWKLAKNIFYMPYWMIYGEVFADQIDPPCGQNETREDGKIIQLPP
CKTGAWIVPAIMACYLLVANILLVNLLIAVFNNTFFEVKSISNQVWKFQRYQLIMTFHERPV
LPPPLHFSHMTMIFQHLCCRWRKHESDPDERDYGLKLFITDDELKKVHDFEEQCIEEYFR
EKDDRFNSSNDERIRVTSERVENMSMRLEEVNEREHSMKASLQTVDIRLAQLEDLIGRM
ATALERLTGLERAESNKIRSRTSSDCTDAAYIVRQSSFNSQEGNTFKLQESIDPAEHPLYS
V
SEQ ID NO: 14 Amino acid sequence of human TRPM3 isoform n
MPEPWGTVYFLGIAQVFSFLFSWWNLEGVMNQADAPRPLNWTIRKLCHAAFLPSVRLLK
AQKSWIERAFYKRECVHIIPSTKDPHRCCCGRLIGQHVGLTPSISVLQNEKNESRLSRNDI
QSEKWSISKHTQLSPTDAFGTIEFQGGGHSNKAMYVRVSFDTKPDLLLHLMTKEWQLEL
PKLLISVHGGLQNFELQPKLKQVFGKGLIKAAMTTGAWIFTGGVNTGVIRHVGDALKDHA
SKSRGKICTIGIAPWGIVENQEDLIGRDVVRPYQTMSNPMSKLTVLNSMHSHFILADNGTT
GKYGAEVKLRRQLEKHISLQKINTRIGQGVPVVALIVEGGPNVISIVLEYLRDTPPVPVVVC
DGSGHASDILAFGHKYSEEGGLINESLRDQLLVTIQKTFTYTRTQAQHLFILMECMKKKEL
ITVFRMGSEGHQDIDLAILTALLKGANASAPDQLSLALAWNRVDIARSQIFIYGQQWPVGS
LEQAMLDALVLDRVDFVKLLIENGVSMHRFLTISRLEELYNTRHGPSNTLYHLVRDVKKRE
YPGFGWIYFKGNLPPDYRISLIDIGLVIEYLMGGAYRCNYTRKRFRTLYHNLFGPKRPKAL
KLLGMEDDIPLRRGRKTTKKREEEVDIDLDDPEINHFPFPFHELMVWAVLMKRQKMALFF
WQHGEEAMAKALVACKLCKAMAHEASENDMVDDISQELNHNSRDFGQLAVELLDQSYK
QDEQLAMKLLTYELKNWSNATCLQLAVAAKHRDFIAHTCSQMLLTDMWMGRLRMRKNS
GLKVILGILLPPSILSLEFKNKDDMPYMSQAQEIHLQEKEAEEPEKPTKEKEEEDMELTAM
LGRNNGESSRKKDEEEVQSKHRLIPLGRKIYEFYNAPIVKFWFYTLAYIGYLMLFNYIVLVK
MERWPSTQEWIVISYIFTLGIEKMREILMSEPGKLLQKVKVWLQEYWNVTDLIAILLFSVG
MILRLQDQPFRSDGRVIYCVNIIYWYIRLLDIFGVNKYLGPYVMMIGKMMIDMMYFVIIMLV
VLMSFGVARQAILFPNEEPSWKLAKNIFYMPYWMIYGEVFADQIDPPCGQNETREDGKII
QLPPCKTGAWIVPAIMACYLLVANILLVNLLIAVFNNTFFEVKSISNQVWKFQRYQLIMTFH
ERPVLPPPLHFSHMTMIFQHLCCRWRKHESDPDERDYGLKLFITDDELKKVHDFEEQCIE
EYFREKDDRFNSSNDERIRVTSERVENMSMRLEEVNEREHSMKASLQTVDIRLAQLEDLI
GRMATALERLTGLERAESNKIRSRTSSDCTDAAYIVRQSSFNSQEGNTFKLQESIDPAEH
PLYSV
SEQ ID NO: 15 Amino acid sequence of human TRPM3 isoform o
MPEPWGTVYFLGIAQVFSFLFSWWNLEGVMNQADAPRPLNWTIRKLCHAAFLPSVRLLK
AQKSWIERAFYKRECVHIIPSTKDPHRCCCGRLIGQHVGLTPSISVLQNEKNESRLSRNDI
QSEKWSISKHTQLSPTDAFGTIEFQGGGHSNKAMYVRVSFDTKPDLLLHLMTKEWQLEL
PKLLISVHGGLQNFELQPKLKQVFGKGLIKAAMTTGAWIFTGGVNTGVIRHVGDALKDHA
SKSRGKICTIGIAPWGIVENQEDLIGRDVVRPYQTMSNPMSKLTVLNSMHSHFILADNGTT
GKYGAEVKLRRQLEKHISLQKINTRCLPFFSLDSRLFYSFWGSCQLDSVGIGQGVPVVALI
VEGGPNVISIVLEYLRDTPPVPVVVCDGSGRASDILAFGHKYSEEGGLINESLRDQLLVTI
QKTFTYTRTQAQHLFIILMECMKKKELITVFRMGSEGHQDIDLAILTALLKGANASAPDQLS
LALAWNRVDIARSQIFIYGQQWPVGSLEQAMLDALVLDRVDFVKLLIENGVSMHRFLTISR
LEELYNTRHGPSNTLYHLVRDVKKREYPGFGWIYFKGNLPPDYRISLIDIGLVIEYLMGGA
YRCNYTRKRFRTLYHNLFGPKRPKALKLLGMEDDIPLRRGRKTTKKREEEVDIDLDDPEI
NHFPFPFHELMVWAVLMKRQKMALFFWQHGEEAMAKALVACKLCKAMAHEASENDMV
DDISQELNHNSRDFGQLAVELLDQSYKQDEQLAMKLLTYELKNWSNATCLQLAVAAKHR
DFIAHTCSQMLLTDMWMGRLRMRKNSGLKVILGILLPPSILSLEFKNKDDMPYMSQAQEI
HLQEKEAEEPEKPTKEKEEEDMELTAMLGRNNGESSRKKDEEEVQSKHRLIPLGRKIYEF
YNAPIVKFWFYTLAYIGYLMLFNYIVLVKMERWPSTQEWIVISYIFTLGIEKMREILMSEPG
KLLQKVKVWLQEYWNVTDLIAILLFSVGMILRLQDQPFRSDGRVIYCVNIIYWYIRLLDIFGV
NKYLGPYVMMIGKMMIDMMYFVIIMLVVLMSFGVARQAILFPNEEPSWKLAKNIFYMPYW
MIYGEVFADQIDPPCGQNETREDGKIIQLPPCKTGAWIVPAIMACYLLVANILLVNLLIAVFN
NTFFEVKSISNQVWKFQRYQLIMTFHERPVLPPPLIIFSHMTMIFQHLCCRWRKHESDPD
ERDYGLKLFITDDELKKVHDFEEQCIEEYFREKDDRFNSSNDERIRVTSERVENMSMRLE
EVNEREHSMKASLQTVDIRLAQLEDLIGRMATALERLTGLERAESNKIRSRTSSDCTDAA
YIVRQSSFNSQEGNTFKLQESIDPAGEETMSPTSPTLMPRMRSHSFYSVNMKDKGGIEK
LESIFKERSLSLHRATSSHSVAKEPKAPAAPANTLAIVPDSRRPSSCIDIYVSAMDELHCDI
DPLDNSVNILGLGEPSFSTPVPSTAPSSSAYATLAPTDRPPSRSIDFEDITSMDTRSFSSD
YTHLPECQNPWDSEPPMYHTIERSKSSRYLATTPFLLEEAPIVKSHSFMFSPSRSYYANF
GVPVKTAEYTSITDCIDTRCVNAPQAIADRAAFPGGLGDKVEDLTCCHPEREAELSHPSS
DSEENEAKGRRATIAISSQEGDNSERTLSNNITVPKIERANSYSAEEPSAPYAHTRKSFSI
SDKLDRQRNTASLRNPFQRSKSSKPEGRGDSLSMRRLSRTSAFQSFESKHN
SEQ ID NO: 16 Amino acid sequence of human TRPM3 isoform p
MPEPWGTVYFLGIAQVFSFLFSWWNLEGVMNQADAPRPLNWTIRKLCHAAFLPSVRLLK
AQKSWIERAFYKRECVHIIPSTKDPHRCCCGRLIGQHVGLTPSISVLQNEKNESRLSRNDI
QSEKWSISKHTQLSPTDAFGTIEFQGGGHSNKAMYVRVSFDTKPDLLLHLMTKEWQLEL
PKLLISVHGGLQNFELQPKLKQVFGKGLIKAAMTTGAWIFTGGVNTGVIRHVGDALKDHA
SKSRGKICTIGIAPWGIVENQEDLIGRDVVRPYQTMSNPMSKLTVLNSMHSHFILADNGTT
GKYGAEVKLRRQLEKHISLQKINTRCLPFFSLDSRLFYSFWGSCQLDSVGIGQGVPVVALI
VEGGPNVISIVLEYLRDTPPVPVVVCDGSGRASDILAFGHKYSEEGGLINESLRDQLLVTI
QKTFTYTRTQAQHLFIILMECMKKKELITVFRMGSEGHQDIDLAILTALLKGANASAPDQLS
LALAWNRVDIARSQIFIYGQQWPVGSLEQAMLDALVLDRVDFVKLLIENGVSMHRFLTISR
LEELYNTRHGPSNTLYHLVRDVKKREYPGFGWIYFKGNLPPDYRISLIDIGLVIEYLMGGA
YRCNYTRKRFRTLYHNLFGPKRDDIPLRRGRKTTKKREEEVDIDLDDPEINHFPFPFHEL
MVWAVLMKRQKMALFFWQHGEEAMAKALVACKLCKAMAHEASENDMVDDISQELNHN
SRDFGQLAVELLDQSYKQDEQLAMKLLTYELKNWSNATCLQLAVAAKHRDFIAHTCSQM
LLTDMWMGRLRMRKNSGLKVILGILLPPSILSLEFKNKDDMPYMSQAQEIHLQEKEAEEP
EKPTKEKEEEDMELTAMLGRNNGESSRKKDEEEVQSKHRLIPLGRKIYEFYNAPIVKFWF
YTLAYIGYLMLFNYIVLVKMERWPSTQEWIVISYIFTLGIEKMREILMSEPGKLLQKVKVWL
QEYWNVTDLIAILLFSVGMILRLQDQPFRSDGRVIYCVNIIYWYIRLLDIFGVNKYLGPYVM
MIGKMMIDMMYFVIIMLVVLMSFGVARQAILFPNEEPSWKLAKNIFYMPYWMIYGEVFAD
QIDPPCGQNETREDGKIIQLPPCKTGAWIVPAIMACYLLVANILLVNLLIAVFNNTFFEVKSI
SNQVWKFQRYQLIMTFHERPVLPPPLIIFSHMTMIFQHLCCRWRKHESDPDERDYGLKLF
ITDDELKKVHDFEEQCIEEYFREKDDRFNSSNDERIRVTSERVENMSMRLEEVNEREHS
MKASLQTVDIRLAQLEDLIGRMATALERLTGLERAESNKIRSRTSSDCTDAAYIVRQSSFN
SQEGNTFKLQESIDPAEHPLYSV
SEQ ID NO: 17 Amino acid sequence of human TRPM3 isoform r
MPEPWGTVYFLGIAQVFSFLFSWWNLEGVMNQADAPRPLNWTIRKLCHAAFLPSVRLLK
AQKSWIERAFYKRECVHIIPSTKDPHRCCCGRLIGQHVGLTPSISVLQNEKNESRLSRNDI
QSEKWSISKHTQLSPTDAFGTIEFQGGGHSNKAMYVRVSFDTKPDLLLHLMTKEWQLEL
PKLLISVHGGLQNFELQPKLKQVFGKGLIKAAMTTGAWIFTGGVNTGVIRHVGDALKDHA
SKSRGKICTIGIAPWGIVENQEDLIGRDVVRPYQTMSNPMSKLTVLNSMHSHFILADNGTT
GKYGAEVKLRRQLEKHISLQKINTRIGQGVPVVALIVEGGPNVISIVLEYLRDTPPVPVVVC
DGSGHASDILAFGHKYSEEGGLINESLRDQLLVTIQKTFTYTRTQAQHLFIILMECMKKKEL
ITVFRMGSEGHQDIDLAILTALLKGANASAPDQLSLALAWNRVDIARSQIFIYGQQWPVGS
LEQAMLDALVLDRVDFVKLLIENGVSMHRFLTISRLEELYNTRHGPSNTLYHLVRDVKKG
NLPPDYRISLIDIGLVIEYLMGGAYRCNYTRKRFRTLYHNLFGPKRDDIPLRRGRKTTKKR
EEEVDIDLDDPEINHFPFPFHELMVWAVLMKRQKMALFFWQHGEEAMAKALVACKLCKA
MAHEASENDMVDDISQELNHNSRDFGQLAVELLDQSYKQDEQLAMKLLTYELKNWSNA
TCLQLAVAAKHRDFIAHTCSQMLLTDMWMGRLRMRKNSGLKVILGILLPPSILSLEFKNKD
DMPYMSQAQEIHLQEKEAEEPEKPTKEKEEEDMELTAMLGRNNGESSRKKDEEEVQSK
HRLIPLGRKIYEFYNAPIVKFWFYTLAYIGYLMLFNYIVLVKMERWPSTQEWIVISYIFTLGIE
KMREILMSEPGKLLQKVKVWLQEYWNVTDLIAILLFSVGMILRLQDQPFRSDGRVIYCVNII
YWYIRLLDIFGVNKYLGPYVMMIGKMMIDMMYFVIIMLVVLMSFGVARQAILFPNEEPSWK
NILLVNLLIAVFNNTFFEVKSISNQVWKFQRYQLIMTFHERPVLPPPLIIFSHMTMIFQHLCC
RWRKHESDPDERDYGLKLFITDDELKKVHDFEEQCIEEYFREKDDRFNSSNDERIRVTSE
RVENMSMRLEEVNEREHSMKASLQTVDIRLAQLEDLIGRMATALERLTGLERAESNKIRS
RTSSDCTDAAYIVRQSSFNSQEGNTFKLQESIDPAEHPLYSV
SEQ ID NO: 18 Amino acid sequence of human TRPM3 isoform t
MPEPWGTVYFLGIAQVFSFLFSWWNLEGVMNQADAPRPLNWTIRKLCHAAFLPSVRLLK
AQKSWIERAFYKRECVHIIPSTKDPHRCCCGRLIGQHVGLTPSISVLQNEKNESRLSRNDI
QSEKWSISKHTQLSPTDAFGTIEFQGGGHSNKAMYVRVSFDTKPDLLLHLMTKEWQLEL
PKLLISVHGGLQNFELQPKLKQVFGKGLIKAAMTTGAWIFTGGVNTGVIRHVGDALKDHA
SKSRGKICTIGIAPWGIVENQEDLIGRDVVRPYQTMSNPMSKLTVLNSMHSHFILADNGTT
GKYGAEVKLRRQLEKHISLQKINTRCLPFFSLDSRLFYSFWGSCQLDSVGIGQGVPVVALI
VEGGPNVISIVLEYLRDTPPVPVVVCDGSGRASDILAFGHKYSEEGGLINESLRDQLLVTI
QKTFTYTRTQAQHLFIILMECMKKKELITVFRMGSEGHQDIDLAILTALLKGANASAPDQLS
LALAWNRVDIARSQIFIYGQQWPVGSLEQAMLDALVLDRVDFVKLLIENGVSMHRFLTISR
LEELYNTRHGPSNTLYHLVRDVKKREYPGFGWIYFKGNLPPDYRISLIDIGLVIEYLMGGA
YRCNYTRKRFRTLYHNLFGPKRPKALKLLGMEDDIPLRRGRKTTKKREEEVDIDLDDPEI
NHFPFPFHELMVWAVLMKRQKMALFFWQHGEEAMAKALVACKLCKAMAHEASENDMV
DDISQELNHNSRDFGQLAVELLDQSYKQDEQLAMKLLTYELKNWSNATCLQLAVAAKHR
DFIAHTCSQMLLTDMWMGRLRMRKNSGLKVILGILLPPSILSLEFKNKDDMPYMSQAQEI
HLQEKEAEEPEKPTKEKEEEDMELTAMLGRNNGESSRKKDEEEVQSKHRLIPLGRKIYEF
YNAPIVKFWFYTLAYIGYLMLFNYIVLVKMERWPSTQEWIVISYIFTLGIEKMREILMSEPG
KLLQKVKVWLQEYWNVTDLIAILLFSVGMILRLQDQPFRSDGRVIYCVNIIYWYIRLLDIFGV
NKYLGPYVMMIGKMMIDMMYFVIIMLVVLMSFGVARQAILFPNEEPSWKLAKNIFYMPYW
MIYGEVFADQIDPPCGQNETREDGKIIQLPPCKTGAWIVPAIMACYLLVANILLVNLLIAVFN
NTFFEVKSISNQVWKFQRYQLIMTFHERPVLPPPLIIFSHMTMIFQHLCCRWRKHESDPD
ERDYGLKLFITDDELKKVHDFEEQCIEEYFREKDDRFNSSNDERIRVTSERVENMSMRLE
EVNEREHSMKASLQTVDIRLAQLEDLIGRMATALERLTGLERAESNKIRSRTSSDCTDAA
YIVRQSSFNSQEGNTFKLQESIDPAEHPLYSV
SEQ ID NO: 19 Amino acid sequence of human TRPM3 isoform v
MYVRVSFDTKPDLLLHLMTKEWQLELPKLLISVHGGLQNFELQPKLKQVFGKGLIKAAMT
TGAWIFTGGVNTGVIRHVGDALKDHASKSRGKICTIGIAPWGIVENQEDLIGRDVVRPYQT
MSNPMSKLTVLNSMHSHFILADNGTTGKYGAEVKLRRQLEKHISLQKINTRIGQGVPVVA
LIVEGGPNVISIVLEYLRDTPPVPVVVCDGSGRASDILAFGHKYSEEGGLINESLRDQLLVT
IQKTFTYTRTQAQHLFIILMECMKKKELITVFRMGSEGHQDIDLAILTALLKGANASAPDQL
SLALAWNRVDIARSQIFIYGQQWPVGSLEQAMLDALVLDRVDFVKLLIENGVSMHRFLTIS
RLEELYNTRHGPSNTLYHLVRDVKKREYPGFGWIYFKGNLPPDYRISLIDIGLVIEYLMGG
AYRCNYTRKRFRTLYHNLFGPKRPKALKLLGMEDDIPLRRGRKTTKKREEEVDIDLDDPEI
NHFPFPFHELMVWAVLMKRQKMALFFWQHGEEAMAKALVACKLCKAMAHEASENDMV
DDISQELNHNSRDFGQLAVELLDQSYKQDEQLAMKLLTYELKNWSNATCLQLAVAAKHR
DFIAHTCSQMLLTDMWMGRLRMRKNSGLKVILGILLPPSILSLEFKNKDDMPYMSQAQEI
HLQEKEAEEPEKPTKEKEEEDMELTAMLGRNNGESSRKKDEEEVQSKHRLIPLGRKIYEF
YNAPIVKFWFYTLAYIGYLMLFNYIVLVKMERWPSTQEWIVISYIFTLGIEKMREILMSEPG
KLLQKVKVWLQEYWNVTDLIAILLFSVGMILRLQDQPFRSDGRVIYCVNIIYWYIRLLDIFGV
NKYLGPYVMMIGKMMIDMMYFVIIMLVVLMSFGVARQAILFPNEEPSWKLAKNIFYMPYW
MIYGEVFADQIDPPCGQNETREDGKIIQLPPCKTGAWIVPAIMACYLLVANILLVNLLIAVFN
NTFFEVKSISNQVWKFQRYQLIMTFHERPVLPPPLIIFSHMTMIFQHLCCRWRKHESDPD
ERDYGLKLFITDDELKKVHDFEEQCIEEYFREKDDRFNSSNDERIRVTSERVENMSMRLE
EVNEREHSMKASLQTVDIRLAQLEDLIGRMATALERLTGLERAESNKIRSRTSSDCTDAA
YIVRQSSFNSQEGNTFKLQESIDPAEHPLYSV
SEQ ID NO: 20 Amino acid sequence of human TRPM3 isoform a
MYVRVSFDTKPDLLLHLMTKEWQLELPKLLISVHGGLQNFELQPKLKQVFGKGLIKAAMT
TGAWIFTGGVNTGVIRHVGDALKDHASKSRGKICTIGIAPWGIVENQEDLIGRDVVRPYQT
MSNPMSKLTVLNSMHSHFILADNGTTGKYGAEVKLRRQLEKHISLQKINTRIGQGVPVVA
LIVEGGPNVISIVLEYLRDTPPVPVVVCDGSGRASDILAFGHKYSEEGGLINESLRDQLLVT
IQKTFTYTRTQAQHLFIILMECMKKKELITVFRMGSEGHQDIDLAILTALLKGANASAPDQL
SLALAWNRVDIARSQIFIYGQQWPVGSLEQAMLDALVLDRVDFVKLLIENGVSMHRFLTIS
RLEELYNTRHGPSNTLYHLVRDVKKGNLPPDYRISLIDIGLVIEYLMGGAYRCNYTRKRFR
TLYHNLFGPKRPKALKLLGMEDDIPLRRGRKTTKKREEEVDIDLDDPEINHFPFPFHELMV
WAVLMKRQKMALFFWQHGEEAMAKALVACKLCKAMAHEASENDMVDDISQELNHNSR
DFGQLAVELLDQSYKQDEQLAMKLLTYELKNWSNATCLQLAVAAKHRDFIAHTCSQMLL
TDMWMGRLRMRKNSGLKVILGILLPPSILSLEFKNKDDMPYMSQAQEIHLQEKEAEEPEK
PTKEKEEEDMELTAMLGRNNGESSRKKDEEEVQSKHRLIPLGRKIYEFYNAPIVKFWFYT
LAYIGYLMLFNYIVLVKMERWPSTQEWIVISYIFTLGIEKMREILMSEPGKLLQKVKVWLQE
YWNVTDLIAILLFSVGMILRLQDQPFRSDGRVIYCVNIIYWYIRLLDIFGVNKYLGPYVMMIG
KMMIDMMYFVIIMLVVLMSFGVARQAILFPNEEPSWKLAKNIFYMPYWMIYGEVFADQIDP
PCGQNETREDGKIIQLPPCKTGAWIVPAIMACYLLVANILLVNLLIAVFNNTFFEVKSISNQV
WKFQRYQLIMTFHERPVLPPPLIIFSHMTMIFQHLCCRWRKHESDPDERDYGLKLFITDD
ELKKVHDFEEQCIEEYFREKDDRFNSSNDERIRVTSERVENMSMRLEEVNEREHSMKAS
LQTVDIRLAQLEDLIGRMATALERLTGLERAESNKIRSRTSSDCTDAAYIVRQSSFNSQEG
NTFKLQESIDPAGEETMSPTSPTLMPRMRSHSFYSVNMKDKGGIEKLESIFKERSLSLHR
ATSSHSVAKEPKAPAAPANTLAIVPDSRRPSSCIDIYVSAMDELHCDIDPLDNSVNILGLGE
PSFSTPVPSTAPSSSAYATLAPTDRPPSRSIDFEDITSMDTRSFSSDYTHLPECQNPWDS
EPPMYHTIERSKSSRYLATTPFLLEEAPIVKSHSFMFSPSRSYYANFGVPVKTAEYTSITD
CIDTRCVNAPQAIADRAAFPGGLGDKVEDLTCCHPEREAELSHPSSDSEENEAKGRRATI
AISSQEGDNSERTLSNNITVPKIERANSYSAEEPSAPYAHTRKSFSISDKLDRQRNTASLR
NPFQRSKSSKPEGRGDSLSMRRLSRTSAFQSFESKHN
SEQ ID NO: 21 Amino acid sequence of human TRPM3 isoform b
MYVRVSFDTKPDLLLHLMTKEWQLELPKLLISVHGGLQNFELQPKLKQVFGKGLIKAAMT
TGAWIFTGGVNTGVIRHVGDALKDHASKSRGKICTIGIAPWGIVENQEDLIGRDVVRPYQT
MSNPMSKLTVLNSMHSHFILADNGTTGKYGAEVKLRRQLEKHISLQKINTRIGQGVPVVA
LIVEGGPNVISIVLEYLRDTPPVPVVVCDGSGRASDILAFGHKYSEEGGLINESLRDQLLVT
IQKTFTYTRTQAQHLFIILMECMKKKELITVFRMGSEGHQDIDLAILTALLKGANASAPDQL
SLALAWNRVDIARSQIFIYGQQWPVGSLEQAMLDALVLDRVDFVKLLIENGVSMHRFLTIS
RLEELYNTRHGPSNTLYHLVRDVKKREYPGFGWIYFKGNLPPDYRISLIDIGLVIEYLMGG
AYRCNYTRKRFRTLYHNLFGPKRPKALKLLGMEDDIPLRRGRKTTKKREEEVDIDLDDPEI
NHFPFPFHELMVWAVLMKRQKMALFFWQHGEEAMAKALVACKLCKAMAHEASENDMV
DDISQELNHNSRDFGQLAVELLDQSYKQDEQLAMKLLTYELKNWSNATCLQLAVAAKHR
DFIAHTCSQMLLTDMWMGRLRMRKNSGLKVILGILLPPSILSLEFKNKDDMPYMSQAQEI
HLQEKEAEEPEKPTKEKEEEDMELTAMLGRNNGESSRKKDEEEVQSKHRLIPLGRKIYEF
YNAPIVKFWFYTLAYIGYLMLFNYIVLVKMERWPSTQEWIVISYIFTLGIEKMREILMSEPG
KLLQKVKVWLQEYWNVTDLIAILLFSVGMILRLQDQPFRSDGRVIYCVNIIYWYIRLLDIFGV
NKYLGPYVMMIGKMMIDMMYFVIIMLVVLMSFGVARQAILFPNEEPSWKLAKNIFYMPYW
MIYGEVFADQIDPPCGQNETREDGKIIQLPPCKTGAWIVPAIMACYLLVANILLVNLLIAVFN
NTFFEVKSISNQVWKFQRYQLIMTFHERPVLPPPLIIFSHMTMIFQHLCCRWRKHESDPD
ERDYGLKLFITDDELKKVHDFEEQCIEEYFREKDDRFNSSNDERIRVTSERVENMSMRLE
EVNEREHSMKASLQTVDIRLAQLEDLIGRMATALERLTGLERAESNKIRSRTSSDCTDAA
YIVRQSSFNSQEGNTFKLQESIDPAGEETMSPTSPTLMPRMRSHSFYSVNMKDKGGIEK
LESIFKERSLSLHRATSSHSVAKEPKAPAAPANTLAIVPDSRRPSSCIDIYVSAMDELHCDI
DPLDNSVNILGLGEPSFSTPVPSTAPSSSAYATLAPTDRPPSRSIDFEDITSMDTRSFSSD
YTHLPECQNPWDSEPPMYHTIERSKSSRYLATTPFLLEEAPIVKSHSFMFSPSRSYYANF
GVPVKTAEYTSITDCIDTRCVNAPQAIADRAAFPGGLGDKVEDLTCCHPEREAELSHPSS
DSEENEAKGRRATIAISSQEGDNSERTLSNNITVPKIERANSYSAEEPSAPYAHTRKSFSI
SDKLDRQRNTASLRNPFQRSKSSKPEGRGDSLSMRRLSRTSAFQSFESKHN
SEQ ID NO: 22 Amino acid sequence of human TRPM3 isoform d
MYVRVSFDTKPDLLLHLMTKEWQLELPKLLISVHGGLQNFELQPKLKQVFGKGLIKAAMT
TGAWIFTGGVNTGVIRHVGDALKDHASKSRGKICTIGIAPWGIVENQEDLIGRDVVRPYQT
MSNPMSKLTVLNSMHSHFILADNGTTGKYGAEVKLRRQLEKHISLQKINTRIGQGVPVVA
LIVEGGPNVISIVLEYLRDTPPVPVVVCDGSGRASDILAFGHKYSEEGGLINESLRDQLLVT
IQKTFTYTRTQAQHLHILMECMKKKELITVFRMGSEGHQDIDLAILTALLKGANASAPDQL
SLALAWNRVDIARSQIFIYGQQWPVGSLEQAMLDALVLDRVDFVKLLIENGVSMHRFLTIS
RLEELYNTRHGPSNTLYHLVRDVKKGNLPPDYRISLIDIGLVIEYLMGGAYRCNYTRKRFR
TLYHNLFGPKRDDIPLRRGRKTTKKREEEVDIDLDDPEINHFPFPFHELMVWAVLMKRQK
MALFFWQHGEEAMAKALVACKLCKAMAHEASENDMVDDISQELNHNSRDFGQLAVELL
DQSYKQDEQLAMKLLTYELKNWSNATCLQLAVAAKHRDFIAHTCSQMLLTDMWMGRLR
MRKNSGLKVILGILLPPSILSLEFKNKDDMPYMSQAQEIHLQEKEAEEPEKPTKEKEEEDM
ELTAMLGRNNGESSRKKDEEEVQSKHRLIPLGRKIYEFYNAPIVKFWFYTLAYIGYLMLFN
YIVLVKMERWPSTQEWIVISYIFTLGIEKMREILMSEPGKLLQKVKVWLQEYWNVTDLIAIL
LFSVGMILRLQDQPFRSDGRVIYCVNIIYWYIRLLDIFGVNKYLGPYVMMIGKMMIDMMYF
VIIMLVVLMSFGVARQAILFPNEEPSWKLAKNIFYMPYWMIYGEVFADQIDPPCGQNETRE
DGKIIQLPPCKTGAWIVPAIMACYLLVANILLVNLLIAVFNNTFFEVKSISNQVWKFQRYQLI
MTFHERPVLPPPLIIFSHMTMIFQHLCCRWRKHESDPDERDYGLKLFITDDELKKVHDFEE
QGIEEYFREKDDRFNSSNDERIRVTSERVENMSMRLEEVNEREHSMKASLQTVDIRLAQ
LEDLIGRMATALERLTGLERAESNKIRSRTSSDCTDAAYIVRQSSFNSQEGNTFKLQESID
PAGEETMSPTSPTLMPRMRSHSFYSVNMKDKGGIEKLESIFKERSLSLHRATSSHSVAKE
PKAPAAPANTLAIVPDSRRPSSCIDIYVSAMDELHCDIDPLDNSVNILGLGEPSFSTPVPST
APSSSAYATLAPTDRPPSRSIDFEDITSMDTRSFSSDYTHLPECQNPWDSEPPMYHTIER
SKSSRYLATTPFLLEEAPIVKSHSFMFSPSRSYYANFGVPVKTAEYTSITDCIDTRCVNAP
QAIADRAAFPGGLGDKVEDLTCCHPEREAELSHPSSDSEENEAKGRRATIAISSQEGDNS
ERTLSNNITVPKIERANSYSAEEPSAPYAHTRKSFSISDKLDRQRNTASLRNPFQRSKSSK
PEGRGDSLSMRRLSRTSAFQSFESKHN
SEQ ID NO: 23 Amino acid sequence of human TRPM3 isoform e
MYVRVSFDTKPDLLLHLMTKEWQLELPKLLISVHGGLQNFELQPKLKQVFGKGLIKAAMT
TGAWIFTGGVNTGVIRHVGDALKDHASKSRGKICTIGIAPWGIVENQEDLIGRDVVRPYQT
MSNPMSKLTVLNSMHSHFILADNGTTGKYGAEVKLRRQLEKHISLQKINTRIGQGVPVVA
LIVEGGPNVISIVLEYLRDTPPVPVVVCDGSGRASDILAFGHKYSEEGGLINESLRDQLLVT
IQKTFTYTRTQAQHLFIILMECMKKKELITVFRMGSEGHQDIDLAILTALLKGANASAPDQL
SLALAWNRVDIARSQIFIYGQQWPVGSLEQAMLDALVLDRVDFVKLLIENGVSMHRFLTIS
RLEELYNTRHGPSNTLYHLVRDVKKREYPGFGWIYFKGNLPPDYRISLIDIGLVIEYLMGG
AYRCNYTRKRFRTLYHNLFGPKRDDIPLRRGRKTTKKREEEVDIDLDDPEINHFPFPFHEL
MVWAVLMKRQKMALFFWQHGEEAMAKALVACKLCKAMAHEASENDMVDDISQELNHN
SRDFGQLAVELLDQSYKQDEQLAMKLLTYELKNWSNATCLQLAVAAKHRDFIAHTCSQM
LLTDMWMGRLRMRKNSGLKVILGILLPPSILSLEFKNKDDMPYMSQAQEIHLQEKEAEEP
EKPTKEKEEEDMELTAMLGRNNGESSRKKDEEEVQSKHRLIPLGRKIYEFYNAPIVKFWF
YTLAYIGYLMLFNYIVLVKMERWPSTQEWIVISYIFTLGIEKMREILMSEPGKLLQKVKVWL
QEYWNVTDLIAILLFSVGMILRLQDQPFRSDGRVIYCVNIIYWYIRLLDIFGVNKYLGPYVM
MIGKMMIDMMYFVIIMLVVLMSFGVARQAILFPNEEPSWKLAKNIFYMPYWMIYGEVFAD
QIDPPCGQNETREDGKIIQLPPCKTGAWIVPAIMACYLLVANILLVNLLIAVFNNTFFEVKSI
SNQVWKFQRYQLIMTFHERPVLPPPLIIFSHMTMIFQHLCCRWRKHESDPDERDYGLKLF
ITDDELKKVHDFEEQCIEEYFREKDDRFNSSNDERIRVTSERVENMSMRLEEVNEREHS
MKASLQTVDIRLAQLEDLIGRMATALERLTGLERAESNKIRSRTSSDCTDAAYIVRQSSFN
SQEGNTFKLQESIDPAGEETMSPTSPTLMPRMRSHSFYSVNMKDKGGIEKLESIFKERSL
SLHRATSSHSVAKEPKAPAAPANTLAIVPDSRRPSSCIDIYVSAMDELHCDIDPLDNSVNIL
GLGEPSFSTPVPSTAPSSSAYATLAPTDRPPSRSIDFEDITSMDTRSFSSDYTHLPECQN
PWDSEPPMYHTIERSKSSRYLATTPFLLEEAPIVKSHSFMFSPSRSYYANFGVPVKTAEY
TSITDCIDTRCVNAPQAIADRAAFPGGLGDKVEDLTCCHPEREAELSHPSSDSEENEAKG
RRATIAISSQEGDNSERTLSNNITVPKIERANSYSAEEPSAPYAHTRKSFSISDKLDRQRN
TASLRNPFQRSKSSKPEGRGDSLSMRRLSRTSAFQSFESKHN
SEQ ID NO: 24 Amino acid sequence of human TRPM3 isoform f
MYVRVSFDTKPDLLLHLMTKEWQLELPKLLISVHGGLQNFELQPKLKQVFGKGLIKAAMT
TGAWIFTGGVNTGVIRHVGDALKDHASKSRGKICTIGIAPWGIVENQEDLIGRDVVRPYQT
MSNPMSKLTVLNSMHSHFILADNGTTGKYGAEVKLRRQLEKHISLQKINTRCLPFFSLDS
RLFYSFWGSCQLDSVGIGQGVPVVALIVEGGPNVISIVLEYLRDTPPVPVVVCDGSGRAS
DILAFGHKYSEEGGLINESLHDQLLVTIQKTFTYTRTQAQHLFIILMECMKKKELITVFRMG
SEGHQDIDLAILTALLKGANASAPDQLSLALAWNRVDIARSQIFIYGQQWPVGSLEQAML
DALVLDRVDFVKLLIENGVSMHRFLTISRLEELYNTRHGPSNTLYHLVRDVKKGNLPPDY
RISLIDIGLVIEYLMGGAYRCNYTRKRFRTLYHNLFGPKRPKALKLLGMEDDIPLRRGRKTT
KKREEEVDIDLDDPEINHFPFPFHELMVWAVLMKRQKMALFFWQHGEEAMAKALVACKL
CKAMAHEASENDMVDDISQELNHNSRDFGQLAVELLDQSYKQDEQLAMKLLTYELKNW
SNATCLQLAVAAKHRDFIAHTCSQMLLTDMWMGRLRMRKNSGLKVILGILLPPSILSLEFK
NKDDMPYMSQAQEIHLQEKEAEEPEKPTKEKEEEDMELTAMLGRNNGESSRKKDEEEV
QSKHRLIPLGRKIYEFYNAPIVKFWFYTLAYIGYLMLFNYIVLVKMERWPSTQEWIVISYIFT
LGIEKMREILMSEPGKLLQKVKVWLQEYWNVTDLIAILLFSVGMILRLQDQPFRSDGRVIY
CVNIIYWYIRLLDIFGVNKYLGPYVMMIGKMMIDMMYFVIIMLVVLMSFGVARQAILFPNEE
PSWKLAKNIFYMPYWMIYGEVFADQIDPPCGQNETREDGKIIQLPPCKTGAWIVPAIMAC
YLLVANILLVNLLIAVFNNTFFEVKSISNQVWKFQRYQLIMTFHERPVLPPPLIIFSHMTMIF
QHLCCRWRKHESDPDERDYGLKLFITDDELKKVHDFEEQCIEEYFREKDDRFNSSNDER
IRVTSERVENMSMRLEEVNEREHSMKASLQTVDIRLAQLEDLIGRMATALERLTGLERAE
SNKIRSRTSSDCTDAAYIVRQSSFNSQEGNTFKLQESIDPAGEETMSPTSPTLMPRMRSH
SFYSVNMKDKGGIEKLESIFKERSLSLHRATSSHSVAKEPKAPAAPANTLAIVPDSRRPSS
CIDIYVSAMDELHCDIDPLDNSVNILGLGEPSFSTPVPSTAPSSSAYATLAPTDRPPSRSID
FEDITSMDTRSFSSDYTHLPECQNPWDSEPPMYHTIERSKSSRYLATTPFLLEEAPIVKSH
SFMFSPSRSYYANFGVPVKTAEYTSITDCIDTRCVNAPQAIADRAAFPGGLGDKVEDLTC
CHPEREAELSHPSSDSEENEAKGRRATIAISSQEGDNSERTLSNNITVPKIERANSYSAEE
PSAPYAHTRKSFSISDKLDRQRNTASLRNPFQRSKSSKPEGRGDSLSMRRLSRTSAFQS
FESKHN
SEQ ID NO: 25 Amino acid sequence of human TRPM3 isoform g
MYVRVSFDTKPDLLLHLMTKEWQLELPKLLISVHGGLQNFELQPKLKQVFGKGLIKAAMT
TGAWIFTGGVNTGVIRHVGDALKDHASKSRGKICTIGIAPWGIVENQEDLIGRDVVRPYQT
MSNPMSKLTVLNSMHSHFILADNGTTGKYGAEVKLRRQLEKHISLQKINTRCLPFFSLDS
RLFYSFWGSCQLDSVGIGQGVPVVALIVEGGPNVISIVLEYLRDTPPVPVVVCDGSGRAS
DILAFGHKYSEEGGLINESLHDQLLVTIQKTFTYTRTQAQHLFIILMECMKKKELITVFRMG
SEGHQDIDLAILTALLKGANASAPDQLSLALAWNRVDIARSQIFIYGQQWPVGSLEQAML
DALVLDRVDFVKLLIENGVSMHRFLTISRLEELYNTRHGPSNTLYHLVRDVKKGNLPPDY
RISLIDIGLVIEYLMGGAYRCNYTRKRFRTLYHNLFGPKRDDIPLRRGRKTTKKREEEVDID
LDDPEINHFPFPFHELMVWAVLMKRQKMALFFWQHGEEAMAKALVACKLCKAMAHEAS
ENDMVDDISQELNHNSRDFGQLAVELLDQSYKQDEQLAMKLLTYELKNWSNATCLQLAV
AAKHRDFIAHTCSQMLLTDMWMGRLRMRKNSGLKVILGILLPPSILSLEFKNKDDMPYMS
QAQEIHLQEKEAEEPEKPTKEKEEEDMELTAMLGRNNGESSRKKDEEEVQSKHRLIPLG
RKIYEFYNAPIVKFWFYTLAYIGYLMLFNYIVLVKMERWPSTQEWIVISYIFTLGIEKMREIL
MSEPGKLLQKVKVWLQEYWNVTDLIAILLFSVGMILRLQDQPFRSDGRVIYCVNIIYWYIRL
LDIFGVNKYLGPYVMMIGKMMIDMMYFVIIMLVVLMSFGVARQAILFPNEEPSWKLAKNIF
YMPYWMIYGEVFADQIDPPCGQNETREDGKIIQLPPCKTGAWIVPAIMACYLLVANILLVN
LLIAVFNNTFFEVKSISNQVWKFQRYQLIMTFHERPVLPPPLIIFSHMTMIFQHLCCRWRK
HESDPDERDYGLKLFITDDELKKVHDFEEQCIEEYFREKDDRFNSSNDERIRVTSERVEN
MSMRLEEVNEREHSMKASLQTVDIRLAQLEDLIGRMATALERLTGLERAESNKIRSRTSS
DCTDAAYIVRQSSFNSQEGNTFKLQESIDPAGEETMSPTSPTLMPRMRSHSFYSVNMKD
KGGIEKLESIFKERSLSLHRATSSHSVAKEPKAPAAPANTLAIVPDSRRPSSCIDIYVSAMD
ELHCDIDPLDNSVNILGLGEPSFSTPVPSTAPSSSAYATLAPTDRPPSRSIDFEDITSMDTR
SFSSDYTHLPECQNPWDSEPPMYHTIERSKSSRYLATTPFLLEEAPIVKSHSFMFSPSRS
YYANFGVPVKTAEYTSITDCIDTRCVNAPQAIADRAAFPGGLGDKVEDLTCCHPEREAEL
SHPSSDSEENEAKGRRATIAISSQEGDNSERTLSNNITVPKIERANSYSAEEPSAPYAHTR
KSFSISDKLDRQRNTASLRNPFQRSKSSKPEGRGDSLSMRRLSRTSAFQSFESKHN
SEQ ID NO: 26 Amino acid sequence of human TRPM3 isoform X18
MGKKWRDAAEMERGCSDREDNAESRRRSRSASRGRFAESWKRLSSKQGSTKRSGLP
SQQTPAQKSWIERAFYKRECVHIIPSTKDPHRCCCGRLIGQHVGLTPSISVLQNEKNESR
LSRNDIQSEKWSISKHTQLSPTDAFGTIEFQGGGHSNKAMYVRVSFDTKPDLLLHLMTKE
WQLELPKLLISVHGGLQNFELQPKLKQVFGKGLIKAAMTTGAWIFTGGVNTGVIRHVGDA
LKDHASKSRGKICTIGIAPWGIVENQEDLIGRDVVRPYQTMSNPMSKLTVLNSMHSHFILA
DNGTTGKYGAEVKLRRQLEKHISLQKINTRIGQGVPVVALIVEGGPNVISIVLEYLRDTPPV
VVVCDGSGHASDILAFGHKYSEEGGLINESLHDOLLVTIQKTFTYTRTQAQHLFIILMEC
MKKKELITVFRMGSEGHQDIDLAILTALLKGANASAPDQLSLALAWNRVDIARSQIFIYGQ
QWPVGSLEQAMLDALVLDRVDFVKLLIENGVSMHRFLTISRLEELYNTRHGPSNTLYHLV
RDVKKGNLPPDYRISLIDIGLVIEYLMGGAYRCNYTRKRFRTLYHNLFGPKRDDIPLRRGR
KTTKKREEEVDIDLDDPEINHFPFPFHELMVWAVLMKRQKMALFFWQHGEEAMAKALVA
CKLCKAMAHEASENDMVDDISQELNHNSRDFGQLAVELLDQSYKQDEQLAMKLLTYELK
NWSNATCLQLAVAAKHRDFIAHTCSQMLLTDMWMGRLRMRKNSGLKVILGILLPPSILSL
EFKNKDDMPYMSQAQEIHLQEKEAEEPEKPTKEKEEEDMELTAMLGRNNGESSRKKDE
EEVQSKHRLIPLGRKIYEFYNAPIVKFWFYTLAYIGYLMLFNYIVLVKMERWPSTQEWIVIS
YIFTLGIEKMREILMSEPGKLLQKVKVWLQEYWNVTDLIAILLFSVGMILRLQDQPFRSDG
RVIYCVNIIYWYIRLLDIFGVNKYLGPYVMMIGKMMIDMMYFVIIMLVVLMSFGVARQAILFP
NEEPSWKLAKNIFYMPYWMIYGEVFADQIDPPCGQNETREDGKIIQLPPCKTGAWIVPAI
MACYLLVANILLVNLLIAVFNNTFFEVKSISNQVWKFQRYQLIMTFHERPVLPPPLIIFSHMT
MIFQHLCCRWRKHESDPDERDYGLKLFITDDELKKVHDFEEQCIEEYFREKDDRFNSSN
DERIRVTSERVENMSMRLEEVNEREHSMKASLQTVDIRLAQLEDLIGRMATALERLTGLE
RAESNKIRSRTSSDCTDAAYIVRQSSFNSQEGNTFKLQESIDPAGEETMSPTSPTLMPRM
RSHSFYSVNMKDKGGIEKLESIFKERSLSLHRATSSHSVAKEPKAPAAPANTLAIVPDSRR
PSSCIDIYVSAMDELHCDIDPLDNSVNILGLGEPSFSTPVPSTAPSSSAYATLAPTDRPPS
RSIDFEDITSMDTRSFSSDYTHLPECQNPWDSEPPMYHTIERSKSSRYLATTPFLLEEAPI
VKSHSFMFSPSRSYYANFGVPVKTAEYTSITDCIDTRCVNAPQAIADRAAFPGGLGDKVE
DLTCCHPEREAELSHPSSDSEENEAKGRRATIAISSQEGDNSERTLSNNITVPKIERANSY
SAEEPSAPYAHTRKSFSISDKLDRQRNTASLRNPFQRSKSSKPEGRGDSLSMRRLSRTS
AFQSFESKHN
SEQ ID NO: 27 Amino acid sequence of human TRPM3 isoform X15
MGKKWRDAAEMERGCSDREDNAESRRRSRSASRGRFAESWKRLSSKQGSTKRSGLP
SQQTPAQKSWIERAFYKRECVHIIPSTKDPHRCCCGRLIGQHVGLTPSISVLQNEKNESR
LSRNDIQSEKWSISKHTQLSPTDAFGTIEFQGGGHSNKAMYVRVSFDTKPDLLLHLMTKE
WQLELPKLLISVHGGLQNFELQPKLKQVFGKGLIKAAMTTGAWIFTGGVNTGVIRHVGDA
LKDHASKSRGKICTIGIAPWGIVENQEDLIGRDVVRPYQTMSNPMSKLTVLNSMHSHFILA
DNGTTGKYGAEVKLRRQLEKHISLQKINTRIGQGVPVVALIVEGGPNVISIVLEYLRDTPPV
PVVVCDGSGHASDILAFGHKYSEEGGLINESLHDQLLVTIQKTFTYTRTQAQHLFIILMEC
MKKKELITVFRMGSEGHQDIDLAILTALLKGANASAPDQLSLALAWNRVDIARSQIFIYGQ
QWPVGSLEQAMLDALVLDRVDFVKLLIENGVSMHRFLTISRLEELYNTRHGPSNTLYHLV
RDVKKREYPGFGWIYFKGNLPPDYRISLIDIGLVIEYLMGGAYRCNYTRKRFRTLYHNLFG
PKRDDIPLRRGRKTTKKREEEVDIDLDDPEINHFPFPFHELMVWAVLMKRQKMALFFWQ
HGEEAMAKALVACKLCKAMAHEASENDMVDDISQELNHNSRDFGQLAVELLDQSYKQD
EQLAMKLLTYELKNWSNATCLQLAVAAKHRDFIAHTCSQMLLTDMWMGRLRMRKNSGL
KVILGILLPPSILSLEFKNKDDMPYMSQAQEIHLQEKEAEEPEKPTKEKEEEDMELTAMLG
RNNGESSRKKDEEEVQSKHRLIPLGRKIYEFYNAPIVKFWFYTLAYIGYLMLFNYIVLVKM
ERWPSTQEWMSYIFTLGIEKMREILMSEPGKLLQKVKVWLQEYWNVTDLIAILLFSVGMI
LRLQDQPFRSDGRVIYCVNIIYWYIRLLDIFGVNKYLGPYVMMIGKMMIDMMYFVIIMLVVL
MSFGVARQAILFPNEEPSWKLAKNIFYMPYWMIYGEVFADQIDPPCGQNETREDGKIIQL
PPCKTGAWIVPAIMACYLLVANILLVNLLIAVFNNTFFEVKSISNQVWKFQRYQLIMTFHER
PVLPPPLIIFSHMTMIFQHLCCRWRKHESDPDERDYGLKLFITDDELKKVHDFEEQCIEEY
FREKDDRFNSSNDERIRVTSERVENMSMRLEEVNEREHSMKASLQTVDIRLAQLEDLIGR
MATALERLTGLERAESNKIRSRTSSDCTDAAYIVRQSSFNSQEGNTFKLQESIDPAGEET
MSPTSPTLMPRMRSHSFYSVNMKDKGGIEKLESIFKERSLSLHRATSSHSVAKEPKAPAA
PANTLAIVPDSRRPSSCIDIYVSAMDELHCDIDPLDNSVNILGLGEPSFSTPVPSTAPSSSA
YATLAPTDRPPSRSIDFEDITSMDTRSFSSDYTHLPECQNPWDSEPPMYHTIERSKSSRY
LATTPFLLEEAPIVKSHSFMFSPSRSYYANFGVPVKTAEYTSITDCIDTRCVNAPQAIADRA
AFPGGLGDKVEDLTCCHPEREAELSHPSSDSEENEAKGRRATIAISSQEGDNSERTLSN
NITVPKIERANSYSAEEPSAPYAHTRKSFSISDKLDRQRNTASLRNPFQRSKSSKPEGRG
DSLSMRRLSRTSAFQSFESKHN
SEQ ID NO: 28 Amino acid sequence of human TRPM3 isoform X13
MGKKWRDAAEMERGCSDREDNAESRRRSRSASRGRFAESWKRLSSKQGSTKRSGLP
SQQTPAQKSWIERAFYKRECVHIIPSTKDPHRCCCGRLIGQHVGLTPSISVLQNEKNESR
LSRNDIQSEKWSISKHTQLSPTDAFGTIEFQGGGHSNKAMYVRVSFDTKPDLLLHLMTKE
WQLELPKLLISVHGGLQNFELQPKLKQVFGKGLIKAAMTTGAWIFTGGVNTGVIRHVGDA
LKDHASKSRGKICTIGIAPWGIVENQEDLIGRDVVRPYQTMSNPMSKLTVLNSMHSHFILA
DNGTTGKYGAEVKLRRQLEKHISLQKINTRIGQGVPVVALIVEGGPNVISIVLEYLRDTPPV
VVVCDGSGHASDILAFGHKYSEEGGLINESLHDQLLVTIQKTFTYTRTQAQHLFIILMEC
MKKKELITVFRMGSEGHQDIDLAILTALLKGANASAPDQLSLALAWNRVDIARSQIFIYGQ
QWPVGSLEQAMLDALVLDRVDFVKLLIENGVSMHRFLTISRLEELYNTRHGPSNTLYHLV
RDVKKREYPGFGWIYFKGNLPPDYRISLIDIGLVIEYLMGGAYRCNYTRKRFRTLYHNLFG
PKRPKALKLLGMEDDIPLRRGRKTTKKREEEVDIDLDDPEINHFPFPFHELMVWAVLMKR
QKMALFFWQHGEEAMAKALVACKLCKAMAHEASENDMVDDISQELNHNSRDFGQLAVE
LLDQSYKQDEQLAMKLLTYELKNWSNATCLQLAVAAKHRDFIAHTCSQMLLTDMWMGR
LRMRKNSGLKVILGILLPPSILSLEFKNKDDMPYMSQAQEIHLQEKEAEEPEKPTKEKEEE
DMELTAMLGRNNGESSRKKDEEEVQSKHRLIPLGRKIYEFYNAPIVKFWFYTLAYIGYLML
FNYIVLVKMERWPSTQEWIVISYIFTLGIEKMREILMSEPGKLLQKVKVWLQEYWNVTDLI
AILLFSVGMILRLQDQPFRSDGRVIYCVNIIYWYIRLLDIFGVNKYLGPYVMMIGKMMIDMM
YFVIIMLVVLMSFGVARQAILFPNEEPSWKLAKNIFYMPYWMIYGEVFADQIDPPCGQNET
REDGKIIQLPPCKTGAWIVPAIMACYLLVANILLVNLLIAVFNNTFFEVKSISNQVWKFQRY
QLIMTFHERPVLPPPLIIFSHMTMIFQHLCCRWRKHESDPDERDYGLKLFITDDELKKVHD
FEEQCIEEYFREKDDRFNSSNDERIRVTSERVENMSMRLEEVNEREHSMKASLQTVDIRL
AQLEDLIGRMATALERLTGLERAESNKIRSRTSSDCTDAAYIVRQSSFNSQEGNTFKLQE
SIDPAGEETMSPTSPTLMPRMRSHSFYSVNMKDKGGIEKLESIFKERSLSLHRATSSHSV
AKEPKAPAAPANTLAIVPDSRRPSSCIDIYVSAMDELHCDIDPLDNSVNILGLGEPSFSTPV
PSTAPSSSAYATLAPTDRPPSRSIDFEDITSMDTRSFSSDYTHLPECQNPWDSEPPMYHT
IERSKSSRYLATTPFLLEEAPIVKSHSFMFSPSRSYYANFGVPVKTAEYTSITDCIDTRCVN
APQAIADRAAFPGGLGDKVEDLTCCHPEREAELSHPSSDSEENEAKGRRATIAISSQEGD
NSERTLSNNITVPKIERANSYSAEEPSAPYAHTRKSFSISDKLDRQRNTASLRNPFQRSKS
SKPEGRGDSLSMRRLSRTSAFQSFESKHN
SEQ ID NO: 29 Amino acid sequence of human TRPM3 isoform X10
MPEPWGTVYFLGIAQVFSFLFSWWNLEGVMNQADAPRPLNWTIRKLCHAAFLPSVRLLK
AQKSWIERAFYKRECVHIIPSTKDPHRCCCGRLIGQHVGLTPSISVLQNEKNESRLSRNDI
QSEKWSISKHTQLSPTDAFGTIEFQGGGHSNKAMYVRVSFDTKPDLLLHLMTKEWQLEL
PKLLISVHGGLQNFELQPKLKQVFGKGLIKAAMTTGAWIFTGGVNTGVIRHVGDALKDHA
SKSRGKICTIGIAPWGIVENQEDLIGRDVVRPYQTMSNPMSKLTVLNSMHSHFILADNGTT
GKYGAEVKLRRQLEKHISLQKINTRCLPFFSLDSRLFYSFWGSCQLDSVGIGQGVPVVALI
VEGGPNVISIVLEYLRDTPPVPVVVCDGSGRASDILAFGHKYSEEGGLINESLRDQLLVTI
QKTFTYTRTQAQHLFIILMECMKKKELITVFRMGSEGHQDIDLAILTALLKGANASAPDQLS
LALAWNRVDIARSQIFIYGQQWPVGSLEQAMLDALVLDRVDFVKLLIENGVSMHRFLTISR
LEELYNTRHGPSNTLYHLVRDVKKREYPGFGWIYFKGNLPPDYRISLIDIGLVIEYLMGGA
YRCNYTRKRFRTLYHNLFGPKRDDIPLRRGRKTTKKREEEVDIDLDDPEINHFPFPFHEL
MVWAVLMKRQKMALFFWQHGEEAMAKALVACKLCKAMAHEASENDMVDDISQELNHN
SRDFGQLAVELLDQSYKQDEQLAMKLLTYELKNWSNATCLQLAVAAKHRDFIAHTCSQM
LLTDMWMGRLRMRKNSGLKVILGILLPPSILSLEFKNKDDMPYMSQAQEIHLQEKEAEEP
EKPTKEKEEEDMELTAMLGRNNGESSRKKDEEEVQSKHRLIPLGRKIYEFYNAPIVKFWF
YTLAYIGYLMLFNYIVLVKMERWPSTQEWIVISYIFTLGIEKMREILMSEPGKLLQKVKVWL
QEYWNVTDLIAILLFSVGMILRLQDQPFRSDGRVIYCVNIIYWYIRLLDIFGVNKYLGPYVM
MIGKMMIDMMYFVIIMLVVLMSFGVARQAILFPNEEPSWKLAKNIFYMPYWMIYGEVFAD
QIDPPCGQNETREDGKIIQLPPCKTGAWIVPAIMACYLLVANILLVNLLIAVFNNTFFEVKSI
SNQVWKFQRYQLIMTFHERPVLPPPLIIFSHMTMIFQHLCCRWRKHESDPDERDYGLKLF
ITDDELKKVHDFEEQCIEEYFREKDDRFNSSNDERIRVTSERVENMSMRLEEVNEREHS
MKASLQTVDIRLAQLEDLIGRMATALERLTGLERAESNKIRSRTSSDCTDAAYIVRQSSFN
SQEGNTFKLQESIDPAGEETMSPTSPTLMPRMRSHSFYSVNMKDKGGIEKLESIFKERSL
SLHRATSSHSVAKEPKAPAAPANTLAIVPDSRRPSSCIDIYVSAMDELHCDIDPLDNSVNIL
GLGEPSFSTPVPSTAPSSSAYATLAPTDRPPSRSIDFEDITSMDTRSFSSDYTHLPECQN
PWDSEPPMYHTIERSKSSRYLATTPFLLEEAPIVKSHSFMFSPSRSYYANFGVPVKTAEY
TSITDCIDTRCVNAPQAIADRAAFPGGLGDKVEDLTCCHPEREAELSHPSSDSEENEAKG
RRATIAISSQEGDNSERTLSNNITVPKIERANSYSAEEPSAPYAHTRKSFSISDKLDRQRN
TASLRNPFQRSKSSKPEGRGDSLSMRRLSRTSAFQSFESKHN
SEQ ID NO: 30 Amino acid sequence of human TRPM3 isoform X4
MGSEGHQDIDLAILTALLKGANASAPDQLSLALAWNRVDIARSQIFIYGQQWPVGSLEQA
MLDALVLDRVDFVKLLIENGVSMHRFLTISRLEELYNTRHGPSNTLYHLVRDVKKGNLPP
DYRISLIDIGLVIEYLMGGAYRCNYTRKRFRTLYHNLFGPKRDDIPLRRGRKTTKKREEEV
DIDLDDPEINHFPFPFHELMVWAVLMKRQKMALFFWQHGEEAMAKALVACKLCKAMAH
EASENDMVDDISQELNHNSRDFGQLAVELLDQSYKQDEQLAMKLLTYELKNWSNATCLQ
LAVAAKHRDFIAHTCSQMLLTDMWMGRLRMRKNSGLKVILGILLPPSILSLEFKNKDDMP
YMSQAQEIHLQEKEAEEPEKPTKEKEEEDMELTAMLGRNNGESSRKKDEEEVQSKHRLI
PLGRKIYEFYNAPIVKFWFYTLAYIGYLMLFNYIVLVKMERWPSTQEWIVISYIFTLGIEKMR
EILMSEPGKLLQKVKVWLQEYWNVTDLIAILLFSVGMILRLQDQPFRSDGRVIYCVNIIYWY
IRLLDIFGVNKYLGPYVMMIGKMMIDMMYFVIIMLVVLMSFGVARQAILFPNEEPSWKLAK
NIFYMPYWMIYGEVFADQIDPPCGQNETREDGKIIQLPPCKTGAWIVPAIMACYLLVANILL
VNLLIAVFNNTFFEVKSISNQVWKFQRYQLIMTFHERPVLPPPLIIFSHMTMIFQHLCCRW
RKHESDPDERDYGLKLFITDDELKKVHDFEEQCIEEYFREKDDRFNSSNDERIRVTSERV
ENMSMRLEEVNEREHSMKASLQTVDIRLAQLEDLIGRMATALERLTGLERAESNKIRSRT
SSDCTDAAYIVRQSSFNSQEGNTFKLQESIDPAGEETMSPTSPTLMPRMRSHSFYSVNM
KDKGGIEKLESIFKERSLSLHRATSSHSVAKEPKAPAAPANTLAIVPDSRRPSSCIDIYVSA
MDELHCDIDPLDNSVNILGLGEPSFSTPVPSTAPSSSAYATLAPTDRPPSRSIDFEDITSM
DTRSFSSDYTHLPECQNPWDSEPPMYHTIERSKSSRYLATTPFLLEEAPIVKSHSFMFSP
SRSYYANFGVPVKTAEYTSITDCIDTRCVNAPQAIADRAAFPGGLGDKVEDLTCCHPERE
AELSHPSSDSEENEAKGRRATIAISSQEGDNSERTLSNNITVPKIERANSYSAEEPSAPYA
HTRKSFSISDKLDRQRNTASLRNPFQRSKSSKPEGRGDSLSMRRLSRTSAFQSFESKHN
SEQ ID NO: 31 Amino acid sequence of human TRPM3 isoform X7
MGSEGHQDIDLAILTALLKGANASAPDQLSLALAWNRVDIARSQIFIYGQQWPVGSLEQA
MLDALVLDRVDFVKLLIENGVSMHRFLTISRLEELYNTRHGPSNTLYHLVRDVKKGNLPP
DYRISLIDIGLVIEYLMGGAYRCNYTRKRFRTLYHNLFGPKRPKALKLLGMEDDIPLRRGR
KTTKKREEEVDIDLDDPEINHFPFPFHELMVWAVLMKRQKMALFFWQHGEEAMAKALVA
CKLCKAMAHEASENDMVDDISQELNHNSRDFGQLAVELLDQSYKQDEQLAMKLLTYELK
NWSNATCLQLAVAAKHRDFIAHTCSQMLLTDMWMGRLRMRKNSGLKVILGILLPPSILSL
EFKNKDDMPYMSQAQEIHLQEKEAEEPEKPTKEKEEEDMELTAMLGRNNGESSRKKDE
EEVQSKHRLIPLGRKIYEFYNAPIVKFWFYTLAYIGYLMLFNYIVLVKMERWPSTQEWIVIS
YIFTLGIEKMREILMSEPGKLLQKVKVWLQEYWNVTDLIAILLFSVGMILRLQDQPFRSDG
RVIYCVNIIYWYIRLLDIFGVNKYLGPYVMMIGKMMIDMMYFVIIMLVVLMSFGVARQAILFP
NEEPSWKLAKNIFYMPYWMIYGEVFADQIDPPCGQNETREDGKIIQLPPCKTGAWIVPAI
MACYLLVANILLVNLLIAVFNNTFFEVKSISNQVWKFQRYQLIMTFHERPVLPPPLIIFSHMT
MIFQHLCCRWRKHESDPDERDYGLKLFITDDELKKVHDFEEQCIEEYFREKDDRFNSSN
DERIRVTSERVENMSMRLEEVNEREHSMKASLQTVDIRLAQLEDLIGRMATALERLTGLE
RAESNKIRSRTSSDCTDAAYIVRQSSFNSQEGNTFKLQESIDPAEHPLYSV
SEQ ID NO: 32 Amino acid sequence of human TRPM3 isoform X3
MGSEGHQDIDLAILTALLKGANASAPDQLSLALAWNRVDIARSQIFIYGQQWPVGSLEQA
MLDALVLDRVDFVKLLIENGVSMHRFLTISRLEELYNTRHGPSNTLYHLVRDVKKGNLPP
DYRISLIDIGLVIEYLMGGAYRCNYTRKRFRTLYHNLFGPKRPKALKLLGMEDDIPLRRGR
KTTKKREEEVDIDLDDPEINHFPFPFHELMVWAVLMKRQKMALFFWQHGEEAMAKALVA
CKLCKAMAHEASENDMVDDISQELNHNSRDFGQLAVELLDQSYKQDEQLAMKLLTYELK
NWSNATCLQLAVAAKHRDFIAHTCSQMLLTDMWMGRLRMRKNSGLKVILGILLPPSILSL
EFKNKDDMPYMSQAQEIHLQEKEAEEPEKPTKEKEEEDMELTAMLGRNNGESSRKKDE
EEVQSKHRLIPLGRKIYEFYNAPIVKFWFYTLAYIGYLMLFNYIVLVKMERWPSTQEWIVIS
YIFTLGIEKMREILMSEPGKLLQKVKVWLQEYWNVTDLIAILLFSVGMILRLQDQPFRSDG
RVIYCVNIIYWYIRLLDIFGVNKYLGPYVMMIGKMMIDMMYFVIIMLVVLMSFGVARQAILFP
NEEPSWKLAKNIFYMPYWMIYGEVFADQIDPPCGQNETREDGKIIQLPPCKTGAWIVPAI
MACYLLVANILLVNLLIAVFNNTFFEVKSISNQVWKFQRYQLIMTFHERPVLPPPLIIFSHMT
MIFQHLCCRWRKHESDPDERDYGLKLFITDDELKKVHDFEEQCIEEYFREKDDRFNSSN
DERIRVTSERVENMSMRLEEVNEREHSMKASLQTVDIRLAQLEDLIGRMATALERLTGLE
RAESNKIRSRTSSDCTDAAYIVRQSSFNSQEGNTFKLQESIDPAGEETMSPTSPTLMPRM
RSHSFYSVNMKDKGGIEKLESIFKERSLSLHRATSSHSVAKEPKAPAAPANTLAIVPDSRR
PSSCIDIYVSAMDELHCDIDPLDNSVNILGLGEPSFSTPVPSTAPSSSAYATLAPTDRPPS
RSIDFEDITSMDTRSFSSDYTHLPECQNPWDSEPPMYHTIERSKSSRYLATTPFLLEEAPI
VKSHSFMFSPSRSYYANFGVPVKTAEYTSITDCIDTRCVNAPQAIADRAAFPGGLGDKVE
DLTCCHPEREAELSHPSSDSEENEAKGRRATIAISSQEGDNSERTLSNNITVPKIERANSY
SAEEPSAPYAHTRKSFSISDKLDRQRNTASLRNPFQRSKSSKPEGRGDSLSMRRLSRTS
AFQSFESKHN
SEQ ID NO: 33 Amino acid sequence of human TRPM3 isoform X6
MGSEGHQDIDLAILTALLKGANASAPDQLSLALAWNRVDIARSQIFIYGQQWPVGSLEQA
MLDALVLDRVDFVKLLIENGVSMHRFLTISRLEELYNTRHGPSNTLYHLVRDVKKREYPG
FGWIYFKGNLPPDYRISLIDIGLVIEYLMGGAYRCNYTRKRFRTLYHNLFGPKRDDIPLRR
GRKTTKKREEEVDIDLDDPEINHFPFPFHELMVWAVLMKRQKMALFFWQHGEEAMAKAL
VACKLCKAMAHEASENDMVDDISQELNHNSRDFGQLAVELLDQSYKQDEQLAMKLLTYE
LKNWSNATCLQLAVAAKHRDFIAHTCSQMLLTDMWMGRLRMRKNSGLKVILGILLPPSIL
SLEFKNKDDMPYMSQAQEIHLQEKEAEEPEKPTKEKEEEDMELTAMLGRNNGESSRKK
DEEEVQSKHRLIPLGRKIYEFYNAPIVKFWFYTLAYIGYLMLFNYIVLVKMERWPSTQEWI
VISYIFTLGIEKMREILMSEPGKLLQKVKVWLQEYWNVTDLIAILLFSVGMILRLQDQPFRS
DGRVIYCVNIIYWYIRLLDIFGVNKYLGPYVMMIGKMMIDMMYFVIIMLVVLMSFGVARQAI
LFPNEEPSWKLAKNIFYMPYWMIYGEVFADQIDPPCGQNETREDGKIIQLPPCKTGAWIV
PAIMACYLLVANILLVNLLIAVFNNTFFEVKSISNQVWKFQRYQLIMTFHERPVLPPPLIIFS
HMTMIFQHLCCRWRKHESDPDERDYGLKLFITDDELKKVHDFEEQCIEEYFREKDDRFN
SSNDERIRVTSERVENMSMRLEEVNEREHSMKASLQTVDIRLAQLEDLIGRMATALERLT
GLERAESNKIRSRTSSDCTDAAYIVRQSSFNSQEGNTFKLQESIDPAEHPLYSV
SEQ ID NO: 34 Amino acid sequence of human TRPM3 isoform X2
MGSEGHQDIDLAILTALLKGANASAPDQLSLALAWNRVDIARSQIFIYGQQWPVGSLEQA
MLDALVLDRVDFVKLLIENGVSMHRFLTISRLEELYNTRHGPSNTLYHLVRDVKKREYPG
FGWIYFKGNLPPDYRISLIDIGLVIEYLMGGAYRCNYTRKRFRTLYHNLFGPKRDDIPLRR
GRKTTKKREEEVDIDLDDPEINHFPFPFHELMVWAVLMKRQKMALFFWQHGEEAMAKAL
VACKLCKAMAHEASENDMVDDISQELNHNSRDFGQLAVELLDQSYKQDEQLAMKLLTYE
LKNWSNATCLQLAVAAKHRDFIAHTCSQMLLTDMWMGRLRMRKNSGLKVILGILLPPSIL
SLEFKNKDDMPYMSQAQEIHLQEKEAEEPEKPTKEKEEEDMELTAMLGRNNGESSRKK
DEEEVQSKHRLIPLGRKIYEFYNAPIVKFWFYTLAYIGYLMLFNYIVLVKMERWPSTQEWI
VISYIFTLGIEKMREILMSEPGKLLQKVKVWLQEYWNVTDLIAILLFSVGMILRLQDQPFRS
DGRVIYCVNIIYWYIRLLDIFGVNKYLGPYVMMIGKMMIDMMYFVIIMLVVLMSFGVARQAI
LFPNEEPSWKLAKNIFYMPYWMIYGEVFADQIDPPCGQNETREDGKIIQLPPCKTGAWIV
PAIMACYLLVANILLVNLLIAVFNNTFFEVKSISNQVWKFQRYQLIMTFHERPVLPPPLIIFS
HMTMIFQHLCCRWRKHESDPDERDYGLKLFITDDELKKVHDFEEQCIEEYFREKDDRFN
SSNDERIRVTSERVENMSMRLEEVNEREHSMKASLQTVDIRLAQLEDLIGRMATALERLT
GLERAESNKIRSRTSSDCTDAAYIVRQSSFNSQEGNTFKLQESIDPAGEETMSPTSPTLM
PRMRSHSFYSVNMKDKGGIEKLESIFKERSLSLHRATSSHSVAKEPKAPAAPANTLAIVP
DSRRPSSCIDIYVSAMDELHCDIDPLDNSVNILGLGEPSFSTPVPSTAPSSSAYATLAPTD
RPPSRSIDFEDITSMDTRSFSSDYTHLPECQNPWDSEPPMYHTIERSKSSRYLATTPFLL
EEAPIVKSHSFMFSPSRSYYANFGVPVKTAEYTSITDCIDTRCVNAPQAIADRAAFPGGLG
DKVEDLTCCHPEREAELSHPSSDSEENEAKGRRATIAISSQEGDNSERTLSNNITVPKIER
ANSYSAEEPSAPYAHTRKSFSISDKLDRQRNTASLRNPFQRSKSSKPEGRGDSLSMRRL
SRTSAFQSFESKHN
SEQ ID NO: 35 Amino acid sequence of human TRPM3 isoform X5
MGSEGHQDIDLAILTALLKGANASAPDQLSLALAWNRVDIARSQIFIYGQQWPVGSLEQA
MLDALVLDRVDFVKLLIENGVSMHRFLTISRLEELYNTRHGPSNTLYHLVRDVKKREYPG
FGWIYFKGNLPPDYRISLIDIGLVIEYLMGGAYRCNYTRKRFRTLYHNLFGPKRPKALKLL
GMEDDIPLRRGRKTTKKREEEVDIDLDDPEINHFPFPFHELMVWAVLMKRQKMALFFWQ
HGEEAMAKALVACKLCKAMAHEASENDMVDDISQELNHNSRDFGQLAVELLDQSYKQD
EQLAMKLLTYELKNWSNATCLQLAVAAKHRDFIAHTCSQMLLTDMWMGRLRMRKNSGL
KVILGILLPPSILSLEFKNKDDMPYMSQAQEIHLQEKEAEEPEKPTKEKEEEDMELTAMLG
RNNGESSRKKDEEEVQSKHRLIPLGRKIYEFYNAPIVKFWFYTLAYIGYLMLFNYIVLVKM
ERWPSTQEWIVISYIFTLGIEKMREILMSEPGKLLQKVKVWLQEYWNVTDLIAILLFSVGMI
LRLQDQPFRSDGRVIYCVNIIYWYIRLLDIFGVNKYLGPYVMMIGKMMIDMMYFVIIMLVVL
MSFGVARQAILFPNEEPSWKLAKNIFYMPYWMIYGEVFADQIDPPCGQNETREDGKIIQL
PPCKTGAWIVPAIMACYLLVANILLVNLLIAVFNNTFFEVKSISNQVWKFQRYQLIMTFHER
PVLPPPLIIFSHMTMIFQHLCCRWRKHESDPDERDYGLKLFITDDELKKVHDFEEQCIEEY
FREKDDRFNSSNDERIRVTSERVENMSMRLEEVNEREHSMKASLQTVDIRLAQLEDLIGR
MATALERLTGLERAESNKIRSRTSSDCTDAAYIVRQSSFNSQEGNTFKLQESIDPAEHPL
YSV
SEQ ID NO: 36 Amino acid sequence of human TRPM3 isoform X1
MGSEGHQDIDLAILTALLKGANASAPDQLSLALAWNRVDIARSQIFIYGQQWPVGSLEQA
MLDALVLDRVDFVKLLIENGVSMHRFLTISRLEELYNTRHGPSNTLYHLVRDVKKREYPG
FGWIYFKGNLPPDYRISLIDIGLVIEYLMGGAYRCNYTRKRFRTLYHNLFGPKRPKALKLL
GMEDDIPLRRGRKTTKKREEEVDIDLDDPEINHFPFPFHELMVWAVLMKRQKMALFFWQ
HGEEAMAKALVACKLCKAMAHEASENDMVDDISQELNHNSRDFGQLAVELLDQSYKQD
EQLAMKLLTYELKNWSNATCLQLAVAAKHRDFIAHTCSQMLLTDMWMGRLRMRKNSGL
KVILGILLPPSILSLEFKNKDDMPYMSQAQEIHLQEKEAEEPEKPTKEKEEEDMELTAMLG
RNNGESSRKKDEEEVQSKHRLIPLGRKIYEFYNAPIVKFWFYTLAYIGYLMLFNYIVLVKM
ERWPSTQEWMSYIFTLGIEKMREILMSEPGKLLQKVKVWLQEYWNVTDLIAILLFSVGMI
LRLQDQPFRSDGRVIYCVNIIYWYIRLLDIFGVNKYLGPYVMMIGKMMIDMMYFVIIMLVVL
MSFGVARQAILFPNEEPSWKLAKNIFYMPYWMIYGEVFADQIDPPCGQNETREDGKIIQL
PPCKTGAWIVPAIMACYLLVANILLVNLLIAVFNNTFFEVKSISNQVWKFQRYQLIMTFHER
PVLPPPLIIFSHMTMIFQHLCCRWRKHESDPDERDYGLKLFITDDELKKVHDFEEQCIEEY
FREKDDRFNSSNDERIRVTSERVENMSMRLEEVNEREHSMKASLQTVDIRLAQLEDLIGR
MATALERLTGLERAESNKIRSRTSSDCTDAAYIVRQSSFNSQEGNTFKLQESIDPAGEET
MSPTSPTLMPRMRSHSFYSVNMKDKGGIEKLESIFKERSLSLHRATSSHSVAKEPKAPAA
PANTLAIVPDSRRPSSCIDIYVSAMDELHCDIDPLDNSVNILGLGEPSFSTPVPSTAPSSSA
YATLAPTDRPPSRSIDFEDITSMDTRSFSSDYTHLPECQNPWDSEPPMYHTIERSKSSRY
LATTPFLLEEAPIVKSHSFMFSPSRSYYANFGVPVKTAEYTSITDCIDTRCVNAPQAIADRA
AFPGGLGDKVEDLTCCHPEREAELSHPSSDSEENEAKGRRATIAISSQEGDNSERTLSN
NITVPKIERANSYSAEEPSAPYAHTRKSFSISDKLDRQRNTASLRNPFQRSKSSKPEGRG
DSLSMRRLSRTSAFQSFESKHN
SEQ ID NO: 37 Amino acid sequence of human TRPM3 isoform A2A3F3
MYVRVSFDTKPDLLLHLMTKEWQLELPKLLISVHGGLQNFELQPKLKQVFGKGLIKAAMT
TGAWIFTGGVNTGVIRHVGDALKDHASKSRGKICTIGIAPWGIVENQEDLIGRDVVRPYQT
MSNPMSKLTVLNSMHSHFILADNGTTGKYGAEVKLRRQLEKHISLQKINTRIGQGVPVVA
LIVEGGPNVISIVLEYLRDTPPVPVVVCDGSGRASDILAFGHKYSEEGGLINESLRDQLLVT
IQKTFTYTRTQAQHLFIILMECMKKKELITVFRMGSEGHQDIDLAILTALLKGANASAPDQL
SLALAWNRVDIARSQIFIYGQQWPVGSLEQAMLDALVLDRVDFVKLLIENGVSMHRFLTIS
RLEELYNTRHGPSNTLYHLVRDVKKGNLPPDYRISLIDIGLVIEYLMGGAYRCNYTRKRFR
TLYHNLFGPKRPKALKLLGMEDDIPLRRGRKTTKKREEEVDIDLDDPEINHFPFPFHELMV
WAVLMKRQKMALFFWQHGEEAMAKALVACKLCKAMAHEASENDMVDDISQELNHNSR
DFGQLAVELLDQSYKQDEQLAMKLLTYELKNWSNATCLQLAVAAKHRDFIAHTCSQMLL
TDMWMGRLRMRKNSGLKVILGILLPPSILSLEFKNKDDMPYMSQAQEIHLQEKEAEEPEK
PTKEKEEEDMELTAMLGRNNGESSRKKDEEEVQSKHRLIPLGRKIYEFYNAPIVKFWFYT
LAYIGYLMLFNYIVLVKMERWPSTQEWIVISYIFTLGIEKMREILMSEPGKLLQKVKVWLQE
YWNVTDLIAILLFSVGMILRLQDQPFRSDGRVIYCVNIIYWYIRLLDIFGVNKYLGPYVMMIG
KMMIDMMYFVIIMLVVLMSFGVARQAILFPNEEPSWKLAKNIFYMPYWMIYGEVFADQIDR
KQVYDSHTPKSAPCGQNETREDGKIIQLPPCKTGAWIVPAIMACYLLVANILLVNLLIAVFN
NTFFEVKSISNQVWKFQRYQLIMTFHERPVLPPPLIIFSHMTMIFQHLCCRWRKHESDPD
ERDYGLKLFITDDELKKVHDFEEQCIEEYFREKDDRFNSSNDERIRVTSERVENMSMRLE
EVNEREHSMKASLQTVDIRLAQLEDLIGRMATALERLTGLERAESNKIRSRTSSDCTDAA
YIVRQSSFNSQEGNTFKLQESIDPAGEETMSPTSPTLMPRMRSHSFYSVNMKDKGGIEK
LESIFKERSLSLHRATSSHSVAKEPKAPAAPANTLAIVPDSRRPSSCIDIYVSAMDELHCDI
DPLDNSVNILGLGEPSFSTPVPSTAPSSSAYATLAPTDRPPSRSIDFEDITSMDTRSFSSD
YTHLPECQNPWDSEPPMYHTIERSKSSRYLATTPFLLEEAPIVKSHSFMFSPSRSYYANF
GVPVKTAEYTSITDCIDTRCVNAPQAIADRAAFPGGLGDKVEDLTCCHPEREAELSHPSS
DSEENEAKGRRATIAISSQEGDNSERTLSNNITVPKIERANSYSAEEPSAPYAHTRKSFSI
SDKLDRQRNTASLRNPFQRSKSSKPEGRGDSLSMRRLSRTSAFQSFESKHN
SEQ ID NO: 38 Nucleotide sequence of exon 1 of the human TRPM3 gene
GGCAAAGGCAGCGCCAACCGAGAGCCGCGCGCAGGCTGGAGGGAGACCCGCGGC
GAGGAGCCAGCGAGAGCGCTCGGCGCTGGGCTGTTTCCCGGCCGAGGGAGGCGAA
CTTCTCATGGGGAAGAAGTGGAGGGATGCGGCGGAAATGGAGCGGGGCTGCTCCG
ACCGCGAGGACAACGCGGAGAGCCGCAGACGCAGCCGGAGCGCCAGCCGGGGCA
GGTTTGCCGAGTCGTGGAAAAGGTTAAGTTCCAAGCAGGGGTCCACCAAACGCTCG
GGACTCCCGTCGCAGCAGACGCCG
SEQ ID NO: 39 Nucleotide sequence of exon 2 of the human TRPM3 gene
ATTCTGGAGCAGGCTGCTTTGACTCCGACCACAGGCTGTTTTGTGCAGGCTGTCCCT
CTTCTTCAAAATCGTGCATCCCCTCCCCGAAGCAGCAGGCAGTGTGCCTCCATTCAG
CCACATTTGGTATGCATGAGCACGGCTGCAGAGAGAGGGGAGGTGGCTGTTTTAAG
AAGGTTCAGGGGCTCAGGCAAGGCTACTTGACTAGTCTTCCAAGTTCCAGGAAGCCT
CTGCCCTAATGGAATTTGCAGGTGTGGAGATGACCATGGGATGCCAGAGCCGTGGG
GGACCGTTTATTTTCTAGGCATTGCTCAGGTTTTCAGTTTCTTGTTTTCCTGGTGGAAT
TTGGAAGGGGTCATGAATCAGGCTGATGCTCCTCGACCCCTAAACTGGACCATCCG
GAAGCTGTGCCACGCAGCCTTTCTTCCATCTGTCAGACTTCTGAAG
SEQ ID NO: 40 Nucleotide sequence of exon 3 of the human TRPM3 gene
AGCAAAATGAAAGCAACAGGAGCTGCTCCGGGGACTGCTTTTGCCAGTACCCAGAAT
CAGTGCTCAG
SEQ ID NO: 41 Nucleotide sequence of exon 4 of the human TRPM3 gene
GCTCAGAAATCCTGGATAGAAAGAGCATTTTATAAAAGAGAATGTGTCCACATCATAC
CCAGCACCAAAGACCCCCATAG
SEQ ID NO: 42 Nucleotide sequence of exon 5 of the human TRPM3 gene
GTGTTGCTGTGGGCGTCTGATAGGCCAGCATGTTGGCCTCACCCCCAGTATCTCCGT
GCTTCAGAATGAGAAAAATGAAAGTCGCCTCTCCCGAAATGACATCCAGTCTGAAAA
GTGGTCCATCAGCAAACACACTCAACTCAGCCCTACGGATGCTTTTGGGACCATTGA
GTTCCAAGGAGGTGGCCATTCCAACAAAGCCATG
SEQ ID NO: 43 Nucleotide sequence of exon 6 of the human TRPM3 gene
TATGTGCGAGTATCTTTTGATACAAAACCTGATCTCCTCTTACACCTGATGACCAAGG
AATGGCAGTTGGAGCTTCCCAAGCTTCTCATCTCTGTCCATGGGGGCCTGCAGAACT
TTGAACTCCAGCCAAAACTCAAGCAAGTCTTTGGGAAAGGGCTCATCAAAGCAGCAA
TGACAACTGGAGCGTGGATATTCACTGGAGGGGTTAACACAG
SEQ ID NO: 44 Nucleotide sequence of exon 7 of the human TRPM3 gene
GTGTTATTCGTCATGTTGGCGATGCCTTGAAGGATCATGCCTCTAAGTCTCGAGGAA
AGATATGCACCATAGGTATTGCCCCCTGGGGAATTGTGGAAAACCAGGAGGACCTCA
TTGGAAGAGAT
SEQ ID NO: 45 Nucleotide sequence of exon 8 of the human TRPM3 gene
GTTGTCCGGCCATACCAGACCATGTCCAATCCCATGAGCAAGCTCACTGTTCTCAAC
AGCATGCATTCCCACTTCATTCTGGCTGACAACGGGACCACTGGAAAATATGGAGCA
GAGGTGAAACTTCGAAGACAACTGGAAAAGCATATTTCACTCCAGAAGATAAACACAA
SEQ ID NO: 46 Nucleotide sequence of exon 9 of the human TRPM3 gene
GATGCCTGCCGTTTTTCTCTCTTGACTCCCGCTTGTTTTATTCATTTTGGGGTAGTTG
CCAGTTAGACTCAGTTG
SEQ ID NO: 47 Nucleotide sequence of exon 10 of the human TRPM3 gene
GAATCGGTCAAGGTGTTCCTGTGGTGGCACTCATAGTGGAAGGAGGACCCAATGTG
ATCTCGATTGTTTTGGAGTACCTTCGAGACACCCCTCCCGTGCCAGTGGTTGTCTGT
GATGGGAGTGGACGGGCATCGGACATCCTGGCCTTTGGGCATAAATACTCAGAAGA
AGGCGG
SEQ ID NO: 48 Nucleotide sequence of exon 11 of the human TRPM3 gene
ACTGATAAATGAATCTTTGAGGGACCAGCTGTTGGTGACTATACAGAAGACTTTCACA
TACACTCGAACCCAAGCTCAGCATCTGTTCATCATCCTCATGGAGTGCATGAAGAAG
AAGGAATTG
SEQ ID NO: 49 Nucleotide sequence of exon 11 a of the human TRPM3 gene
GCAGAATCTCACCAGGCCTGCCTAAACTACCCATCCCTGGCTGACCTCAGTGCCCCC
TAGTCTCTCCATTCCAGTTGAAACAGGACAGCTGGATGACTCCCATTAAGGAGCAAC
TCTCATAATGTAACCTCCCACCCAATCGGAAACCCACATTACCCTCGCTGTGCTTCCC
AAG
SEQ ID NO: 50 Nucleotide sequence of exon 12 of the human TRPM3 gene
ATTACGGTATTTCGGATGGGATCAGAAGGACACCAGGACATTGATTTGGCTATCCTG
ACAGCTTTACTCAAAG
SEQ ID NO: 51 Nucleotide sequence of exon 13 of the human TRPM3 gene
GAGCCAATGCCTCGGCCCCAGACCAACTGAGCTTAGCTTTAGCCTGGAACAGAGTC
GACATCGCTCGCAGCCAGATCTTTATTTACGGGCAACAGTGGCCG
SEQ ID NO: 52 Nucleotide sequence of exon 14 of the human TRPM3 gene
GTGGGATCTCTGGAGCAAGCCATGTTGGATGCCTTAGTTCTGGACAGAGTGGATTTT
GTGAAATTACTCATAGAGAATGGAGTAAGCATGCACCGTTTTCTCACCATCTCCAGAC
TAGAGGAATTGTACAATACG
SEQ ID NO: 53 Nucleotide sequence of exon 15 of the human TRPM3 gene
AGACATGGGCCCTCAAATACATTGTACCACTTGGTCAGGGATGTCAAAAAG
SEQ ID NO: 54 Nucleotide sequence of exon 16 of the human TRPM3 gene
CGAGAGTATCCAGGTTTCGGTTGGATCTATTTTAAG
SEQ ID NO: 55 Nucleotide sequence of exon 17 of the human TRPM3 gene
GGGAACCTGCCCCCAGACTACAGAATCAGCCTGATTGACATCGGCCTGGTGATCGA
GTACCTGATGGGCGGGGCTTATCGCTGCAACTACACGCGCAAGCGCTTCCGGACCC
TCTACCACAACCTCTTCGGCCCCAAGAGG
SEQ ID NO: 56 Nucleotide sequence of exon 18 of the human TRPM3 gene
CCCAAAGCCTTGAAACTGCTGGGAATGGAG
SEQ ID NO: 57 Nucleotide sequence of exon 19 of the human TRPM3 gene
GATGATATTCCCTTGAGGCGAGGAAGAAAGACAACCAAGAAACGTGAAGAAGAGGT
GGACATTGACTTGGATGATCCTGAGATCAACCACTTCCCCTTCCCTTTCCATGAGCTC
ATGGTGTGGGCTGTTCTCATGAAGCGGCAGAAGATGGCCCTGTTCTTCTGGCAGCA
CGGTGAGGAGGCCATGGCCAAGGCCCTGGTGGCCTGCAAGCTCTGCAAAGCCATG
GCTCATGAGGCCTCTGAGAACGACATGGTTGACGACATTTCCCAGGAGCTGAATCAC
AATTCCAG
SEQ ID NO: 58 Nucleotide sequence of exon 20 of the human TRPM3 gene
AGACTTTGGCCAGCTGGCTGTGGAGCTCCTGGACCAGTCCTACAAGCAGGACGAAC
AGCTGGCCATGAAACTGCTGACGTATGAGCTGAAGAACTGGAGCAACGCCACGTGC
CTGCAGCTTGCCGTGGCTGCCAAACACCGCGACTTCATCGCGCACACGTGCAGCCA
GATGCTGCTCACCGACATGTGGATGGGCCGGCTCCGCATGCGCAAGAACTCAGGCC
TCAAG
SEQ ID NO: 59 Nucleotide sequence of exon 21 of the human TRPM3 gene
GTAATTCTGGGAATTCTACTTCCTCCTTCAATTCTCAGCTTGGAGTTCAAGAACAAAG
ACGACATGCCCTATATGTCTCAGGCCCAGGAAATCCACCTCCAAGAGAAGGAGGCA
GAAGAACCAGAGAAGCCCACAAAGGAAAAAGAGGAAGAGGACATGGAGCTCACA
SEQ ID NO: 60 Nucleotide sequence of exon 22 of the human TRPM3 gene
GCAATGTTGGGACGAAACAACGGGGAGTCCTCCAGGAAGAAGGATGAAGAGGAAGT
TCAGAGCAAGCACCGGTTAATCCCCCTCGGCAGAAAAATCTATGAATTCTACAATGC
ACCCATCGTGAAGTTCTGGTTCTACACA
SEQ ID NO: 61 Nucleotide sequence of exon 23 of the human TRPM3 gene
CTGGCGTATATCGGATACCTGATGCTCTTCAACTATATCGTGTTAGTGAAGATGGAAC
GCTGGCCGTCCACCCAGGAATGGATCGTAATCTCCTATATTTTCACCCTGGGAATAG
AAAAGATGAGAGAG
SEQ ID NO: 62 Nucleotide sequence of exon 24 of the human TRPM3 gene
ATTCTGATGTCAGAGCCAGGGAAGTTGCTACAGAAAGTGAAGGTATGGCTGCAGGA
GTACTGGAATGTCACGGACCTCATCGCCATCCTTCTGTTTTCTGTCGGAATGATCCTT
CGTCTCCAAGACCAGCCCTTCAGGAGTGACGGGAGGGTCATCTACTGCGTGAACAT
CATTTACTGGTATATCCGTCTCCTAGACATCTTCGGCGTGAACAAGTATTTGGGCCCG
TATGTAATGATGATTGGAAAAATG
SEQ ID NO: 63 Nucleotide sequence of exon 25 of the human TRPM3 gene
ATGATAGACATGATGTACTTTGTCATCATTATGCTGGTGGTTCTGATGAGCTTTGGGG
TCGCCAGGCAAGCCATCCTTTTTCCCAATGAGGAGCCATCATGGAAACTGGCCAAGA
ACATCTTCTACATGCCCTATTGGATGATTTATGGGGAAGTGTTTGCGGACCAGATAGA
CC
SEQ ID NO: 64 Nucleotide sequence of exon 26 of the human TRPM3 gene
CTCCCTGTGGACAGAATGAGACCCGAGAGGATGGTAAAATAATCCAGCTGCCTCCCT
GCAAGACAGGAGCTTGGATCGTGCCGGCCATCATGGCCTGCTACCTCTTAGTGGCA
AACATCTTGCTGGTCAACCTCCTCATTGCTGTCTTTAA
SEQ ID NO: 65 Nucleotide sequence of exon 27 of the human TRPM3 gene
CAATACATTTTTTGAAGTAAAATCGATATCCAACCAAGTCTGGAAGTTTCAGAGGTAT
CAGCTCATCATGACTTTCCATGAAAGGCCAGTTCTGCCCCCACCACTGATCATCTTCA
GCCACATGACCATGATATTCCAGCACCTGTGCTGCCGATGGAGGAAACACGAGAGC
GACCCGGATGAAAGGGACTACGGCCTGA
SEQ ID NO: 66 Nucleotide sequence of exon 28 of the human TRPM3 gene
AACTCTTCATAACCGATGATGAGCTCAAGAAAGTACATGACTTTGAAGAGCAATGCAT
AGAAGAATACTTCAGAGAAAAGGATGATCGGTTCAACTCATCTAATGATGAGAGGATA
CGGGTGACTTCAGAAAG
SEQ ID NO: 67 Nucleotide sequence of exon 29 of the human TRPM3 gene
GGTGGAGAACATGTCTATGCGGCTGGAGGAAGTCAACGAGAGAGAGCACTCCATGA
AGGCTTCACTCCAGACCGTGGACATCCGGCTGGCGCAGCTGGAAGACCTTATCGGG
CGCATGGCCACGGCCCTGGAGCGCCTGACAGGTCTGGAGCGGGCCGAGTCCAACA
AAATCCGCTCGAGGACCTCGTCAGACTGCACGGACGCCGCCTACATTGTCCGTCAG
AGCAGCTTCAACAGCCAGGAAGGGAACACCTTCAAGCTCCAAGAGAGTATAGACCCT
GCAGGTGAGGAGACCATGTCCCCAACTTCTCCAACCTTAATGCCCCGTATGCGAAGC
CATTCTTTCTATTCGGTCAATATGAAAGACAAAGGTGGTATAGAAAAGTTGGAAAGTA
TTTTTAAAGAAAGGTCCCTGAGCCTACACCGGGCTACTAGTTCCCACTCTGTAGCAAA
AGAACCCAAAGCTCCTGCAGCCCCTGCCAACACCTTGGCCATTGTTCCTGATTCCAG
AAGACCATCATCGTGTATAGACATCTATGTCTCTGCTATGGATGAGCTCCACTGTGAT
ATAGACCCTCTGGACAATTCCGTGAACATCCTTGGGCTGGGCGAGCCAAGCTTTTCA
ACTCCAGTACCTTCCACAGCCCCTTCAAGTAGTGCCTATGCAACACTTGCACCCACA
GACAGACCTCCAAGCCGGAGCATTGATTTTGAGGACATCACCTCCATGGACACTAGA
TCTTTTTCTTCAGACTACACCCACCTCCCAGAATGCCAAAACCCCTGGGACTCAGAG
CCTCCGATGTACCACACCATTGAGCGTTCCAAAAGTAGCCGCTACCTAGCCACCACA
ceCTTTCTTCTAGAAGAGGCTCCCATTGTGAAATCTCATAGCTTTATGTTTTCCCCCT
CAAGGAGCTATTATGCCAACTTTGGGGTGCCTGTAAAAACAGCAGAATACACAAGTA
TTACAGACTGTATTGACACAAGGTGTGTCAATGCCCCTCAAGCAATTGCGGACAGAG
CTGCCTTCCCTGGAGGTCTTGGAGACAAAGTGGAGGACTTAACTTGCTGCCATCCAG
AGCGAGAAGCAGAACTGAGTCACCCCAGCTCTGACAGTGAGGAGAATGAGGCCAAA
GGCCGCAGAGCCACCATTGCAATATCCTCCCAGGAGGGTGATAACTCAGAGAGAAC
CCTGTCCAACAACATCACTGTTCCCAAGATAGAGCGCGCCAACAGCTACTCGGCAGA
GGAGCCAAGTGCGCCATATGCACACACCAGGAAGAGCTTCTCCATCAGTGACAAACT
CGACAGGCAGCGGAACACAGCAAGCCTGCGAAATCCCTTCCAGAGAAGCAAGTCCT
CCAAGCCGGAGGGCCGAGGGGACAGCCTGTCCATGAGGAGACTGTCCAGAACATC
GGCTTTCCAAAGCTTTGAAAGCAAGCACAACTAAACCTTCTTAATATCCGCCACAGAA
GGCTCAAGAATCCAGCCCTAAAATTCTCTCCAACTCCAGTTTTTCCCCTTTCCTTGAA
TCATACCTGCTTTATTCTTAGCTGAGCAAAACAAGCAATGCTTTGGGAGGTGTTAACT
CAAAGGTGACTTCTGGGCCACAGATCAAGAAAGCATTTGATCTGACCCAGTGCCAGA
CACAGGGGATTTAAGGCATGTTCACACTTGCTGGGCAGGGAGGGGGAAGAGAGGGA
GAAGGAAGGGTTAGAGATGAATGTGTATCCGCAGTCACAGCAGAAAGCCATGAGAG
CAGGGGAAACAAGGGGCTTCGAGCACGCTCCATGCCAGGAGGCATCTGTTGATTTC
TGACCATTATCAAGAGTTGTAGGATGCAGGGCTAAATTGCAAAATAAAATAAAATAGC
CAGCGTACACAATGAGATATTCTAAACTTCCATTCTGTTTTCTTTTCACATTGGCTCCA
TCACTGGTGACTGATGAAGAGCATCCTCTTTATTCAGTATAAGCCGGCAGCAAGCAG
TTCTACCTAACGTCCCACATCCTTCTCATGCCAACACTTCTGTAATTGATCATTATAAA
GAAAAAACAAGGTAACAGTCATAGTTCACCTGTCTCTTATCTATTCACTTCTGGTGCC
ACAACTGTTTATCCTTTTTTGAAGAAAATAAGGGAACAGAAATGCCTTTTTGTATTGCA
ATCGAAATGAAAGAAGAGTTGATGTTAAAAAAACAAAAGTCAAGTGATTTATTATATAC
AGTGGGCGTTCAAGTCTAGTCGAGCAAGCTCAGGAGAATGTAATTAAATAATTTTATA
TTTTTTAATTTATTTTGTATCTCACCTGTCATGGATGAATTCATTCACTGAATATGTAAT
ATTGAACTTAAAAAAAAAAAAAAAAAAACAGTGGACAGAACTAGCCTGAAATTGCCAT
GTGGGATGTTCTGTTCTCACCGTTCATGTCGTGTGTTGTGCTGCTGTGTGACCGTCA
TCATTTGCATGGCTCCACCACGTCCTCCTGAACACCTCCAAGACGTAACTGCCAATTT
TTCACACCACTCATCACATGACCATTTTGTATATGAATATATGGTTATATGTGGATATT
TTCATACCTAGAGTTTTGCCATCTAATCTGAGCTCAGCATAAATTTAATTGGAGTTTTT
CAAGGGCTGGTATCTTTTCTATGGAAATGTTCCAAAGTACTTTTTAAGGAAGTTTCAAA
ACTATAAATAACCTTAGATTTGACTGGGAGTTTGGTATCAACCCAAAGCCATCAGTTG
CAAGCATACCATGAATATTTTTCTTATAGACAGAGCTATAAAAAAAGATACAGTAGACC
AAAATAGAAAGCAAATAAACCATATAAAGAGTTCTATAGTATATGCCACTTATACACAT
GTATATGCATTTATGCAGAGACACATATTTATATATAAAATAAATGGACATAAAGAGCT
TTCTTAAGAGGCTTGGACTTTTCTATCACCTTGAAGTATAATCAAGATTTTTTTTAATAT
ACAAGAAGTTCCATTGAAAGAGTTCATTATACTATTAGACCAAATGTTGAAATTATGAA
TTTTTTAAATTCCTATGACTTTTCCTCATATCAAACTAGAGTTGTCAAAATGACTCACT
GCTCGCCTTCTGCCCTTTTTTGTCTGAAATCACTCACTATCAGGACATCTCATTTAGT
CTCGCTTTTCCTAAACAAAGTTTTTACACCACGTACAATCTGAATAGACACGCAACTTT
AAACCAGCTAGTGTTACAGACTCTAAAGTAGAGGCATAATCTTCTAGATCAGAATCTG
GATTGGAAGTGGGATAAAGAAATCTTGACCCCAAATCTTTGGATTCCTATTTGGCTCA
TTAACGTTTAAAAAAGAATAAAGCCTTTAAGGAAAACAATCTATTGTTCCTGGCCTGA
GATACTACCTGGTTCTTTTGTTTAATTATAATTGAAAACACCAAGTTTTACTTTATTTTG
TGAAATCAAGTAAATACATGGCCTTTAAATATAAGATAAAAAACAAAATAAAAACTCTT
CTGAATAAGCATTTCAGGTATAAAAATTGTGATGAGGTCACATATGTAAAGAATTAAAC
AATTCCTTTTTCACTCCTTGGCATTTAGGTGAATTGCTAACAAACACCCAGACTCCCTT
ATTTACAGCTGTTCTAGAAACACTTAACATTTTATTGGGCCCCACGCTGGCTATTTCC
CTCTCAGCAAACATGCACTGGGTCTGAGAATAAATCAGTCGTTCTTAAACTTCCTTGA
GTCATGAATCCCTACGAGGCTCTACGATAACTCTAGGCTCTCTCTCCAGAAAGAAAAA
AAAAATGTACATAATGCAAAGTTGCAATATAACTTGAAAGGGTTCATGAGCCCCTGGT
TCTATATTGATTATGAGGGAAAGGAACCATGTTTGCTCGTAGTTAATGCAGTTGTAAT
AGGCACTTTACATTTGAGACCATCTCTCTGTGATTCTCAGGAAGGCCAAATCTCAGCA
CCAGAGTTTGAACAAAGATTTTGTTATTTTTGTTCTTTAAATTAGATGTTGCCAATTTAA
TGCACTTACATCCTAACATGTTGATGATTTTAAGTATCAAGCACAATCACAATGCTGAA
GTCTCCCTGAAGGGTCTATAGAGGAATCCAAGAAGGAGGTGAAAACATTATTCATTG
ACATCAAACACCTCCAAGGCCTCATTCTGGGCCAGATTGGAAGTGAGGGAAAGAAAT
TTTCAGCTCAAGTGTTTCCATTCATCGTGATTTCAGAGAGAAAAGGATAGGAACTGGG
AAACGAAATACTCAACAGTCCTGATCTTGCAATTACCTGATTCTTTTGTTGTGCATGAA
AATTAAGTTCTAACGTCTGTGAAACGTGTGAAATATATAAGCCAAACTTAAATGACTGT
AGACACAAACTGTGTAACTTCCATGAGCCATTTCCTTCCTGTGCACATGTATGTGTGT
TATTTTTCAGTCCATTTCCTTCCATTTCTGGGTTCTCTGTGCAGCAATGTGGCATTGAT
TCTGTGGCCTTGTGCAGTCAGCTTATGCTCAGCCCATTGATTCCACCACATCCCTGAT
CGAGGGGACGTTAATTACCTGTCTTTGTGAATTAAGCAGTTTATGAGCAGCAAGTTAC
TGAGGACCGTAAATAAAGTCATAGAGAGCTCAAGGAAGTAATAGCTTCGGTTTAATCA
TCTGCTTCAAAGGTGCCAATTTTTACATACTACTATATGTAATGATTTGTTCTAAATTG
CTTGTTCCTTTTGAAAAAAATTTAAGGATGTTTTATGAAAATGACCATGGAGGTCTCCT
GCTTAACCAATATACTTTATATTTATAGTTAGAGGGCCCCTCCTTGATAGATTTGAATG
AGGAGCTGGAAAGCTTGGGTACAATTTTCATGACTAGTTTGTATTATCTCTGCAAGCC
AAAAGGTGTATTAGCAGTCAGAATACAGCACTTTACAGAACTGGATTTTTTAAATGTAT
AAGTTTCCAGGTCTAGGATATTATTTAGCACTTTGAAAGTTCCCCTTTTAAAATATATA
TATAAGAATAATCAAACGAGAAAAATGTACTCATAAAAACACAAAATGTGTTTCTTCTG
AATGTTTCAAATGTATTCTAAAATATGGTTTCTATAGATAATATTATGGTAGATAACTG
GATAGTACTTTTTTACTTTCTGAGTCTGTACAGTCTAATCTTCCTGAATGTTTGGGCTG
TTTAATTTCTTTAAAGATGATAAATATTACCCGGTCATCCATCATGCTCTATCTATAGG
CTATACCATTCATATGTTGGGCAATGCTGTATTATAAAACACATGTAAACATGTATGTA
ACCCATGTTTAACCCAGCGTAGTTGTAGCATGTGGTTGTATTAATGAACGTTACAGGA
GAGCTTTATAATCATTTATAAATCTGTAACATTCAATAAAGAAACACATGTTGCAATGA
TTAATTATGTTCCCAATTCCATGTTATTTAGCATCTTTGTGAACCGGGCATTTTTGTTC
CTGATCTGCTTGATGCTTTTCATGTTCCTTGGACCACTGTAAAATATGTGTGTAAGCT
GATGTATATCCTCTAATGAGTCCTGCGAGCTGTGGTAGGCAAGTTGTGATTTTGTGAA
AGATGAACTGGCATGTTATGCAAAAGACTATATGACAAATGAACTTAAAATGATATTTT
AAACTCCAGTTGAATTAGCTGTTTACATCTGCCAACCCCATGCAGAAGCAGTGTACAG
TATTTTATAAATATGTTGATTTAGTAACACTATGTTGTTTCTAGATAAAGTGATAGTAGT
GTGTAGGTGGTTATATATTTGGCCTAGATATTGCATTATAACTCACTATCCTTGAACAA
TAACAATGTGACTGAAATGAAGTAATCTCTCTCCAAACCTGGTCTTAATTTTGTGTCAA
CAAAATGCATGCACTGCTTTATGCATTTCTTTTGACTTTTCTAAGGCACTTAACATTTT
ACTATTGCCTGCTGTGACGCTTAAGCCAGTTAAGAATTCACTCAAACAGTTGGGCTTC
TTACTATGGGGATTATGTTCAGTTTTGAAAAATTAAAAACAAAAAAAAAGTAACCAAGG
GATTTTTAAATCTTTTTTTTAAGCACTGAACAAATAATATGAAATAAAGTATTAACTGCA
GGTTGTATAGCCATGGGACCCAGAGGTGGCAAGCCAACACCTCTTGGGTCTGCCAA
GGTCTAGGTGTTGTGCATCTGTGATATATGCAAGACACATTTCTGATTCTTCAGAGCT
ACTGGGTGCAAATTGCTAAGCCGGAGATGAGAGCGAAAAGTCCATTTGTGATAGTGC
AGAAGTGCGCAGATCGCTGTGAATGTGTGATCCAATGATATGACAAGAGACCTCCCT
CCTTGTCTTGTGGCTTCTTCACCTTGGGGGTGGGAGGCGTGTGTTTGGGTGTCTGAT
GAAGCACTGGGTGGTTCTAAAGAGTTTACTCTTTTAGAAAATCATCATTACTAGAAGC
CATTCAACATCTGTTAAAGAGAGGAGCCAATTTGCACAGTTTTGAAAATGACTGCTCC
TAAAATGAGTAGACAAAACTTTCCTCTCAGCCTGCATTGACCTAAGCTTTCTCTTCTTT
TTACAAGTTGGATATTCTGTCTCTCAGATTGGAGTCAACCAAAAGAAAGGCTGGTTCC
AATTAAGGAAAAGCAATTTTTTTCTTTTAATTCCTCTCAGAAGGTAGTTGTGTTACCTT
TTCTCTGATGATTTCCTTGCACTCATCACTGCTAATGAAAGCAACGTGACTGTTTCTA
CAGGTCATCTAAGAGAGGATTTTGGTGACCAGGTAATCCAGATGGCAATAAGAATGA
GGTGAATTTTCCCTTCCTTACCTTACCTAGTCTACCAGCCTCCTTAAACCTGCAGGCC
CTTGTGCGTTTGCACAATGTTTGTGTGCTGGAAACTGAAAAGACATGTGTATGGCTAG
AAGTCCATAGGCTTTGTAAAGAGTGTCAGTGCCAATCAGGACCCATGCCTTCCTCTG
GAGGAGACAGGTGGCTGAGGCCTATGGGGCAAAGGGGGAAATTCAGCCTTGTGCCT
GGATGTGCGGCTTTCTTTCTCCCCTCTTCTTGCTTCTCTTCTCTTAGTCTAAGACTAG
CTGCTTTTTCACCAGACCAACAGACCCTTGCCCACATTACCTTTATACCTTGTTGACC
CTCGCACAGTAAGCTGAAACAGCGGCCGCTTGGAAAAAGGCAAACATTTAGACTTTG
TCCCAGGCCTGATCTTCCCACCAACTTAGCCTCCTCCTGCCTCTACCCCGGCTGGTG
TTCACATTGACCTCGTCCAATTTTTGCCTCTCTAAATCTCAAGAGTCCTACACATCTTC
ACCGCAGCTTCCCACACATCATTAAAACCAGCCATCTGCACATAGACTGAGAGGCTG
CAAATTCAAACACATGGAGGGGCCGGATCCAGAAAACACAAATGTGTGAACTGGACT
GGTGAAAGTCAACAGAAGTAGGGTTTGTAAGTAAGGCATGACAGAATACACATCCCA
CCACAAACACCTTCAAATTCAGCTTTCCAAACACTGTCCTAGCCAAACAAACATGTCT
GCCAGCAGCATTCAGTCCCAAAGGCAATAGTTTGGGACCTCTGATTTAGAGCCAACA
GGATCAGATTCTTGATAGTGACTTGGTGAGTGTACACACAAAATACACTGAGAACCG
AGAGGGGGGAAAAGAAACTCGCCTTATTCAGAGTTACTCTTGTTCTGAATGATATATG
CATGTATATTTGTTAGCCACATAATTCTAAACTTGAACACAGAGTCTTTGTGAGAGAG
ATTCAGAAGAAGAGTACCATAGCATGGTATGAAGACTTACCTGCTTCCTGTCACTACT
GCGTCTGACCTGAGTTTATATTGCTGCTACCGCTGTGTAAAAACAGTACGTGCAGTAT
ACTGTACTCATAGCCATTTCATAATATAGGACCTAGACTCTCATTTGTAAACGTTCTAA
CCCTAGTTTATAATGGGGGAAATTATTCAGATATCATTTTTAAATGAATTCCTACAAAT
ACAATGGTTTTAAAATTGGGTTTGCTGAATATATATACATCTTTTCCTTCTTTGTGTATG
GATGGACCACGGTATGCATATCTATCGACACTATAAATATATATAAATCCTTAGAAGA
ATTGTTCTTTTTTAAAGGTCTATTATTGGATTCACAATGCACTTTGAGTTTGATTGTTTA
GATATACAATTCAGAGCAGAAGTTGGCAGATACCACGGGAAAAACTTTCTGAAATTTC
TGTAACAAATGTTTTGCAATAAAAAAAAAAACTCAGTAGTTTAAAACA
SEQ ID NO: 68 Nucleotide sequence of exon 29 of the human TRPM3 gene
where exon 30 is used
GGTGGAGAACATGTCTATGCGGCTGGAGGAAGTCAACGAGAGAGAGCACTCCATGA
AGGCTTCACTCCAGACCGTGGACATCCGGCTGGCGCAGCTGGAAGACCTTATCGGG
CGCATGGCCACGGCCCTGGAGCGCCTGACAGGTCTGGAGCGGGCCGAGTCCAACA
AAATCCGCTCGAGGACCTCGTCAGACTGCACGGACGCCGCCTACATTGTCCGTCAG
AGCAGCTTCAACAGCCAGGAAGGGAACACCTTCAAGCTCCAAGAGAGTATAGACCCT
GCAG
SEQ ID NO: 69 Nucleotide sequence of exon 30 of the human TRPM3 gene
AGCATCCTCTTTATTCAGTATAAGCCGGCAGCAAGCAGTTCTACCTAACGTCCCACAT
CCTTCTCATGCCAACACTTCTGTAATTGATCATTATAAAGAAAAAACAAGGTAACAGTC
ATAGTTCACCTGTCTCTTATCTATTCACTTCTGGTGCCACAACTGTTTATCCTTTTTTG
AAGAAAATAAGGGAACAGAAATGCCTTTTTGTATTGCAATCGAAATGAAAGAAGAGTT
GATGTTAAAAAAACAAAAGTCAAGTGATTTATTATATACAGTGGGCGTTCAAGTCTAG
TCGAGCAAGCTCAGGAGAATGTAATTAAATAATTTTATATTTTTTAATTTATTTTGTATC
TCACCTGTCATGGATGAATTCATTCACTGAATATGTAATATTGAACTTAAAAAAAAAAA
AAAAAAAACAGTGGACAGAACTAGCCTGAAATTGCCATGTGGGATGTTCTGTTCTCAC
CGTTCATGTCGTGTGTTGTGCTGCTGTGTGACCGTCATCATTTGCATGGCTCCACCA
CGTCCTCCTGAACACCTCCAAGACGTAACTGCCAATTTTTCACACCACTCATCACATG
ACCATTTTGTATATGAATATATGGTTATATGTGGATATTTTCATACGTAGAGTTTTGCC
ATCTAATCTGAGCTCAGCATAAATTTAATTGGAGTTTTTCAAGGGCTGGTATCTTTTCT
ATGGAAATGTTCCAAAGTACTTTTTAAGGAAGTTTCAAAACTATAAATAACCTTAGATT
TGACTGGGAGTTTGGTATCAACCCAAAGCCATCAGTTGCAAGCATACCATGAATATTT
TTCTTATAGACAGAGCTATAAAAAAAGATACAGTAGACCAAAATAGAAAGCAAATAAA
CCATATAAAGAGTTCTATAGTATATGCCACTTATACACATGTATATGCATTTATGCAGA
GACACATATTTATATATAAAATAAATGGACATAAAGAGCTTTCTTAAGAGGCTTGGACT
TTTCTATCACCTTGAAGTATAATCAAGATTTTTTTTAATATACAAGAAGTTCCATTGAAA
GAGTTCATTATACTATTAGACCAAATGTTGAAATTATGAATTTTTTAAATTCCTATGACT
TTTCCTCATATCAAACTAGAGTTGTCAAAATGACTCACTGCTCGCCTTCTGCCCTTTTT
TGTCTGAAATCACTCACTATCAGGACATCTCATTTAGTCTCGCTTTTCCTAAACAAAGT
TTTTACACCACGTACAATCTGAATAGACACGCAACTTTAAACCAGCTAGTGTTACAGA
CTCTAAAGTAGAGGCATAATCTTCTAGATCAGAATCTGGATTGGAAGTGGGATAAAGA
AATCTTGACCCCAAATCTTTGGATTCCTATTTGGCTCATTAACGTTTAAAAAAGAATAA
AGCCTTTAAGGAAAACAATCTATTGTTCCTGGCCTGAGATACTACCTGGTTCTTTTGT
TTAATTATAATTGAAAACACCAAGTTTTACTTATTTGTGAAATCAAGTAAATACATGG
CCTTTAAATATAAGATAAAAAACAAAATAAAAACTCTTCTGAATAAGCATTTCAGGTAT
AAAAATTGTGATGAGGTCACATATGTAAAGAATTAAACAATTGCTTTTTCACTCCTTGG
CATTTAGGTGAATTGCTAACAAACACCCAGACTCCCTTATTTACAGCTGTTCTAGAAA
CACTTAACATTTTATTGGGCCCCACGCTGGCTATTTCCCTCTCAGCAAACATGCACTG
GGTCTGAGAATAAATCAGTCGTTCTTAAACTTCCTTGAGTCATGAATCCCTACGAGGC
TCTACGATAACTCTAGGCTCTCTCTCCAGAAAGAAAAAAAAAATGTACATAATGCAAA
GTTGCAATATAACTTGAAAGGGTTCATGAGCCCCTGGTTCTATATTGATTATGAGGGA
AAGGAACCATGTTTGCTCGTAGTTAATGCAGTTGTAATAGGCACTTTACATTTGAGAC
CATCTCTCTGTGATTCTCAGGAAGGCCAAATCTCAGCACCAGAGTTTGAACAAAGATT
TTGTTATTTTTGTTCTTTAAATTAGATGTTGCCAATTTAATGCACTTACATCCTAACATG
TTGATGATTTTAAGTATCAAGCACAATCACAATGCTGAAGTCTCCCTGAAGGGTCTAT
AGAGGAATCCAAGAAGGAGGTGAAAACATTATTCATTGACATCAAACACCTCCAAGG
ceTCATTcTGGGCCAGATTGGAAGTGAGGGAAAGAAATTTTCAGCTCAAGTGTTTCC
ATTCATCGTGATTTCAGAGAGAAAAGGATAGGAACTGGGAAACGAAATACTCAACAG
TCCTGATCTTGCAATTACGTGATTGTTTTGTTGTGGATGAAAATTAAGTTGTAAGGTGT
GTGAAACGTGTGAAATATATAAGCCAAACTTAAATGACTGTAGACACAAACTGTGTAA
GTTGGATGAGGGATTTGGTTGGTGTGGAGATGTATGTGTGTTATTTTTGAGTGGATTTG
CTTCCATTTCTGGGTTCTCTGTGCAGCAATGTGGCATTGATTCTGTGGCCTTGTGCAG
TCAGCTTATGCTCAGCCCATTGATTCCACCACATCCCTGATCGAGGGGACGTTAATTA
CCTGTCTTTGTGAATTAAGCAGTTTATGAGCAGCAAGTTACTGAGGACCGTAAATAAA
GTCATAGAGAGCTCAAGGAAGTAATAGCTTCGGTTTAATCATCTGCTTCAAAGGTGCC
AATTTTTAGATAGTAGTATATGTAATGATTTGTTGTAAATTGGTTGTTGGTTTTGAAAAA
AATTTAAGGATGTTTTATGAAAATGAGGATGGAGGTGTGGTGGTTAAGGAATATAGTTT
ATATTTATAGTTAGAGGGCCCCTCCTTGATAGATTTGAATGAGGAGCTGGAAAGCTTG
GGTAGAATTTTGATGAGTAGTTTGTATTATGTGTGGAAGGGAAAAGGTGTATTAGGAG
TGAGAATAGAGGAGTTTAGAGAAGTGGATTTTTTAAATGTATAAGTTTGGAGGTGTAGG
ATATTATTTAGGAGTTTGAAAGTTGGGGTTTTAAAATATATATATAAGAATAATGAAAGG
AGAAAAATGTACTCATAAAAACACAAAATGTGTTTCTTCTGAATGTTTCAAATGTATTC
TAAAATATGGTTTGTATAGATAATATTATGGTAGATAAGTGGATAGTAGTTTTTTAGTTT
CTGAGTCTGTACAGTCTAATCTTCCTGAATGTTTGGGCTGTTTAATTTCTTTAAAGATG
ATAAATATTACCCGGTCATCCATCATGCTCTATCTATAGGCTATACCATTCATATGTTG
GGCAATGCTGTATTATAAAACACATGTAAACATGTATGTAACCCATGTTTAACCCAGC
GTAGTTGTAGCATGTGGTTGTATTAATGAACGTTACAGGAGAGCTTTATAATCATTTAT
AAATCTGTAACATTCAATAAAGAAACACATGTTGCAATGATTAATTATGTTCCCAATTC
GATGTTATTTAGGATGTTTGTGAAGGGGGGATTTTTGTTGGTGATGTGGTTGATGGTTT
TCATGTTCCTTTGGACCACTGTAAAATATGTGTGTAAGCTGATGTATATCCTCTAATGA
GTGGTGGGAGGTGTGGTAGGGAAGTTGTGATTTTGTGAAAGATGAAGTGGGATGTTA
TGGAAAAGAGTATATGAGAAATGAAGTTAAAATGATATTTTAAAGTGGAGTTGAATTAG
GTGTTTAGATGTGGGAAGGGGATGGAGAAGGAGTGTAGAGTATTTTATAAATATGTTG
ATTTAGTAACACTATGTTGTTTCTAGATAAAGTGATACTAGTGTGTAGGTGGTTATATA
TTTGGCCTAGATATTGCATTATAACTCACTATCCTTGAACAATAACAATGTGACTGAAA
TGAAGTAATGTGTGTGGAAAGGTGGTGTTAATTTTGTGTGAAGAAAATGGATGGAGTG
CTTTATGCATTTCTTTTGACTTTTCTAAGGCACTTAACATTTTACTATTGCCTGCTGTG
ACGCTTAAGCCAGTTAAGAATTCACTCAAACAGTTGGGCTTCTTACTATGGGGATTAT
GTTCAGTTTTGAAAAATTAAAAACAAAAAAAAAGTAACCAAGGGATTTTTAAATCTTTTT
TTTAAGCACTGAACAAATAATATGAAATAAAGTATTAACTGCAGGTTGTATAGCCATG
GGACCCAGAGGTGGCAAGCCAACACCTCTTGGGTCTGCCAAGGTCTAGGTGTTGTG
CATCTGTGATATATGCAAGACACATTTCTGATTCTTCAGAGCTACTGGGTGCAAATTG
CTAAGCCGGAGATGAGAGCGAAAAGTCCATTTGTGATAGTGCAGAAGTGCGCAGATC
GCTGTGAATGTGTGATCCAATGATATGACAAGAGACCTCCCTCCTTGTCTTGTGGCTT
CTTCACCTTGGGGGTGGGAGGCGTGTGTTTGGGTGTCTGATGAAGCACTGGGTGGT
TCTAAAGAGTTTACTCTTTTAGAAAATCATCATTACTAGAAGCCATTCAACATCTGTTA
AAGAGAGGAGCCAATTTGCACAGTTTTGAAAATGACTGCTCCTAAAATGAGTAGACAA
AACTTTCCTCTCAGCCTGCATTGACCTAAGCTTTCTCTTCTTTTTACAAGTTGGATATT
CTGTCTCTCAGATTGGAGTCAACCAAAAGAAAGGCTGGTTCCAATTAAGGAAAAGCA
ATTTTTTTCTTTTAATTCCTCTCAGAAGGTAGTTGTGTTACCTTTTCTCTGATGATTTCC
TTGCACTCATCACTGCTAATGAAAGCAACGTGACTGTTTCTACAGGTCATCTAAGAGA
GGATTTTGGTGACCAGGTAATCCAGATGGCAATAAGAATGAGGTGAATTTTCCCTTCC
TTACCTTACCTAGTCTACCAGCCTCCTTAAACCTGCAGGCCCTTGTGCGTTTGCACAA
TGTTTGTGTGCTGGAAACTGAAAAGACATGTGTATGGCTAGAAGTCCATAGGCTTTGT
AAAGAGTGTCAGTGCCAATCAGGACCCATGCCTTCCTCTGGAGGAGACAGGTGGCT
GAGGCCTATGGGGCAAAGGGGGAAATTCAGCCTTGTGCCTGGATGTGCGGCTTTCT
TTCTCCCCTCTTCTTGCTTCTCTTCTCTTAGTCTAAGACTAGCTGCTTTTTCACCAGAC
CAACAGACCCTTGCCCACATTACCTTTATACCTTGTTGACCCTCGCACAGTAAGCTGA
AACAGCGGCCGCTTGGAAAAAGGCAAACATTTAGACTTTGTCCCAGGCCTGATCTTC
CCACCAACTTAGCCTCCTCCTGCCTCTACCCCGGCTGGTGTTCACATTGACCTCGTC
CAATTTTTGCCTCTCTAAATCTCAAGAGTCCTACACATCTTCACCGCAGCTTCCCACA
CATCATTAAAACCAGCCATCTGCACATAGACTGAGAGGCTGCAAATTCAAACACATG
GAGGGGCCGGATCCAGAAAACACAAATGTGTGAACTGGACTGGTGAAAGTCAACAG
AAGTAGGGTTTGTAAGTAAGGCATGACAGAATACACATCCCACCACAAACACCTTCAA
ATTCAGCTTTCCAAACACTGTCCTAGCCAAACAAACATGTCTGCCAGCAGCATTCAGT
CCCAAAGGCAATAGTTTGGGACCTCTGATTTAGAGCCAACAGGATCAGATTCTTGATA
GTGACTTGGTGAGTGTACACACAAAATACACTGAGAACCGAGAGGGGGGAAAAGAA
ACTCGCCTTATTCAGAGTTACTCTTGTTCTGAATGATATATGCATGTATATTTGTTAGC
CACATAATTCTAAACTTGAACACAGAGTCTTTGTGAGAGAGATTCAGAAGAAGAGTAC
CATAGCATGGTATGAAGACTTACCTGCTTCCTGTCACTACTGCGTCTGACCTGAGTTT
ATATTGCTGCTACCGCTGTGTAAAAACAGTACGTGCAGTATACTGTACTCATAGCCAT
TTCATAATATAGGACCTAGACTCTCATTTGTAAACGTTCTAACCCTAGTTTATAATGGG
GGAAATTATTCAGATATCATTTTTAAATGAATTCCTACAAATACAATGGTTTTAAAATTG
GGTTTGCTGAATATATATACATCTTTTCCTTCTTTGTGTATGGATGGACCACGGTATG
CATATCTATCGACACTATAAATATATATAAATCCTTAGAAGAATTGTTCTTTTTTAAAGG
TCTATTATTGGATTCACAATGCACTTTGAGTTTGATTGTTTAGATATACAATTCAGAGC
AGAAGTTGGCAGATACCACGGGAAAAACTTTCTGAAATTTCTGTAACAAATGTTTTGC
AATAAAAAAAAAAACTCAGTAGTTTAAAACATGA

The material in the XML file named “PB66994US02.XML[,]” created on Jul. 12, 2022 and having a size of 134,165 bytes is incorporated herein by reference in its entirety.

1. A method of treatment or prevention of a disorder selected from migraine, trigeminal neuralgia, cluster headache, postherpetic neuralgia, chemotherapy-induced neuropathy, complex regional pain syndrome, HIV sensory neuropathy, peripheral nerve injury and phantom limb pain, which comprises administering a subject in need thereof a therapeutically effective amount of an inhibitor of human TRPM3.

2. A method according to claim 1, wherein the subject is human.

3. A method according to claim 2, wherein the inhibitor of human TRPM3 is an inhibitor of a human TRPM3 variant having the sequence set out in SEQ ID NO:2, or a processed version of this variant lacking the initial methionine residue.

4. A method according to claim 3, wherein the inhibitor of human TRPM3 is an inhibitor of a mutated version of human TRPM3 having a gain of function mutation.

5. A method according to claim 4, wherein the mutated version of human TRPM3 has one or more of the following amino acid substitutions is R1670Q, A1645V, V990M and P10900 (numbering based on SEQ ID NO: 2).

6. A method according to claim 2, wherein the use is the treatment of migraine.

7. A method according to claim 6, wherein the subject is a human subject whose migraines are not responsive to CGRP inhibition.

8. A method according to claim 7, wherein the human subject has failed treatment with an antagonist of CGRP.

9. A method according to claim 6, wherein the subject is a human subject whose migraines are responsive to therapy with a triptan.

10. A method according to claim 9, wherein the human subject has migraines that have previously responded to treatment with a triptan.

11. A method according to claim 10, wherein the triptan is selected from the group consisting of: almotriptan, eletriptan, frovatriptan, naratriptan, rizatriptan, sumatriptan and zolmitriptan.

12. A method according to claim 6, wherein the percentage of patients that are pain free 2 hours after administration of the inhibitor of human TRPM3 is higher for a population of patients receiving the inhibitor of human TRPM3 compared to a population of patients receiving placebo.

13. A method according to claim 6, wherein the percentage of patients that are pain free 2 hours after administration of the inhibitor of human TRPM3 and do not use rescue medication or relapse within 24 hours after administration of the inhibitor of human TRPM3 is higher compared to a population of patients receiving placebo.

14. A method according to claim 6, wherein the inhibitor of human TRPM3 is administered in combination with at least one other therapeutic agent selected from: a triptan, an ergot, a non-steroidal anti-inflammatory drug, an acetaminophen containing product, a butalbital containing product, an anti-emetic, caffeine, dexamethasone, ubrogepant and lasmiditan.

15. A method according to claim 6, wherein the inhibitor of human TRPM3 is administered in combination with a triptan and a non-steroidal anti-inflammatory drug.

16. A method according to claim 6, wherein the inhibitor of human TRPM3 is administered in combination with sumatriptan.

17. A method according to claim 6, wherein the inhibitor of human TRPM3 is administered in combination with a non-steroidal anti-inflammatory drug.

18. A method according to any claim 2, wherein the use is the prevention of migraine.

19. A method according to claim 18, wherein the subject is a human subject whose migraines are not responsive to CGRP inhibition.

20. A method according to claim 19, wherein the human subject has failed treatment with an antagonist of CGRP.

21. A method according to claim 18, wherein the subject is a human subject whose migraines are responsive to therapy with a triptan.

22. A method according to claim 21, wherein the human subject has migraines that have previously responded to treatment with a triptan.

23. A method according to claim 22, wherein the triptan is selected from the group consisting of: almotriptan, eletriptan, frovatriptan, naratriptan, rizatriptan, sumatriptan and zolmitriptan.

24. A method according to claim 18, wherein, following administration to a population of patients, the reduction in mean monthly migraine days is greater for a population of patients receiving the inhibitor of TRPM3 compared to placebo.

25. A method according to claim 18, wherein the 50% responder rate is higher for a population of patients receiving the inhibitor of TRPM3 compared to placebo.

26. A method according to claim 18, wherein the inhibitor of human TRPM3 is administered in combination with at least one other therapeutic agent selected from: botulinum toxin A, a CGRP inhibitor, an anticonvulsant, a β-blocker, an antidepressant and a non-steroidal anti-inflammatory drug.

27. A method according to claim 18, wherein the inhibitor of human TRPM3 is administered in combination with a therapeutic agent selected from: valproate, divalproex sodium, amitriptyline, topiramate, venlafaxine, metoprolol, propranolol and timolol.

28. A method according to claim 2, wherein the use is the treatment of cluster headache.

29. A method according to claim 28, wherein the subject is a human subject whose headaches are not responsive to CGRP inhibition.

30. A method according to claim 29, wherein the human subject has failed treatment with an antagonist of CGRP.

31. A method according to claim 28, wherein the subject is a human subject whose headaches are responsive to therapy with a triptan.

32. A method according to claim 31, wherein the human subject has migraines that have previously responded to treatment with a triptan.

33. A method according to claim 32, wherein the triptan is selected from the group consisting of: almotriptan, eletriptan, frovatriptan, naratriptan, rizatriptan, sumatriptan and zolmitriptan.

34. A method of treatment of medication overuse headache in a human subject, which comprises administering a subject in need thereof a therapeutically effective amount of an inhibitor of human TRPM3.

35. A method for measuring PACAP in a sample comprising incubating a cell line expressing the PAC1 receptor with the sample and measuring cAMP signalling in the cell line.

36. A method for identifying an inhibitor of human TRPM3, comprising measuring release of PACAP from dorsal root ganglia or trigeminal ganglia, or from primary cultures of cells isolated from dorsal root ganglia or trigeminal ganglia, following challenge with an agonist of human TRPM3 in the presence or absence of a test inhibitor, wherein the test inhibitor is identified as an inhibitor for human TRPM3 if PACAP production is reduced in the presence of the test inhibitor compared to PACAP production in the absence of the test inhibitor, and wherein PACAP production is measured according to the method defined in claim 35.

37. A method according to claim 36, wherein the agonist of human TRPM3 is pregnenolone sulfate, a racemate of 2-(3,4-dihydroquinolin-1(2H)-yl)N-(5-methylisoxazol-3-yl)-2-phenylacetamide, (R)-2-(3,4-dihydroquinolin-1(2H)-yl)-N-(5-methylisoxazol-3-yl)propenamide or (S)-2-(3,4-dihydroquinolin-1(2H)-yl)-N-(5-methylisoxazol-3-yl)propenamide.

38. (R)-2-(3,4-dihydroquinolin-1(2H)-yl)-N-(5-methylisoxazol-3-yl)propenamide or a salt thereof.
